(19) United States
(12) Patent Application Publication
(10) Pub. No.: US 2024/0123076 A1
(43) Pub. Date: Apr. 18, 2024

(54) UNSATURATED DENDRIMERS COMPOSITIONS, RELATED FORMULATIONS, AND METHODS OF USE THEREOF

(71) Applicant: The Board of Regents of The University of Texas System, Austin, TX (US)

(72) Inventors: Sang M. LEE, Dallas, TX (US); Daniel J. SIEGWART, Dallas, TX (US)

(73) Assignee: The Board of Regents of The University of Texas System, Austin, TX (US)

(21) Appl. No.: 18/264,576

(22) PCT Filed: Dec. 9, 2021
(86) PCT No.: PCT/US2021/062717

§ 371 Date: Aug. 7, 2023

Related U.S. Application Data
(60) Provisional application No. 63/146,858, filed on Feb. 8, 2021.

(51) Int. Cl. A61K 47/59; A61K 47/54; A61K 47/60

(52) U.S. Cl. CPC A61K 47/593; A61K 47/542; A61K 47/543; A61K 47/545; A61K 47/60

(57) ABSTRACT

Described herein are novel lipid compositions comprising unsaturated dendrimers and methods of synthesis of unsaturated dendrimers. The lipid composition can comprise an ionizable cationic lipid, a phospholipid, and a selective organ targeting lipid. Also described herein are pharmaceutical formulations comprising an unsaturated dendrimer, a lipid composition, and a therapeutic agent. Further described in here are methods of mRNA delivery comprising a lipid composition and a therapeutic agent. Further described herein are high-potency dosage forms of a therapeutic formulated with a lipid composition.

This application claims the benefit of priority to U.S. Provisional Application No. 63/146,858, filed Feb. 8, 2021, the entire contents of which are hereby incorporated by reference.

### BACKGROUND

Beyond functioning as a link between the genetic code of DNA and functional proteins, messenger mRNA (mRNA) has emerged as a versatile tool to produce proteins for therapeutic applications towards cancer, vaccines, and other areas. However, the major challenge of RNA therapeutics remains efficacious delivery. mRNAs are incapable of passing through cellular membranes on their own due to their physiochemical attributes and propensity for degradation. Methods are needed to encapsulate and deliver mRNA inside cells. To address this challenge, lipid nanoparticles (LNPs) represent the leading concept for mRNA delivery. LNPs are composed of multiple lipids, including ionizable amino lipids, which acquire charge during endosomal maturation and allow endosomal escape of RNA into the cytoplasm to enable delivery of the genetic material. LNPs were initially established as carriers for siRNAs and have increasingly been explored for delivery of mRNA.

### SUMMARY

Described herein are ionizable amino lipid platforms created using modular dendrimer growth reactions and producing dendrimers with unsaturation. Described herein are lipid designs consisting of an ionizable amine core, ester-based degradable linker, and alkyl thiol tail periphery or unsaturated alkyl thiol tail periphery. In some embodiments, incorporate unsaturation into the ionizable lipid, we synthesized alkenyl thiols and inserted them as the hydrophobic tail domain, mimicking natural fatty acids. These newly synthesized lipids were formulated into LNPs and compared to their saturated parents. In doing so, we aimed to understand why unsaturation may be important for LNPs and explore the potential applications of unsaturated LNPs. Modular reactions were utilized to create a chemically diverse library of unsaturated amino lipids. Our synthetic route towards the ionizable lipid involves a nucleophilic amine addition to an ester-based linker, followed by a Michael addition with the thiols. Previous studies solely involved alkyl thiols because unsaturated thiols are not commercially available. Thus, we hypothesized bridging this gap by synthesizing unsaturated thiols from alkenyl alcohols and terpenes found in nature. However, initial attempts for the unsaturated thiols proved difficult, resulting in undesired products and low yields.

One aspect of the disclosure provides a (e.g., unsaturated) dendrimer of a generation (g) having a structural formula:

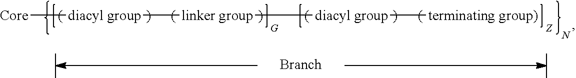

or a pharmaceutically acceptable salt thereof, wherein:
(a) the core comprises a structural formula (X_{Core}):

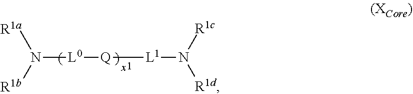

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

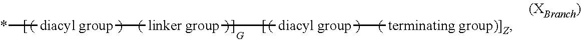

wherein:
   * indicates a point of attachment of the branch to the core;
   g is 1, 2, 3, or 4;
   Z=2^{(g-1)};
   G=0, when g=1; or G=Σᵢ₌₀^{i=g-2}2ⁱ, when g≠1;
(c) each diacyl group independently comprises a structural formula

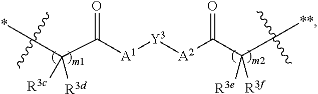

wherein:
   * indicates a point of attachment of the diacyl group at the proximal end thereof,
   ** indicates a point of attachment of the diacyl group at the distal end thereof,
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein: R⁴ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula

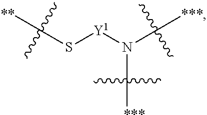

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is R independently at each occurrence selected from C₆-C₂₂ alkenyl, C₆-C₂₂ alkadienyl, and C₆-C₂₂ alkatrienyl.

Described herein are lipid compositions comprising an unsaturated dendrimer as described herein, and one or more lipids selected from an ionizable cationic lipid, a zwitterionic lipid, a phospholipid, a steroid or a steroid derivative thereof, and a polymer-conjugated (e.g., polyethylene glycol (PEG)-conjugated) lipid. Further described herein are pharmaceutical compositions comprising a therapeutic agent coupled to a lipid composition comprising a dendrimer as described herein. Another aspect of the disclosure are methods for delivering a therapeutic agent into a cell, the method comprising: contacting the cell with the therapeutic agent coupled to a lipid composition of described herein, thereby delivering the therapeutic agent into the cell.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A shows the synthesis reaction scope of alkenyl thiols using non-allylic and allylic alcohols.

FIG. 1B shows the synthesis reaction scope of ionizable amino lipids using 7 different amine cores with yields of isolated products reported.

FIG. 2A shows a bar graph of lipid series for in vitro expression assays of Luc mRNA delivery to IGROV-I cells and displaying 4A3-4T as the most potent.

FIG. 2B shows heat map of the in vitro Luciferase assay data revealed differs based on positions and configuration of the unsaturation showing 4A3-derived lipids performed best across the lipid series.

FIG. 3A shows ex vivo imaging of whole body images 6 hours after i.v. administration of LNPs with the highest mRNA expression in eight carbon series (0.25 Luc mg kg⁻¹).

FIG. 3B shows ex vivo organs imaged 6 hours after administration of LNPs.

FIG. 3C shows a graph of quantified total radiance of the liver.

FIG. 3D shows a presentation of the 4-component standard LNP in vivo formulation method.

FIG. 4A shows a visual assay representation showing the emission changes based on fusion.

FIG. 4B shows a graph of percent lipid fusion with the model endosomal membrane.

FIG. 5A shows images for optimization of Cit SORT lipid.

FIG. 5B shows images an evaluation of cross-over mix using identified lipid percentages.

FIG. 5C shows a graph of quantified average luminescence of the liver after 6 hours.

FIG. 5D shows a visual representation of the formulation.

FIG. 5E shows a table of details for the base LNP and SORT LNP formulations (Total lipids/mRNA ratio=40; wt/wt).

FIG. 6 shows a table for molar ratio and molar percentage of the formulations (total lipids/mRNA=40; wt/wt).

FIG. 7A shows images of whole body and ex vivo imaging of C57BL/6 mice injected with LNPs carrying Luc mRNA (0.25 mg/kg).

FIG. 7B shows graphs for liver (left) and spleen (right) luminescence quantification.

FIG. 8 shows graphs of luminescence quantification for liver (left) and spleen (middle and right). The graph on the left depicts total luminescence quantification of the liver from the SORT formulations. The graph in the middle depicts average luminesce quantification of the 4A3-Cit SORT formulations. The graph on the right depicts total luminescence quantification of the 4A3-Cit SORT formulations.

FIG. 9 shows a table for physical characterization data of the general LNP base formulations and the mRNA encapsulation efficiency.

FIG. 10 shows a table for physical characterization of the SORT LNP formulations and the mRNA encapsulation efficiency.

FIG. 11 shows a graph for IGROV-1 cell viability data of 24 hours following treatment with 25 ng of Luciferase mRNA in various LNP formulations.

FIG. 12 shows ex vivo imaging of C57BL/6 mice 6 hours after IV injection with LNPs carrying Cy5 Luc mRNA (0.25 mg/kg) and its distribution primary to the liver. There was no statistical difference between ROI values 4A3-SC8 and 4A3-Cit (two-tailed unpair t-test).

FIG. 13 shows a graph of average and total luminescence quantification of the Luc mRNA expression in the liver (0.25 mg/kg). Data are presented as mean±s.d. and statistical significance was analyzed by the two-tailed unpaired t-test in relation to the 5A2-SC8 results.

FIG. 14A shows representative confocal images of cellular uptake and colocalization of IGROVI cells and Cy5 Luc mRNA-loaded LNPs 4 h after incubation at 63×.

FIG. 14B shows a graph of the mean intensity of Cy5 Luc mRNA signal at 4 h and 24 h after incubation plotted as an average of randomly measured spots.

FIG. 14C shows a graph of Pearson's correlation coefficient of the Cy5 Luc mRNA and lysosome organelles at 4 h and 24 h after incubation plotted as an average of randomly measured spots.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “disease,” as used herein, generally refers to an abnormal physiological condition that affects part or all of a subject, such as an illness (e.g., primary ciliary dyskinesia) or another abnormality that causes defects in the action of cilia in, for example, the lining the respiratory tract (lower and upper, sinuses, Eustachian tube, middle ear), in a variety of lung cells, in the fallopian tube, or flagella of sperm cells.

The term “polynucleotide” or “nucleic acid” as used herein generally refers to a polymeric form of nucleotides of any length, either ribonucleotides or deoxyribonucleotides, that comprise purine and pyrimidine bases, purine and pyrimidine analogues, chemically or biochemically modified, natural or non-natural, or derivatized nucleotide bases. Polynucleotides include sequences of deoxyribonucleic acid (DNA), ribonucleic acid (RNA), or DNA copies of ribonucleic acid (cDNA), all of which can be recombinantly produced, artificially synthesized, or isolated and purified from natural sources. The polynucleotides and nucleic acids may exist as single-stranded or double-stranded. The backbone of the polynucleotide can comprise sugars and phosphate groups, as may typically be found in RNA or DNA, or analogues or substituted sugar or phosphate groups. A polynucleotide may comprise naturally occurring or non-naturally occurring nucleotides, such as methylated nucleotides and nucleotide analogues (or analogs).

The term “polyribonucleotide,” as used herein, generally refers to polynucleotide polymers that comprise ribonucleic acids. The term also refers to polynucleotide polymers that comprise chemically modified ribonucleotides. A polyribonucleotide can be formed of D-ribose sugars, which can be found in nature.

The term “polypeptides,” as used herein, generally refers to polymer chains comprised of amino acid residue monomers which are joined together through amide bonds (peptide bonds). A polypeptide can be a chain of at least three amino acids, a protein, a recombinant protein, an antigen, an epitope, an enzyme, a receptor, or a structure analogue or combinations thereof. As used herein, the abbreviations for the L-enantiomeric amino acids that form a polypeptide are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). X or Xaa can indicate any amino acid.

The term “engineered,” as used herein, generally refers to polynucleotides, vectors, and nucleic acid constructs that have been genetically designed and manipulated to provide a polynucleotide intracellularly. An engineered polynucleotide can be partially or fully synthesized in vitro. An engineered polynucleotide can also be cloned. An engineered polyribonucleotide can contain one or more base or sugar analogues, such as ribonucleotides not naturally-found in messenger RNAs. An engineered polyribonucleotide can contain nucleotide analogues that exist in transfer RNAs (tRNAs), ribosomal RNAs (rRNAs), guide RNAs (gRNAs), small nuclear RNA (snRNA), small nucleolar RNA (snoRNA), SmY RNA, spliced leader RNA (SL RNA), CRISPR RNA, long noncoding RNA (lncRNA), microRNA (miRNA), or another suitable RNA.

### Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof, in a divalent context “phosphate” means —OP(O)(OH)O— or a deprotonated form thereof, “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, for example, the formula

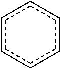

includes

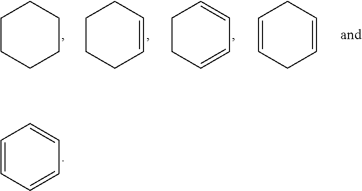

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

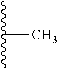

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

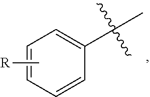

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

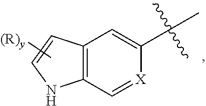

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H—R, wherein R is alkyl as this term is defined above.

When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

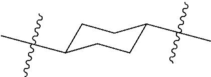 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C—CH, —C—CCH₃, and —CH₂C—CCH₃ are non-limiting examples of alkynyl groups.

An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

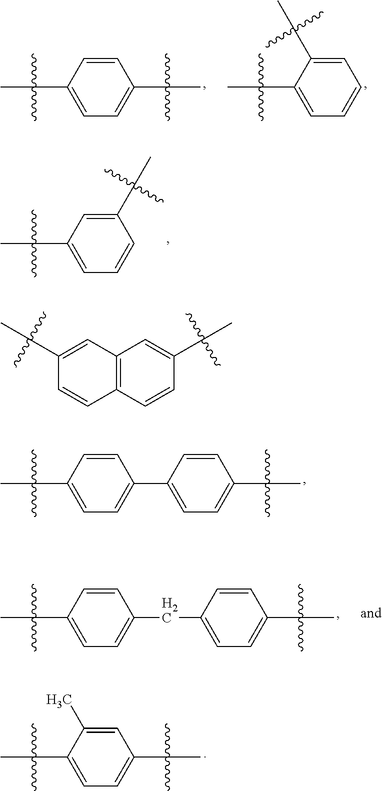

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

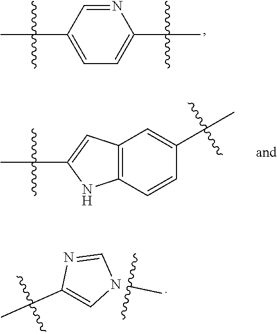

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to an divalent cyclic group, with two carbon atoms, two nitrogen atoms, or one carbon atom and one nitrogen atom as the two points of attachment, said atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

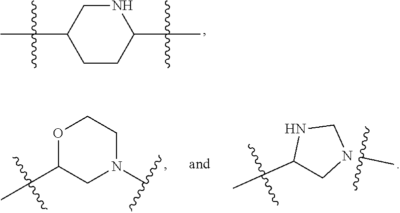

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules.

Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity.

Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The term “molar percentage” or “molar %” as used herein in connection with lipid composition(s) generally refers to the molar proportion of that component lipid relative to compared to all lipids formulated or present in the lipid composition.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### Compositions

### Unsaturated Dendrimers

In some embodiments, the ionizable cationic lipid is a dendrimer of the formula Core [symbol] Branch)_{N}. In some embodiments, the ionizable cationic lipid is a dendrimer of the formula

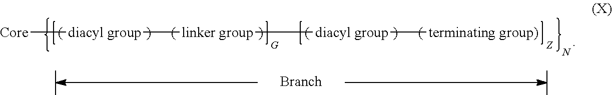

In some embodiments, the ionizable cationic lipid is a dendrimer of a generation (g) having a structural formula:

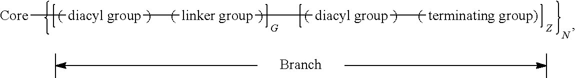

or a pharmaceutically acceptable salt thereof, wherein:
(a) the core comprises a structural formula (X_{Core}):

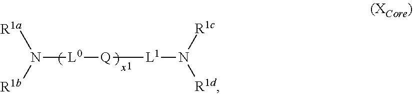

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

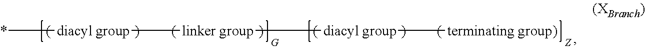

wherein:
   * indicates a point of attachment of the branch to the core;
   g is 1, 2, 3, or 4;
   Z=2^{(g-1)};
   G=0, when g=1; or G=Σᵢ₌₀^{i=g-2}2ⁱ, when g≠1;
(c) each diacyl group independently comprises a structural formula

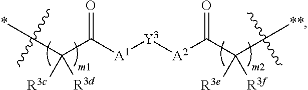

wherein:
   * indicates a point of attachment of the diacyl group at the proximal end thereof,
   ** indicates a point of attachment of the diacyl group at the distal end thereof,
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein: R⁴ is hydrogen or optionally substituted (e.g., C₁-C₆) alkyl;
   m¹ and m² are each independently at each occurrence 1, 2, or 3; and
      R^{3c}, R^{3d}, R^{3e}, and R^{3f} are each independently at each occurrence hydrogen or an optionally substituted (e.g., C₁-C₈) alkyl; and
(d) each linker group independently comprises a structural formula

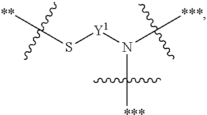

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkenylthiol, and optionally substituted (e.g., C₁-C₁₈, such as C₄-C₁₈) alkenylthiol.

In some embodiments of X_{Core}, Q is independently at each occurrence a covalent bond, —O—, —S—, —NR²—, or —CR^{3a}R^{3b}. In some embodiments of X_{Core} Q is independently at each occurrence a covalent bond.

In some embodiments of X_{Core} Q is independently at each occurrence an —O—. In some embodiments of X_{Core} Q is independently at each occurrence a —S—. In some embodiments of X_{Core} Q is independently at each occurrence a —NR² and R² is independently at each occurrence R^{1g} or -L²-NR^{1e}R^{1f}. In some embodiments of X_{Core} Q is independently at each occurrence a —CR^{3a}R^{3b}R^{3a}, and R^{3a} and R^{3b} are each independently at each occurrence hydrogen or an optionally substituted alkyl (e.g., C₁-C₆, such as C₁-C₃).

In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl. In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen. In some embodiments of X_{Core}, R^{1a}, R^{1b}, R^{1c}, R^{1d}, R^{1e}, R^{1f}, and R^{1g} (if present) are each independently at each occurrence a point of connection to a branch an optionally substituted alkyl (e.g., C₁-C₁₂).

In some embodiments of X_{Core}, L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or, alternatively, part of L¹ form a heterocycloalkyl (e.g., C₄-C₆ and containing one or two nitrogen atoms and, optionally, an additional heteroatom selected from oxygen and sulfur) with one of R^{1c} and R^{1d}. In some embodiments of X_{Core}, L⁰, L¹, and L² are each independently at each occurrence can be a covalent bond. In some embodiments of X_{Core}, L⁰, L¹, and L² are each independently at each occurrence can be a hydrogen. In some embodiments of X_{Core}, L⁰, L¹, and L² are each independently at each occurrence can be an alkylene (e.g., C₁-C₁₂, such as C₁-C₆ or C₁-C₃). In some embodiments of X_{Core}, L⁰, L¹, and L² are each independently at each occurrence can be a heteroalkylene (e.g., C₁-C₁₂, such as C₁-C₆ or C₁-C₆). In some embodiments of X_{Core}, L⁰, L¹, and L² are each independently at each occurrence can be a heteroalkylene (e.g., C₂-C₅ alkyleneoxide, such as oligo(ethyleneoxide)). In some embodiments of X_{Core}, L⁰, L¹, and L² are each independently at each occurrence can be a [alkylene]-[heterocycloalkyl]-[alkylene] [(e.g., C₁-C₆) alkylene]-[(e.g., C₄-C₆) heterocycloalkyl]-[(e.g., C₁-C₆) alkylene]. In some embodiments of X_{Core}, L⁰, L¹, and L² are each independently at each occurrence can be a [alkylene]-(arylene)-[alkylene] [(e.g., C₁-C₆) alkylene]-(arylene)-[(e.g., C₁-C₆) alkylene]. In some embodiments of X_{Core}, L⁰, L¹, and L² are each independently at each occurrence can be a [alkylene]-(arylene)-[alkylene] (e.g., [(e.g., C₁-C₆) alkylene]-phenylene-[(e.g., C₁-C₆) alkylene]). In some embodiments of X_{Core}, L⁰, L¹, and L² are each independently at each occurrence can be a heterocycloalkyl (e.g., C₄-C₆ heterocycloalkyl). In some embodiments of X_{Core}, L⁰, L¹, and L² are each independently at each occurrence can be an arylene (e.g., phenylene). In some embodiments of X_{Core}, part of L¹ form a heterocycloalkyl with one of R^{1c} and R^{1d}. In some embodiments of X_{Core}, part of L¹ form a heterocycloalkyl (e.g., C₄-C₆ heterocycloalkyl) with one of R^{1c} and R^{1d} and the heterocycloalkyl can contain one or two nitrogen atoms and, optionally, an additional heteroatom selected from oxygen and sulfur.

In some embodiments of X_{Core}, L⁰, L¹, and L² are each independently at each occurrence selected from a covalent bond, C₁-C₆ alkylene (e.g., C₁-C₃ alkylene), C₂-C₁₂ (e.g., C₂-C₅) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene] (e.g.,

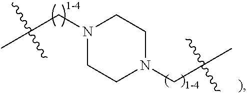

and [(C₁-C₄) alkylene]-phenylene-[(C₁-C₄) alkylene] (e.g.,

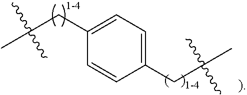

In some embodiments of X_{Core}, L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments of X_{Core}, L⁰, L¹, and L² are each independently at each occurrence C₁-C₆ alkylene (e.g., C₁-C₃ alkylene). In some embodiments, L⁰, L¹, and L² are each independently at each occurrence C₂-C₁₂ (e.g., C₂-C₅) alkyleneoxide (e.g., —(C₁-C₃ alkylene-O)₁₋₄—(C₁-C₃ alkylene)). In some embodiments of X_{Core}, L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

In some embodiments of X_{Core}, x¹ is 0, 1, 2, 3, 4, 5, or 6. In some embodiments of X_{Core}, x¹ is 0. In some embodiments of X_{Core}, x¹ is 1. In some embodiments of X_{Core} x¹ is 2. In some embodiments of X_{Core}, x¹ is 0, 3. In some embodiments of X_{Core} x¹ is 4. In some embodiments of X_{Core} x¹ is 5. In some embodiments of X_{Core}, x is 6.

In some embodiments of X_{Core}, the core comprises a structural formula:

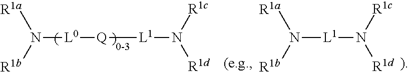

In some embodiments of X_{Core}, the core comprises a structural formula:

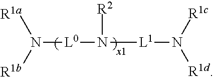

In some embodiments of X_{Core}, the core comprises a structural formula:

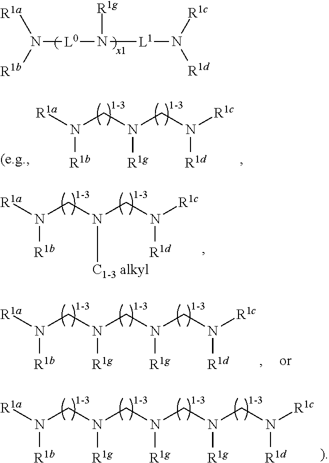

In some embodiments of X_{Core}, the core comprises a structural formula:

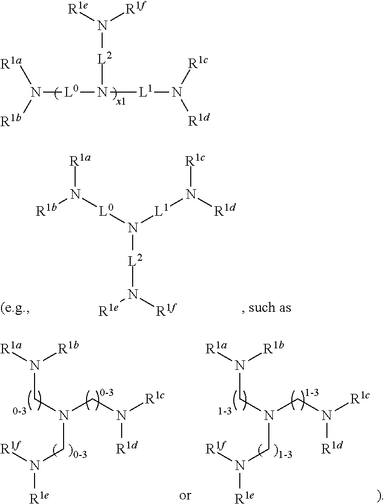

In some embodiments of X_{Core}, the core comprises a structural formula:

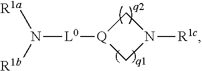

wherein Q¹ is —NR²— or —CR^{3a}R^{3b}—; q¹ and q² are each independently 1 or 2. In some embodiments of X_{Core}, the core comprises a structural formula:

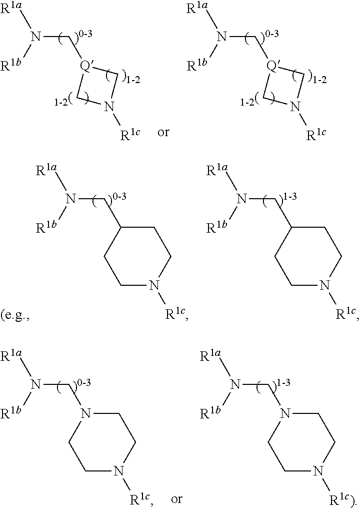

In some embodiments of X_{Core}, the core comprises a structural formula

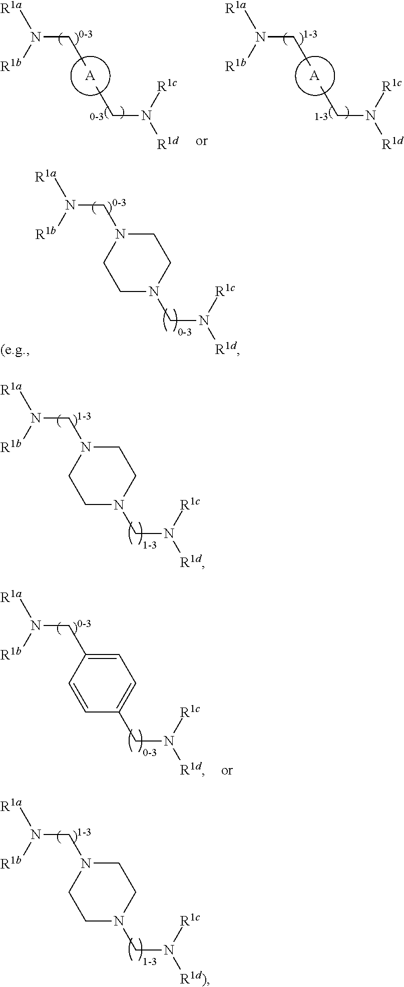

wherein ring A is an optionally substituted aryl or an optionally substituted (e.g., C₃-C₁₂, such as C₃-C₅) heteroaryl. In some embodiments of X_{Core}, the core comprises has a structural formula

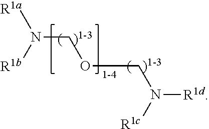

In some embodiments of X_{Core}, the core comprises a structural formula set forth in Table 1 and pharmaceutically acceptable salts thereof, wherein * indicates a point of attachment of the core to a branch of the plurality of branches. In some embodiments, the example cores of Table 1 are not limiting of the stereoisomers (i.e. enantiomers, diastereomers) listed.

TABLE 1
Example core structures

| ID # | Structure |
|---|---|
| 1A1 | 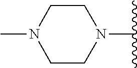 [SMILES: *N1CCN(C)CC1] |
| 1A2 | 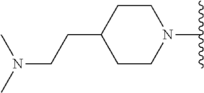 [SMILES: *N1CCC(CCN(C)C)CC1] |
| 1A3 | 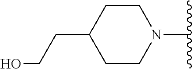 [SMILES: *N1CCC(CCO)CC1] |
| 1A4 | 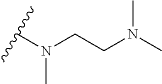 [SMILES: *N(C)CCN(C)C] |
| 1A5 | 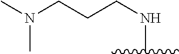 [SMILES: *NCCCN(C)C] |
| 2A1 | 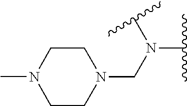 [SMILES: *N(*)CN1CCN(C)CC1] |
| 2A2 | 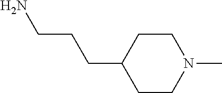 |
| 2A3 | 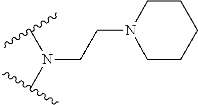 [SMILES: *N(*)CCN1CCCCC1] |
| 2A4 | 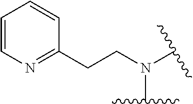 [SMILES: *N(*)CCc1ccccn1] |
| 2A5 | 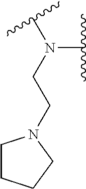 [SMILES: *N(*)CCN1CCCC1] |
| 2A6 | 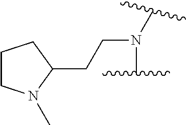 [SMILES: *N(*)CCC1CCCN1C] |
| 2A7 | 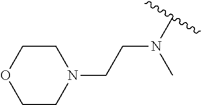 [SMILES: *N(C)CCN1CCOCC1] |
| 2A8 | 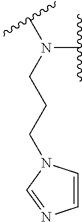 [SMILES: *N(*)CCCn1ccnc1] |
| 2A9 | 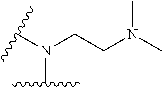 [SMILES: *N(*)CCN(C)C] |
| 2A9V | 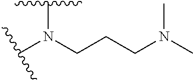 [SMILES: *N(*)CCCN(C)C] |
| 2A10 | 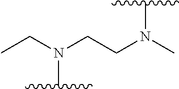 [SMILES: *N(C)CCN(*)CC] |
| 2A11 | 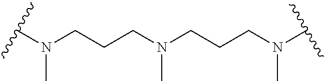 [SMILES: *N(C)CCCN(C)CCCN(*)C] |
| 2A12 | 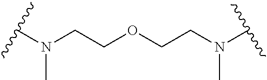 [SMILES: *N(C)CCOCCN(*)C] |
| 3A1 | 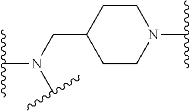 [SMILES: *N(*)CC1CCN(*)CC1] |
| 3A2 | 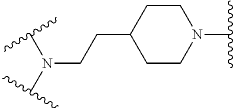 [SMILES: *N(*)CCC1CCN(*)CC1] |
| 3A3 | 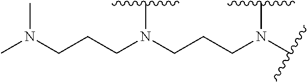 [SMILES: *N(*)CCCN(*)CCCN(C)C] |
| 3A4 | 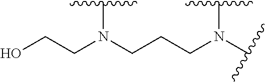 [SMILES: *N(*)CCCN(*)CCO] |
| 3A5 | 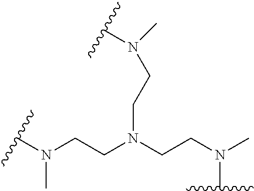 [SMILES: *N(C)CCN(CCN(*)C)CCN(*)C] |
| 4A1 | 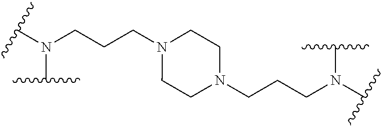 [SMILES: *N(*)CCCN1CCN(CCCN(*)*)CC1] |
| 4A2 | 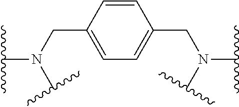 [SMILES: *N(*)Cc1ccc(CN(*)*)cc1] |
| 4A3 | 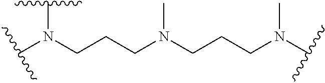 [SMILES: *N(*)CCCN(C)CCCN(*)C] |
| 4A4 | 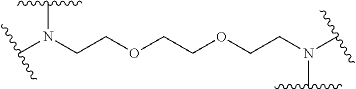 [SMILES: *N(*)CCOCCOCCN(*)*] |
| 5A1 | 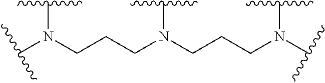 [SMILES: *N(*)CCCN(*)CCCN(*)*] |
| 5A2 | 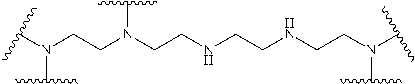 [SMILES: *N(*)CCNCCNCCN(*)CCN(*)*] |
| 5A3 | 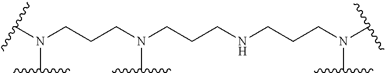 [SMILES: *N(*)CCCNCCCN(*)CCCN(*)*] |
| 5A4 | 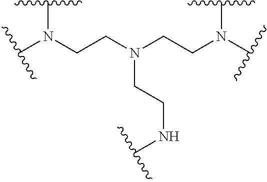 [SMILES: *NCCN(CCN(*)*)CCN(*)*] |
| 5A5 | 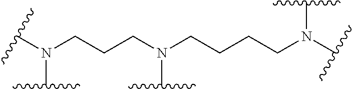 [SMILES: *N(*)CCCCN(*)CCCN(*)*] |
| 6A1 | 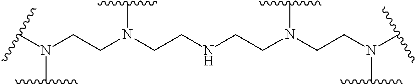 [SMILES: *N(*)CCN(*)CCNCCN(*)CCN(*)*] |
| 6A2 | 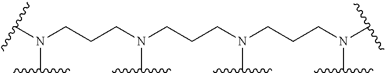 [SMILES: *N(*)CCCN(*)CCCN(*)CCCN(*)*] |
| 6A3 | 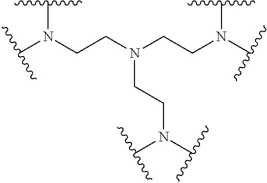 [SMILES: *N(*)CCN(CCN(*)*)CCN(*)*] |
| 6A4 | 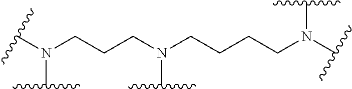 [SMILES: *N(*)CCCCN(*)CCCN(*)*] |
| 1H1 | 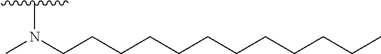 [SMILES: *N(C)CCCCCCCCCCCC] |
| 1H2 | 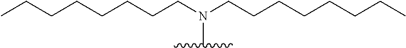 [SMILES: *N(CCCCCCCC)CCCCCCCC] |
| 1H3 | 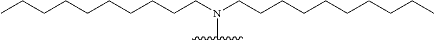 [SMILES: *N(CCCCCCCCCC)CCCCCCCCCC] |
| 2H1 | 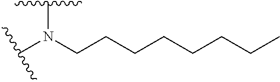 [SMILES: *N(*)CCCCCCCC] |
| 2H2 | 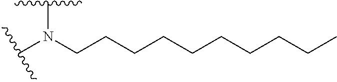 [SMILES: *N(*)CCCCCCCCCC] |
| 2H3 | 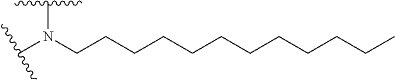 [SMILES: *N(*)CCCCCCCCCCCC] |
| 2H4 | 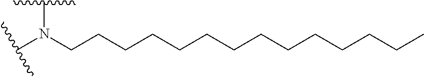 [SMILES: *N(*)CCCCCCCCCCCCCC] |
| 2H5 | 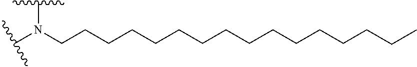 [SMILES: *N(*)CCCCCCCCCCCCCCCC] |
| 2H6 | 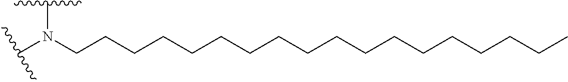 [SMILES: *N(*)CCCCCCCCCCCCCCCCCC] |

In some embodiments of X_{Core}, the core comprises a structural formula selected from the group consisting of:

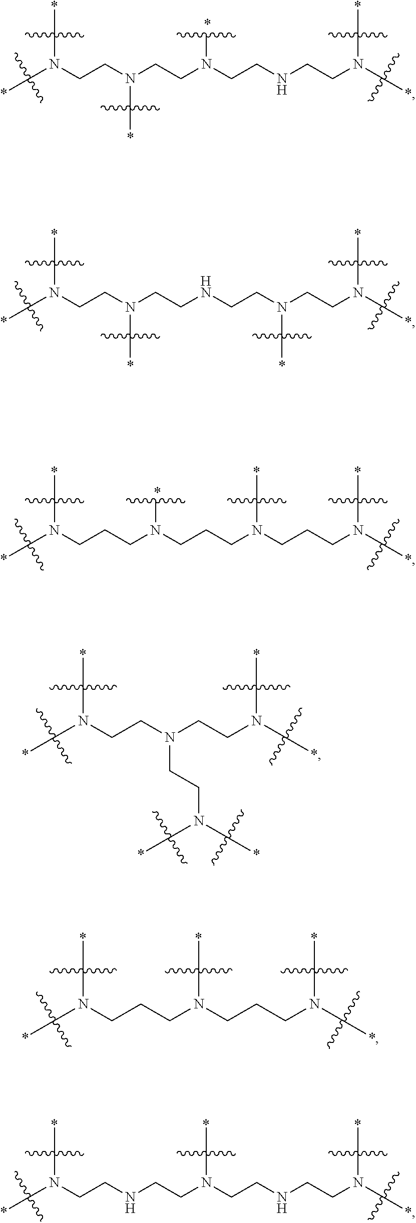

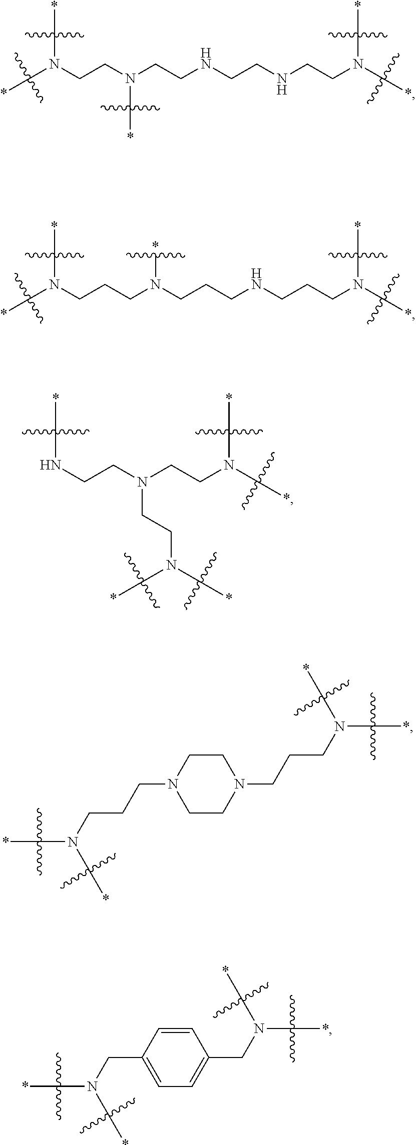

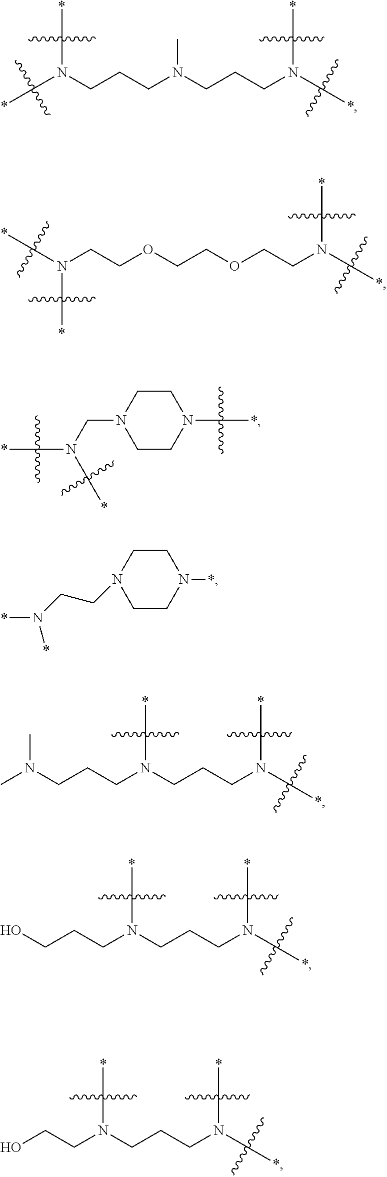

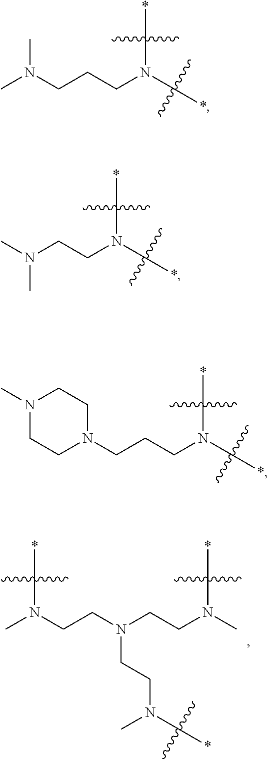

and pharmaceutically acceptable salts thereof, wherein * indicates a point of attachment of the core to a branch of the plurality of branches.

In some embodiments, the plurality (N) of branches comprises at least 3 branches, at least 4 branches, at least 5 branches. In some embodiments, the plurality (N) of branches comprises at least 3 branches. In some embodiments, the plurality (N) of branches comprises at least 4 branches. In some embodiments, the plurality (N) of branches comprises at least 5 branches.

In some embodiments of X_{Branch}, g is 1, 2, 3, or 4. In some embodiments of X_{Branch}, g is 1. In some embodiments of X_{Branch}, g is 2. In some embodiments of X_{Branch}, g is 3. In some embodiments of X_{Branch}, g is 4.

In some embodiments of X_{Branch}, Z=2^{(g-1)} and when g=1, G=0. In some embodiments of X_{Branch}, Z=2^{(g-1)} and G=Σᵢ₌₀^{i=g-2}2ⁱ, when g≠1.

In some embodiments of X_{Branch}, g=1, G=0, Z=1, and each branch of the plurality of branches comprises a structural formula each branch of the plurality of branches comprises a structural formula * [symbol] diacyl group [symbol] terminating group).

In some embodiments of X_{Branch}, g=2, G=1, Z=2, and each branch of the plurality of branches comprises a structural formula

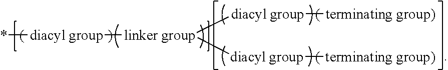

In some embodiments of X_{Branch}, g=3, G=3, Z=4, and each branch of the plurality of branches comprises a structural formula

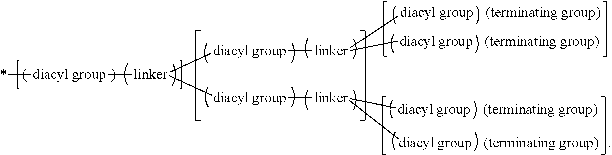

In some embodiments of X_{Branch}, g=4, G=7, Z=8, and each branch of the plurality of branches comprises a structural formula

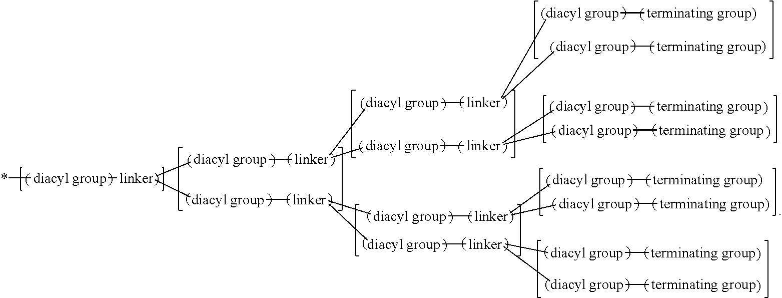

In some embodiments, the dendrimers described herein with a generation (g)=1 has structure the structure:

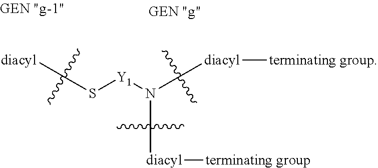

In some embodiments, the dendrimers described herein with a generation (g)=1 has structure the structure:

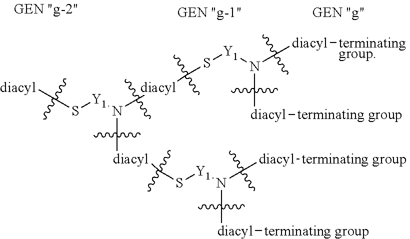

The example formulation of the dendrimers described herein for generations 1 to 4 is shown in Table 2. The number of diacyl groups, linker groups, and terminating groups can be calculated based on g.

TABLE 2
Formulation of Dendrimer Groups Based on Generation (g)

| | g = 1 | g = 2 | g = 3 | g = 4 | |
|---|---|---|---|---|---|
| # of diacyl grp | 1 | 1 + 2 = 3 | 1 + 2 + | 1 + 2 + 2² + | 1 + 2 + |
| | | | 2² = 7 | 2³ = 15 | . . . + 2g⁻¹ |
| # of linker grp | 0 | 1 | 1 + 2 | 1 + 2 + 2² | 1 + 2 + |
| | | | | | . . . + 2g⁻² |
| # of terminating | 1 | 2 | 2² | 2³ | 2^{(g-1)} |
| grp | | | | | |

In some embodiments, the diacyl group independently comprises a structural formula

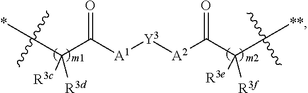

* indicates a point of attachment of the diacyl group at the proximal end thereof, and ** indicates a point of attachment of the diacyl group at the distal end thereof.

In some embodiments of the diacyl group of X_{Branch}, Y³ is independently at each occurrence an optionally substituted; alkylene, an optionally substituted alkenylene, or an optionally substituted arenylene. In some embodiments of the diacyl group of X_{Branch}, Y³ is independently at each occurrence an optionally substituted alkylene (e.g., C₁-C₁₂). In some embodiments of the diacyl group of X_{Branch}, Y³ is independently at each occurrence an optionally substituted alkenylene (e.g., C₁-C₁₂). In some embodiments of the diacyl group of X_{Branch}, Y³ is independently at each occurrence an optionally substituted arenylene (e.g., C₁-C₁₂).

In some embodiments of the diacyl group of X_{Branch}, A¹ and A² are each independently at each occurrence —O—, —S—, or —NR⁴—. In some embodiments of the diacyl group of X_{Branch}, A¹ and A² are each independently at each occurrence —O—. In some embodiments of the diacyl group of X_{Branch}, A¹ and A² are each independently at each occurrence —S—. In some embodiments of the diacyl group of X_{Branch}, A¹ and A² are each independently at each occurrence —NR⁴— and R⁴ is hydrogen or optionally substituted alkyl (e.g., C₁-C₆). In some embodiments of the diacyl group of X_{Branch}, m¹ and m² are each independently at each occurrence 1, 2, or 3. In some embodiments of the diacyl group of X_{Branch}, m¹ and m² are each independently at each occurrence 1. In some embodiments of the diacyl group of X_{Branch}, m¹ and m² are each independently at each occurrence 2. In some embodiments of the diacyl group of X_{Branch}, m¹ and m² are each independently at each occurrence 3. In some embodiments of the diacyl group of X_{Branch}, R^{3c}, R^{3d}, R^{3e}, and R^{3f} are each independently at each occurrence hydrogen or an optionally substituted alkyl. In some embodiments of the diacyl group of X_{Branch}, R^{3c}, R^{3d}, R^{3e}, and R^{3f} are each independently at each occurrence hydrogen. In some embodiments of the diacyl group of X_{Branch}, R^{3c}, R^{3d}, R^{3e}, and R^{3f} are each independently at each occurrence an optionally substituted (e.g., C₁-C₈) alkyl.

In some embodiments of the diacyl group, A¹ is —O— or —NH—. In some embodiments of the diacyl group, A₁ is —O—. In some embodiments of the diacyl group, A² is —O— or —NH—. In some embodiments of the diacyl group, A² is —O—. In some embodiments of the diacyl group, Y³ is C₁-C₁₂ (e.g., C₁-C₆, such as C₁-C₃) alkylene.

In some embodiments of the diacyl group, the diacyl group independently at each occurrence comprises a structural formula

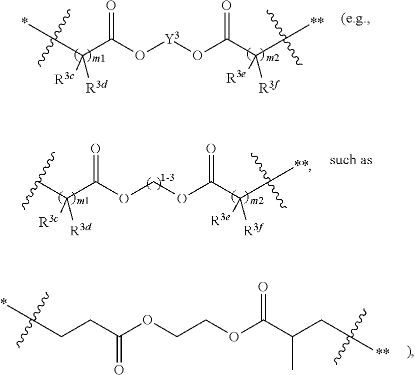

optionally R^{3c}, R^{3d}, R^{3e}, and R^{3f} are each independently at each occurrence hydrogen or C₁-C₃ alkyl.

In some embodiments, linker group independently comprises a structural formula

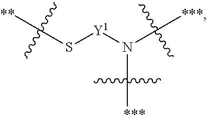

** indicates a point of attachment of the linker to a proximal diacyl group, and *** indicates a point of attachment of the linker to a distal diacyl group.

In some embodiments of the linker group of X_{Branch} if present, Y₁ is independently at each occurrence an optionally substituted alkylene, an optionally substituted alkenylene, or an optionally substituted arenylene. In some embodiments of the linker group of X_{Branch} if present, Y₁ is independently at each occurrence an optionally substituted alkylene (e.g., C₁-C₁₂). In some embodiments of the linker group of X_{Branch} if present, Y₁ is independently at each occurrence an optionally substituted alkenylene (e.g., C₁-C₁₂). In some embodiments of the linker group of X_{Branch} if present, Y₁ is independently at each occurrence an optionally substituted arenylene (e.g., C₁-C₁₂).

In some embodiments of the terminating group of X_{Branch}, each terminating group is independently selected from optionally substituted alkenylthiol. In some embodiments of the terminating group of X_{Branch}, each terminating group is an optionally substituted alkenylthiol (e.g., C₁-C₁₈, such as C₄-C₁₈). In some embodiments of the terminating group of X_{Branch}, each terminating group is optionally substituted alkenylthiol (e.g., C₁-C₁₈, such as C₄-C₁₈).

In some embodiments of the terminating group of X_{Branch}, each terminating group is independently C₁-C₁₈ alkenylthiol and or alkenyl moiety is optionally substituted with one or more substituents each independently selected from halogen, C₆-C₁₂ aryl, C₁-C₁₂ alkylamino, C₄-C₆ N-heterocycloalkyl, —OH, —C(O)OH, —C(O)N(C₁-C₃ alkyl)-(C₁-C₆ alkylene)-(C₁-C₁₂ alkylamino), —C(O)N(C₁-C₃ alkyl)-(C₁-C₆ alkylene)-(C₄-C₆ N-heterocycloalkyl), —C(O)—(C₁-C₁₂ alkylamino), and —C(O)—(C₄-C₆ N-heterocycloalkyl), and the C₄-C₆ N-heterocycloalkyl moiety of any of the preceding substituents is optionally substituted with C₁-C₃ alkyl or C₁-C₃ hydroxyalkyl.

In some embodiments of the terminating group of X_{Branch}, each terminating group is independently C₁-C₁₈ (e.g., C₄-C₁₈) alkenylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino

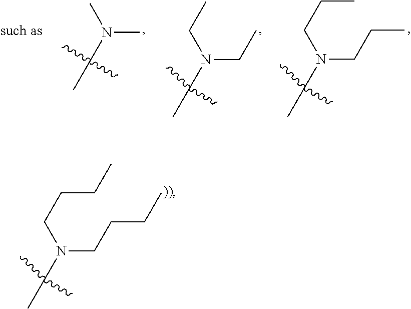

C₄-C₆ N-heterocycloalkyl (e.g., N-pyrrolidinyl

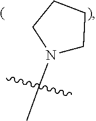 [SMILES: *N1CCCC1]

piperidinyl

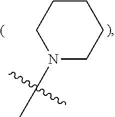 [SMILES: *N1CCCCC1]

### N-azepanyl

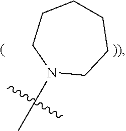 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino)) (e.g.,

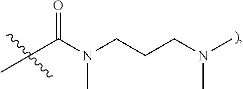 [SMILES: *C(=O)N(C)CCCN(C)C]

—C(O)N(C₁-C₃ alkyl)-(C₁-C₆ alkylene)-(C₄-C₆ N-heterocycloalkyl) (e.g.,

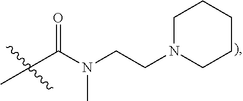 [SMILES: *C(=O)N(C)CCN1CCCCC1]

—C(O)—(C₁-C₁₂ alkyl-amino (e.g., mono- or di-alkylamino)), and —C(O)—(C₄-C₆ N-heterocycloalkyl)

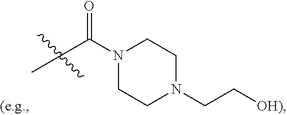

wherein the C₄-C₆ N-heterocycloalkyl moiety of any of the preceding substituents is optionally substituted with C₁-C₃ alkyl or C₁-C₃ hydroxyalkyl. In some embodiments of the terminating group of X_{Branch}, each terminating group is independently C₁-C₁₈ (e.g., C₄-C₁₈) alkenylthiol, wherein the alkenyl moiety is optionally substituted with one substituent —OH. In some embodiments of the terminating group of X_{Branch}, each terminating group is independently C₁-C₁₈ (e.g., C₄-C₁₈) alkenylthiol, wherein the alkenyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino

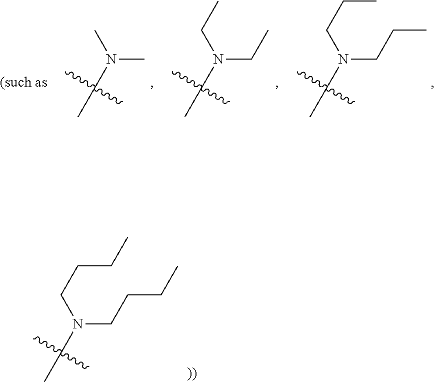

and C₄-C₆ N-heterocycloalkyl (e.g., N-pyrrolidinyl

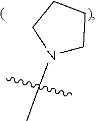 [SMILES: *N1CCCC1]

### N-piperidinyl

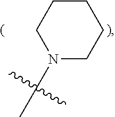 [SMILES: *N1CCCCC1]

### N-azepanyl

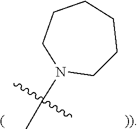 [SMILES: *N1CCCCCC1]

In some embodiments of the terminating group of X_{Branch}, each terminating group is independently C₁-C₁₈ (e.g., C₄-C₁₈) alkenylthiol. In some embodiments of the terminating group of X_{Branch}, each terminating group is independently C₁-C₁₈ (e.g., C₄-C₁₈) alkenylthiol.

In some embodiments, a method for preparing an unsaturated thiol compound having structural formula I**:

R—SH (I**)

from a compound having structural formula II**:

R—OH (II**),

wherein R is a C₆-C₂₂ alkenyl, C₆-C₂₂ alkadienyl, or C₆-C₂₂ alkatrienyl; and

wherein the method provides the unsaturated thiol compound in a yield of at least 40%, 50%, 60%, 70%, 80%, or 90%.

In some embodiments, R is a C₆-C₂₂ alkenylthiol having one, two or three double bond(s). In some embodiments, R is a C₆-C₂₂ alkenylthiol having one double bond. In some embodiments, R is a C₆-C₂₂ alkenyl thiol having two double bonds. In some embodiments, R is a C₆-C₂₂ alkenyl thiol having three double bonds. In some embodiments, R is a C₆-C₁₆ alkenylthiol having one double bond. In some embodiments, R is a C₆-C₁₆ alkenylthiol having two double bonds. In some embodiments, R is a C₆-C₁₆ alkenylthiol having three double bonds. In some embodiments, R is a C₆-C₁₄ alkenylthiol having one double bond. In some embodiments, R is a C₆-C₁₄ alkenylthiol having two double bonds. In some embodiments, R is a C₆-C₁₄ alkenylthiol having three double bonds. In some embodiments, R is a C₆-C₁₀ alkenylthiol having one double bond. In some embodiments, R is a C₆-C₁₀ alkenylthiol having two double bonds. In some embodiments, R is a C₆-C₁₀ alkenylthiol having three double bonds.

In some embodiments, R has a structural formula:

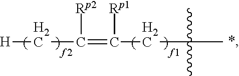

wherein:
R^{p1} and R^{p2} are each independently H or C₁-C₆ alkyl;
f1 is 1, 2, 3, or 4; and
f2 is 0, 1, 2, or 3.

In some embodiments, —CR^{p2}=CR^{p1}— is a cis bond. In some embodiments, —CR^{p2}=CR^{p1}— is a trans bond. In some embodiments, R^{p1} is H. In some embodiments, R^{p1} is C₁-C₆ alkyl. In some embodiments, R^{p1} is C₁-C₃ alkyl. In some embodiments, R^{p2} is H. In some embodiments, R^{p2} is C₁-C₆ alkyl. In some embodiments, R^{p2} is C₁-C₃ alkyl. In some embodiments, f1 is 1. In some embodiments, f1 is 2. In some embodiments, f1 is 3. In some embodiments, f1 is 4. In some embodiments, f2 is 0. In some embodiments, f2 is 1. In some embodiments, f2 is 2. In some embodiments, f2 is 3. In some embodiments, f1+f2≥3. In some embodiments, f1+f2 is 3. In some embodiments, f1+f2 is 4. In some embodiments, f1+f2 is 5. In some embodiments, f1+f2 is 6.

In some embodiments, R has a structural formula:

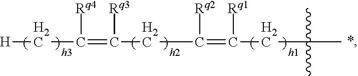

wherein:
R^{q1}, R^{q2}, R^{q3}, and R^{q4} are each independently H or C₁-C₆ alkyl;
h1 is 1, 2, 3, or 4;
h2 is 1 or 2; and
h3 is 0, 1, 2, or 3.

In some embodiments, —CR^{q2}=CR^{q1}— is a cis bond. In some embodiments, —CR^{q2}=CR^{q1}— is a trans bond. In some embodiments, —CR^{q4}=CR^{q3}— is a cis bond. In some embodiments, —CR^{q4}=CR^{q3}— is a trans bond. In some embodiments, R^{q1} is H. In some embodiments, R^{q1} is C₁-C₆alkyl. In some embodiments, R^{q1} is C₁-C₃ alkyl. In some embodiments, R^{q1} is methyl. In some embodiments, R^{q2} is H. In some embodiments, R^{q2} is C₁-C₆ alkyl. In some embodiments, R^{q2} is C₁-C₃ alkyl. In some embodiments, R^{q2} is methyl. In some embodiments, R^{q3} is H. In some embodiments, R^{q3} is C₁-C₆alkyl. In some embodiments, R^{q3} is C₁-C₃ alkyl. In some embodiments, R^{q3} is methyl. In some embodiments, R^{q4} is H. In some embodiments, R^{q4} is C₁-C₆ alkyl. In some embodiments, R^{q4} is C₁-C₃ alkyl. In some embodiments, R^{q4} is methyl. In some embodiments, h1 is 1. In some embodiments, h1 is 2. In some embodiments, h1 is 3. In some embodiments, h1 is 4. In some embodiments, h2 is 1. In some embodiments, h2 is 2. In some embodiments, h3 is 0. In some embodiments, h3 is 1. In some embodiments, h3 is 2. In some embodiments, h3 is 3. In some embodiments, h1+h2+h3≥3. In some embodiments, h1+h2+h3 is 3. In some embodiments, h1+h2+h3 is 4. In some embodiments, h1+h2+h3 is 5. In some embodiments, h1+h2+h3 is 6.

In some embodiments, R has a structural formula:

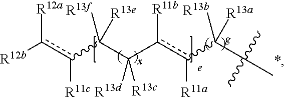

wherein:
indicates the point of attachment to the sulfur;
e is 0, 1, 2, 3, 4, 5, or 6;
g is 1, 2, or 3 (optionally g is 1);
x is independently at each occurrence 0, 1, 2, or 3 and
R^{11a}, R^{11b}, R^{11c}, R^{12a}, R^{12b}, R^{13a}, R^{13b}, R^{13c}, R^{13d}, R^{13e}, and R^{13f} are each independently at each occurrence H or C₁-C₆ alkyl.

In some embodiments, R has the structural formula

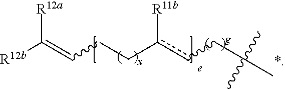

In some embodiments, R has the structural formula

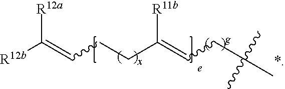

In some embodiments, R has the structural formula

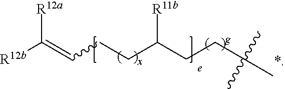

In some embodiments, e is 0. In some embodiments, e is 1. In some embodiments, e is 2. In some embodiments, e is 3. In some embodiments, e is 4. In some embodiments, e is 5. In some embodiments, e is 6. In some embodiments, g is 1. In some embodiments, g is 2. In some embodiments, g is 3. In some embodiments, x is 0. In some embodiments, x is 1. In some embodiments, x is 2. In some embodiments, x is 3. In some embodiments, R^{11a} is H. In some embodiments, R^{11a} is C₁-C₆ alkyl. In some embodiments, R^{11a} is C₁-C₃ alkyl. In some embodiments, R^{11a} is methyl. In some embodiments, R^{11b} is H. In some embodiments, R^{11b} is C₁-C₆ alkyl. In some embodiments, R^{11b} is C₁-C₃ alkyl. In some embodiments, R^{11b} is methyl. In some embodiments, R^{11c} is H. In some embodiments, R^{11c} is C₁-C₆ alkyl. In some embodiments, R^{11c} is C₁-C₃ alkyl. In some embodiments, R^{11c} is methyl. In some embodiments, R^{12a} is H. In some embodiments, R^{12a} is C₁-C₆ alkyl. In some embodiments, R^{12a} is C₁-C₃ alkyl. In some embodiments, R^{12a} is methyl. In some embodiments, R^{12b} is H. In some embodiments, R^{12b} is C₁-C₆ alkyl. In some embodiments, R^{12b} is C₁-C₃ alkyl. In some embodiments, R^{12b} is methyl. In some embodiments, R^{13a} is H. In some embodiments, R^{13a} is C₁-C₆ alkyl. In some embodiments, R^{13a} is C₁-C₃ alkyl. In some embodiments, R^{13a} is methyl. In some embodiments, R^{13b} is H. In some embodiments, R^{13b} is C₁-C₆alkyl. In some embodiments, R^{13b} is C₁-C₃ alkyl. In some embodiments, R^{13b} is methyl. In some embodiments, R^{13c} is H. In some embodiments, R^{13c} is C₁-C₆ alkyl. In some embodiments, R^{13c} is C₁-C₃ alkyl. In some embodiments, R^{13c} is methyl. In some embodiments, R^{13d} is H. In some embodiments, R^{13d} is C₁-C₆ alkyl. In some embodiments, R^{13d} is C₁-C₃ alkyl. In some embodiments, R^{13d} is methyl. In some embodiments, R^{13e} is H. In some embodiments, R^{13e} is C₁-C₆ alkyl. In some embodiments, R^{13e} is C₁-C₃ alkyl. In some embodiments, R^{13e} is methyl. In some embodiments, R^{13f} is H. In some embodiments, R^{13f} is C₁-C₆ alkyl. In some embodiments, R^{13f} is C₁-C₃ alkyl. In some embodiments, R^{13f} is methyl.

In some embodiments of the terminating group of X_{Branch}, each terminating group is independently a structural set forth in Table 3. In some embodiments, the dendrimers described herein can comprise a terminating group or pharmaceutically acceptable salt, or thereof selected in Table 3. In some embodiments, the example terminating group of Table 3 are not limiting of the stereoisomers (i.e. enantiomers, diastereomers) listed.

TABLE 3
Example terminating group/peripheries structures

| ID # | Structure |
|---|---|
| 2C | 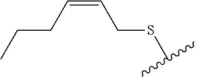 [SMILES: *SC/C=C\CCC] |
| 2T | 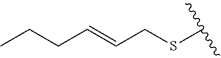 [SMILES: *SC/C=C/CCC] |
| 3C | 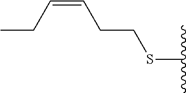 [SMILES: *SCC/C=C\CC] |
| 3T | 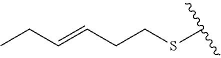 [SMILES: *SCC/C=C/CC] |
| 4C | 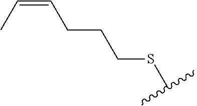 [SMILES: *SCCC/C=C\C] |
| 4T | 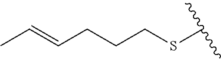 [SMILES: *SCCC/C=C/C] |
| 5 | 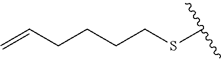 [SMILES: *SCCCCC=C] |
| 8/2 | 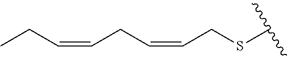 [SMILES: *SC/C=C\C/C=C\CC] |
| Citronellol (Cit) | 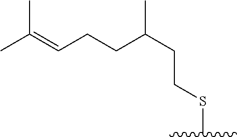 [SMILES: *SCCC(C)CCC=C(C)C] |
| Nerol (Nerol) | 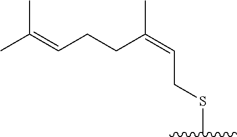 [SMILES: *SC/C=C(/C)CCC=C(C)C] |
| Farnesol (Far) | 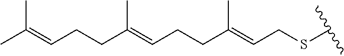 [SMILES: *SC/C=C(\C)CC/C=C(\C)CCC=C(C)C] |

In some embodiments, the dendrimer of Formula (X) is selected from those set forth in Table 4 and pharmaceutically acceptable salts thereof.

TABLE 4
Example unsaturated lipo-dendrimers

| ID # | Structure |
|---|---|
| 6A3-2C | 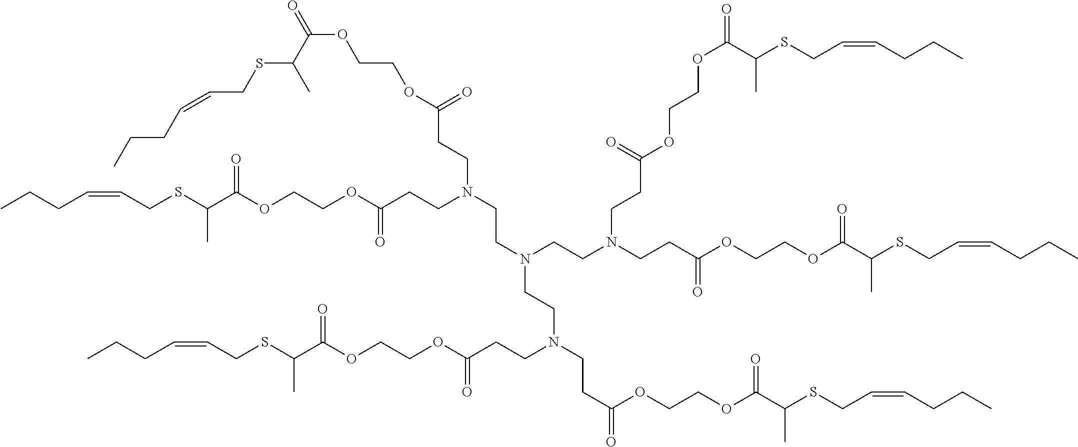 [SMILES: CCC/C=C\CSC(C)C(=O)OCCOC(=O)CCN(CCC(=O)OCCOC(=O)C(C)SC/C=C\CCC)CCN(CCN(CCC(=O)OCCOC(=O)C(C)SC/C=C\CCC)CCC(=O)OCCOC(=O)C(C)SC/C=C\CCC)CCN(CCC(=O)OCCOC(=O)C(C)SC/C=C\CCC)CCC(=O)OCCOC(=O)C(C)SC/C=C\CCC] |
| 2A2-Cit | 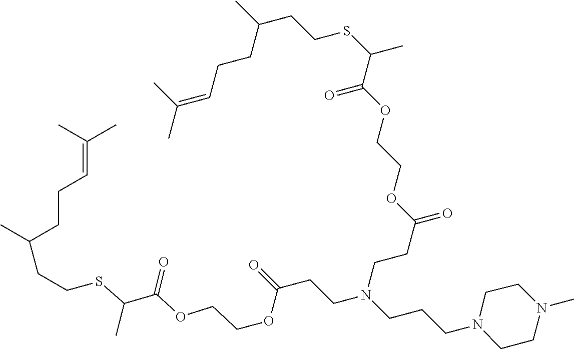 [SMILES: CC(C)=CCCC(C)CCSC(C)C(=O)OCCOC(=O)CCN(CCCN1CCN(C)CC1)CCC(=O)OCCOC(=O)C(C)SCCC(C)CCC=C(C)C] |
| 3A4-3T | 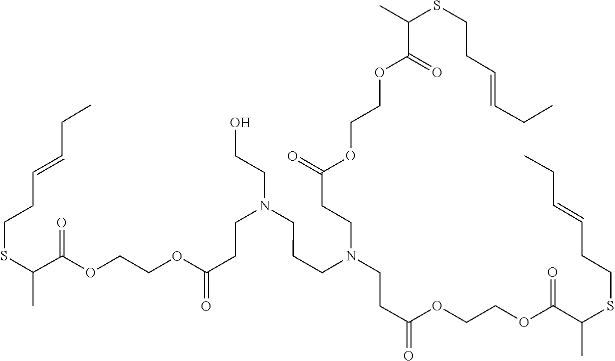 [SMILES: CC/C=C/CCSC(C)C(=O)OCCOC(=O)CCN(CCO)CCCN(CCC(=O)OCCOC(=O)C(C)SCC/C=C/CC)CCC(=O)OCCOC(=O)C(C)SCC/C=C/CC] |
| 3A4-5 | 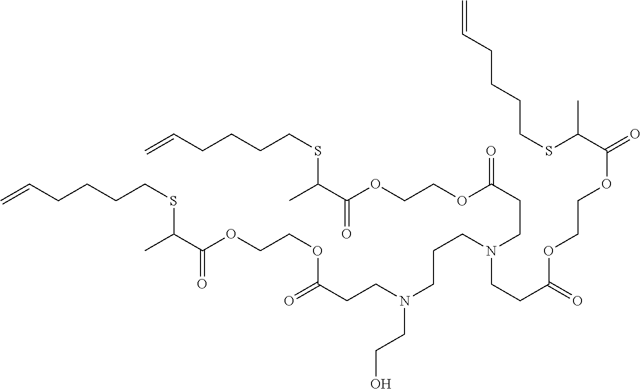 [SMILES: C=CCCCCSC(C)C(=O)OCCOC(=O)CCN(CCO)CCCN(CCC(=O)OCCOC(=O)C(C)SCCCCC=C)CCC(=O)OCCOC(=O)C(C)SCCCCC=C] |
| 4A3-4C | 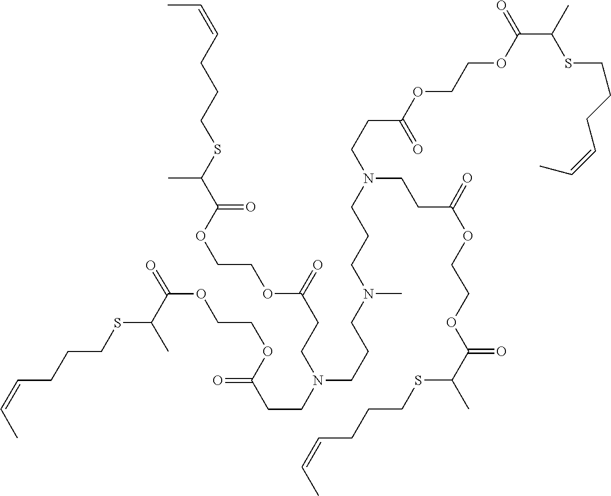 [SMILES: C/C=C\CCCSC(C)C(=O)OCCOC(=O)CCN(CCCN(C)CCCN(CCC(=O)OCCOC(=O)C(C)SCCC/C=C\C)CCC(=O)OCCOC(=O)C(C)SCCC/C=C\C)CCC(=O)OCCOC(=O)C(C)SCCC/C=C\C] |
| 3A4-3C | 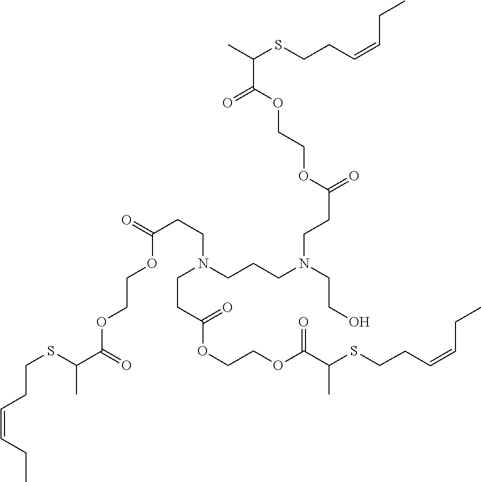 [SMILES: CC/C=C\CCSC(C)C(=O)OCCOC(=O)CCN(CCO)CCCN(CCC(=O)OCCOC(=O)C(C)SCC/C=C\CC)CCC(=O)OCCOC(=O)C(C)SCC/C=C\CC] |
| 6A3-Far | 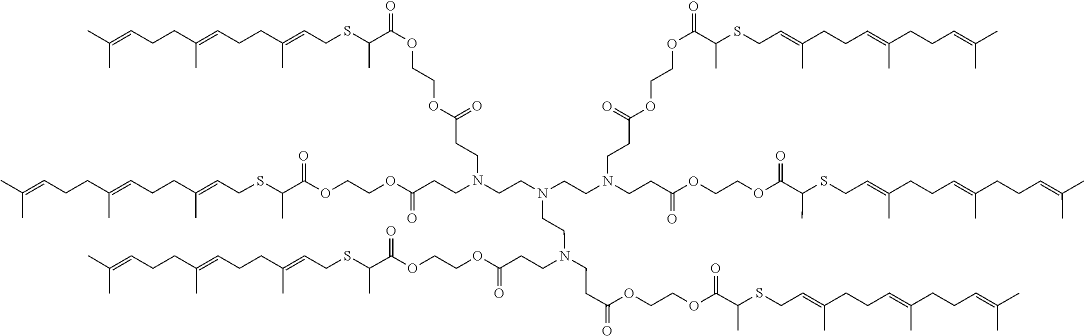 [SMILES: CC(C)=CCC/C(C)=C/CC/C(C)=C/CSC(C)C(=O)OCCOC(=O)CCN(CCC(=O)OCCOC(=O)C(C)SC/C=C(\C)CC/C=C(\C)CCC=C(C)C)CCN(CCN(CCC(=O)OCCOC(=O)C(C)SC/C=C(\C)CC/C=C(\C)CCC=C(C)C)CCC(=O)OCCOC(=O)C(C)SC/C=C(\C)CC/C=C(\C)CCC=C(C)C)CCN(CCC(=O)OCCOC(=O)C(C)SC/C=C(\C)CC/C=C(\C)CCC=C(C)C)CCC(=O)OCCOC(=O)C(C)SC/C=C(\C)CC/C=C(\C)CCC=C(C)C] |
| 2A2-3C | 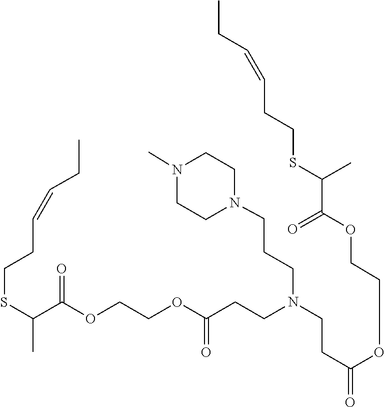 [SMILES: CC/C=C\CCSC(C)C(=O)OCCOC(=O)CCN(CCCN1CCN(C)CC1)CCC(=O)OCCOC(=O)C(C)SCC/C=C\CC] |
| 3A4-8/2 | 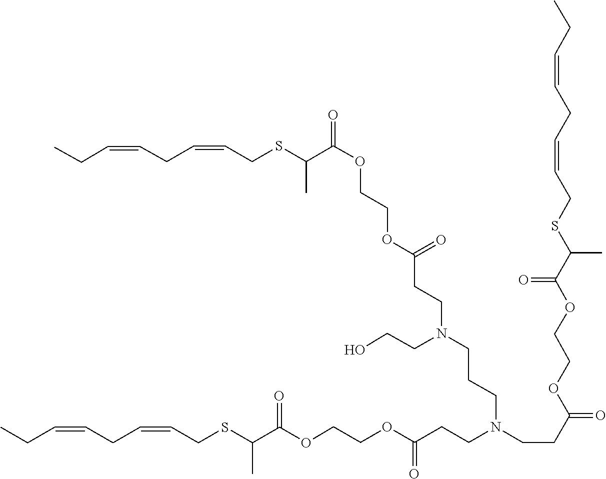 [SMILES: CC/C=C\C/C=C\CSC(C)C(=O)OCCOC(=O)CCN(CCO)CCCN(CCC(=O)OCCOC(=O)C(C)SC/C=C\C/C=C\CC)CCC(=O)OCCOC(=O)C(C)SC/C=C\C/C=C\CC] |
| 6A3-4T | 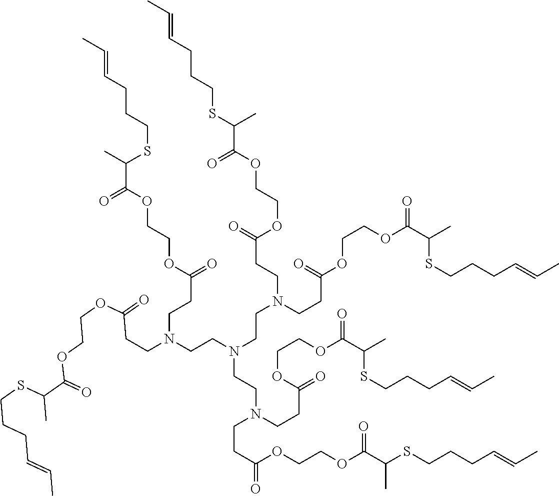 [SMILES: C/C=C/CCCSC(C)C(=O)OCCOC(=O)CCN(CCC(=O)OCCOC(=O)C(C)SCCC/C=C/C)CCN(CCN(CCC(=O)OCCOC(=O)C(C)SCCC/C=C/C)CCC(=O)OCCOC(=O)C(C)SCCC/C=C/C)CCN(CCC(=O)OCCOC(=O)C(C)SCCC/C=C/C)CCC(=O)OCCOC(=O)C(C)SCCC/C=C/C] |
| 4A1-2T | 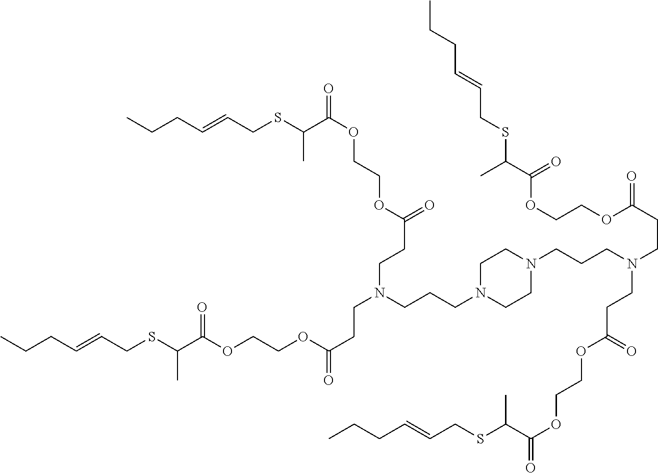 [SMILES: CCC/C=C/CSC(C)C(=O)OCCOC(=O)CCN(CCCN1CCN(CCCN(CCC(=O)OCCOC(=O)C(C)SC/C=C/CCC)CCC(=O)OCCOC(=O)C(C)SC/C=C/CCC)CC1)CCC(=O)OCCOC(=O)C(C)SC/C=C/CCC] |
| 6A3-4C | 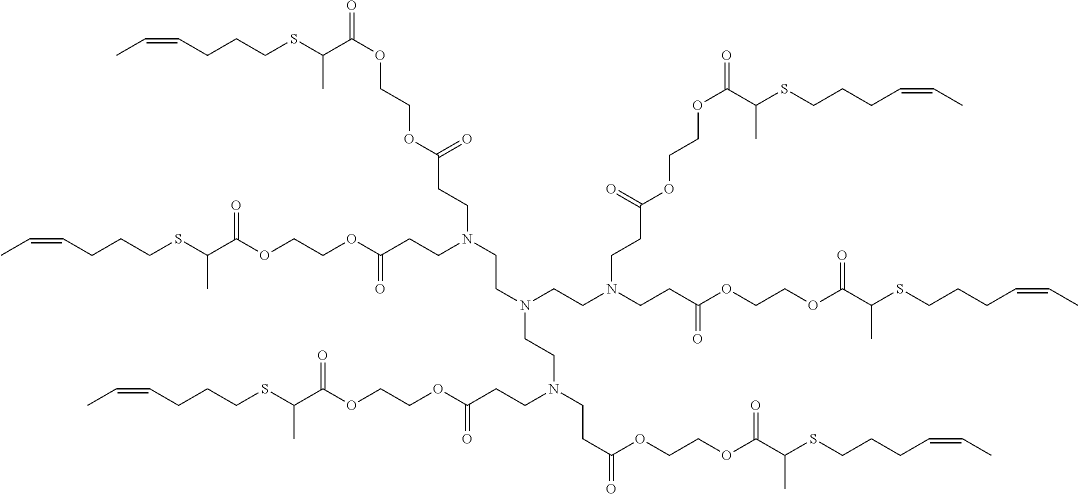 [SMILES: C/C=C\CCCSC(C)C(=O)OCCOC(=O)CCN(CCC(=O)OCCOC(=O)C(C)SCCC/C=C\C)CCN(CCN(CCC(=O)OCCOC(=O)C(C)SCCC/C=C\C)CCC(=O)OCCOC(=O)C(C)SCCC/C=C\C)CCN(CCC(=O)OCCOC(=O)C(C)SCCC/C=C\C)CCC(=O)OCCOC(=O)C(C)SCCC/C=C\C] |
| 6A3-5 | 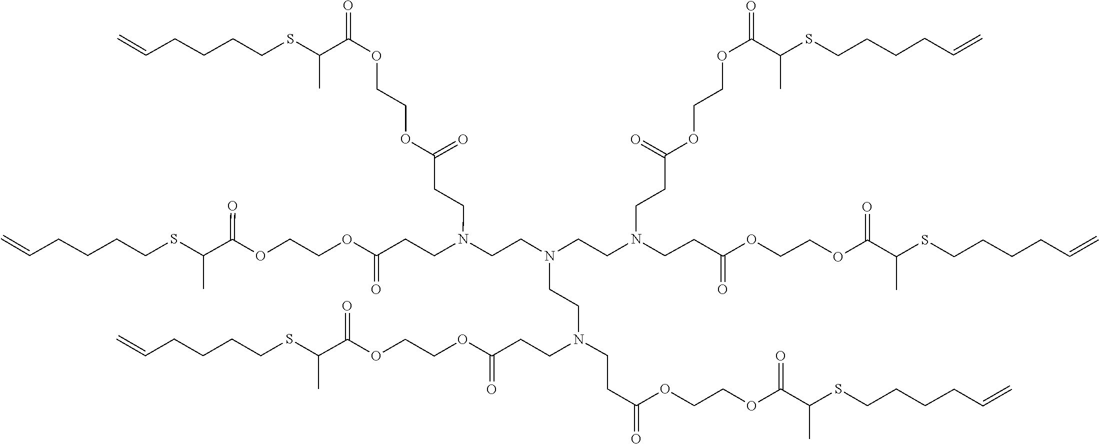 [SMILES: C=CCCCCSC(C)C(=O)OCCOC(=O)CCN(CCC(=O)OCCOC(=O)C(C)SCCCCC=C)CCN(CCN(CCC(=O)OCCOC(=O)C(C)SCCCCC=C)CCC(=O)OCCOC(=O)C(C)SCCCCC=C)CCN(CCC(=O)OCCOC(=O)C(C)SCCCCC=C)CCC(=O)OCCOC(=O)C(C)SCCCCC=C] |
| 6A3-3T | 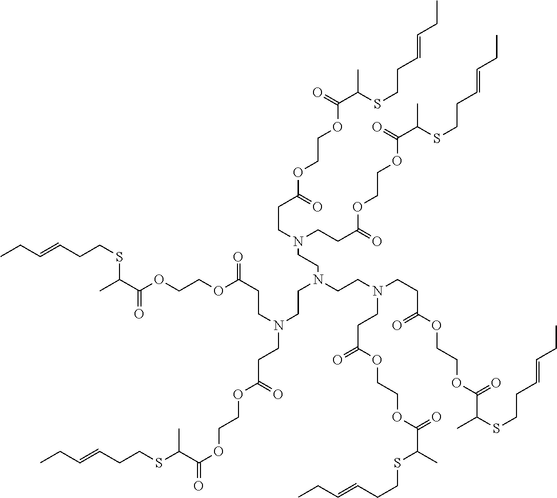 [SMILES: CC/C=C/CCSC(C)C(=O)OCCOC(=O)CCN(CCC(=O)OCCOC(=O)C(C)SCC/C=C/CC)CCN(CCN(CCC(=O)OCCOC(=O)C(C)SCC/C=C/CC)CCC(=O)OCCOC(=O)C(C)SCC/C=C/CC)CCN(CCC(=O)OCCOC(=O)C(C)SCC/C=C/CC)CCC(=O)OCCOC(=O)C(C)SCC/C=C/CC] |
| 6A3-Ne | 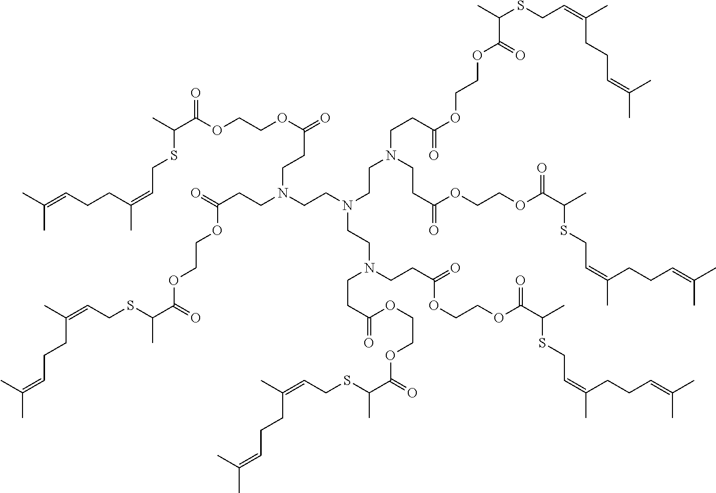 [SMILES: CC(C)=CCC/C(C)=C\CSC(C)C(=O)OCCOC(=O)CCN(CCC(=O)OCCOC(=O)C(C)SC/C=C(/C)CCC=C(C)C)CCN(CCN(CCC(=O)OCCOC(=O)C(C)SC/C=C(/C)CCC=C(C)C)CCC(=O)OCCOC(=O)C(C)SC/C=C(/C)CCC=C(C)C)CCN(CCC(=O)OCCOC(=O)C(C)SC/C=C(/C)CCC=C(C)C)CCC(=O)OCCOC(=O)C(C)SC/C=C(/C)CCC=C(C)C] |
| 6A3-3C | 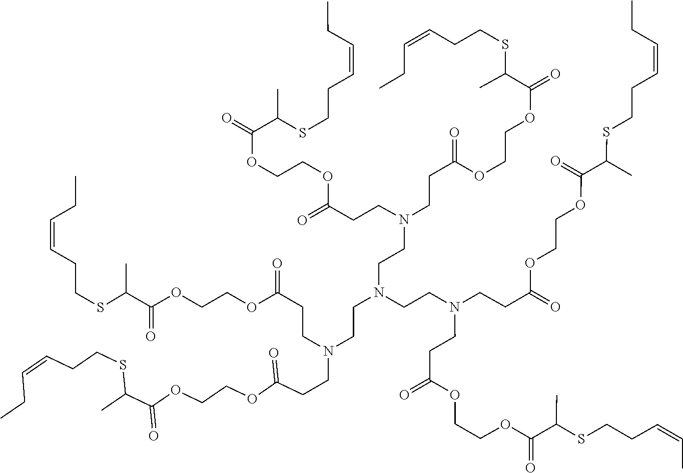 [SMILES: C/C=C\CCSC(C)C(=O)OCCOC(=O)CCN(CCC(=O)OCCOC(=O)C(C)SCC/C=C\CC)CCN(CCN(CCC(=O)OCCOC(=O)C(C)SCC/C=C\CC)CCC(=O)OCCOC(=O)C(C)SCC/C=C\CC)CCN(CCC(=O)OCCOC(=O)C(C)SCC/C=C\CC)CCC(=O)OCCOC(=O)C(C)SCC/C=C\CC] |
| 2A2-5 | 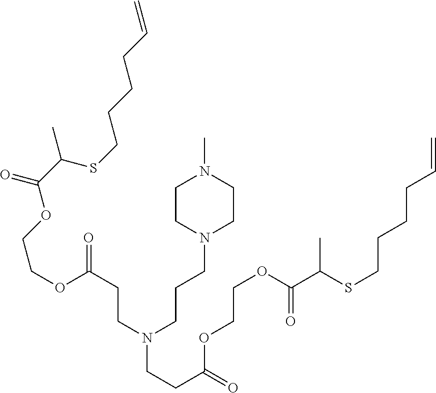 [SMILES: C=CCCCCSC(C)C(=O)OCCOC(=O)CCN(CCCN1CCN(C)CC1)CCC(=O)OCCOC(=O)C(C)SCCCCC=C] |
| 2A9V-5 | 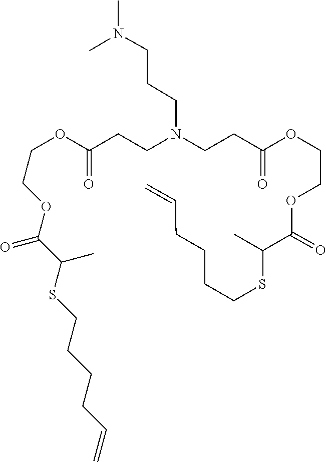 [SMILES: C=CCCCCSC(C)C(=O)OCCOC(=O)CCN(CCCN(C)C)CCC(=O)OCCOC(=O)C(C)SCCCCC=C] |
| 4A1-5 | 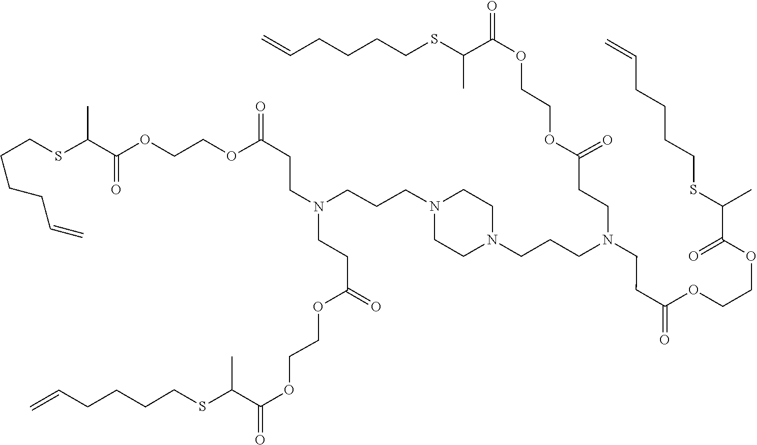 [SMILES: C=CCCCCSC(C)C(=O)OCCOC(=O)CCN(CCCN1CCN(CCCN(CCC(=O)OCCOC(=O)C(C)SCCCCC=C)CCC(=O)OCCOC(=O)C(C)SCCCCC=C)CC1)CCC(=O)OCCOC(=O)C(C)SCCCCC=C] |
| 3A4-SC8 | 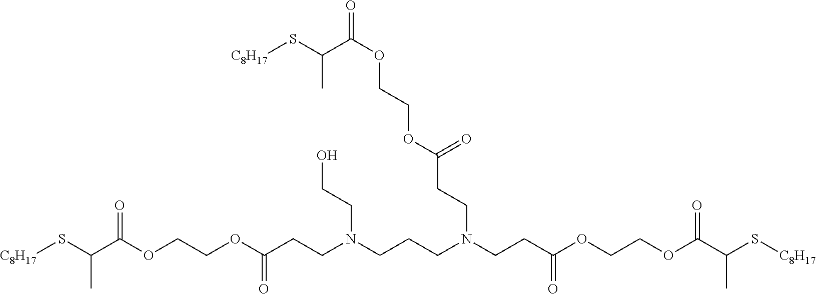 |
| 6A3-2T | 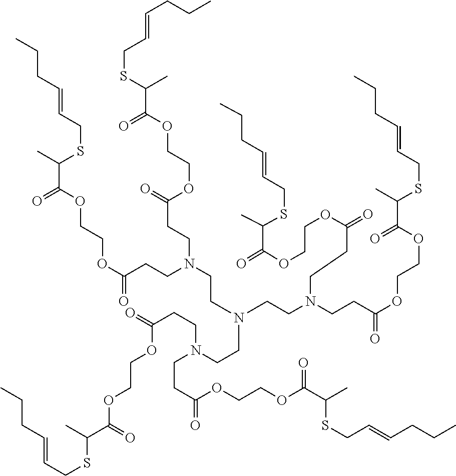 |
| 3A4-Cit | 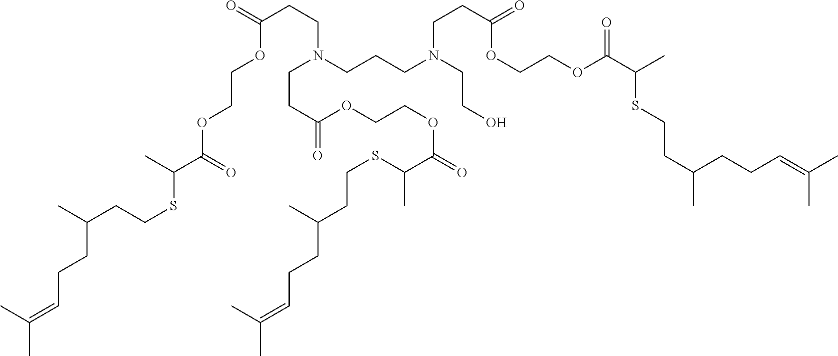 [SMILES: CC(C)=CCCC(C)CCSC(C)C(=O)OCCOC(=O)CCN(CCO)CCCN(CCC(=O)OCCOC(=O)C(C)SCCC(C)CCC=C(C)C)CCC(=O)OCCOC(=O)C(C)SCCC(C)CCC=C(C)C] |
| 4A1-3T | 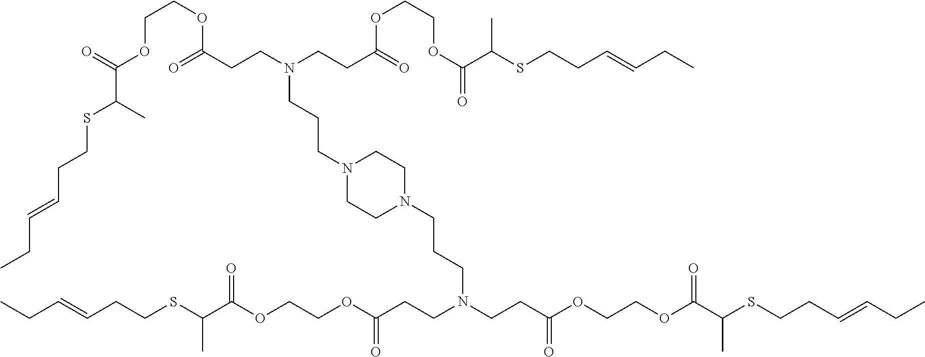 [SMILES: CC/C=C/CCSC(C)C(=O)OCCOC(=O)CCN(CCCN1CCN(CCCN(CCC(=O)OCCOC(=O)C(C)SCC/C=C/CC)CCC(=O)OCCOC(=O)C(C)SCC/C=C/CC)CC1)CCC(=O)OCCOC(=O)C(C)SCC/C=C/CC] |
| 4A1-4T | 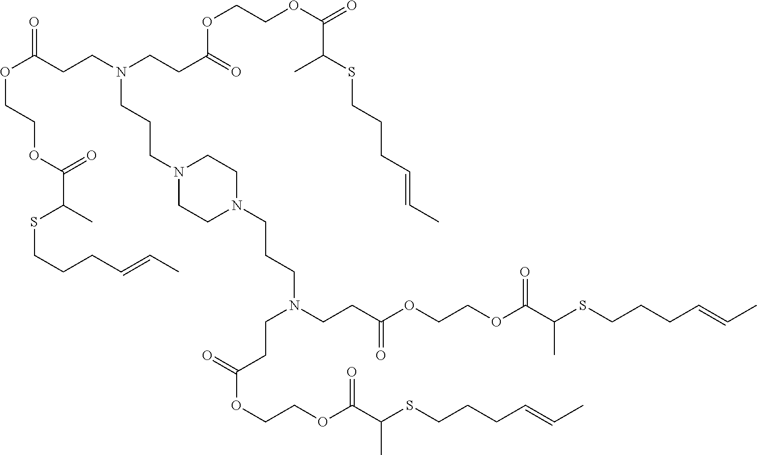 [SMILES: C/C=C/CCCSC(C)C(=O)OCCOC(=O)CCN(CCCN1CCN(CCCN(CCC(=O)OCCOC(=O)C(C)SCCC/C=C/C)CCC(=O)OCCOC(=O)C(C)SCCC/C=C/C)CC1)CCC(=O)OCCOC(=O)C(C)SCCC/C=C/C] |
| 4A1-Cit | 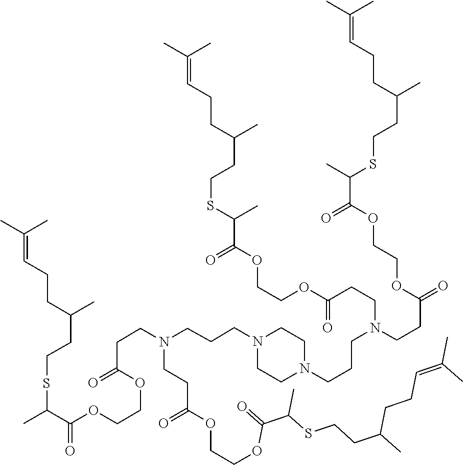 [SMILES: CC(C)=CCCC(C)CCSC(C)C(=O)OCCOC(=O)CCN(CCCN1CCN(CCCN(CCC(=O)OCCOC(=O)C(C)SCCC(C)CCC=C(C)C)CCC(=O)OCCOC(=O)C(C)SCCC(C)CCC=C(C)C)CC1)CCC(=O)OCCOC(=O)C(C)SCCC(C)CCC=C(C)C] |
| 4A1-3C | 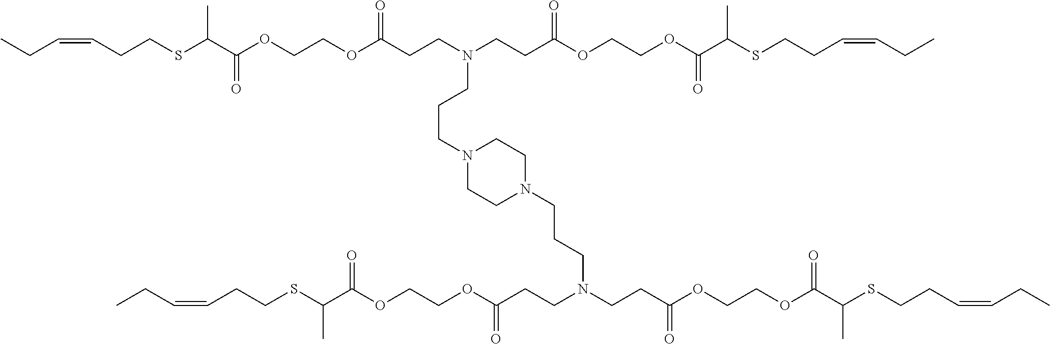 [SMILES: CC/C=C\CCSC(C)C(=O)OCCOC(=O)CCN(CCCN1CCN(CCCN(CCC(=O)OCCOC(=O)C(C)SCC/C=C\CC)CCC(=O)OCCOC(=O)C(C)SCC/C=C\CC)CC1)CCC(=O)OCCOC(=O)C(C)SCC/C=C\CC] |
| 6A3-8/2 | 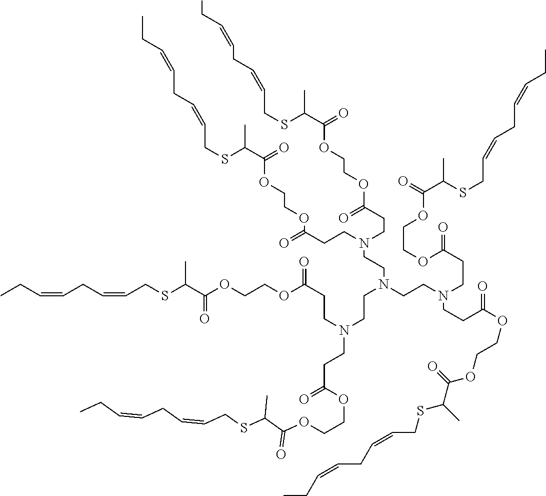 [SMILES: CC/C=C\C/C=C\CSC(C)C(=O)OCCOC(=O)CCN(CCC(=O)OCCOC(=O)C(C)SC/C=C\C/C=C\CC)CCN(CCN(CCC(=O)OCCOC(=O)C(C)SC/C=C\C/C=C\CC)CCC(=O)OCCOC(=O)C(C)SC/C=C\C/C=C\CC)CCN(CCC(=O)OCCOC(=O)C(C)SC/C=C\C/C=C\CC)CCC(=O)OCCOC(=O)C(C)SC/C=C\C/C=C\CC] |
| 4A3-3T | 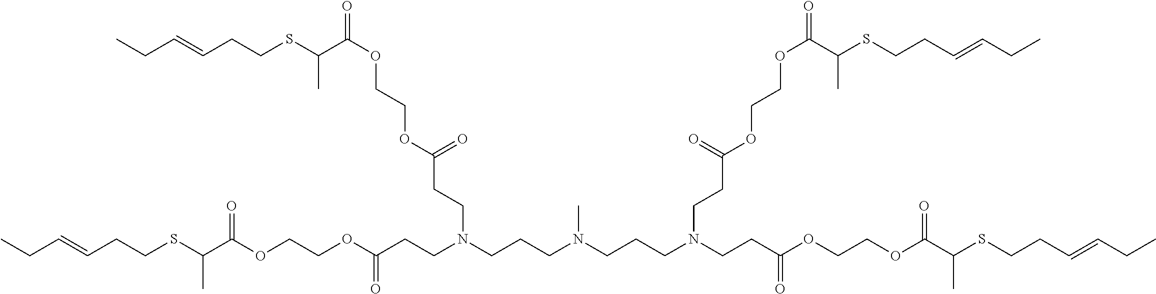 [SMILES: CC/C=C/CCSC(C)C(=O)OCCOC(=O)CCN(CCCN(C)CCCN(CCC(=O)OCCOC(=O)C(C)SCC/C=C/CC)CCC(=O)OCCOC(=O)C(C)SCC/C=C/CC)CCC(=O)OCCOC(=O)C(C)SCC/C=C/CC] |
| 4A3-Cit | 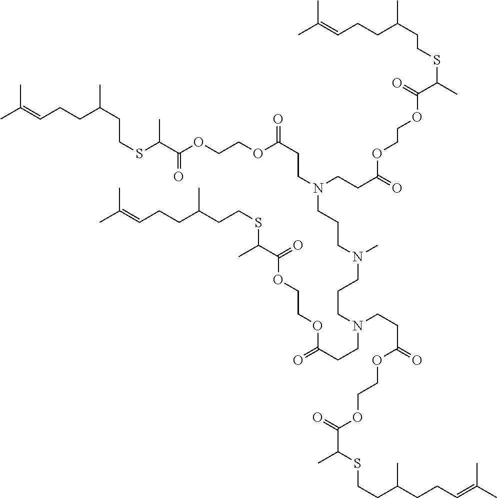 [SMILES: CC(C)=CCCC(C)CCSC(C)C(=O)OCCOC(=O)CCN(CCCN(C)CCCN(CCC(=O)OCCOC(=O)C(C)SCCC(C)CCC=C(C)C)CCC(=O)OCCOC(=O)C(C)SCCC(C)CCC=C(C)C)CCC(=O)OCCOC(=O)C(C)SCCC(C)CCC=C(C)C] |
| 4A1-8/2 | 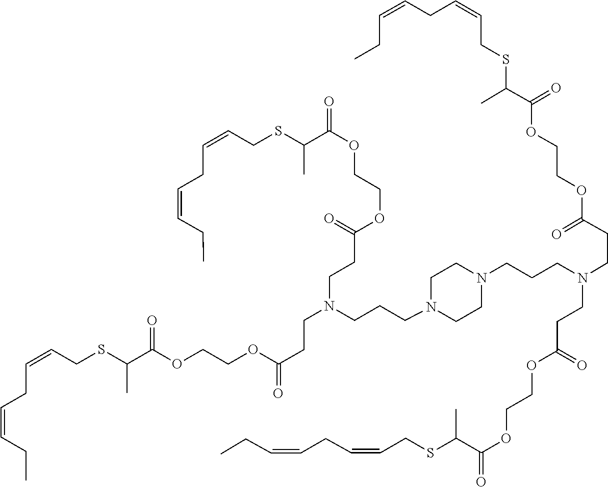 [SMILES: CC/C=C\C/C=C\CSC(C)C(=O)OCCOC(=O)CCN(CCCN1CCN(CCCN(CCC(=O)OCCOC(=O)C(C)SC/C=C\C/C=C\CC)CCC(=O)OCCOC(=O)C(C)SC/C=C\C/C=C\CC)CC1)CCC(=O)OCCOC(=O)C(C)SC/C=C\C/C=C\CC] |
| 4A3-3C | 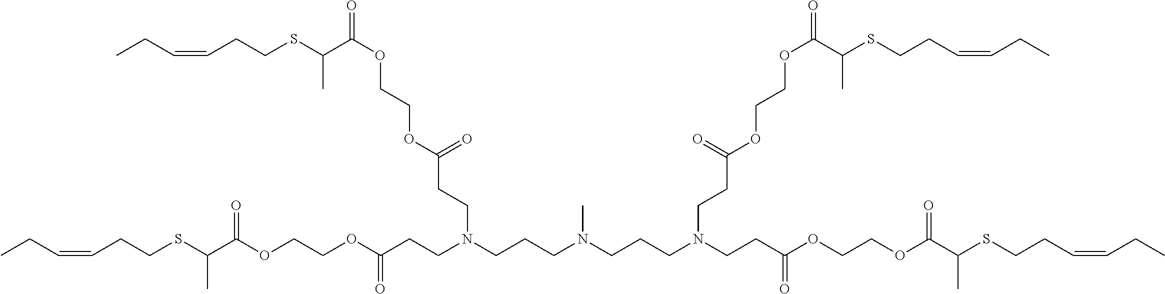 [SMILES: CC/C=C\CCSC(C)C(=O)OCCOC(=O)CCN(CCCN(C)CCCN(CCC(=O)OCCOC(=O)C(C)SCC/C=C\CC)CCC(=O)OCCOC(=O)C(C)SCC/C=C\CC)CCC(=O)OCCOC(=O)C(C)SCC/C=C\CC] |
| 4A3-2T | 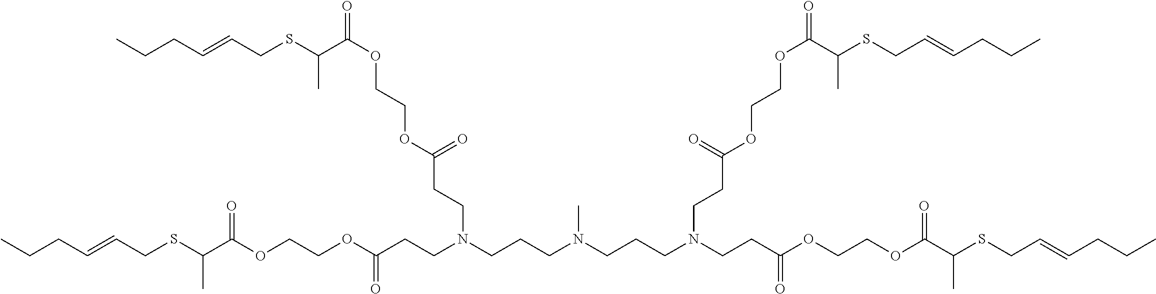 [SMILES: CCC/C=C/CSC(C)C(=O)OCCOC(=O)CCN(CCCN(C)CCCN(CCC(=O)OCCOC(=O)C(C)SC/C=C/CCC)CCC(=O)OCCOC(=O)C(C)SC/C=C/CCC)CCC(=O)OCCOC(=O)C(C)SC/C=C/CCC] |
| 4A3-5 | 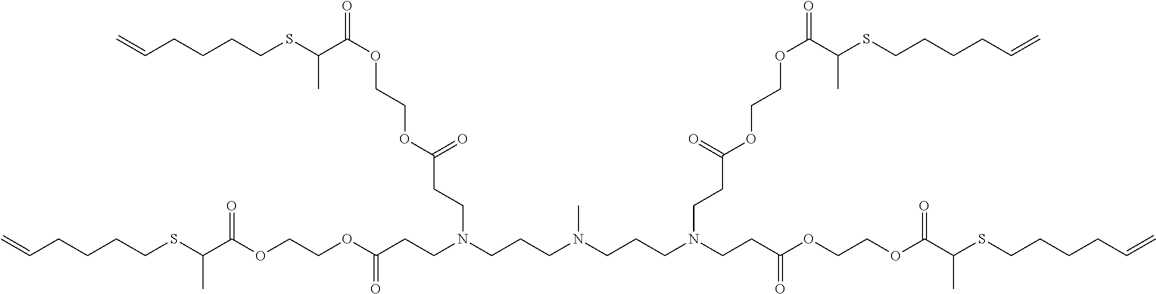 [SMILES: C=CCCCCSC(C)C(=O)OCCOC(=O)CCN(CCCN(C)CCCN(CCC(=O)OCCOC(=O)C(C)SCCCCC=C)CCC(=O)OCCOC(=O)C(C)SCCCCC=C)CCC(=O)OCCOC(=O)C(C)SCCCCC=C] |
| 4A3-8/2 | 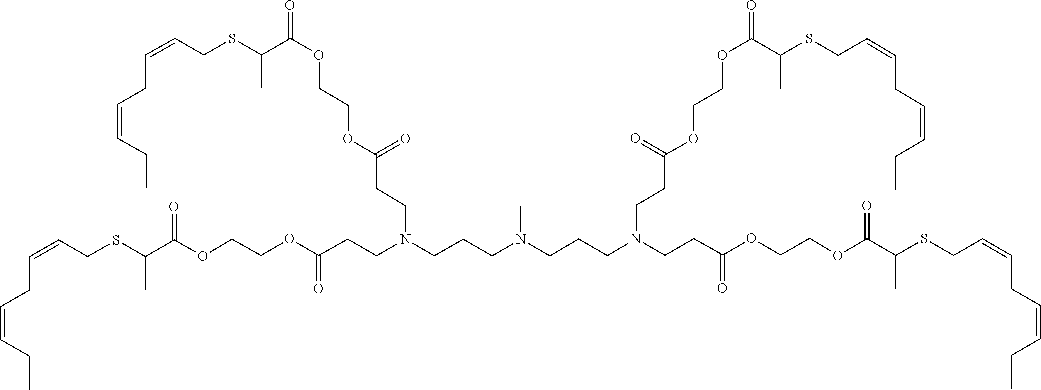 [SMILES: CC/C=C\C/C=C\CSC(C)C(=O)OCCOC(=O)CCN(CCCN(C)CCCN(CCC(=O)OCCOC(=O)C(C)SC/C=C\C/C=C\CC)CCC(=O)OCCOC(=O)C(C)SC/C=C\C/C=C\CC)CCC(=O)OCCOC(=O)C(C)SC/C=C\C/C=C\CC] |
| 4A3-2C | 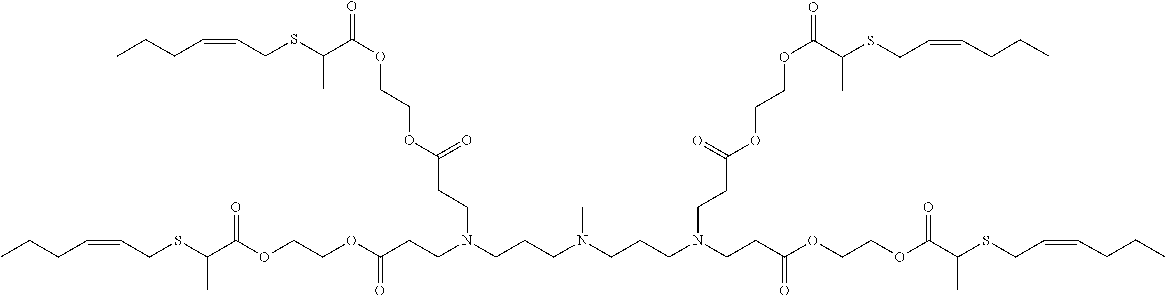 [SMILES: CCC/C=C\CSC(C)C(=O)OCCOC(=O)CCN(CCCN(C)CCCN(CCC(=O)OCCOC(=O)C(C)SC/C=C\CCC)CCC(=O)OCCOC(=O)C(C)SC/C=C\CCC)CCC(=O)OCCOC(=O)C(C)SC/C=C\CCC] |
| 4A3-4T | 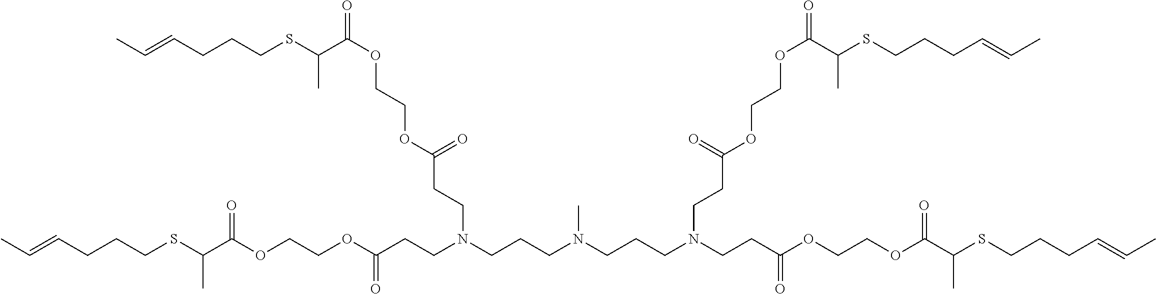 [SMILES: C/C=C/CCCSC(C)C(=O)OCCOC(=O)CCN(CCCN(C)CCCN(CCC(=O)OCCOC(=O)C(C)SCCC/C=C/C)CCC(=O)OCCOC(=O)C(C)SCCC/C=C/C)CCC(=O)OCCOC(=O)C(C)SCCC/C=C/C] |

### Synthesis of Unsaturated Dendrimers

In some embodiments, the ionizable cationic lipid is an unsaturated dendrimer described herein. In some embodiments, the method of synthesizing an unsaturated dendrimer can be supplemented using procedural techniques set forth in: Zhou et al., Modular degradable dendrimers enable small RNAs to extend survival in an aggressive liver cancer model. PNAS. 113, 520-526, 2016 and WO2017/048789A1. In some embodiments, the method of synthesizing an unsaturated dendrimer can be supplemented using procedural techniques set forth in: Lee et al., A Systematic Study of Unsaturation in Lipid Nanoparticles Lead to Improved mRNA Transfection In Vivo. Angew. Chem. Int. Ed. 60, 2021.

In some embodiments, allylic alcohol conversion to bromide and subsequent reaction with NaSH provided thiols at 48% to 91% yield. In some embodiments, the method for preparing an unsaturated thiol compound having structural formula I,

R—SH (I),

wherein (a) contacting a compound having structural formula II,

R—OH (II)

with a halogenating agent to form an activated halogenated compound, and (b) contacting the activated halogenated compound with a thiolate compound to yield said unsaturated compound having structural formula I. In some embodiments, contacting said activated halogenated compound in (b) with the thiolate compound can require 1 equivalent to about 2 equivalents. In some embodiments, the method provides the unsaturated thiol compound in a yield of about 40%, 50%, 60%, 70%, 80%, or 90%. In some embodiments, R of Formula I is

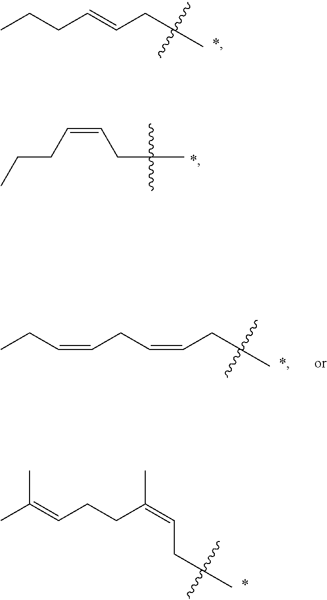

wherein * indicates the point of attachment to the sulfur. In some embodiments, R of formula I is

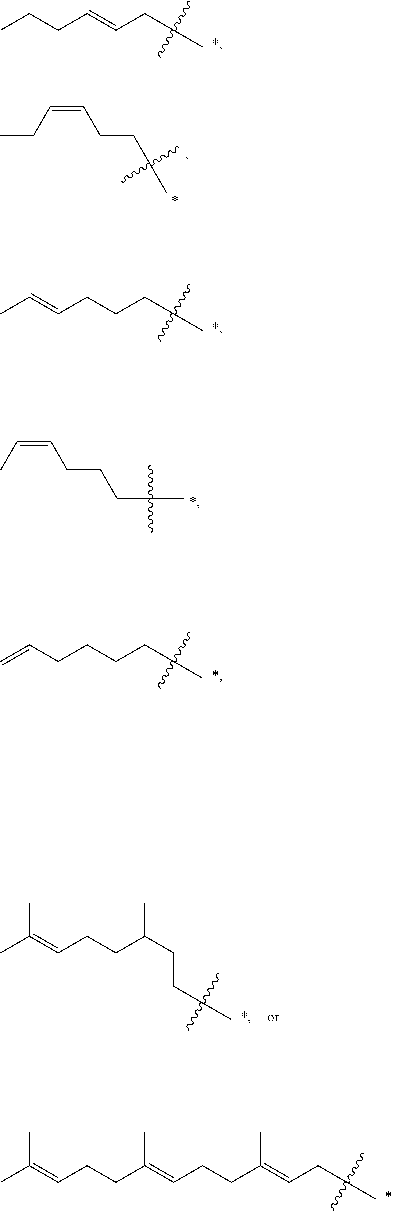

wherein * indicates the point of attachment to the sulfur.

In some embodiments, tosyl protection of the alcohol, for non-allylic alcohols and farnesol, then subsequent treatment with NaSH afforded the desired thiols at 19% to 67% yields. In some embodiments, the method for preparing an unsaturated thiol compound having structural formula I,

R—SH (I),

from a compound having structural formula II,

R—OH (II),

wherein R is a C₆-C₂₂ alkenyl, C₆-C₂₂ alkadienyl, or C₆-C₂₂ alkatrienyl.

In some embodiments, R is a C₆-C₂₂ alkenyl. In some embodiments, C₆-C₂₂ alkenyl is a linear chain. In some embodiments, C₆-C₂₂ alkenyl is a branched chain. In some embodiments, C₆-C₂₂ alkenyl has one double bond. In some embodiments, C₆-C₂₂ alkenyl has at least two double bonds. In some embodiments, C₆-C₂₂ alkenyl has at least 3 double bonds. In some embodiments, C₆-C₂₂ alkenyl has multiple double bonds. In some embodiments, R is a C₆-C₂₂ alkadienyl. In some embodiments, C₆-C₂₂ alkadienyl is a linear chain. In some embodiments, C₆-C₂₂ alkadienyl is a branched chain. In some embodiments, R is a C₆-C₂₂ alkatrienyl. In some embodiments, C₆-C₂₂ alkatrienyl is a linear chain. In some embodiments, C₆-C₂₂ alkatrienyl is a branched chain. In some embodiments, the double bonds of the C₆-C₂₂ alkenyl, C₆-C₂₂ alkadienyl, or C₆-C₂₂ alkatrienyl is conjugated. In some embodiments, the double bonds of the C₆-C₂₂ alkenyl, C₆-C₂₂ alkadienyl, or C₆-C₂₂ alkatrienyl is unconjugated. In some embodiments, the method provides the unsaturated thiol compound in a yield of about 40%, 50%, 60%, 70%, 80%, or 90%.

In some embodiments, R of formula I has the structural formula:

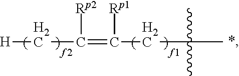

wherein:
Rₚ₁ and R^{p2} are each independently H or C₁-C₆ alkyl;
f1 is 1, 2, 3, or 4;
f2 is 0, 1, 2, or 3; and
wherein * indicates the point of attachment to the sulfur.

In some embodiments, R^{p1} is H. In some embodiments, R^{p1} is C₁-C₆ alkyl. In some embodiments, R^{p1} is C₁-C₃ alkyl. In some embodiments, R^{p2} is H. In some embodiments, R^{p2} is C₁-C₆ alkyl. In some embodiments, R^{p2} is C₁-C₃ alkyl. In some embodiments, —CR^{p1}=R^{p2}— is a cis bond. In some embodiments, —CR^{p1}=R^{p2}— is a trans bond. In some embodiments, f1 is 1. In some embodiments, f1 is 2. In some embodiments, f1 is 3. In some embodiments, f1 is 4. In some embodiments, f2 is 0. In some embodiments, f2 is 1. In some embodiments, f2 is 2. In some embodiments, f2 is 3. In some embodiments, f1+f2≥3. In some embodiments, f1+f2 is 3. In some embodiments, f1+f2 is 4. In some embodiments, f1+f2 is 5. In some embodiments, f1+f2 is 6.

In some embodiments, R of formula I has the structural formula:

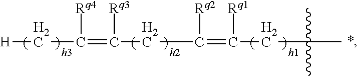

wherein:
R^{q1}, R^{q2}, R^{q3}, and R^{q4} are each independently H or C₁-C₆ alkyl;
h1 is 1, 2, 3, or 4;
h2 is 1 or 2;
h3 is 0, 1, 2, or 3; and
wherein * indicates the point of attachment to the sulfur.

In some embodiments, R^{q1} is H. In some embodiments, R^{q1} is C₁-C₆ alkyl. In some embodiments, R^{q1} is C₁-C₃ alkyl. In some embodiments, R^{q1} is methyl. In some embodiments, R^{q2} is H. In some embodiments, R^{q2} is C₁-C₆ alkyl. In some embodiments, R^{q2} is C₁-C₃ alkyl. In some embodiments, R^{q2} is methyl. In some embodiments, R^{q3} is H. In some embodiments, R^{q3} is C₁-C₆ alkyl. In some embodiments, R^{q3} is C₁-C₃ alkyl. In some embodiments, R^{q3} is methyl. In some embodiments, R^{q4} is H. In some embodiments, R^{q4} is C₁-C₆ alkyl. In some embodiments, R^{q4} is C₁-C₃ alkyl. In some embodiments, R^{q4} is methyl. In some embodiments, —CR^{q2}=CR^{q1}— is a cis bond. In some embodiments, —CR^{q2}=CR^{q1}— is a trans bond. In some embodiments, h1 is 1. In some embodiments, h1 is 2. In some embodiments, h1 is 3. In some embodiments, h1 is 4. In some embodiments, h2 is 1. In some embodiments, h2 is 2. In some embodiments, h3 is 0. In some embodiments, h3 is 1. In some embodiments, h3 is 2. In some embodiments, h3 is 3. In some embodiments, h1+h2+h3≥3. In some embodiments, h1+h2+h3 is 4. In some embodiments, h1+h2+h3 is 5. In some embodiments, h1+h2+h3 is 6.

In some embodiments, R of formula I has the structural formula:

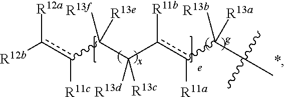

wherein:
e is 0, 1, 2, 3, 4, 5, or 6;
g is 1, 2, or 3;
x is independently at each occurrence 0, 1, 2, or 3;
R^{11a}, R^{11b}, R^{11c}, R^{12a}, R^{12b}, R^{13a}, R^{13b}, R^{13c}, R^{13d}, R^{13e}, and R^{13f} are each independently at each occurrence H or C₁-C₆ alkyl; and
wherein * indicates the point of attachment to the sulfur.

In some embodiments, R^{11a} is H. In some embodiments, R^{11a} is C₁-C₆ alkyl. In some embodiments, R^{11a} is C₁-C₃ alkyl. In some embodiments, R^{11b} is H. In some embodiments, R^{11b} is C₁-C₆ alkyl. In some embodiments, R^{11b} is C₁-C₃ alkyl. In some embodiments, R^{11c} is H. In some embodiments, R^{11c} is C₁-C₆ alkyl. In some embodiments, R^{11c} is C₁-C₃ alkyl. In some embodiments, R^{12a} is H. In some embodiments, R^{12a} is C₁-C₆ alkyl. In some embodiments, R^{12a} is C₁-C₃ alkyl. In some embodiments, R^{12b} is H. In some embodiments, R^{12b} is C₁-C₆ alkyl. In some embodiments, R^{12b} is C₁-C₃ alkyl. In some embodiments, R^{13a} is H. In some embodiments, R^{13a} is C₁-C₆ alkyl. In some embodiments, R^{13a} is C₁-C₃ alkyl. In some embodiments, R^{13b} is H. In some embodiments, R^{13b} is C₁-C₆ alkyl. In some embodiments, R^{13b} is C₁-C₃ alkyl. In some embodiments, R^{13c} is H. In some embodiments, R^{13c} is C₁-C₆ alkyl. In some embodiments, R^{13c} is C₁-C₃ alkyl. In some embodiments, R^{13d} is H. In some embodiments, R^{13d} is C₁-C₆ alkyl. In some embodiments, R^{13d} is C₁-C₃ alkyl. In some embodiments, R^{13e} is H. In some embodiments, R^{13e} is C₁-C₆ alkyl. In some embodiments, R^{13e} is C₁-C₃ alkyl. In some embodiments, R^{13f} is H. In some embodiments, R^{13f} is C₁-C₆ alkyl. In some embodiments, R^{13f} is C₁-C₃ alkyl. In some embodiments, e is 0. In some embodiments, e is 1. In some embodiments, e is 2. In some embodiments, e is 3. In some embodiments, e is 4. In some embodiments, e is 5. In some embodiments, e is 6. In some embodiments, g is 1. In some embodiments, g is 2. In some embodiments, g is 3. In some embodiments, x is 0. In some embodiments, x is 1. In some embodiments, x is 2. In some embodiments, x is 3. In some embodiments, R has the structural formula of

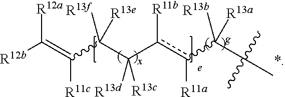

In some embodiments, R has the structural formula of

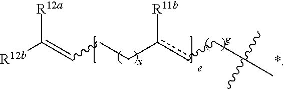

In some embodiments, R has the structural formula of

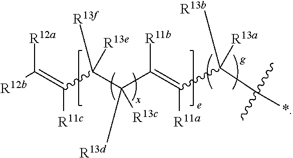

In some embodiments, R has the structural formula of

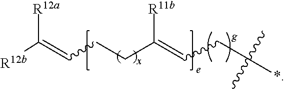

In some embodiments, R has the structural formula of

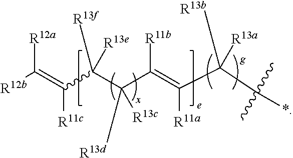

In some embodiments, R has the structural formula of

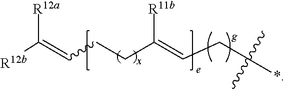

In some embodiments, R has the structural formula of

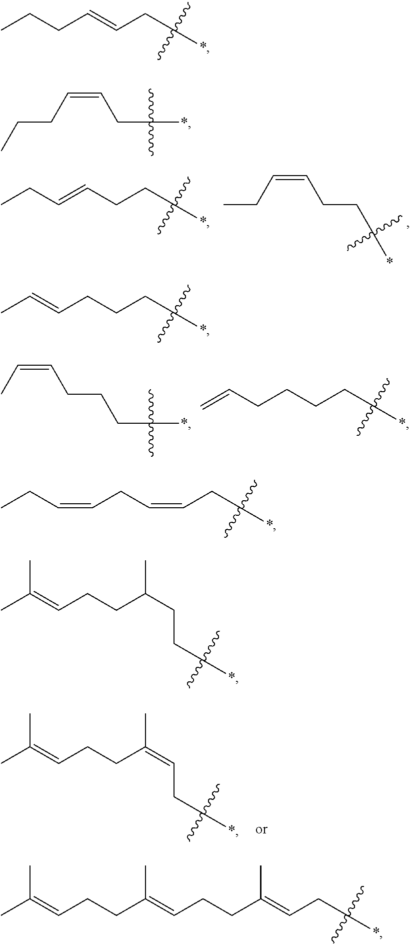

wherein * indicates the point of attachment to the sulfur.

### Lipid Formulations

In some embodiments, provided herein is a lipid composition comprising an unsaturated dendrimer (such as one described herein) and one or more lipids. The one or more lipids may be selected from an ionizable cationic lipid (such as one described herein), a zwitterionic lipid (such as one described herein), a phospholipid (such as one described herein), a steroid or a steroid derivative thereof (such as one described herein), and a polymer-conjugated (e.g., polyethylene glycol (PEG)-conjugated) lipid (such as one described herein).

In some embodiments, the lipid composition of the present disclosure comprises 1-2 ionizable lipids and 1-2 phospholipids, totaling to 3 components. In some embodiments, a 3 component lipid formulation comprises 1 ionizable lipid and 2 phospholipids. In some embodiments, a 3 component lipid formulation comprises 2 ionizable lipid and 1 phospholipid. In some embodiments, an ionizable lipid in a 3 component lipid formulation may be selected from ionizable cationic lipid (such as an unsaturated dendrimer, saturated dendrimer, LF92, and other cationic lipids described herein). In some embodiments, a phospholipid in a a 3 component lipid formulation may be selected from a phospholipid described herein or a zwitterionic lipid.

In some embodiments, provided herein is a lipid composition comprising 4 component formulation. In some embodiments, a 4 component lipid composition comprises an ionizable lipid, a phospholipid, a steroid, and a polymer-conjugated lipid. In some embodiments, an ionizable lipid in a 4 component lipid may be selected from ionizable cationic lipid (such as an unsaturated dendrimer, saturated dendrimer, LF92, and other cationic lipids described herein). In some embodiments, a phospholipid in a 4 component lipid may be selected from a phospholipid described herein or a zwitterionic lipid. In some embodiments, a steroid in a 4 component lipid may be selected from a steroid (such as one described herein) or a steroid derivative (such as one described herein). In some embodiments, a polymer-conjugated lipid in a 4 component lipid may be selected from a polymer-conjugated lipid (such as PEG-lipid described herein).

The present disclosure provides a (e.g., pharmaceutical) composition comprising a polynucleotide coupled to a lipid composition, wherein the polynucleotide encodes a dynein axonemal intermediate chain 1 (DNAI1) protein; and wherein the lipid composition comprises a (e.g., ionizable) cationic lipid. The polynucleotide may be a polynucleotide as disclosed hereinabove or disclosed elsewhere herein. The polynucleotide may comprise a nucleic acid sequence (e.g., an open reading frame (ORF) sequence) having at least about 70% sequence identity to a sequence over at least 1,000 bases (e.g., nucleotide residues 1 to 1,000) of SEQ ID NO: 15.

### Ionizable Cationic Lipids

In some embodiments of the lipid composition of the present application, the lipid composition comprises an ionizable cationic lipid. In some embodiments, the ionizable cationic lipid is an unsaturated dendrimer (such as one described herein). In some embodiments, the ionizable cationic lipid is a saturated dendrimer (such as one described herein). In some embodiments, the ionizable cationic lipid is cationic lipid having a structural formula (I′) (such as described herein). In some embodiments, the ionizable cationic lipid is cationic lipid having a structural formula (D-I′) (such as described herein).

In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some embodiments of the lipid composition of the present application, the ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C₆-C₂₄ alkyl or alkenyl groups, but may have at least 1 or more that 6 tails. In some embodiments, these cationic ionizable lipids are dendrimers, which are a polymer exhibiting regular dendritic branching, formed by the sequential or generational addition of branched layers to or from a core and are characterized by a core, at least one interior branched layer, and a surface branched layer. (See Petar R. Dvornic and Donald A. Tomalia in Chem. in Britain, 641-645, August 1994.) In some embodiments, the term “dendrimer” as used herein is intended to include, but is not limited to, a molecular architecture with an interior core, interior layers (or “generations”) of repeating units regularly attached to this initiator core, and an exterior surface of terminal groups attached to the outermost generation. A “dendron” is a species of dendrimer having branches emanating from a focal point which is or can be joined to a core, either directly or through a linking moiety to form a larger dendrimer. In some embodiments, the dendrimer structures have radiating repeating groups from a central core which doubles with each repeating unit for each branch. In some embodiments, the dendrimers described herein may be described as a small molecule, medium-sized molecules, lipids, or lipid-like material. These terms may be used to described compounds described herein which have a dendron like appearance (e.g. molecules which radiate from a single focal point). For clarity, as used herein, the term “dendrimer” is intended to include, but is not limited to, dendron and dendron-like structures.

While dendrimers are polymers, dendrimers may be preferable to traditional polymers because they have a controllable structure, a single molecular weight, numerous and controllable surface functionalities, and traditionally adopt a globular conformation after reaching a specific generation. Dendrimers can be prepared by sequentially reactions of each repeating unit to produce monodisperse, tree-like and/or generational structure polymeric structures. Individual dendrimers consist of a central core molecule, with a dendritic wedge attached to one or more functional sites on that central core. The dendrimeric surface layer can have a variety of functional groups disposed thereon including anionic, cationic, hydrophilic, or lipophilic groups, according to the assembly monomers used during the preparation.

Modifying the functional groups and/or the chemical properties of the core, repeating units, and the surface or terminating groups, their physical properties can be modulated. Some properties which can be varied include, but are not limited to, solubility, toxicity, immunogenicity and bioattachment capability. Dendrimers are often described by their generation or number of repeating units in the branches. A dendrimer consisting of only the core molecule is referred to as Generation 0, while each consecutive repeating unit along all branches is Generation 1, Generation 2, and so on until the terminating or surface group. In some embodiments, half generations are possible resulting from only the first condensation reaction with the amine and not the second condensation reaction with the thiol.

Preparation of dendrimers requires a level of synthetic control achieved through series of stepwise reactions comprising building the dendrimer by each consecutive group. Dendrimer synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. Thus, in some embodiments, the dendrimers of G1-G10 generation are specifically contemplated. In some embodiments, the dendrimers comprise 1, 2, 3, 4, 5, 6, 7, 8, 9, or 10 repeating units, or any range derivable therein. In some embodiments, the dendrimers used herein are G0, G1, G2, or G3. However, the number of possible generations (such as 11, 12, 13, 14, 15, 20, or 25) may be increased by reducing the spacing units in the branching polymer.

Additionally, dendrimers have two major chemical environments: the environment created by the specific surface groups on the termination generation and the interior of the dendritic structure which due to the higher order structure can be shielded from the bulk media and the surface groups. Because of these different chemical environments, dendrimers have found numerous different potential uses including in therapeutic applications.

In some embodiments of the lipid composition of the present application, the dendrimers are assembled using the differential reactivity of the acrylate and methacrylate groups with amines and thiols. The dendrimers may include secondary or tertiary amines and thioethers formed by the reaction of an acrylate group with a primary or secondary amine and a methacrylate with a mercapto group. Additionally, the repeating units of the dendrimers may contain groups which are degradable under physiological conditions. In some embodiments, these repeating units may contain one or more germinal diethers, esters, amides, or disulfides groups. In some embodiments, the core molecule is a monoamine which allows dendritic polymerization in only one direction. In other embodiments, the core molecule is a polyamine with multiple different dendritic branches which each may comprise one or more repeating units. The dendrimer may be formed by removing one or more hydrogen atoms from this core. In some embodiments, these hydrogen atoms are on a heteroatom such as a nitrogen atom. In some embodiments, the terminating group is a lipophilic groups such as a long chain alkyl or alkenyl group. In other embodiments, the terminating group is a long chain haloalkyl or haloalkenyl group. In other embodiments, the terminating group is an aliphatic or aromatic group containing an ionizable group such as an amine (—NH₂) or a carboxylic acid (—CO₂H). In still other embodiments, the terminating group is an aliphatic or aromatic group containing one or more hydrogen bond donors such as a hydroxide group, an amide group, or an ester.

The cationic ionizable lipids of the present application may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Cationic ionizable lipids may occur as racemates and racemic mixtures, single enantiomers, diastereomeric mixtures and individual diastereomers. In some embodiments, a single diastereomer is obtained. The chiral centers of the cationic ionizable lipids of the present application can have the S or the R configuration. Furthermore, it is contemplated that one or more of the cationic ionizable lipids may be present as constitutional isomers. In some embodiments, the compounds have the same formula but different connectivity to the nitrogen atoms of the core. Without wishing to be bound by any theory, it is believed that such cationic ionizable lipids exist because the starting monomers react first with the primary amines and then statistically with any secondary amines present. Thus, the constitutional isomers may present the fully reacted primary amines and then a mixture of reacted secondary amines.

Chemical formulas used to represent cationic ionizable lipids of the present application will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given formula, and regardless of which one is most prevalent, all tautomers of a given chemical formula are intended.

The cationic ionizable lipids of the present application may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the cationic ionizable lipids of the present application are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a cationic ionizable lipids provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

In some embodiments of the lipid composition of the present application, the ionizable cationic lipid is a dendrimer or dendron. In some embodiments, the ionizable cationic lipid comprises an ammonium group which is positively charged at physiological pH and contains at least two hydrophobic groups. In some embodiments, the ammonium group is positively charged at a pH from about 6 to about 8. In some embodiments, the ionizable cationic lipid is a dendrimer or dendron. In some embodiments, the ionizable cationic lipid comprises at least two C₆-C₂₄ alkyl or alkenyl groups.

### Dendrimers of Formula (I)

In some embodiments, the ionizable cationic lipid comprises at least two C₈-C₂₄ alkyl groups. In some embodiments, the ionizable cationic lipid is a dendrimer further defined by the formula:

Core-Repeating Unit-Terminating Group (I)
wherein the core is linked to the repeating unit by removing one or more hydrogen atoms from the core and replacing the atom with the repeating unit and wherein:
   the core has the formula:

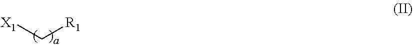

wherein:
   X₁ is amino or alkylamino_{(C≤12)}, dialkylamino_{(C≤12)}, heterocycloalkyl_{(C≤12)}, heteroaryl_{(C≤12)}, or a substituted version thereof;
   R₁ is amino, hydroxy, or mercapto, or alkylamino_{(C≤12)}, dialkylamino_{(C≤12)}, or a substituted version of either of these groups; and
   a is 1, 2, 3, 4, 5, or 6; or
the core has the formula:

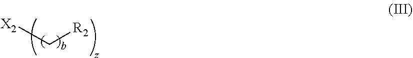

wherein:
   X₂ is N(R₅)_{y};
   R₅ is hydrogen, alkyl_{(C≤18)}, or substituted alkyl_{(C≤18)}; and
   y is 0, 1, or 2, provided that the sum of y and z is 3;
   R₂ is amino, hydroxy, or mercapto, or alkylamino_{(C≤12)}, dialkylamino_{(C≤12)}, or a substituted version of either of these groups;
   b is 1, 2, 3, 4, 5, or 6; and
   z is 1, 2, 3; provided that the sum of z and y is 3; or
the core has the formula:

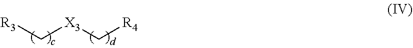

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})(CH₂CH₂N(R_{c}))ₑR_{d},

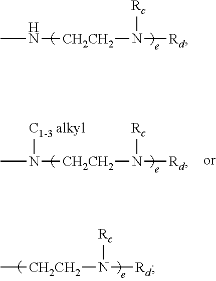

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker; the degradable diacyl group has the formula:

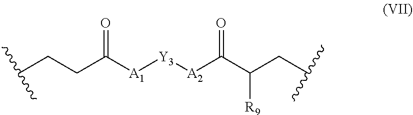

wherein:
   A₁ and A₂ are each independently —O—, —S—, or —NRₐ—, wherein:
   Rₐ is hydrogen, alkyl_{(C≤6)}, or substituted alkyl_{(C≤6)};
   Y₃ is alkanediyl_{(C≤12)}, alkenediyl_{(C≤12)}, arenediyl_{(C≤12)}, or a substituted version of any of these groups; or a group of the formula:

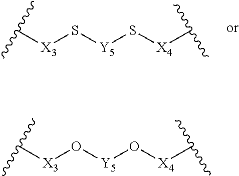

wherein:
   X₃ and X₄ are alkanediyl_{(C≤12)}, alkenediyl_{(C≤12)}, arenediyl_{(C≤12)}, or a substituted version of any of these groups;
   Y₅ is a covalent bond, alkanediyl_{(C≤12)}, alkenediyl_{(C≤12)}, arenediyl_{(C≤12)}, or a substituted version of any of these groups; and
   R₉ is alkyl_{(C≤8)} or substituted alkyl_{(C≤18)}; the linker group has the formula:

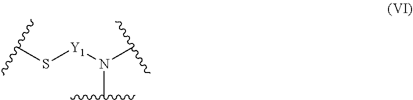

wherein:
   Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula:

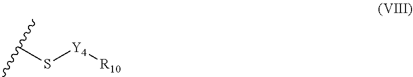

wherein:
   Y₄ is alkanediyl_{(C≤18)} or an alkanediyl_{(C≤18)} wherein one or more of the hydrogen atoms on the alkanediyl_{(C≤18)} has been replaced with —OH, —F, —Cl, —Br, —I, —SH, —OCH₃, —OCH₂CH₃, —SCH₃, or —OC(O)CH₃;
   R₁₀ is hydrogen, carboxy, hydroxy, or
   aryl_{(C≤12)}, alkylamino_{(C≤12)}, dialkylamino_{(C≤12)}, N-heterocycloalkyl_{(C≤12)}, —C(O)N(R₁₁)-alkanediyl_{(C≤6)}-heterocycloalkyl_{(C≤12)}, —C(O)-alkylamino_{(C≤12)}, —C(O)-dialkylamino_{(C≤12)}, —C(O)—N-heterocycloalkyl_{(C≤12)}, wherein:
   R₁₁ is hydrogen, alkyl_{(C≤6)}, or substituted alkyl_{(C≤6)};
   wherein the final degradable diacyl in the chain is attached to a terminating group;
   n is 0, 1, 2, 3, 4, 5, or 6;
      or a pharmaceutically acceptable salt thereof. In some embodiments, the terminating group is further defined by the formula:

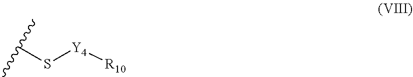

wherein:
Y₄ is alkanediyl_{(C≤18)}; and
R₁₀ is hydrogen. In some embodiments, A₁ and A₂ are each independently —O— or —NRₐ—.

In some embodiments, the core is further defined by the formula:

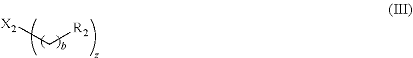

wherein:
X₂ is N(R₅)_{y};
   R₅ is hydrogen or alkyl_{(C≤18)}, or substituted alkyl_{(C≤18)}; and
   y is 0, 1, or 2, provided that the sum of y and z is 3;
R₂ is amino, hydroxy, or mercapto, or alkylamino_{(C≤12)}, dialkylamino_{(C≤12)}, or a substituted version of either of these groups;
b is 1, 2, 3, 4, 5, or 6; and
z is 1, 2, 3; provided that the sum of z and y is 3.

In some embodiments, the core is further defined by the formula:

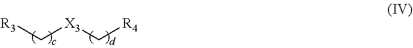

wherein:
X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤8)}, heteroarenediyl_{(C≤8)}, heterocycloalkanediyl_{(C≤8)}, or a substituted version of any of these groups;
R₃ and R₄ are each independently amino, hydroxy, or mercapto, or alkylamino_{(C≤12)}, dialkylamino_{(C≤12)}, or a substituted version of either of these groups; or a group of the formula: —N(R_{f})(CH₂CH₂N(R_{c}))ₑR_{d},

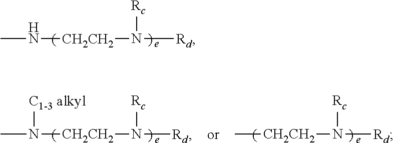

wherein:
   e and f are each independently 1, 2, or 3; provided that the sum of e and f is 3;
   R_{c}, R_{d}, and R_{f} are each independently hydrogen, alkyl_{(C≤6)}, or substituted alkyl_{(C≤6)};
c and d are each independently 1, 2, 3, 4, 5, or 6.

In some embodiments, the the terminating group is represented by the formula:

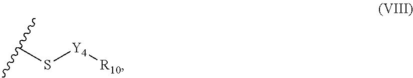

wherein:
Y₄ is alkanediyl_{(C≤18)}; and
R₁₀ is hydrogen.

In some embodiments, the core is further defined as:

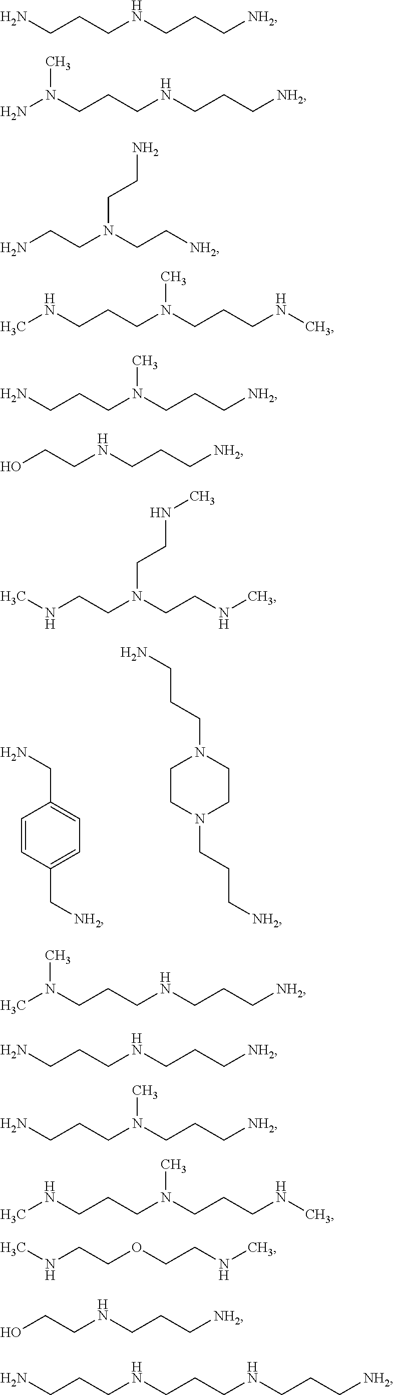

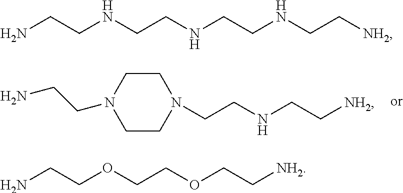

In some embodiments, the degradable diacyl is further defined as:

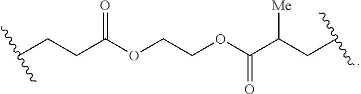 [SMILES: *CCC(=O)OCCOC(=O)C(C)C*]

In some embodiments, the linker is further defined as

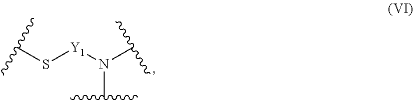

wherein Y₁ is alkanediyl_{(C≤8)} or substituted alkanediyl_{(C≤18)}.

In some embodiments, the dendrimer is further defined as:

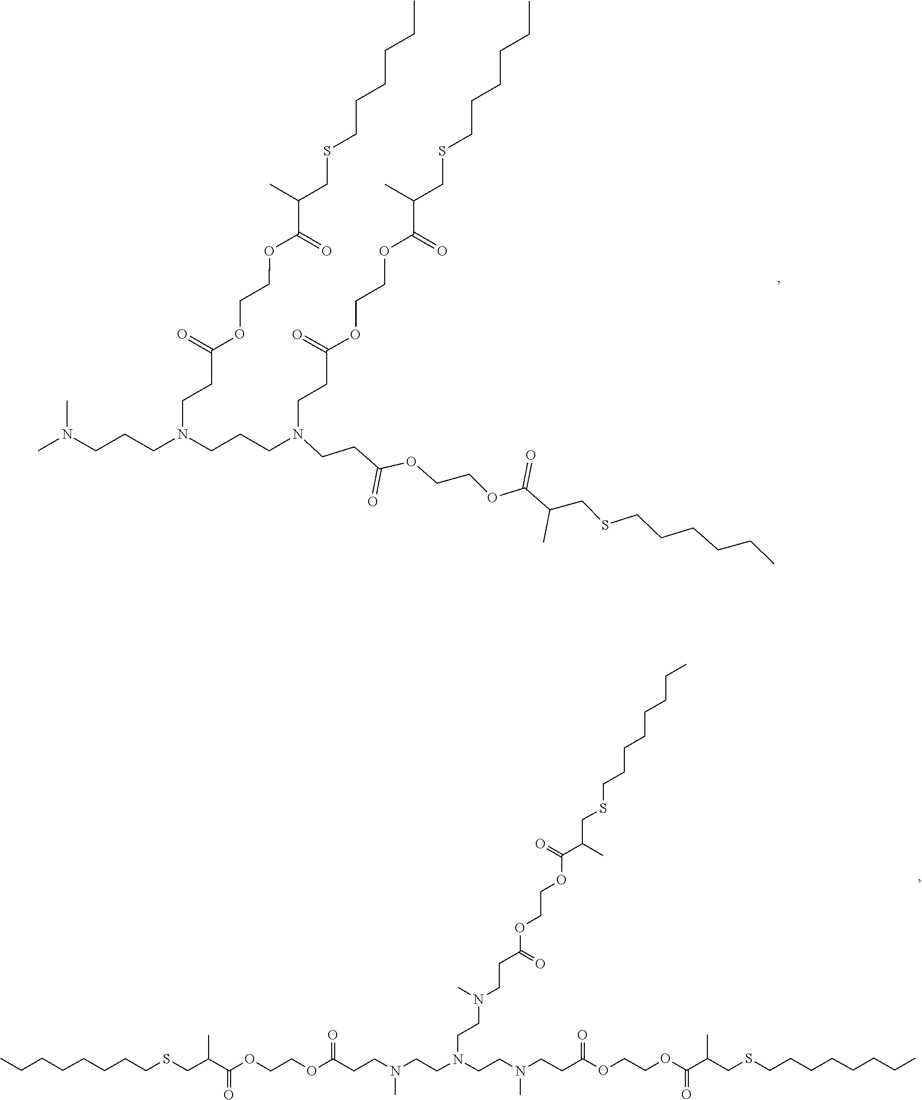

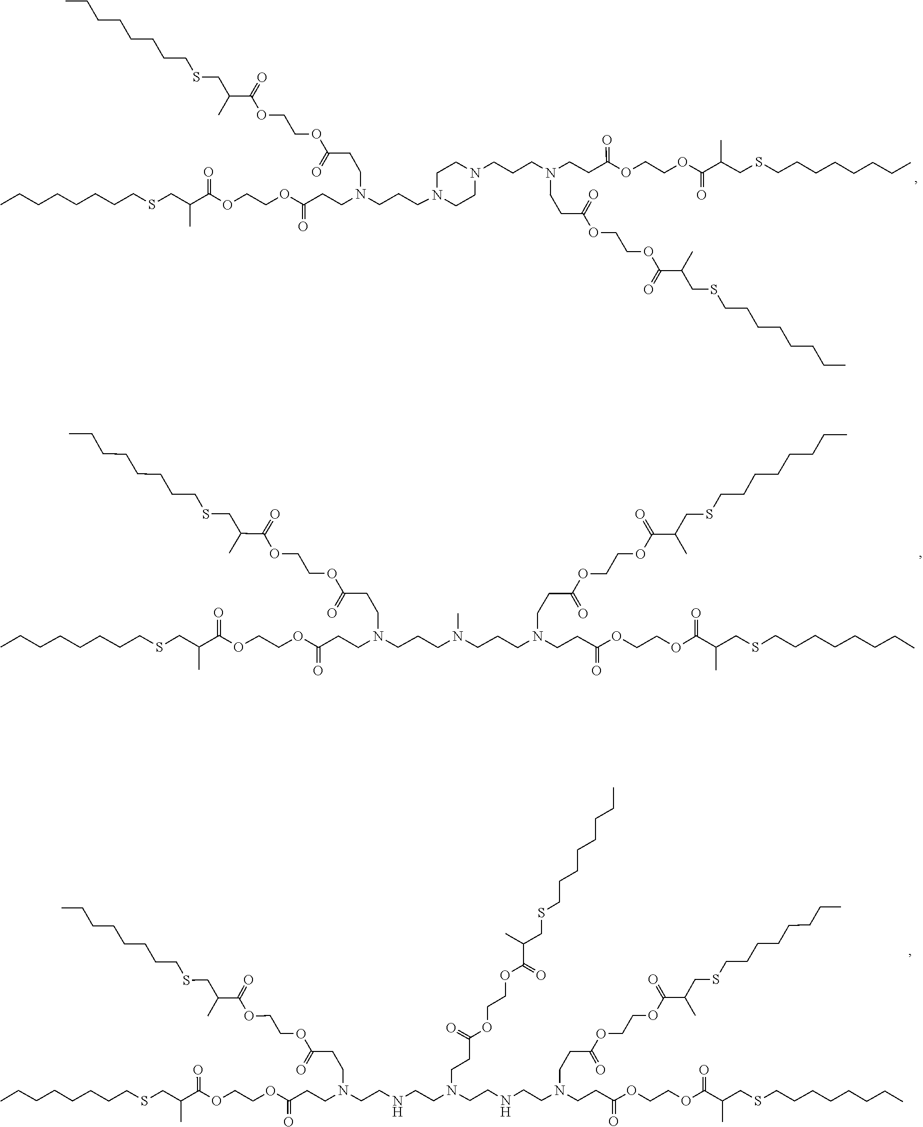

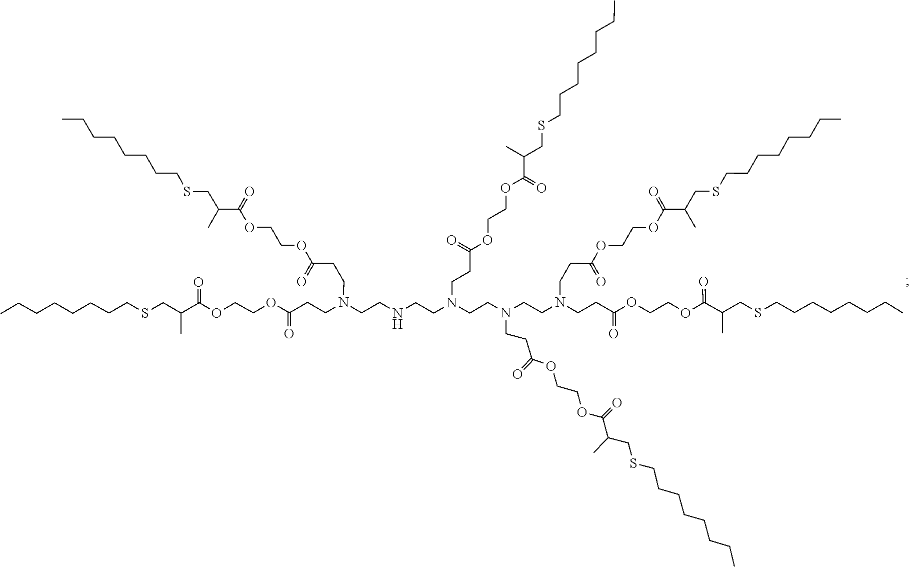 [SMILES: CCCCCCCCSCC(C)C(=O)OCCOC(=O)CCN(CCNCCN(CCC(=O)OCCOC(=O)C(C)CSCCCCCCCC)CCN(CCC(=O)OCCOC(=O)C(C)CSCCCCCCCC)CCN(CCC(=O)OCCOC(=O)C(C)CSCCCCCCCC)CCC(=O)OCCOC(=O)C(C)CSCCCCCCCC)CCC(=O)OCCOC(=O)C(C)CSCCCCCCCC]

or a pharmaceutically acceptable salt thereof.

In some embodiments, an ionizable cationic lipid in the lipid composition comprises lipophilic and cationic components, wherein the cationic component is ionizable. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some aspects of the present disclosure, composition containing compounds containing lipophilic and cationic components, wherein the cationic component is ionizable, are provided. In some embodiments, ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C₆-C₂₄ alkyl or alkenyl groups, but may have at least 1 or more that 6 tails. In some embodiments, these cationic ionizable lipids are dendrimers, which are a polymer exhibiting regular dendritic branching, formed by the sequential or generational addition of branched layers to or from a core and are characterized by a core, at least one interior branched layer, and a surface branched layer. (See Petar R. Dvornic and Donald A. Tomalia in Chem. in Britain, 641-645, August 1994.) In other embodiments, the term “dendrimer” as used herein is intended to include, but is not limited to, a molecular architecture with an interior core, interior layers (or “generations”) of repeating units regularly attached to this initiator core, and an exterior surface of terminal groups attached to the outermost generation. A “dendron” is a species of dendrimer having branches emanating from a focal point which is or can be joined to a core, either directly or through a linking moiety to form a larger dendrimer. In some embodiments, the dendrimer structures have radiating repeating groups from a central core which doubles with each repeating unit for each branch. In some embodiments, the dendrimers described herein may be described as a small molecule, medium-sized molecules, lipids, or lipid-like material. These terms may be used to described compounds described herein which have a dendron like appearance (e.g. molecules which radiate from a single focal point).

While dendrimers are polymers, dendrimers may be preferable to traditional polymers because they have a controllable structure, a single molecular weight, numerous and controllable surface functionalities, and traditionally adopt a globular conformation after reaching a specific generation. Dendrimers can be prepared by sequentially reactions of each repeating unit to produce monodisperse, tree-like and/or generational structure polymeric structures. Individual dendrimers consist of a central core molecule, with a dendritic wedge attached to one or more functional sites on that central core. The dendrimeric surface layer can have a variety of functional groups disposed thereon including anionic, cationic, hydrophilic, or lipophilic groups, according to the assembly monomers used during the preparation.

Modifying the functional groups and/or the chemical properties of the core, repeating units, and the surface or terminating groups, their physical properties can be modulated. Some properties which can be varied include, but are not limited to, solubility, toxicity, immunogenicity and bioattachment capability. Dendrimers are often described by their generation or number of repeating units in the branches. A dendrimer consisting of only the core molecule is referred to as Generation 0, while each consecutive repeating unit along all branches is Generation 1, Generation 2, and so on until the terminating or surface group. In some embodiments, half generations are possible resulting from only the first condensation reaction with the amine and not the second condensation reaction with the thiol.

Preparation of dendrimers requires a level of synthetic control achieved through series of stepwise reactions comprising building the dendrimer by each consecutive group. Dendrimer synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. Thus, in some embodiments, the dendrimers of G1-G10 generation are specifically contemplated. In some embodiments, the dendrimers comprise 1, 2, 3, 4, 5, 6, 7, 8, 9, or 10 repeating units, or any range derivable therein. In some embodiments, the dendrimers used herein are G0, G1, G2, or G3. However, the number of possible generations (such as 11, 12, 13, 14, 15, 20, or 25) may be increased by reducing the spacing units in the branching polymer.

Additionally, dendrimers have two major chemical environments: the environment created by the specific surface groups on the termination generation and the interior of the dendritic structure which due to the higher order structure can be shielded from the bulk media and the surface groups. Because of these different chemical environments, dendrimers have found numerous different potential uses including in therapeutic applications.

In some embodiments, the dendrimers that may be used in the present compositions are assembled using the differential reactivity of the acrylate and methacrylate groups with amines and thiols. The dendrimers may include secondary or tertiary amines and thioethers formed by the reaction of an acrylate group with a primary or secondary amine and a methacrylate with a mercapto group. Additionally, the repeating units of the dendrimers may contain groups which are degradable under physiological conditions.

In some embodiments, these repeating units may contain one or more germinal diethers, esters, amides, or disulfides groups. In some embodiments, the core molecule is a monoamine which allows dendritic polymerization in only one direction. In other embodiments, the core molecule is a polyamine with multiple different dendritic branches which each may comprise one or more repeating units. The dendrimer may be formed by removing one or more hydrogen atoms from this core. In some embodiments, these hydrogen atoms are on a heteroatom such as a nitrogen atom. In some embodiments, the terminating group is a lipophilic groups such as a long chain alkyl or alkenyl group. In other embodiments, the terminating group is a long chain haloalkyl or haloalkenyl group. In other embodiments, the terminating group is an aliphatic or aromatic group containing an ionizable group such as an amine (—NH₂) or a carboxylic acid (—C(O)OH).

In still other embodiments, the terminating group is an aliphatic or aromatic group containing one or more hydrogen bond donors such as a hydroxide group, an amide group, or an ester.

### Dendrimers of Formula (X)

In some embodiments, the ionizable cationic lipid is a dendrimer of the formula Core [symbol] Branch)_{N}. In some embodiments, the ionizable cationic lipid is a dendrimer of the formula

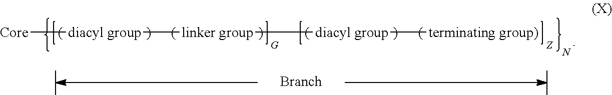

In some embodiments, the ionizable cationic lipid is a dendrimer of a generation (g) having a structural formula:

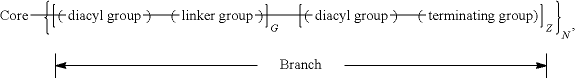

or a pharmaceutically acceptable salt thereof, wherein:
(a) the core comprises a structural formula (X_{Core}):

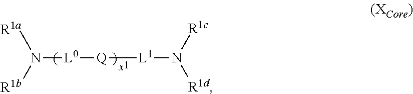

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1d}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

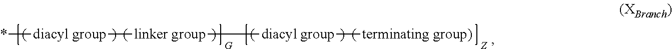

wherein:
   indicates a point of attachment of the branch to the core;
   g is 1, 2, 3, or 4;
   Z=2^{(g-1)};
   G=0, when g=1; or G=Σᵢ₌₀^{i=g-2}2ⁱ, when g≠1;
(c) each diacyl group independently comprises a structural formula

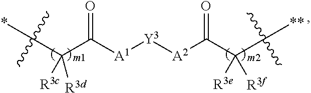

wherein:
   * indicates a point of attachment of the diacyl group at the proximal end thereof,
   ** indicates a point of attachment of the diacyl group at the distal end thereof,
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein: R⁴ is hydrogen or optionally substituted (e.g., C₁-C₆) alkyl;
   m¹ and m² are each independently at each occurrence 1, 2, or 3; and
   R^{3c}, R^{3d}, R^{3e}, and R^{3f} are each independently at each occurrence hydrogen or an optionally substituted (e.g., C₁-C₆) alkyl; and
(d) each linker group independently comprises a structural formula

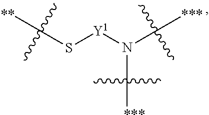

wherein:
   ** indicates a point of attachment of the linker to a proximal diacyl group;
   *** indicates a point of attachment of the linker to a distal diacyl group; and
   Y¹ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

In some embodiments of X_{Core}, Q is independently at each occurrence a covalent bond, —O—, —S—, —NR₂—, or —CR^{3a}R^{3b}. In some embodiments of X_{Core} Q is independently at each occurrence a covalent bond. In some embodiments of X_{Core} Q is independently at each occurrence an —O—. In some embodiments of X_{Core} Q is independently at each occurrence a —S—. In some embodiments of X_{Core} Q is independently at each occurrence a —NR² and R² is independently at each occurrence R^{1g} or -L²-NR^{1e}R^{1f}. In some embodiments of X_{Core} Q is independently at each occurrence a —CR^{3a}R^{3b}R^{3a}, and R^{3a} and R^{3b} are each independently at each occurrence hydrogen or an optionally substituted alkyl (e.g., C₁-C₆, such as C₁-C₃).

In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl. In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen. In some embodiments of X_{Core}, R^{1a}, R^{1b}, R^{1c}, R^{1d}, R^{1e}, R^{1f}, and R^{1g} (if present) are each independently at each occurrence a point of connection to a branch an optionally substituted alkyl (e.g., C₁-C₁₂).

In some embodiments of X_{Core}, L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or, alternatively, part of L¹ form a heterocycloalkyl (e.g., C₄-C₆ and containing one or two nitrogen atoms and, optionally, an additional heteroatom selected from oxygen and sulfur) with one of R^{1c} and R^{1d}. In some embodiments of X_{Core}, L⁰, L¹, and L² are each independently at each occurrence can be a covalent bond. In some embodiments of X_{Core}, L⁰, L¹, and L² are each independently at each occurrence can be a hydrogen. In some embodiments of X_{Core}, L⁰, L¹, and L² are each independently at each occurrence can be an alkylene (e.g., C₁-C₁₂, such as C₁-C₆ or C₁-C₃). In some embodiments of X_{Core}, L⁰, L¹, and L² are each independently at each occurrence can be a heteroalkylene (e.g., C₁-C₁₂, such as C₁-C₈ or C₁-C₆). In some embodiments of X_{Core}, L⁰, L¹, and L² are each independently at each occurrence can be a heteroalkylene (e.g., C₂-C₈ alkyleneoxide, such as oligo(ethyleneoxide)). In some embodiments of X_{Core}, L⁰, L¹, and L² are each independently at each occurrence can be a [alkylene]-[heterocycloalkyl]-[alkylene] [(e.g., C₁-C₆) alkylene]-[(e.g., C₄-C₆) heterocycloalkyl]-[(e.g., C₁-C₆) alkylene]. In some embodiments of X_{Core}, L⁰, L¹, and L² are each independently at each occurrence can be a [alkylene]-(arylene)-[alkylene] [(e.g., C₁-C₆) alkylene]-(arylene)-[(e.g., C₁-C₆) alkylene]. In some embodiments of X_{Core}, L⁰, L¹, and L² are each independently at each occurrence can be a [alkylene]-(arylene)-[alkylene] (e.g., [(e.g., C₁-C₆) alkylene]-phenylene-[(e.g., C₁-C₆) alkylene]). In some embodiments of X_{Core}, L⁰, L¹, and L² are each independently at each occurrence can be a heterocycloalkyl (e.g., C₄-C₆ heterocycloalkyl). In some embodiments of X_{Core}, L⁰, L¹, and L² are each independently at each occurrence can be an arylene (e.g., phenylene). In some embodiments of X_{Core}, part of L¹ form a heterocycloalkyl with one of R^{1c} and R^{1d}. In some embodiments of X_{Core}, part of L¹ form a heterocycloalkyl (e.g., C₄-C₆ heterocycloalkyl) with one of R^{1c} and R^{1d} and the heterocycloalkyl can contain one or two nitrogen atoms and, optionally, an additional heteroatom selected from oxygen and sulfur.

In some embodiments of X_{Core}, L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

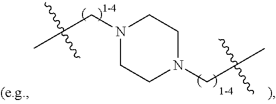

and [(C₁-C₄) alkylene]-phenylene-[(C₁-C₄) alkylene]

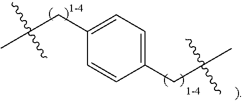

In some embodiments of X_{Core}, L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments of X_{Core}, L⁰, L¹, and L² are each independently at each occurrence C₁-C₆ alkylene (e.g., C₁-C₃ alkylene). In some embodiments, L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)). In some embodiments of X_{Core}, L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

In some embodiments of X_{Core}, x¹ is 0, 1, 2, 3, 4, 5, or 6. In some embodiments of X_{Core}, x¹ is 0. In some embodiments of X_{Core}, x¹ is 1. In some embodiments of X_{Core} x¹ is 2. In some embodiments of X_{Core}, x¹ is 0, 3. In some embodiments of X_{Core} x¹ is 4. In some embodiments of X_{Core} x¹ is 5. In some embodiments of X_{Core}, x¹ is 6.

In some embodiments of X_{Core}, the core comprises a structural formula:

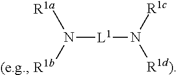

In some embodiments of X_{Core}, the core comprises a structural formula:

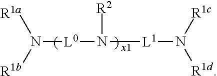

In some embodiments of X_{Core}, the core comprises a structural formula:

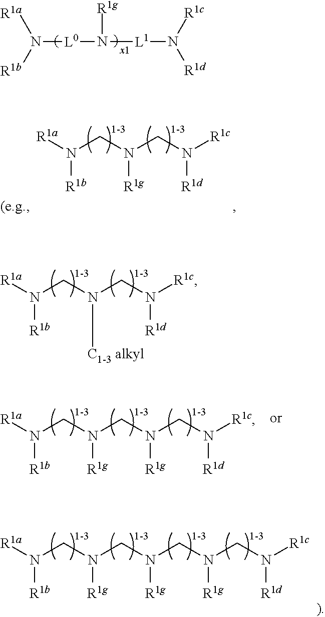

In some embodiments of X_{core}, the core comprises a structural formula:

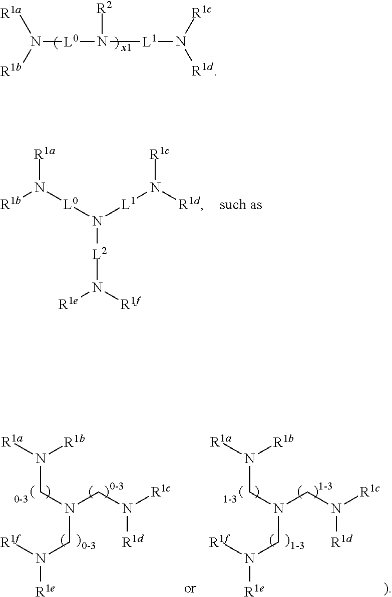

In some embodiments of X_{Core}, the core comprises a structural formula:

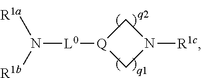

wherein Q′ is —NR²— or —CR^{3a}R^{3b}—; q¹ and q² are each independently 1 or
2. In some embodiments of X_{Core}, the core comprises a structural formula:

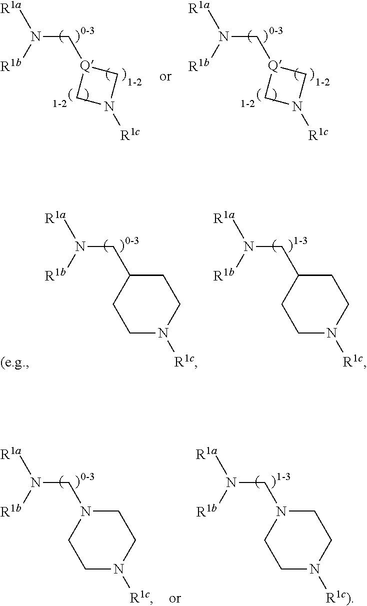

In some embodiments of X_{Core}, the core comprises a structural formula

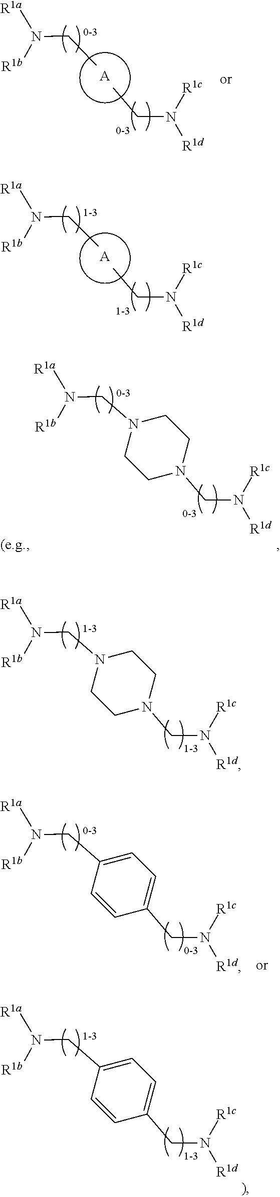

wherein ring A is an optionally substituted aryl or an optionally substituted (e.g., C₃-C₁₂, such as C₃-C₅) heteroaryl. In some embodiments of X_{Core}, the core comprises has a structural formula

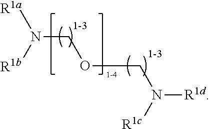

In some embodiments of X_{Core}, the core comprises a structural formula set forth in Table 1 and pharmaceutically acceptable salts thereof, wherein * indicates a point of attachment of the core to a branch of the plurality of branches. In some embodiments, the example cores of Table 1 are not limiting of the stereoisomers (i.e. enantiomers, diastereomers) listed.

In some embodiments of X_{Core}, the core comprises a structural formula selected from the group consisting of:

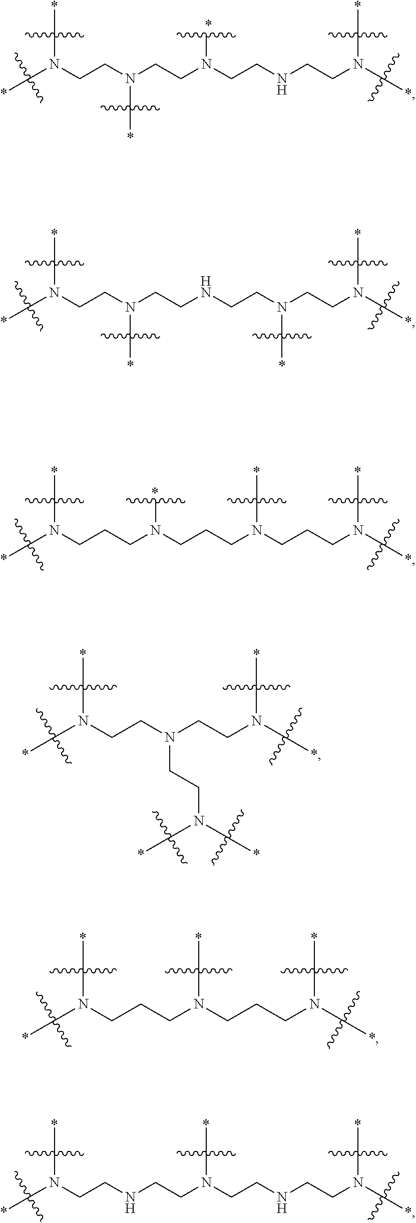

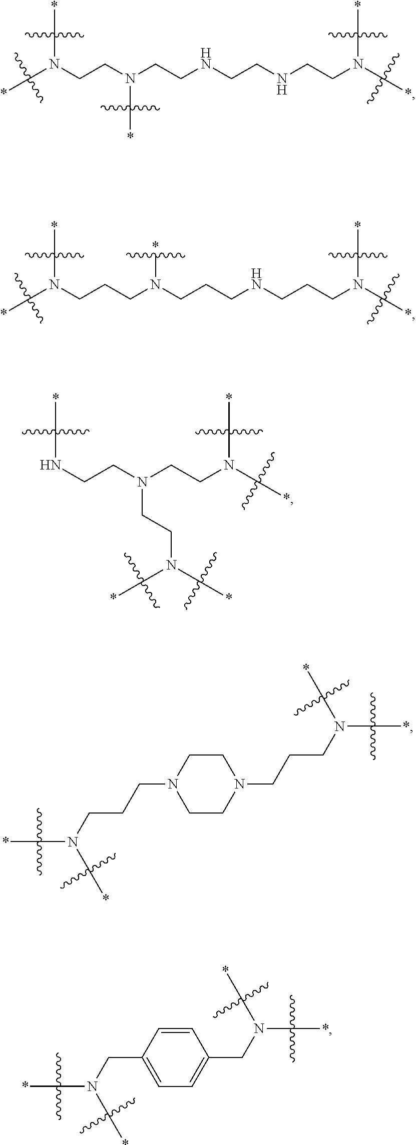

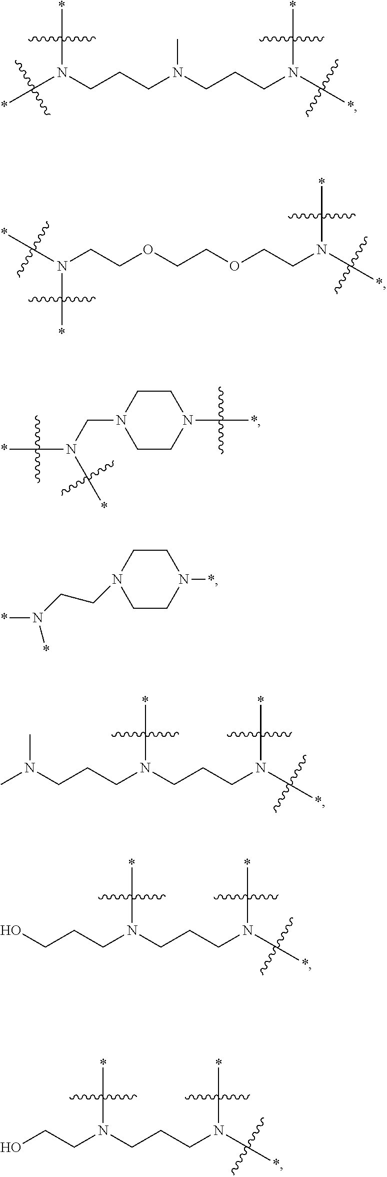

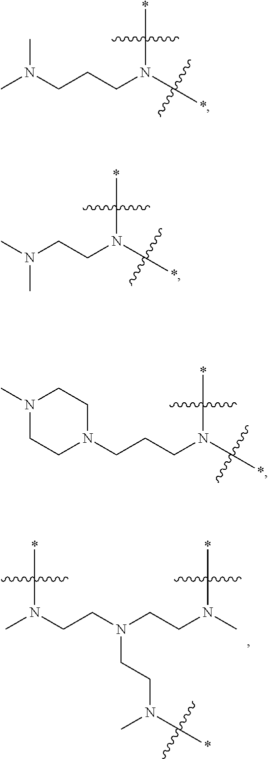

and pharmaceutically acceptable salts thereof, wherein * indicates a point of attachment of the core to a branch of the plurality of branches.

In some embodiments, the plurality (N) of branches comprises at least 3 branches, at least 4 branches, at least 5 branches. In some embodiments, the plurality (N) of branches comprises at least 3 branches. In some embodiments, the plurality (N) of branches comprises at least 4 branches. In some embodiments, the plurality (N) of branches comprises at least 5 branches.

In some embodiments of X_{Branch}, g is 1, 2, 3, or 4. In some embodiments of X_{Branch}, g is 1. In some embodiments of X_{Branch}, g is 2. In some embodiments of X_{Branch}, g is 3. In some embodiments of X_{Branch}, g is 4.

In some embodiments of X_{Branch}, Z=2^{(g-1)} and when g=1, G=0. In some embodiments of X_{Branch}, Z=2^{(g-1)} and G=Σ=ᵢ₌₀^{i=g-2}2ⁱ, when g≠1.

In some embodiments of X_{Branch}, g=1, G=0, Z=1, and each branch of the plurality of branches comprises a structural formula each branch of the plurality of branches comprises a structural formula *∵diacyl group [symbol] terminating group)

In some embodiments of X_{Branch}, g=2, G=1, Z=2, and each branch of the plurality of branches comprises a structural formula

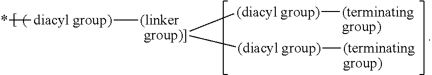

In some embodiments of X_{Branch}, g=3, G=3, Z=4, and each branch of the plurality of branches comprises a structural formula

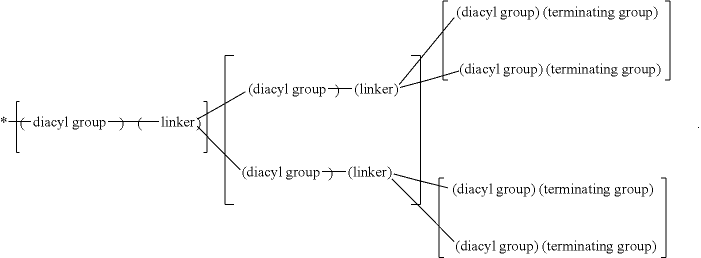

In some embodiments of X_{Branch}, g=4, G=7, Z=8, and each branch of the plurality of branches comprises a structural formula

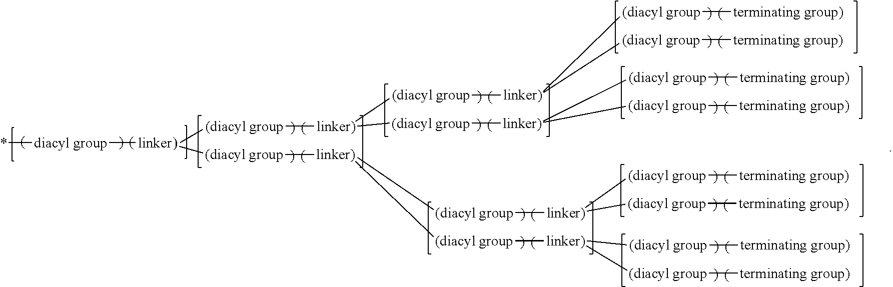

In some embodiments the dendrimers described herein with a generation (g)=1 has structure the structure:

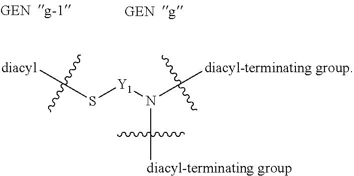

In some embodiments, the dendrimers described herein with a generation (g)=1 has structure the structure:

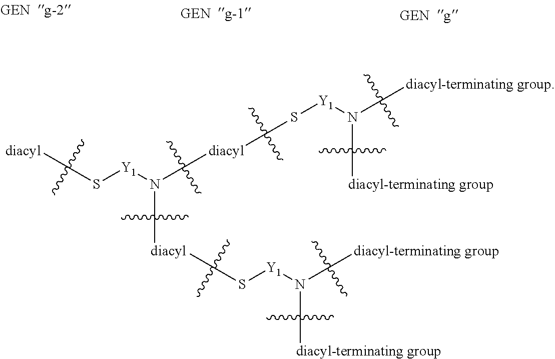

The example formulation of the dendrimers described herein for generations 1 to 4 is shown in Table 2. The number of diacyl groups, linker groups, and terminating groups can be calculated based on g.

In some embodiments, the diacyl group independently comprises a structural formula

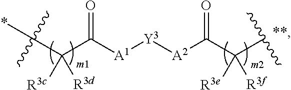

* indicates a point of attachment of the diacyl group at the proximal end thereof, and ** indicates a point of attachment of the diacyl group at the distal end thereof.

In some embodiments of the diacyl group of X_{Branch}, Y³ is independently at each occurrence an optionally substituted; alkylene, an optionally substituted alkenylene, or an optionally substituted arenylene. In some embodiments of the diacyl group of X_{Branch}, Y³ is independently at each occurrence an optionally substituted alkylene (e.g., C₁-C₁₂). In some embodiments of the diacyl group of X_{Branch}, Y³ is independently at each occurrence an optionally substituted alkenylene (e.g., C₁-C₁₂). In some embodiments of the diacyl group of X_{Branch}, Y³ is independently at each occurrence an optionally substituted arenylene (e.g., C₁-C₁₂).

In some embodiments of the diacyl group of X_{Branch}, A¹ and A² are each independently at each occurrence —O—, —S—, or —NR⁴—. In some embodiments of the diacyl group of X_{Branch}, A¹ and A² are each independently at each occurrence —O—. In some embodiments of the diacyl group of X_{Branch}, A¹ and A² are each independently at each occurrence —S—. In some embodiments of the diacyl group of X_{Branch}, A¹ and A² are each independently at each occurrence —NR⁴— and R⁴ is hydrogen or optionally substituted alkyl (e.g., C₁-C₆). In some embodiments of the diacyl group of X_{Branch}, m¹ and m² are each independently at each occurrence 1, 2, or 3. In some embodiments of the diacyl group of X_{Branch}, m¹ and m² are each independently at each occurrence 1. In some embodiments of the diacyl group of X_{Branch}, m¹ and m² are each independently at each occurrence 2. In some embodiments of the diacyl group of X_{Branch}, m¹ and m² are each independently at each occurrence 3. In some embodiments of the diacyl group of X_{Branch}, R^{3c}, R^{3d}, R^{3e}, and R^{3f} are each independently at each occurrence hydrogen or an optionally substituted alkyl. In some embodiments of the diacyl group of X_{Branch}, R^{3c}, R^{3d}, R^{3e}, and R^{3f} are each independently at each occurrence hydrogen. In some embodiments of the diacyl group of X_{Branch}, R^{3c}, R^{3d}, R^{3e}, and R^{3f} are each independently at each occurrence an optionally substituted (e.g., C₁-C₈) alkyl.

In some embodiments of the diacyl group, A¹ is —O— or —NH—. In some embodiments of the diacyl group, A¹ is —O—. In some embodiments of the diacyl group, A² is —O— or —NH—. In some embodiments of the diacyl group, A² is —O—. In some embodiments of the diacyl group, Y³ is C₁-C₁₂ (e.g., C₁-C₆, such as C₁-C₃) alkylene.

In some embodiments of the diacyl group, the diacyl group independently at each occurrence comprises a structural formula

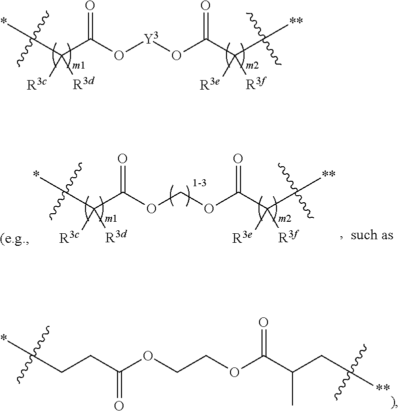

and optionally R^{3c}, R^{3d}, R^{3e}, and R^{3f} are each independently at each occurrence hydrogen or C₁-C₃ alkyl.

In some embodiments, linker group independently comprises a structural formula

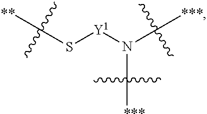

** indicates a point of attachment of the linker to a proximal diacyl group, and *** indicates a point of attachment of the linker to a distal diacyl group.

In some embodiments of the linker group of X_{Branch} if present, Y₁ is independently at each occurrence an optionally substituted alkylene, an optionally substituted alkenylene, or an optionally substituted arenylene. In some embodiments of the linker group of X_{Branch} if present, Y₁ is independently at each occurrence an optionally substituted alkylene (e.g., C₁-C₁₂). In some embodiments of the linker group of X_{Branch} if present, Y₁ is independently at each occurrence an optionally substituted alkenylene (e.g., C₁-C₁₂). In some embodiments of the linker group of X_{Branch} if present, Y₁ is independently at each occurrence an optionally substituted arenylene (e.g., C₁-C₁₂).

In some embodiments of the terminating group of X_{Branch}, each terminating group is independently selected from optionally substituted alkylthiol and optionally substituted alkenylthiol. In some embodiments of the terminating group of X_{Branch}, each terminating group is an optionally substituted alkylthiol (e.g., C₁-C₁₈, such as C₄-C₁₈). In some embodiments of the terminating group of X_{Branch}, each terminating group is optionally substituted alkenylthiol (e.g., C₁-C₁₈, such as C₄-C₁₈).

In some embodiments of the terminating group of X_{Branch}, each terminating group is independently C₁-C₁₈ alkenylthiol or C₁-C₁₈ alkylthiol, and the alkyl or alkenyl moiety is optionally substituted with one or more substituents each independently selected from halogen, C₆-C₁₂ aryl, C₁-C₁₂ alkylamino, C₄-C₆ N-heterocycloalkyl, —OH, —C(O)OH, —C(O)N(C₁-C₃ alkyl)-(C₁-C₆ alkylene)-(C₁-C₁₂ alkylamino), —C(O)N(C₁-C₃ alkyl)-(C₁-C₆ alkylene)-(C₄-C₆ N-heterocycloalkyl), —C(O)—(C₁-C₁₂ alkylamino), and —C(O)—(C₄-C₆ N-heterocycloalkyl), and the C₄-C₆ N-heterocycloalkyl moiety of any of the preceding substituents is optionally substituted with C₁-C₃ alkyl or C₁-C₃ hydroxyalkyl.

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino

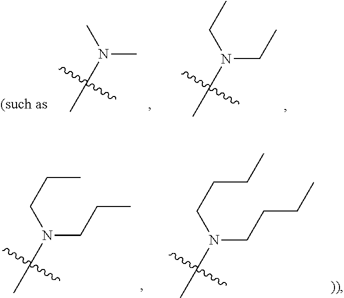

C₄-C₆ N-heterocycloalkyl (e.g., N-pyrrolidinyl

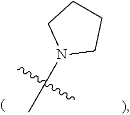 [SMILES: *N1CCCC1]

### N-piperidinyl

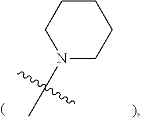 [SMILES: *N1CCCCC1]

### N-azepanyl

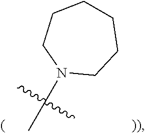 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

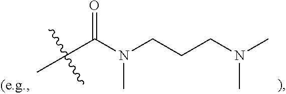 [SMILES: *C(=O)N(C)CCCN(C)C]

—C(O)N(C₁-C₃ alkyl)-(C₁-C₆ alkylene)-(C₄-C₆ N-heterocycloalkyl)

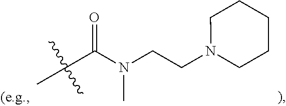 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

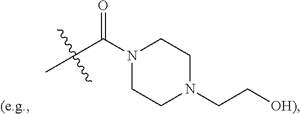

wherein the C₄-C₆ N-heterocycloalkyl moiety of any of the preceding substituents is optionally substituted with C₁-C₃ alkyl or C₁-C₃ hydroxyalkyl. In some embodiments of the terminating group of X_{Branch}, each terminating group is independently C₁-C₁₈ (e.g., C₄-C₁₈) alkylthiol, wherein the alkyl moiety is optionally substituted with one substituent —OH. In some embodiments of the terminating group of X_{Branch}, each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino

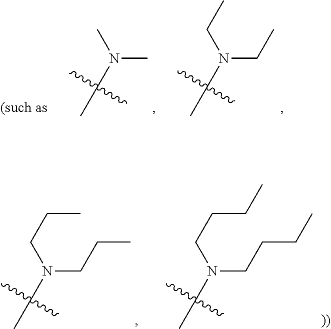

and C₄-C₆ N-heterocycloalkyl (e.g., N-pyrrolidinyl

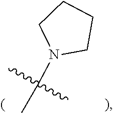 [SMILES: *N1CCCC1]

### N-piperidinyl

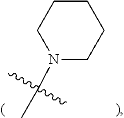 [SMILES: *N1CCCCC1]

### N-azepanyl

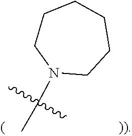 [SMILES: *N1CCCCCC1]

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol. In some embodiments of the terminating group of X_{Branch}, each terminating group is independently C₁-C₁₈ (e.g., C₄-C₁₈) alkylthiol.

In some embodiments of the terminating group of X_{Branch}, each terminating group is independently a structural set forth in Table 5. In some embodiments, the dendrimers described herein can comprise a terminating group or pharmaceutically acceptable salt, or thereof selected in Table 5. In some embodiments, the example terminating group of Table 5 are not limiting of the stereoisomers (i.e. enantiomers, diastereomers) listed.

TABLE 5
Example terminating group/peripheries structures

| ID # | Structure |
|---|---|
| SC1 | 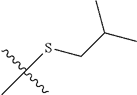 [SMILES: *SCC(C)C] |
| SC2 | 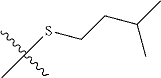 [SMILES: *SCCC(C)C] |
| SC3 | 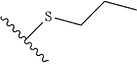 [SMILES: *SCCC] |
| SC4 | 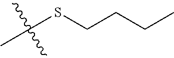 [SMILES: *SCCCC] |
| SC5 | 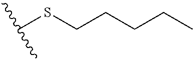 [SMILES: *SCCCCC] |
| SC6 | 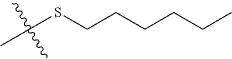 [SMILES: *SCCCCCC] |
| SC7 | 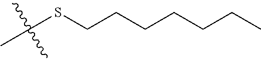 [SMILES: *SCCCCCCC] |
| SC8 | 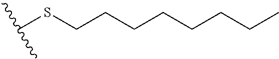 [SMILES: *SCCCCCCCC] |
| SC9 | 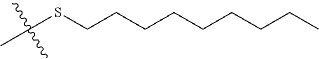 [SMILES: *SCCCCCCCCC] |
| SC10 | 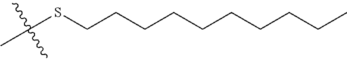 [SMILES: *SCCCCCCCCCC] |
| SC11 | 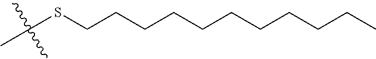 [SMILES: *SCCCCCCCCCCC] |
| SC12 | 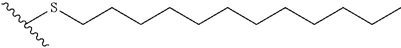 [SMILES: *SCCCCCCCCCCCC] |
| SC14 | 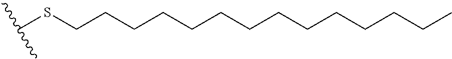 [SMILES: *SCCCCCCCCCCCCCC] |
| SC16 | 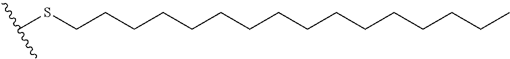 [SMILES: *SCCCCCCCCCCCCCCCC] |
| SC18 | 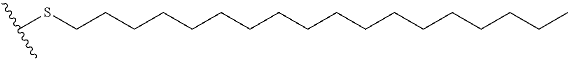 [SMILES: *SCCCCCCCCCCCCCCCCCC] |
| SC19 | 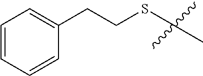 [SMILES: *SCCc1ccccc1] |
| SO1 | 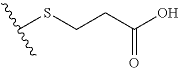 [SMILES: *SCCC(=O)O] |
| SO2 | 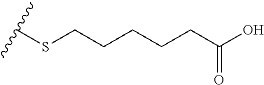 [SMILES: *SCCCCCC(=O)O] |
| SO3 | 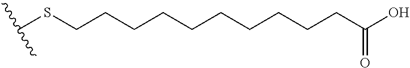 [SMILES: *SCCCCCCCCCCC(=O)O] |
| SO4 | 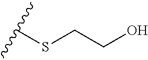 [SMILES: *SCCO] |
| SO5 | 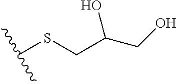 [SMILES: *SCC(O)CO] |
| SO6 | 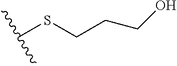 [SMILES: *SCCCO] |
| SO7 | 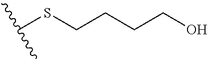 [SMILES: *SCCCCO] |
| SO8 | 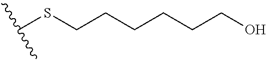 [SMILES: *SCCCCCCO] |
| SO9 | 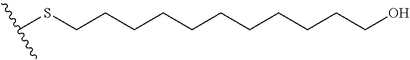 [SMILES: *SCCCCCCCCCCCO] |
| SN1 | 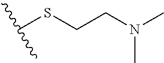 [SMILES: *SCCN(C)C] |
| SN2 | 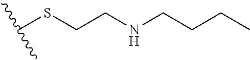 [SMILES: *SCCNCCCC] |
| SN3 | 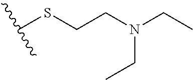 [SMILES: *SCCN(CC)CC] |
| SN4 | 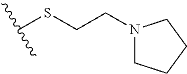 [SMILES: *SCCN1CCCC1] |
| SN5 | 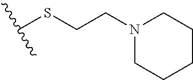 [SMILES: *SCCN1CCCCC1] |
| SN6 | 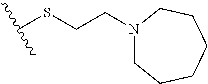 [SMILES: *SCCN1CCCCCC1] |
| SN7 | 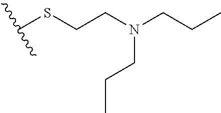 [SMILES: *SCCN(CCC)CCC] |
| SN8 | 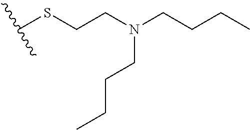 [SMILES: *SCCN(CCCC)CCCC] |
| SN9 | 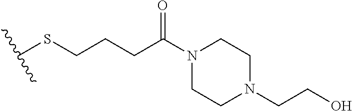 [SMILES: *SCCCC(=O)N1CCN(CCO)CC1] |
| SN10 | 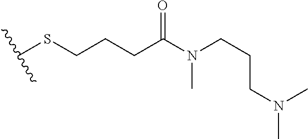 [SMILES: *SCCCC(=O)N(C)CCCN(C)C] |
| SN11 | 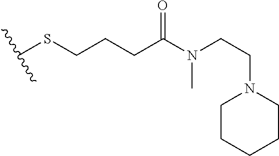 [SMILES: *SCCCC(=O)N(C)CCN1CCCCC1] |

In some embodiments, the dendrimer of Formula (X) is selected from those set forth in Table 6 and pharmaceutically acceptable salts thereof.

TABLE 6
Example ionizable cationic lipo-dendrimers

| ID # | Structure |
|---|---|
| 2A2- SC14 | 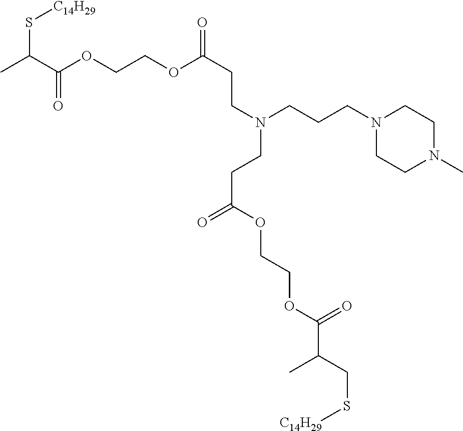 |
| 2A6- SC14 | 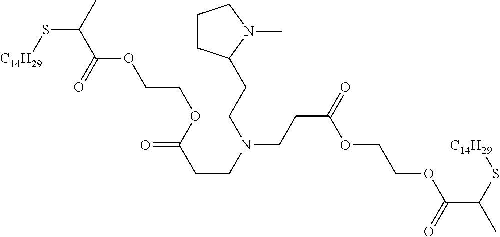 |
| 2A9- SC14 | 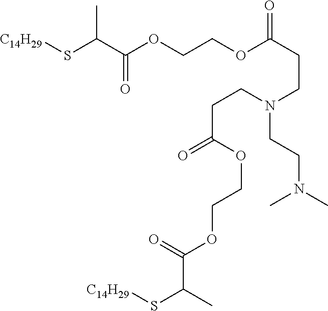 |
| 3A3- SC10 | 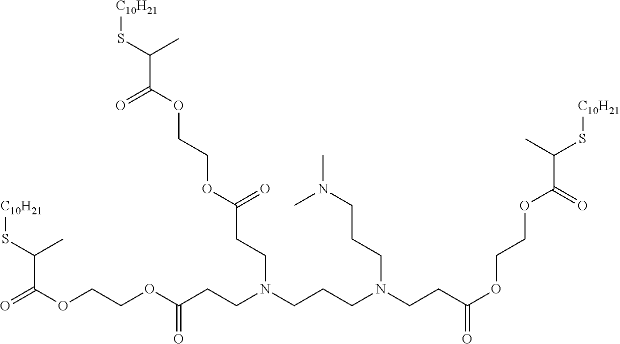 |
| 3A3- SC14 | 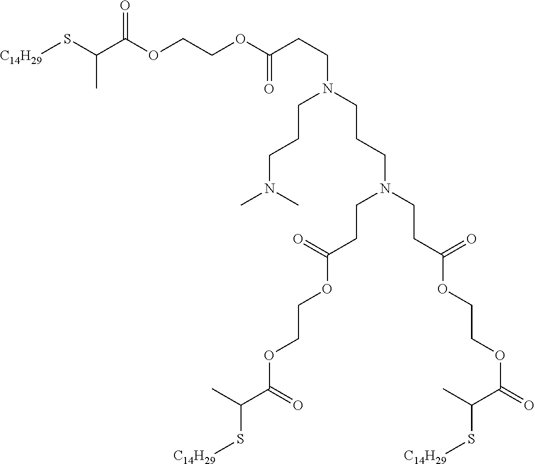 |
| 3A5- SC10 | 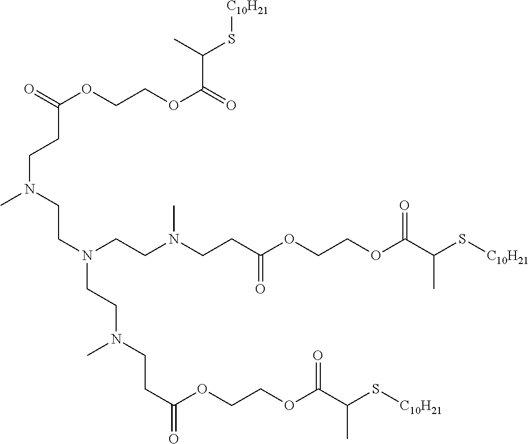 |
| 3A5- SC14 | 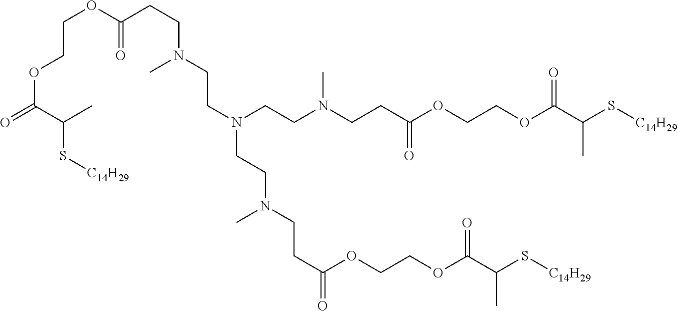 |
| 4A1- SC12 | 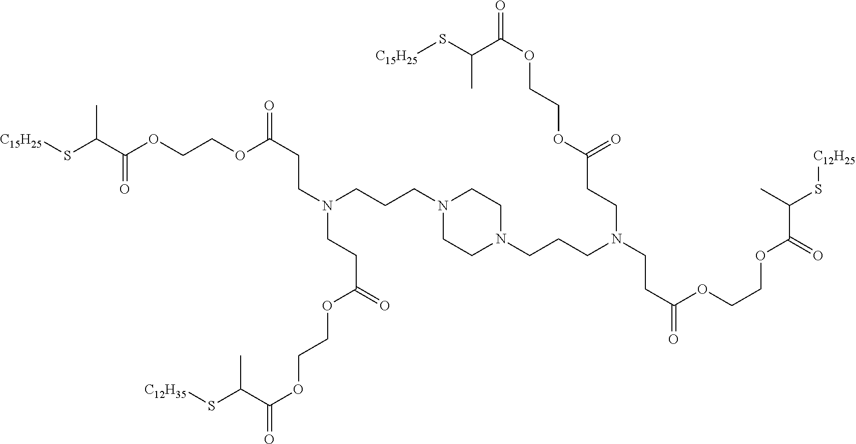 |
| 4A3- SC12 | 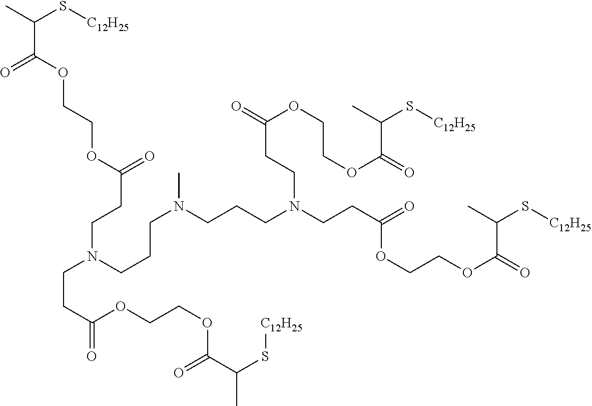 |
| 5A1- SC12 | 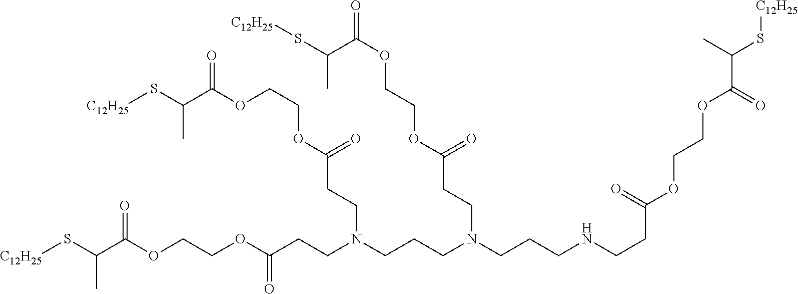 |
| 5A1- SC8 | 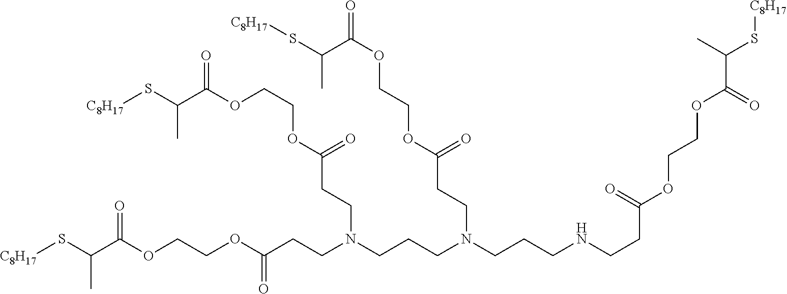 |
| 5A2- SC12 | 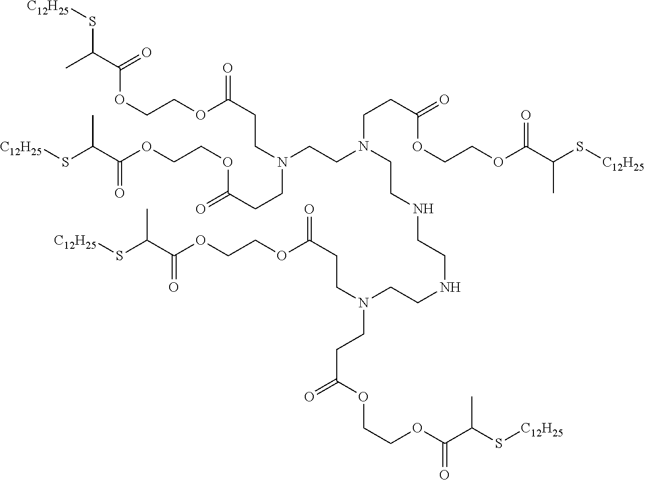 |
| 5A3- SC12 | 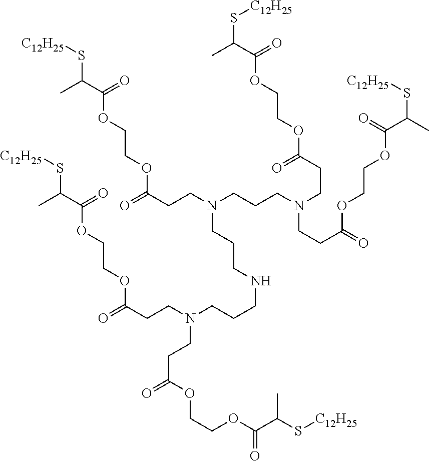 |
| 5A3- SC8 | 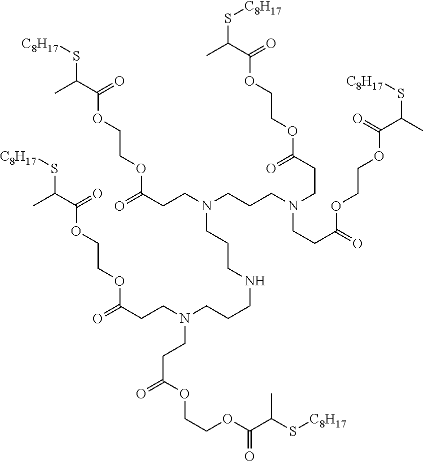 |
| 5A4- SC12 | 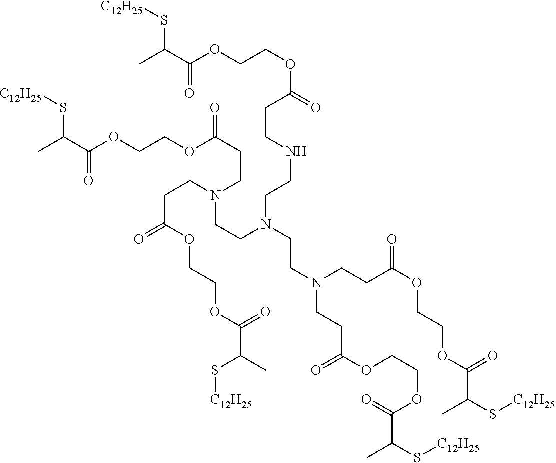 |
| 5A4- SC8 | 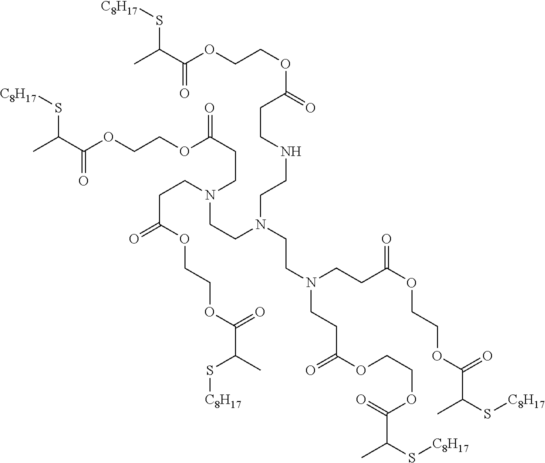 |
| 5A5- SC8 | 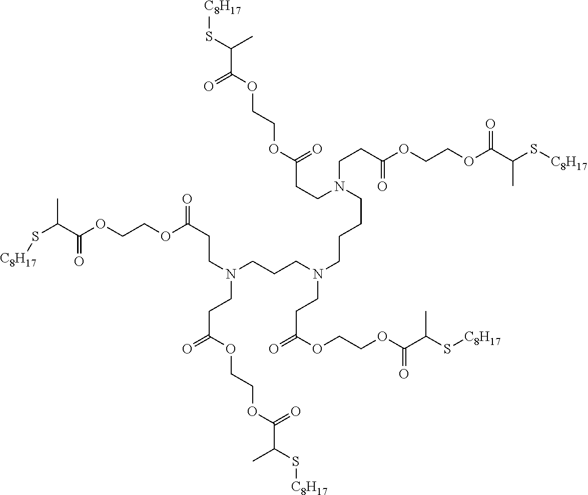 |
| 5A5- SC12 | 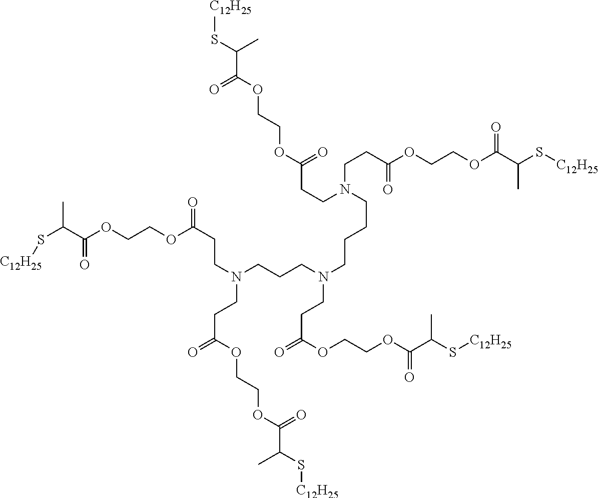 |
| 6A1- SC12 | 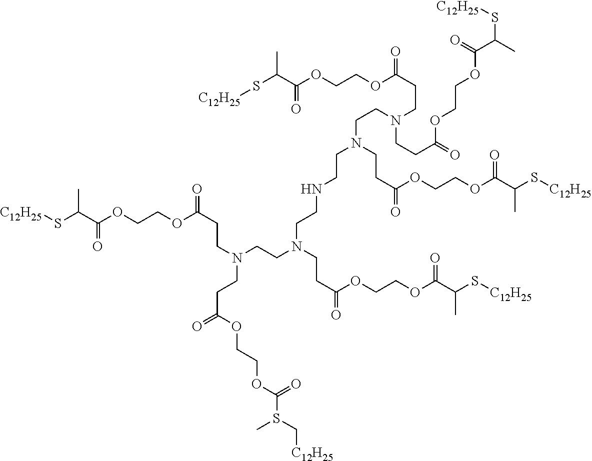 |
| 6A1- SC10 | 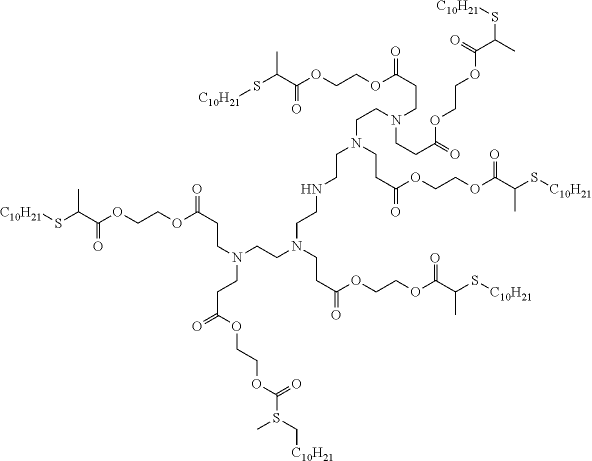 |
| 6A2- SC8 | 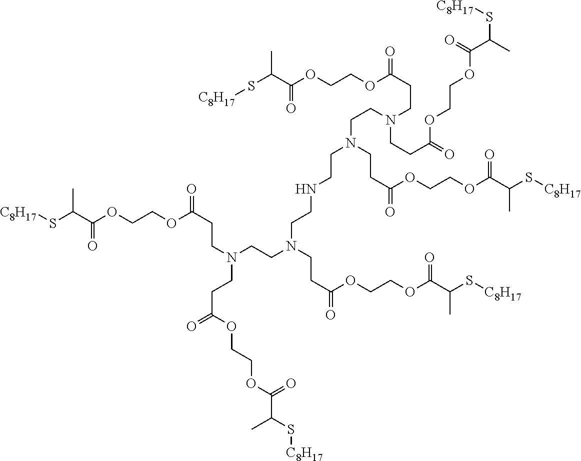 |
| 6A2- SC12 | 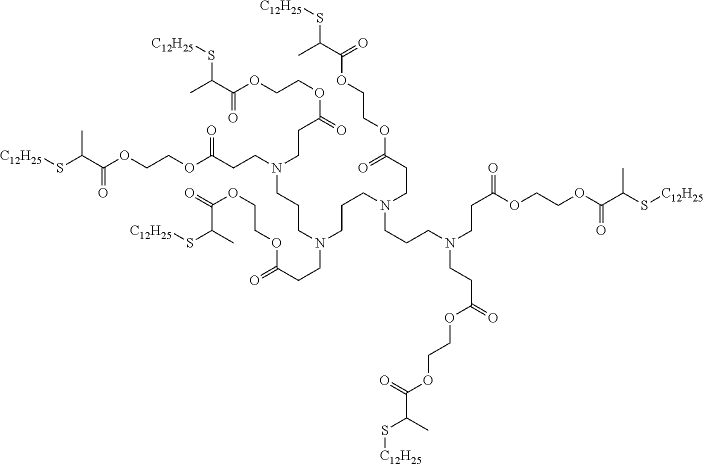 |
| 6A3- SC8 | 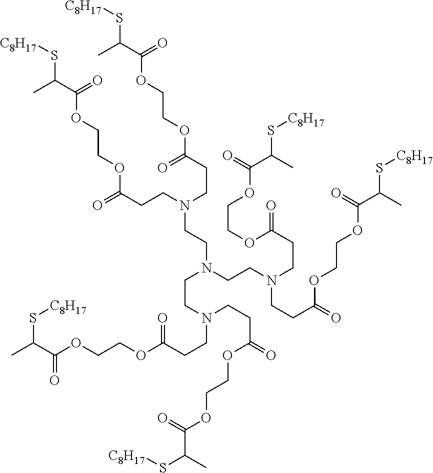 |
| 6A3- SC12 | 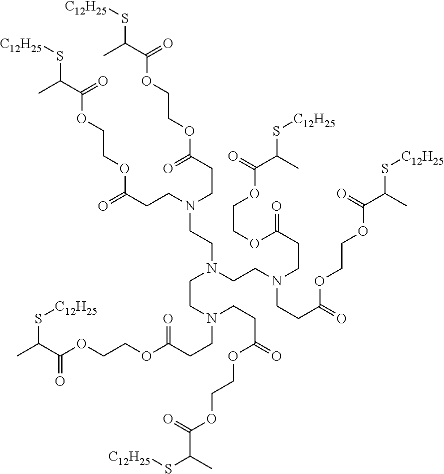 |
| 6A4- SC8 | 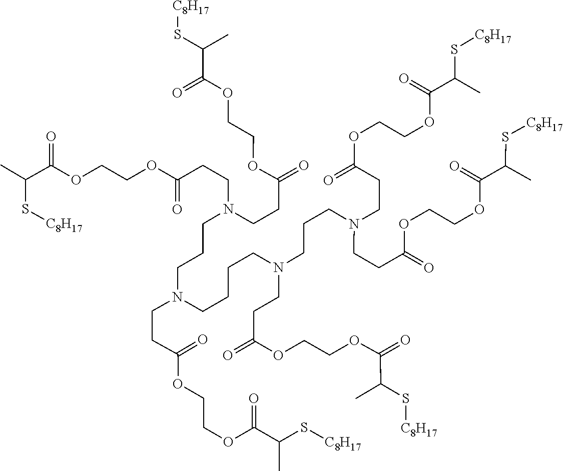 |
| 6A4- SC12 | 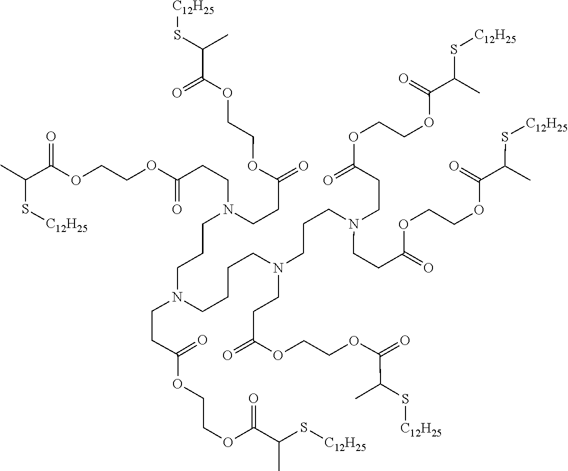 |
| 2A2- g2- SC12 | 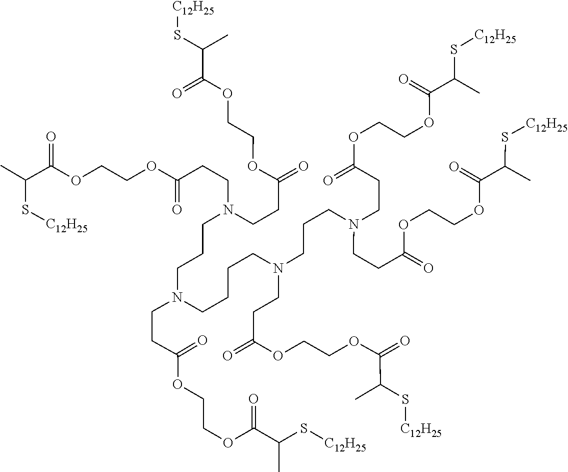 |
| 2A2- g2- SC12 | 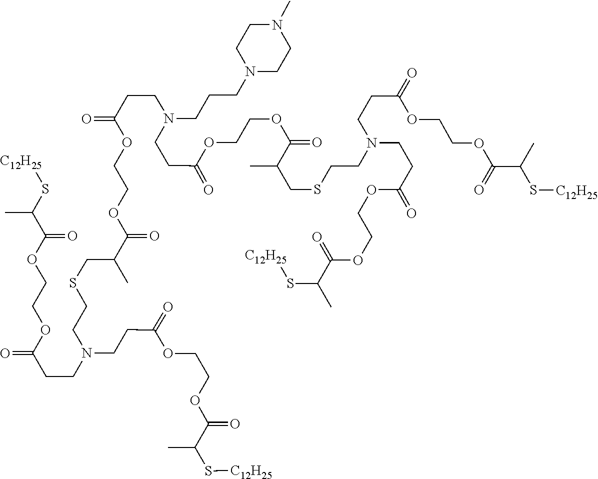 |
| 2A2- g2- SC8 | 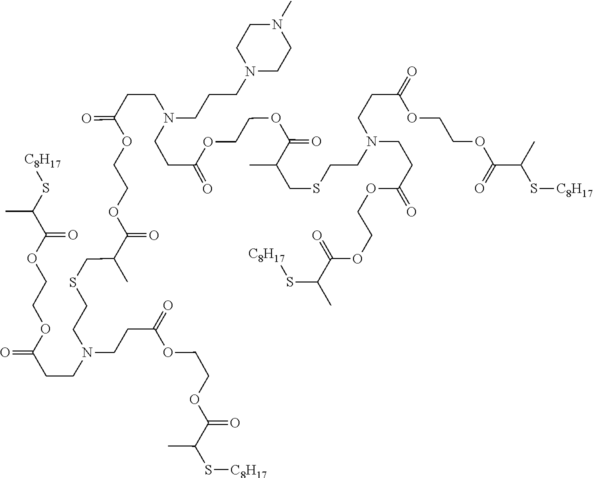 |
| 2A11- g2- SC12 | 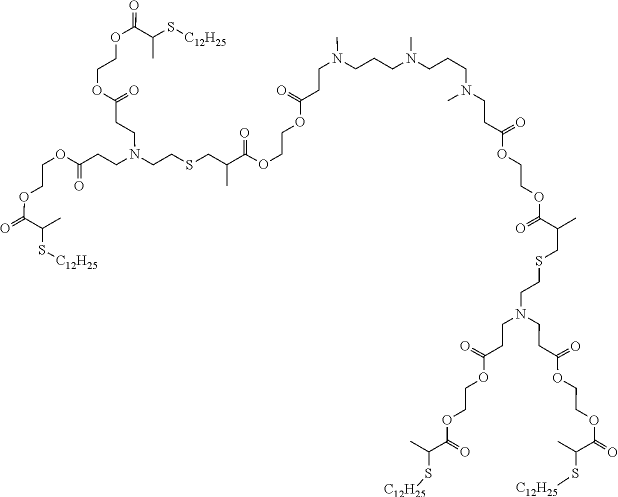 |
| 2A11- g2- SC8 | 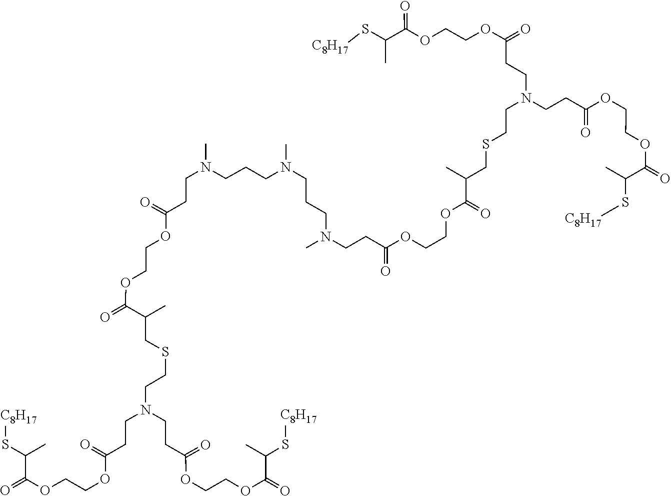 |
| 3A3- g2- SC12 | 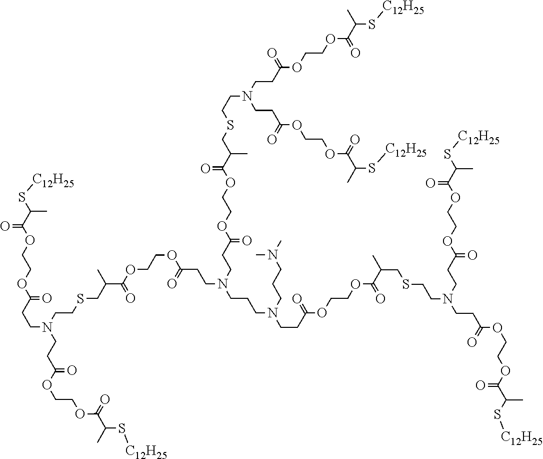 |
| 3A3- g2- SC8 | 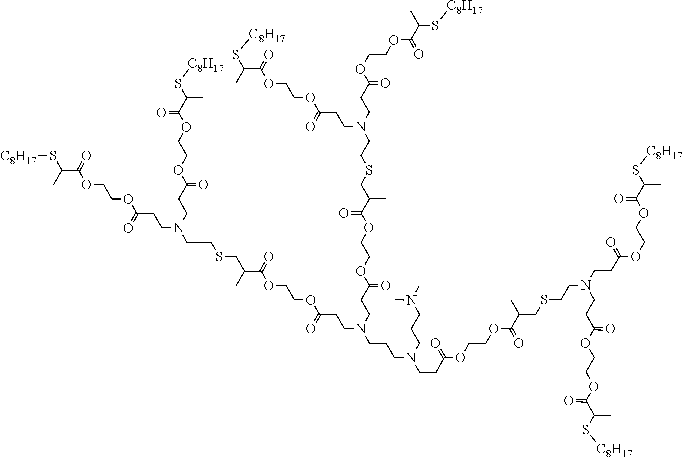 |
| 3A5- g2- SC12 | 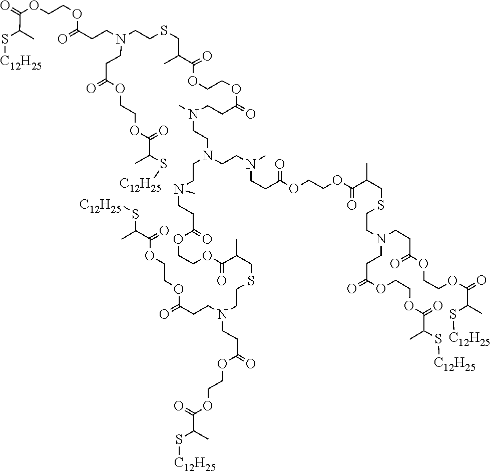 |
| 2A11- g3- SC12 | 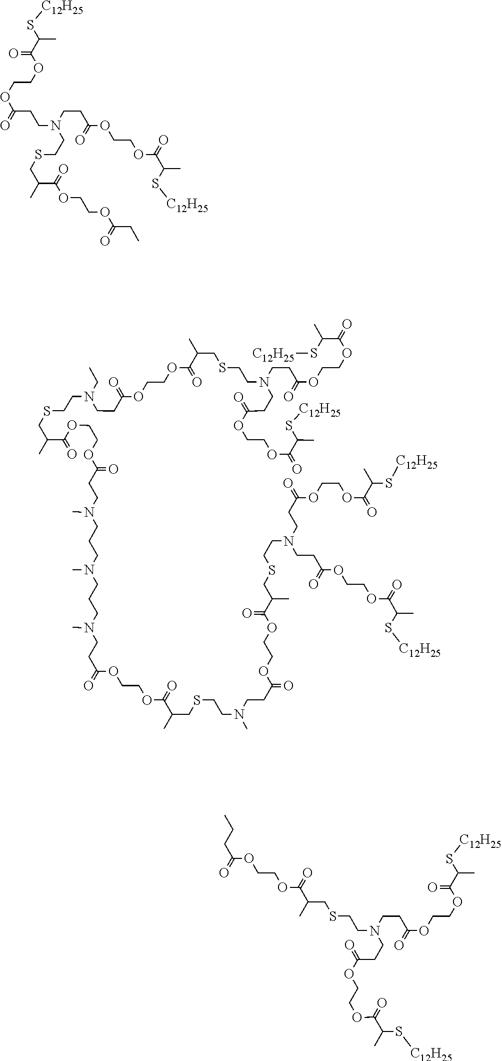 |
| 2A11- g3- SC8 | 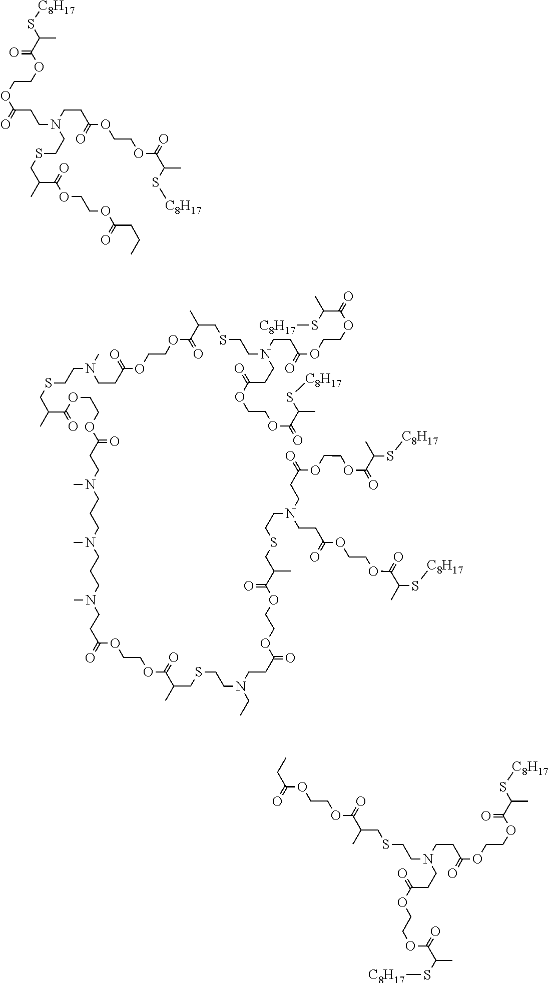 |
| 1A2- G4- SC12 | 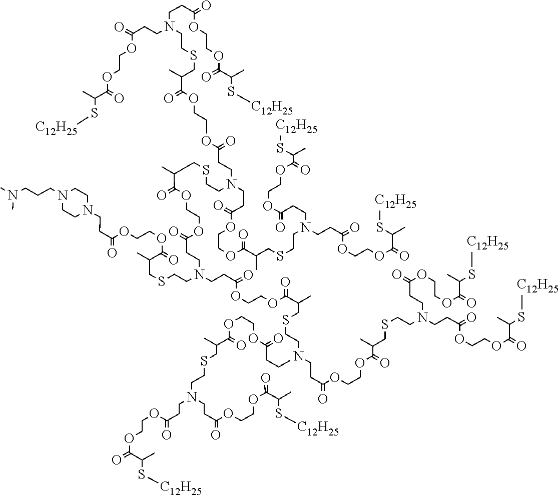 |
| 4A1- g2- SC12 | 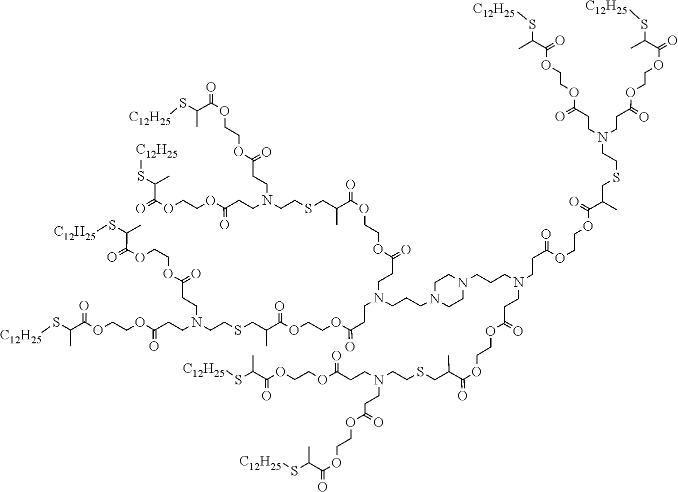 |
| 1A2- G4- SC8 | 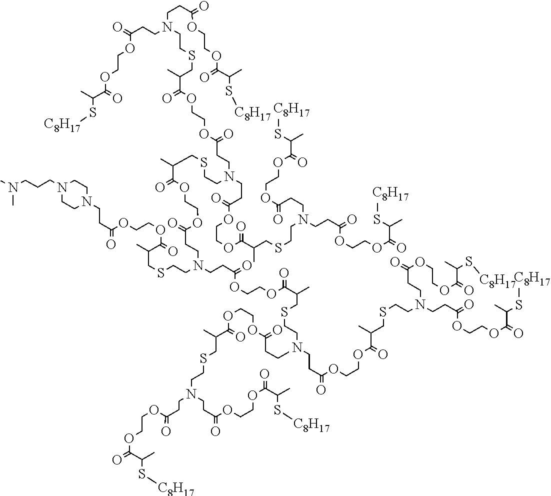 |
| 4A1- SC5 | 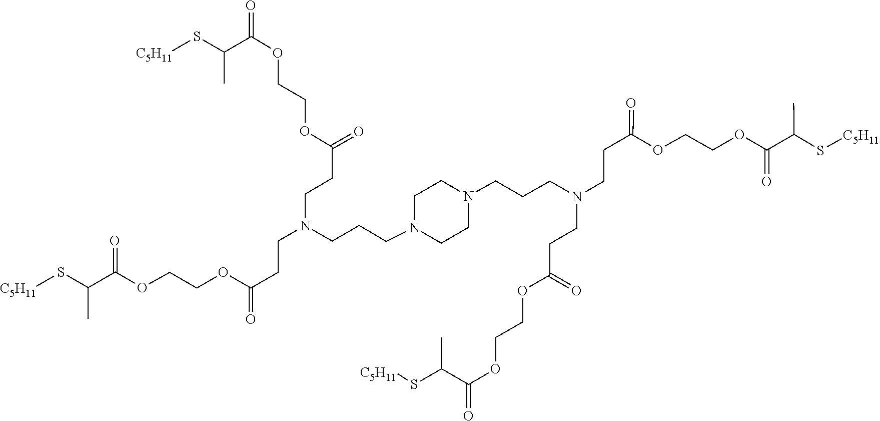 |
| 4A1- g2- SC8 | 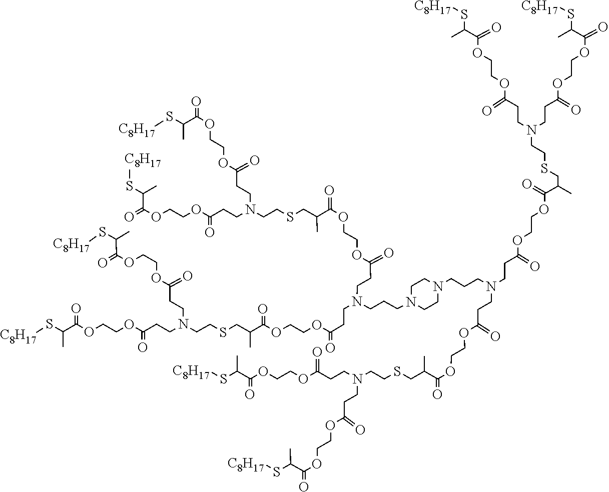 |
| 5A2- SC8-1 | 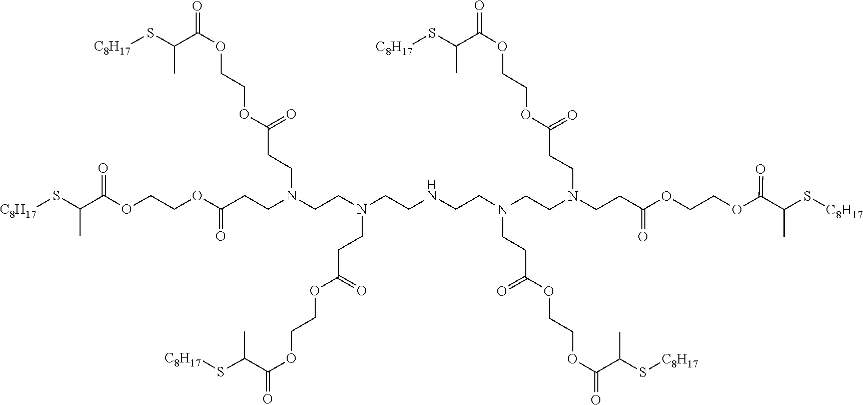 |
| 4A3- g2- SC12 | 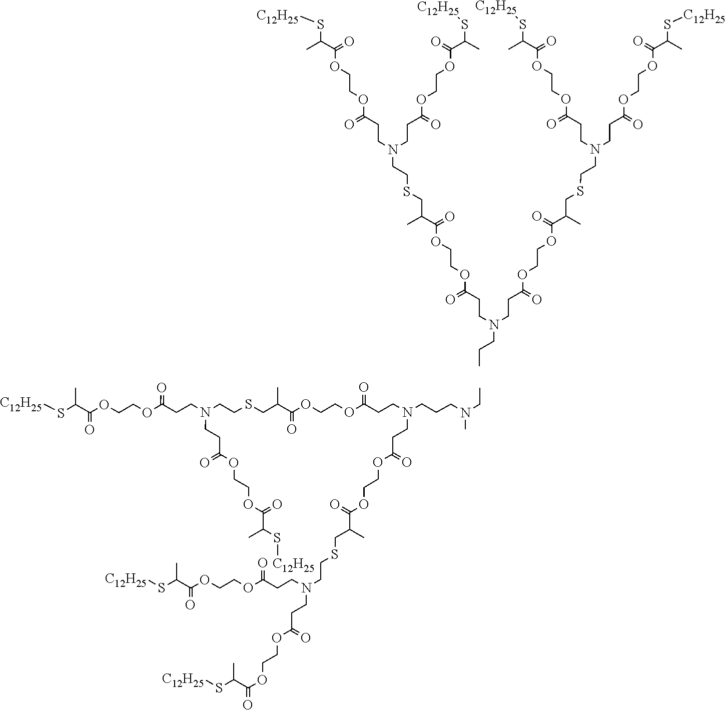 |
| 5A2- SC8 | 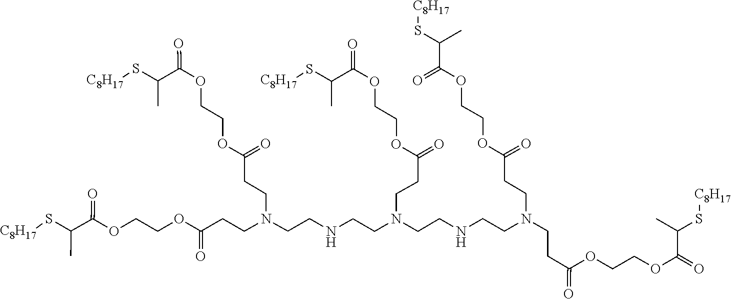 |
| 4A3- g2- SC8 | 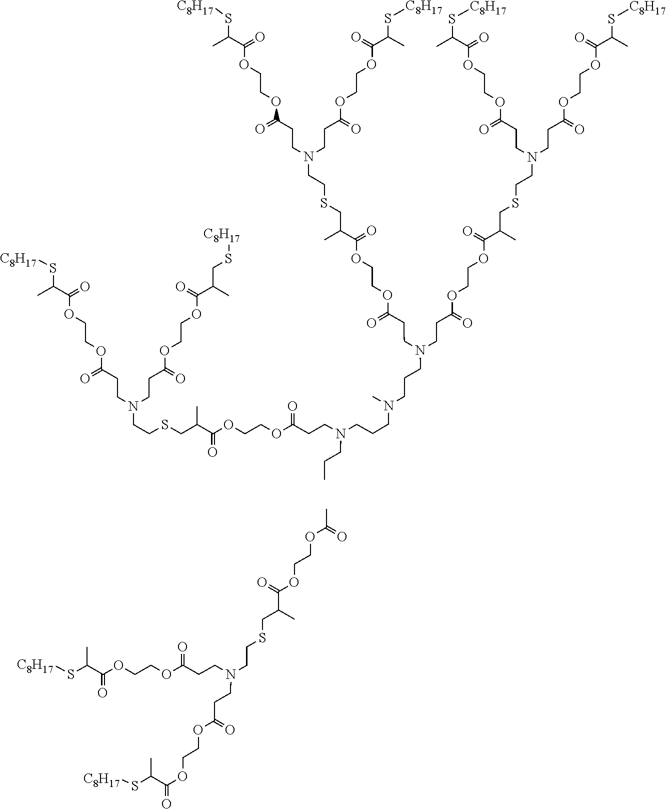 |
| 1A2- g3- SC12 | 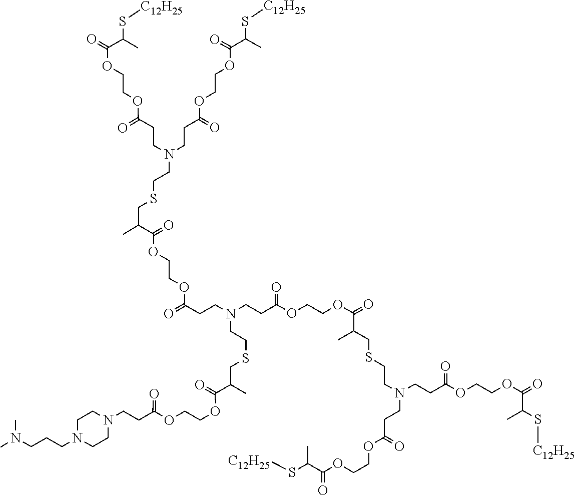 |
| 1A2- g3- SC8 | 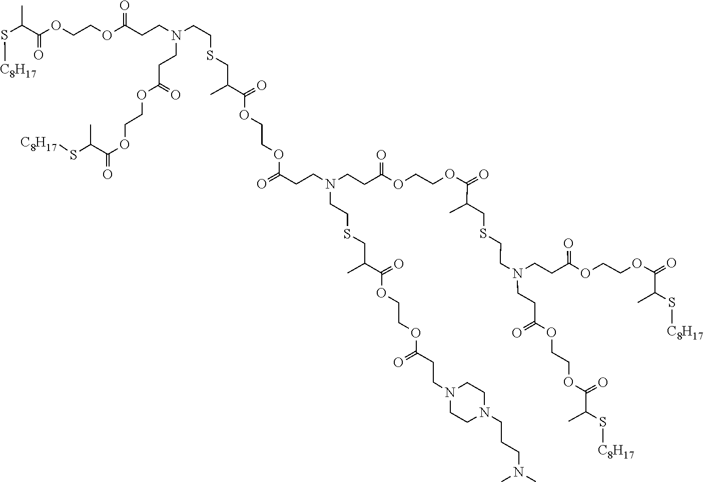 |
| 2A2- g3- SC12 | 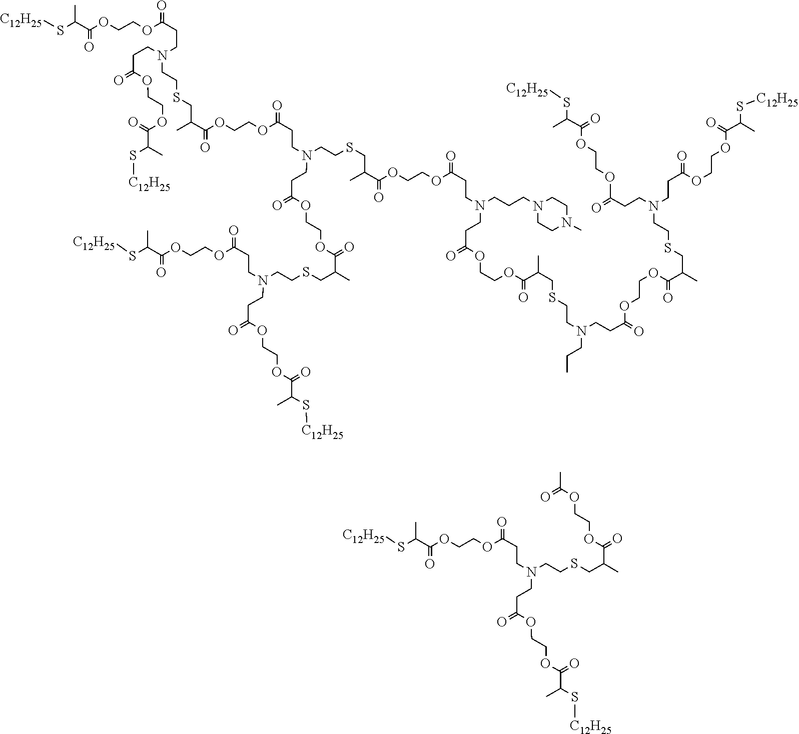 |
| 2A2- g3- SC8 | 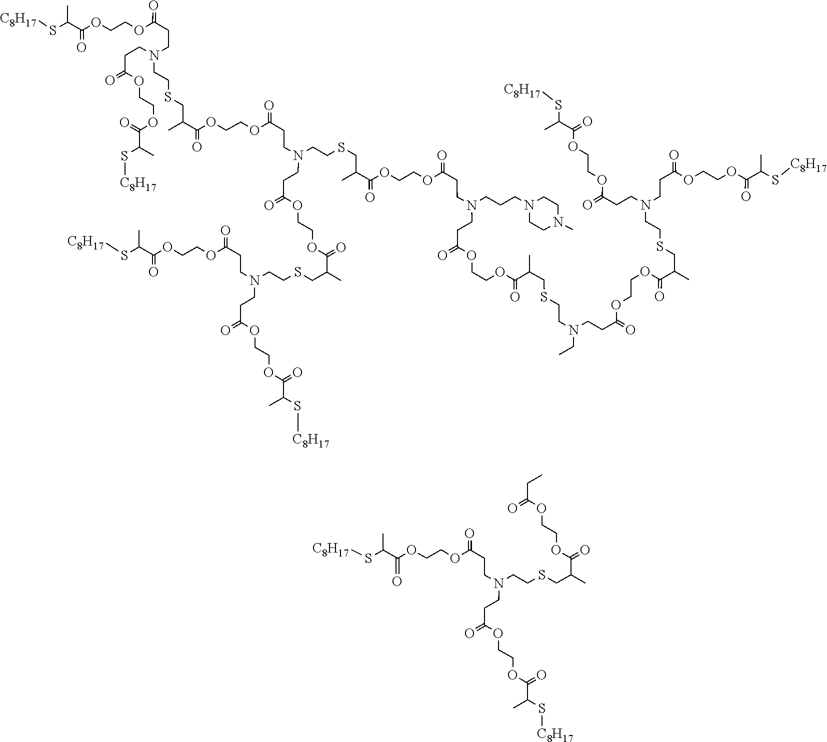 |
| 5A2- 2-SC8 | 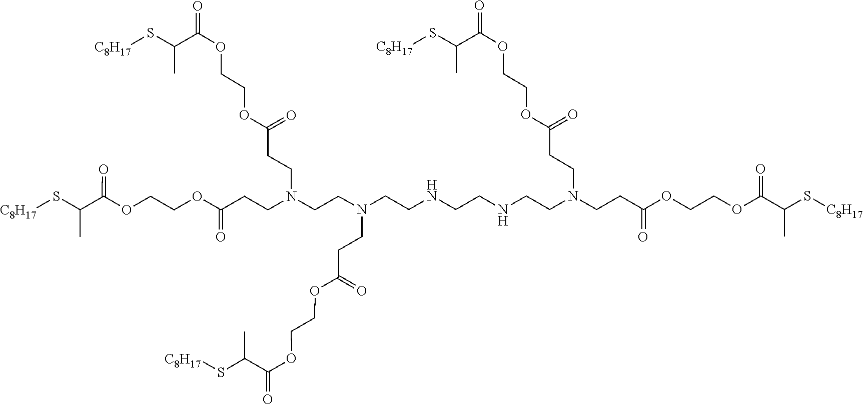 |
| 4A1- SC8 | 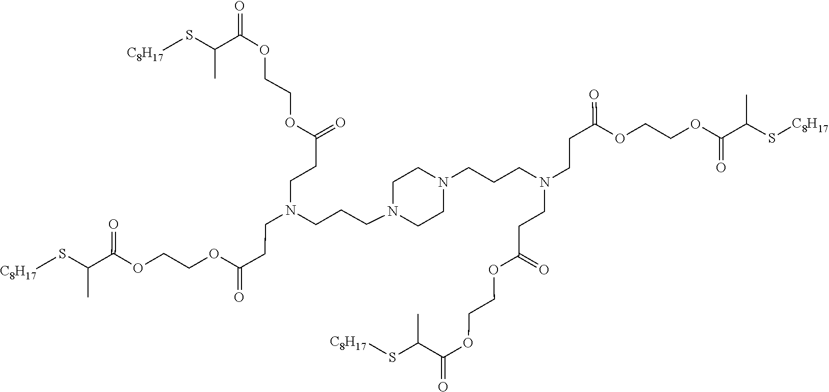 |
| 4A3- SC6 | 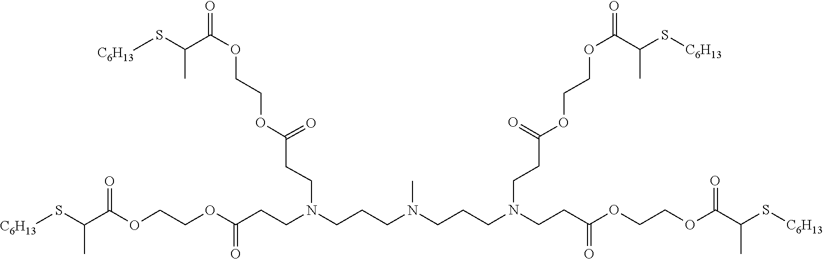 |
| 4A3- SC7 | 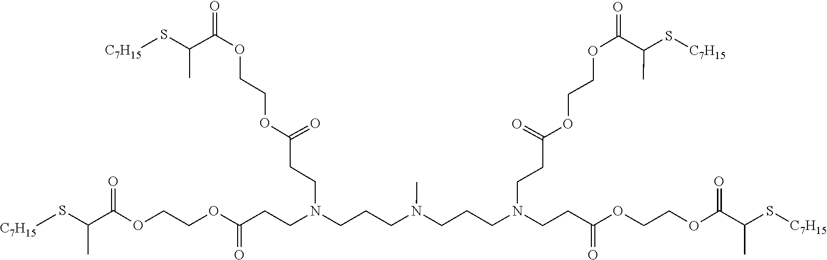 |
| 4A3- SC8 | 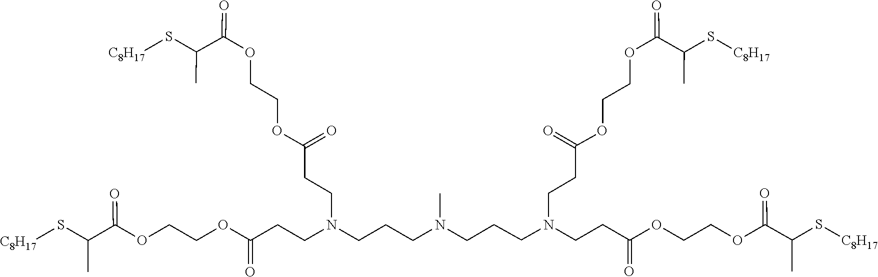 |
| 5A4- SC5 | 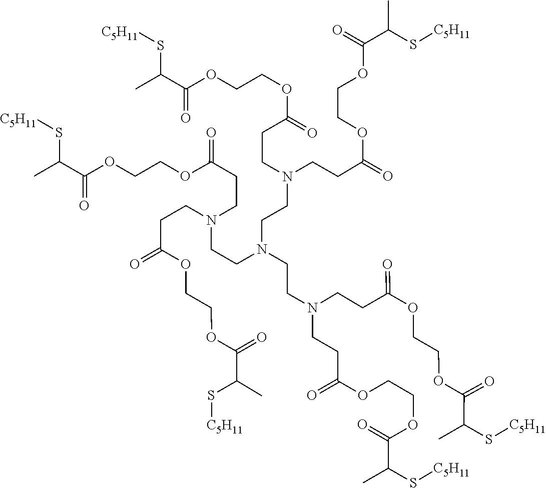 |
| 5A4- SC6 | 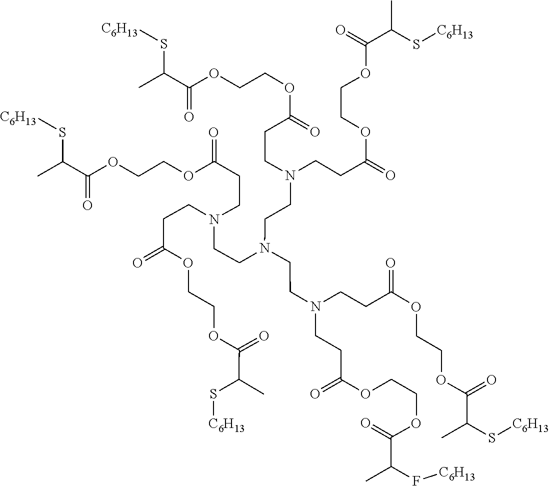 |
| 5A4- SC8-1 | 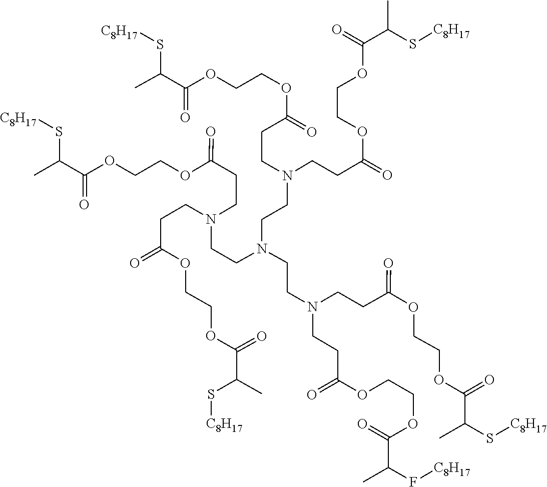 |
| 5A2- SC8 | 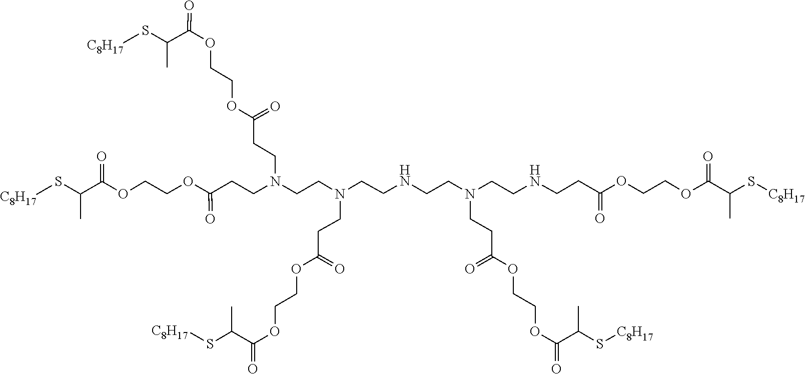 |
| 3A5- SC14 | 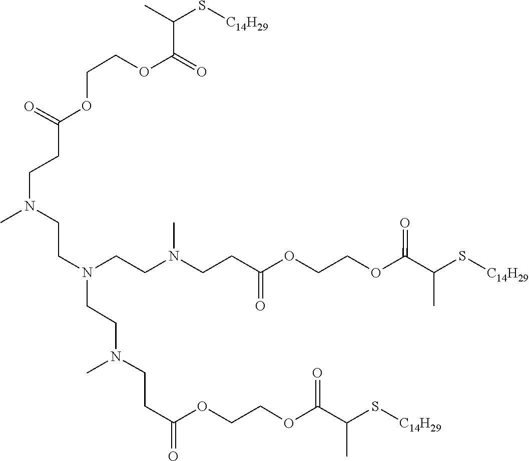 |
| 6A3- SC12 | 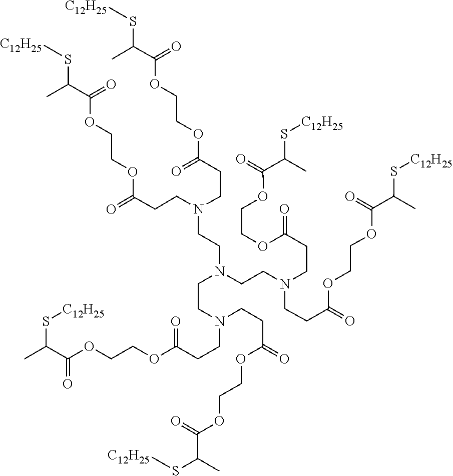 |
| 5A3- SC8 | 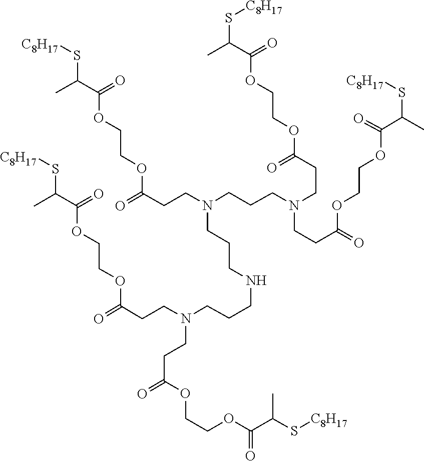 |
| 3A5- g2- SC8 | 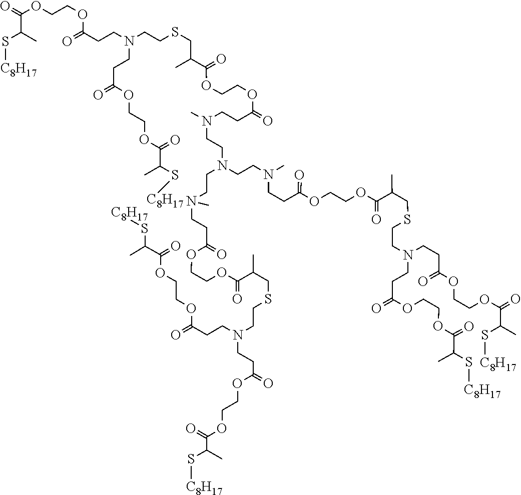 |
| 3A4- SC6 | 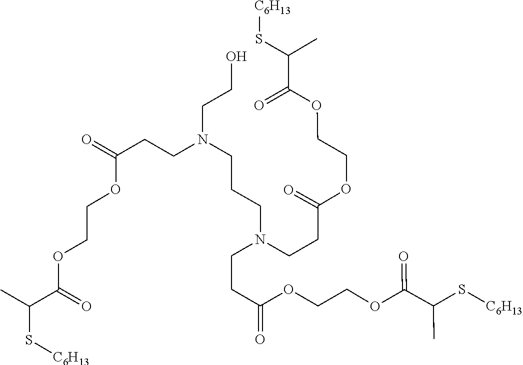 |
| 2A2- SC8 | 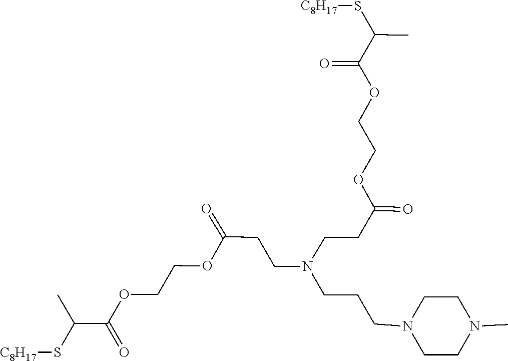 |
| 6A3- SC6 | 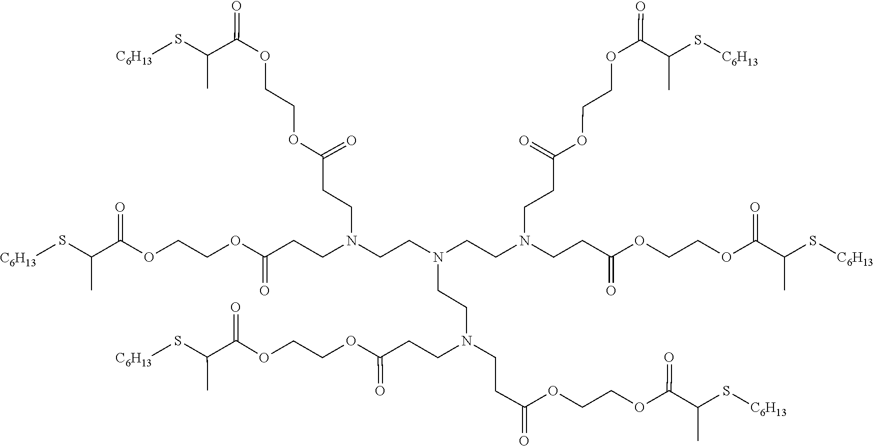 |
| 4A1- SC6 | 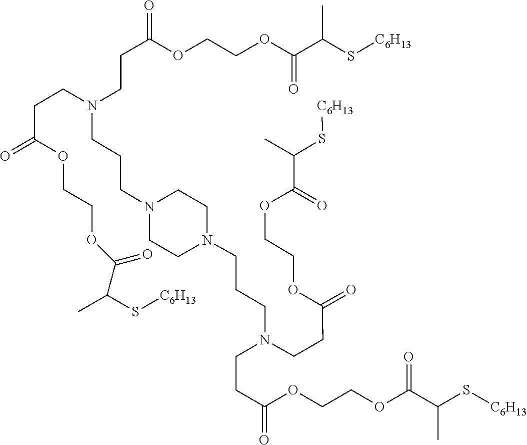 |

LF92

In some embodiments, the lipid composition of the present disclosure comprises a cationic lipid having a structural formula (I′):

wherein:

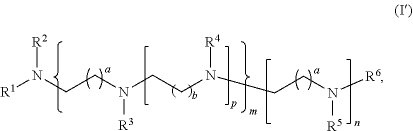

a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and
R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
wherein one or more of the nitrogen atoms indicated in formula (I′) may be protonated to provide a cationic lipid.

In some embodiments of the cationic lipid of formula (I′), a is 1. In some embodiments of the cationic lipid of formula (I′), b is 2. In some embodiments of the cationic lipid of formula (I′), m is 1. In some embodiments of the cationic lipid of formula (I′), n is 1. In some embodiments of the cationic lipid of formula (I′), R¹, R², R³, R⁴, R⁵, and R⁶ are each independently H or —CH₂CH(OH)R⁷. In some embodiments of the cationic lipid of formula (I′), R¹, R², R³, R⁴, R⁵, and R⁶ are each independently H or

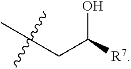

In some embodiments of the cationic lipid of formula (I′), R¹, R², R³, R⁴, R⁵, and R⁶ are each independently H

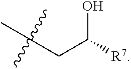

In some embodiments of the cationic lipid of formula (I′), R⁷ is C₃-C₁₈ alkyl (e.g., C₆-C₁₂ alkyl).

In some embodiments, the cationic lipid of formula (I′) is 13,16,20-tris(2-hydroxydodecyl)-13,16,20,23-tetraazapentatricontane-11,25-diol:

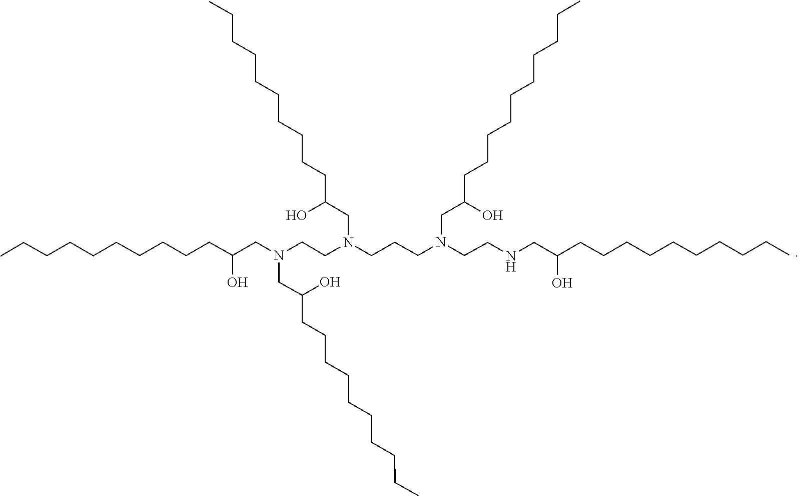 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

In some embodiments, the cationic lipid of formula (I′) is (11R,25R)-13,16,20-tris((R)-2-hydroxydodecyl)-13,16,20,23-tetraazapentatricontane-11,25-diol:

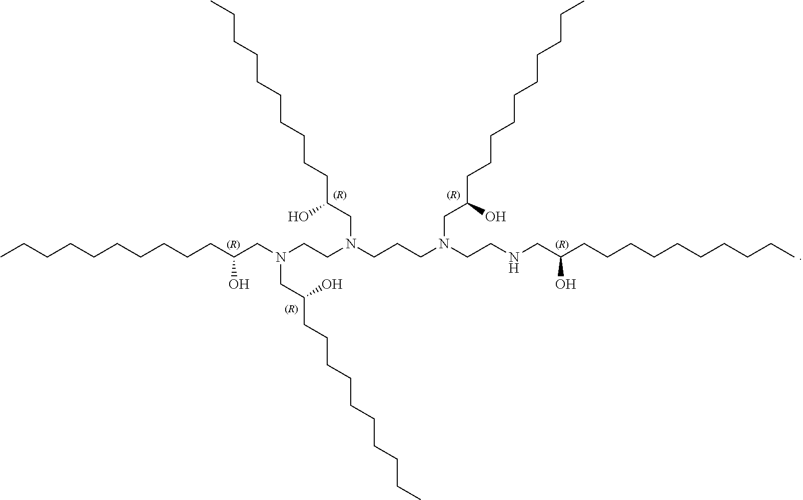

In some embodiments of the LF92 lipid composition, a lipid of the lipid composition can be in a particular amount or molar percentage. In some embodiments, the lipid composition comprises the cationic lipid of formula (I′) at a molar percentage of no more than 50% (e.g., no more than 45%). In some embodiments, the LF92 lipid composition further comprises a phospholipid. In some embodiments, the phospholipid is present in the LF92 lipid composition at a molar percentage of at least about 10%, 15%, 20%, or 25%. In some embodiments, the phospholipid is present in the LF92 lipid composition at a molar percentage of at most about 40%, 35%, or 30%. In some embodiments, the phospholipid is present in the LF92 lipid composition at a molar percentage of about 10%, 15%, 20%, 25%, 30%, 35%, or 40%, or any range between any two of the foregoing. In some embodiments, the phospholipid is present in the LF92 lipid composition at a molar percentage of 10% to 40%, or 20% to 40%. In some embodiments, lipid composition further comprises a steroid or steroid derivative. In some embodiments, the lipid composition further comprises a polymer-conjugated lipid (e.g., poly(ethylene glycol) (PEG)-conjugated lipid).

### Other Ionizable Cationic Lipids

In some embodiments of the lipid composition, the cationic lipid comprises a structural formula (D-I′):
1.

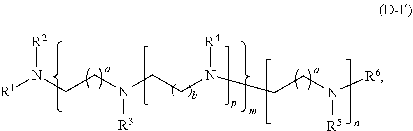

2. wherein:
3. a is 1 and b is 2, 3, or 4; or, alternatively, b is 1 and a is 2, 3, or 4;
4. m is 1 and n is 1; or, alternatively, m is 2 and n is 0; or, alternatively, m is 2 and n is 1; and
5. R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
6. provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
7. wherein one or more of the nitrogen atoms indicated in formula (D-I′) may be protonated to provide a cationic lipid.

In some embodiments of the cationic lipid of formula (D-I′), a is 1. In some embodiments of the cationic lipid of formula (D-I′), b is 2. In some embodiments of the cationic lipid of formula (D-I′), m is 1. In some embodiments of the cationic lipid of formula (D-I′), n is 1. In some embodiments of the cationic lipid of formula (D-I′), R¹, R², R³, R⁴, R⁵, and R⁶ are each independently H or —CH₂CH(OH)R⁷. In some embodiments of the cationic lipid of formula (D-I′), R¹, R², R³, R⁴, R⁵, and R⁶ are each independently H or

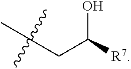

In some embodiments of the cationic lipid of formula (D-I′), R¹, R², R³, R⁴, R⁵, and R⁶ are each independently H or

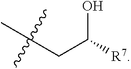

In some embodiments of the cationic lipid of formula (D-I′), R⁷ is C₃-C₁₈ alkyl (e.g., C₆-C₁₂ alkyl).

In some embodiments, the cationic lipid of formula (D-I′) is 13,16,20-tris(2-hydroxydodecyl)-13,16,20,23-tetraazapentatricontane-11,25-diol:

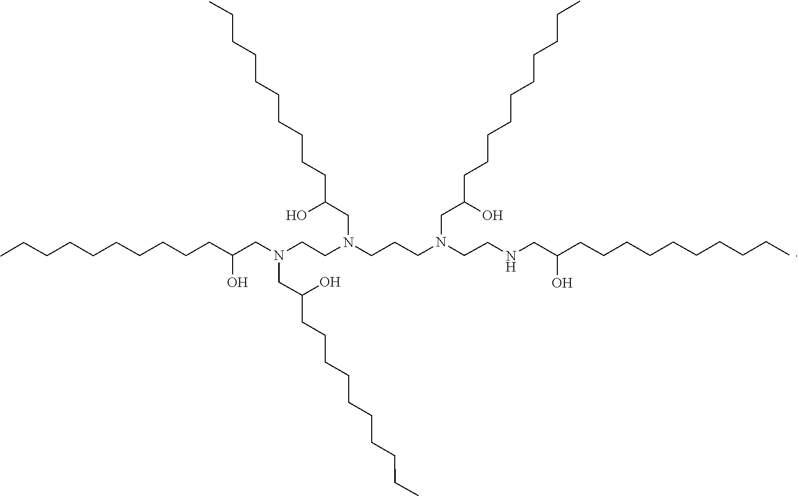 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

In some embodiments, the cationic lipid of formula (D-I′) is (11R,25R)-13,16,20-tris((R)-2-hydroxydodecyl)-13,16,20,23-tetraazapentatricontane-11,25-diol:

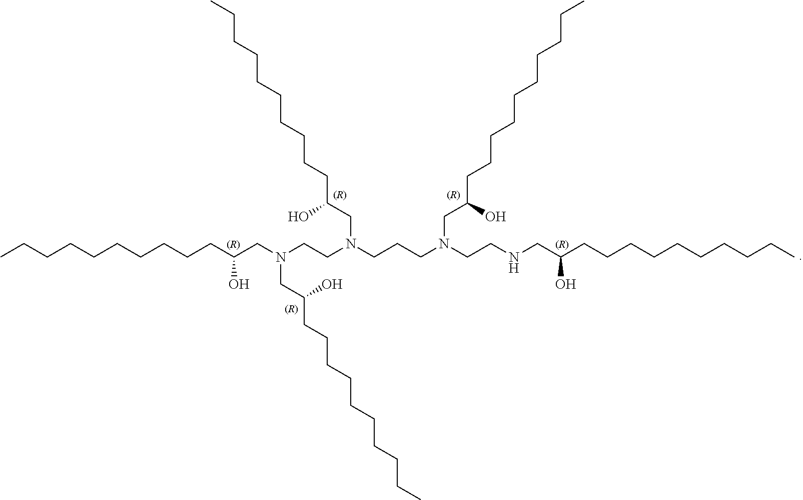

Additional cationic lipids that can be used in the compositions and methods of the present application include those cationic lipids as described in J. McClellan, M. C. King, Cell 2010, 141, 210-217, and International Patent Publication WO 2010/144740, WO 2013/149140, WO 2016/118725, WO 2016/118724, WO 2013/063468, WO 2016/205691, WO 2015/184256, WO 2016/004202, WO 2015/199952, WO 2017/004143, WO 2017/075531, WO 2017/117528, WO 2017/049245, WO 2017/173054 and WO 2015/095340, which are incorporated herein by reference for all purposes. Examples of those ionizable cationic lipids include but are not limited to those as shown in Table 7.

TABLE 7
Example ionizable cationic lipids

| # | Structure of example ionizable cationic lipid |
|---|---|
| 1 | 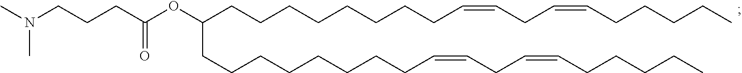 [SMILES: CCCCC/C=C\C/C=C\CCCCCCCCC(CCCCCCCC/C=C\C/C=C\CCCCC)OC(=O)CCCN(C)C] |
| 2 | 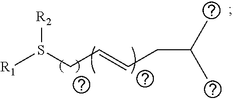 |
| 3 | 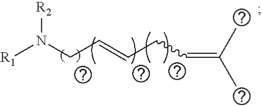 |
| 4 | 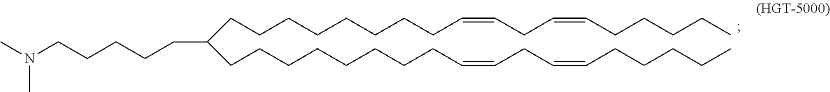 [SMILES: CCCCC/C=C\C/C=C\CCCCCCCCC(CCCCCCCC/C=C\C/C=C\CCCCC)CCCCCN(C)C] |
| 5 | 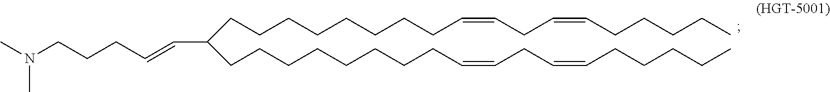 [SMILES: CCCCC/C=C\C/C=C\CCCCCCCCC(/C=C/CCCN(C)C)CCCCCCCC/C=C\C/C=C\CCCCC] |
| 6 | 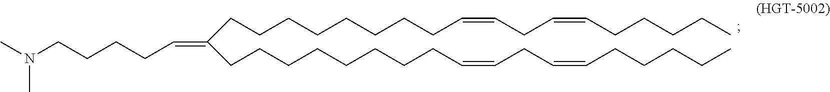 [SMILES: CCCCC/C=C\C/C=C\CCCCCCCCC(=CCCCCN(C)C)CCCCCCCC/C=C\C/C=C\CCCCC] |
| 7 | 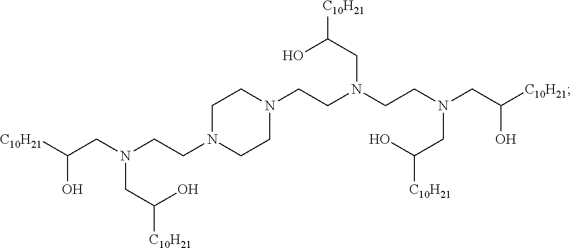 |
| 8 | 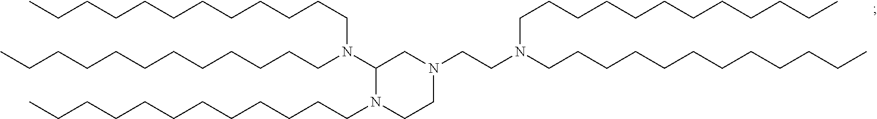 [SMILES: CCCCCCCCCCCCN(CCCCCCCCCCCC)CCN1CCN(CCCCCCCCCCCC)C(N(CCCCCCCCCCCC)CCCCCCCCCCCC)C1] |
| 9 | 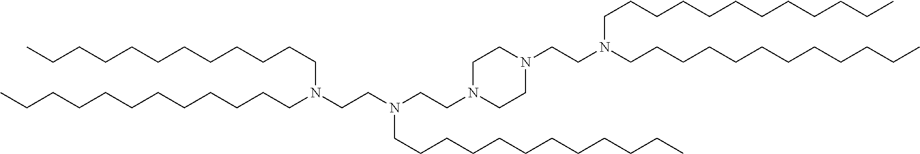 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1] |
| 10 | 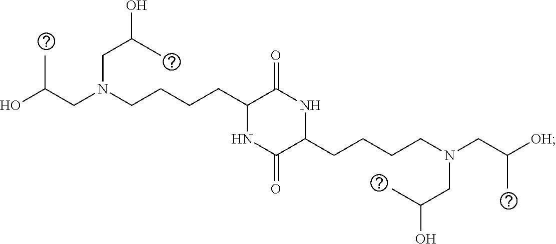 [SMILES: CC(O)CN(CCCCC1NC(=O)C(CCCCN(CC(C)O)CC(C)O)NC1=O)CC(C)O] |
| 11 | 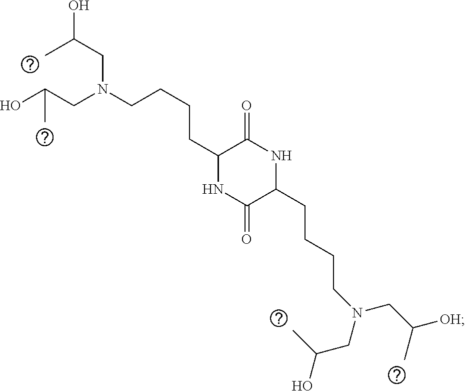 [SMILES: CC(O)CN(CCCCC1NC(=O)C(CCCCN(CC(C)O)CC(C)O)NC1=O)CC(C)O] |
| 12 | 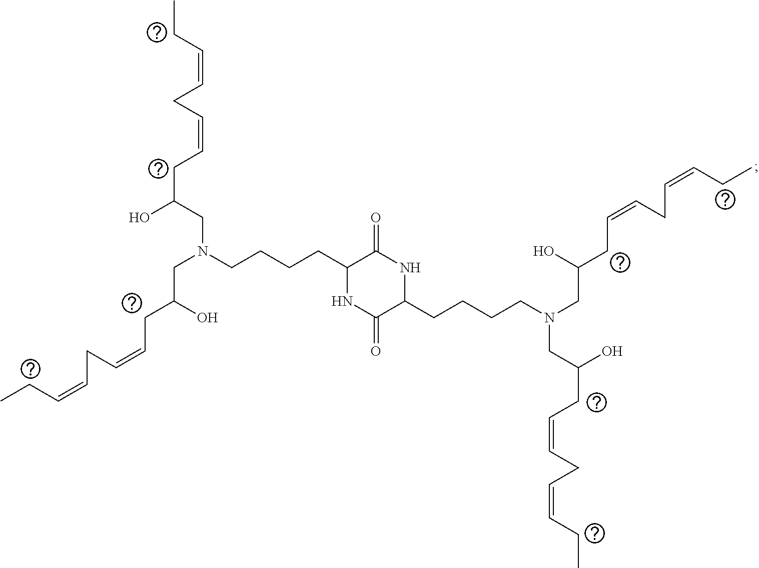 [SMILES: CC/C=C\C/C=C\CC(O)CN(CCCCC1NC(=O)C(CCCCN(CC(O)C/C=C\C/C=C\CC)CC(O)C/C=C\C/C=C\CC)NC1=O)CC(O)C/C=C\C/C=C\CC] |
| 13 | 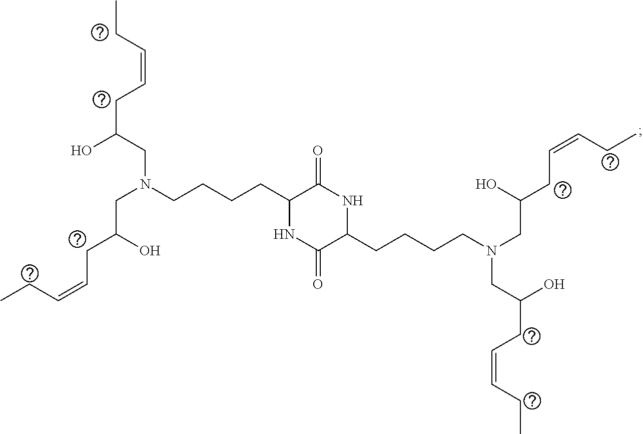 [SMILES: CC/C=C\CC(O)CN(CCCCC1NC(=O)C(CCCCN(CC(O)C/C=C\CC)CC(O)C/C=C\CC)NC1=O)CC(O)C/C=C\CC] |
| 14 | 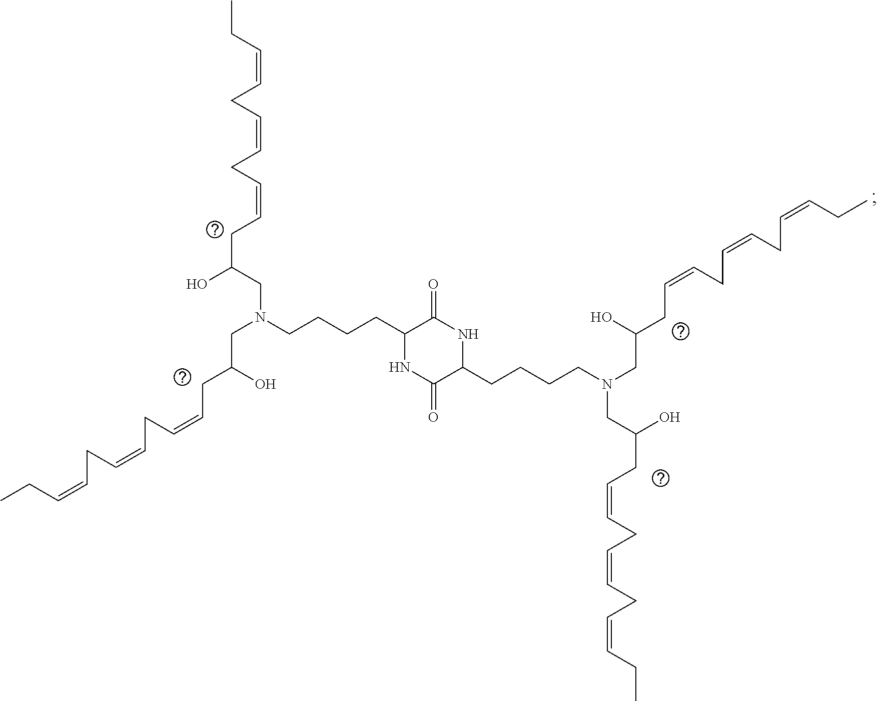 [SMILES: CC/C=C\C/C=C\C/C=C\CC(O)CN(CCCCC1NC(=O)C(CCCCN(CC(O)C/C=C\C/C=C\C/C=C\CC)CC(O)C/C=C\C/C=C\C/C=C\CC)NC1=O)CC(O)C/C=C\C/C=C\C/C=C\CC] |
| 15 | 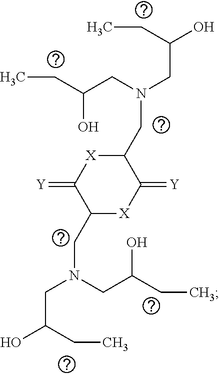 |
| 16 | 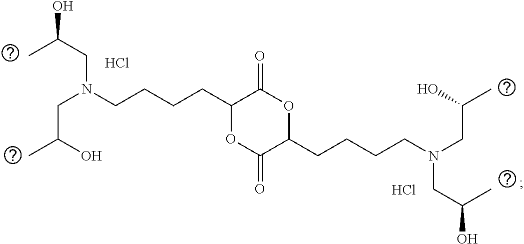 [SMILES: CC(O)CN(CCCCC1OC(=O)C(CCCCN(C[C@@H](C)O)C[C@@H](C)O)OC1=O)C[C@@H](C)O ; Cl ; Cl] |
| 17 | 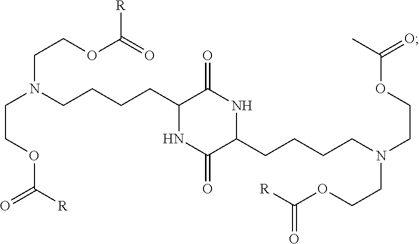 |
| 18 | 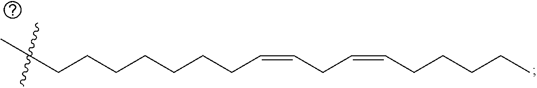 [SMILES: *CCCCCCC/C=C\C/C=C\CCCCC] |
| 19 | 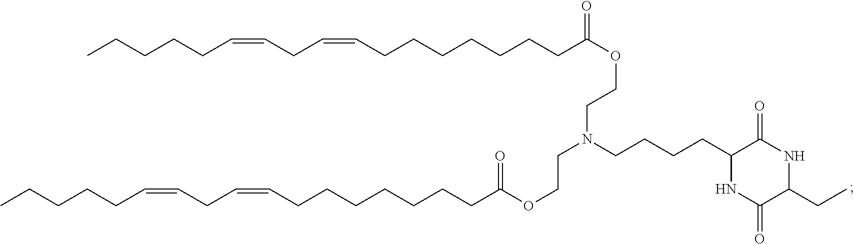 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC] |
| 20 | 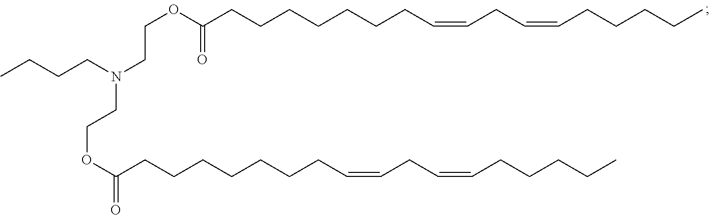 [SMILES: CCCCC/C=C\C/C=C\CCCCCCCC(=O)OCCN(CCCC)CCOC(=O)CCCCCCC/C=C\C/C=C\CCCCC] |
| 21 | 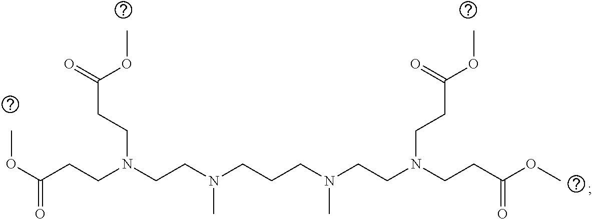 [SMILES: COC(=O)CCN(CCC(=O)OC)CCN(C)CCCN(C)CCN(CCC(=O)OC)CCC(=O)OC] |
| 22 | 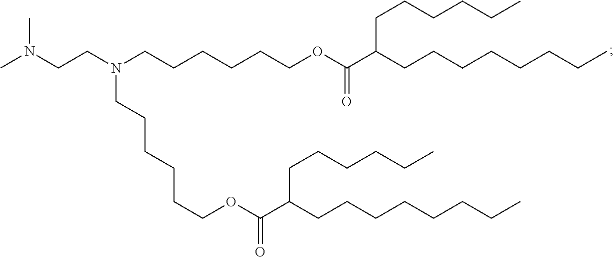 [SMILES: CCCCCCCCC(CCCCCC)C(=O)OCCCCCCN(CCCCCCOC(=O)C(CCCCCC)CCCCCCCC)CCN(C)C] |
| 23 | 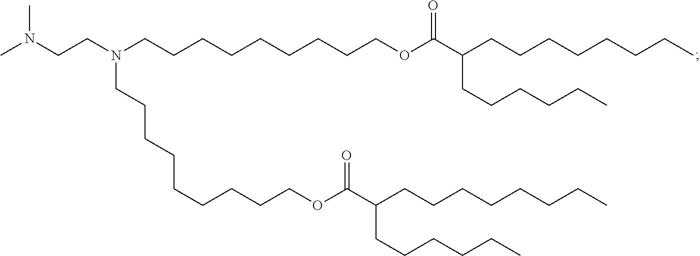 [SMILES: CCCCCCCCC(CCCCCC)C(=O)OCCCCCCCCCN(CCCCCCCCCOC(=O)C(CCCCCC)CCCCCCCC)CCN(C)C] |
| 24 | 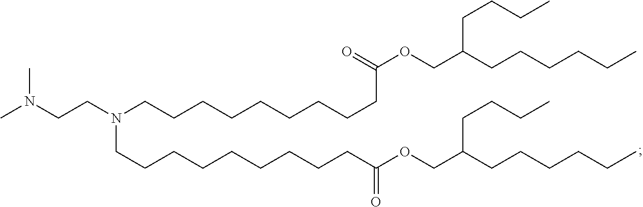 [SMILES: CCCCCCC(CCCC)COC(=O)CCCCCCCCCN(CCCCCCCCCC(=O)OCC(CCCC)CCCCCC)CCN(C)C] |
| 25 | 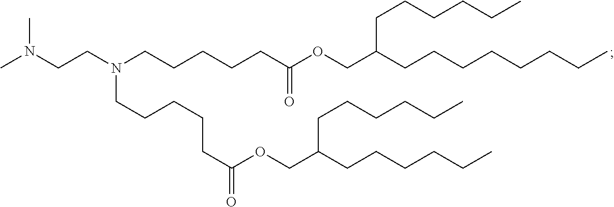 [SMILES: CCCCCCCCC(CCCCCC)COC(=O)CCCCCN(CCCCCC(=O)OCC(CCCCCC)CCCCCC)CCN(C)C] |
| 26 | 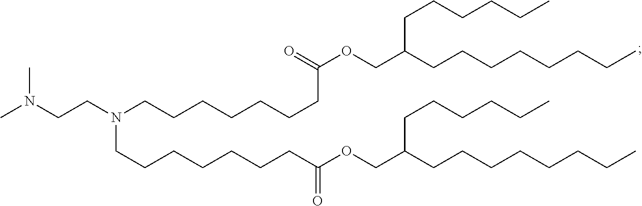 [SMILES: CCCCCCCCC(CCCCCC)COC(=O)CCCCCCCN(CCCCCCCC(=O)OCC(CCCCCC)CCCCCCCC)CCN(C)C] |
| 27 | 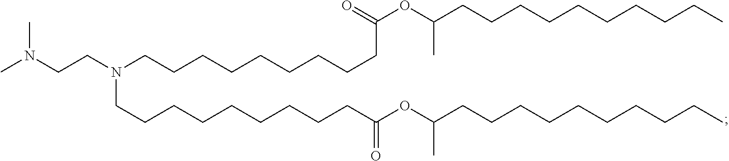 [SMILES: CCCCCCCCCCC(C)OC(=O)CCCCCCCCCN(CCCCCCCCCC(=O)OC(C)CCCCCCCCCC)CCN(C)C] |
| 28 | 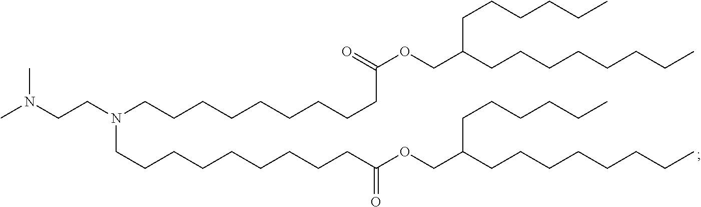 [SMILES: CCCCCCCCC(CCCCCC)COC(=O)CCCCCCCCCN(CCCCCCCCCC(=O)OCC(CCCCCC)CCCCCCCC)CCN(C)C] |
| 29 | 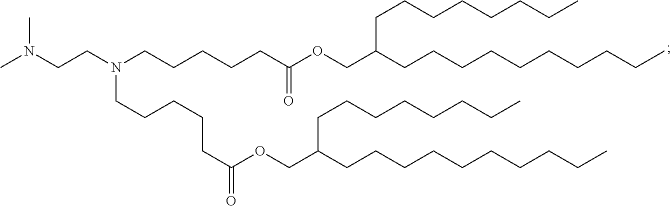 [SMILES: CCCCCCCCCCC(CCCCCCCC)COC(=O)CCCCCN(CCCCCC(=O)OCC(CCCCCCCC)CCCCCCCCCC)CCN(C)C] |
| 30 | 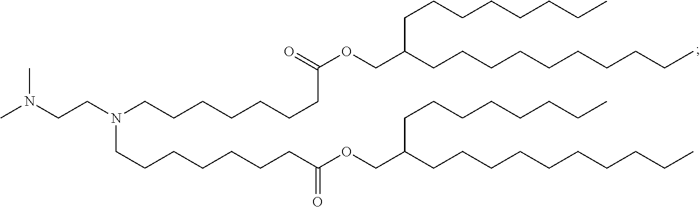 [SMILES: CCCCCCCCCCC(CCCCCCCC)COC(=O)CCCCCCCN(CCCCCCCC(=O)OCC(CCCCCCCC)CCCCCCCCCC)CCN(C)C] |
| 31 | 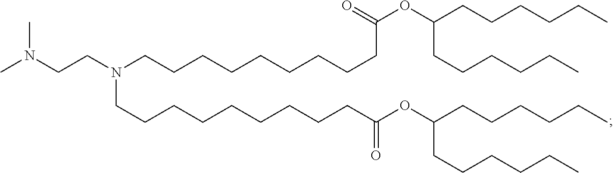 [SMILES: CCCCCCC(CCCCCC)OC(=O)CCCCCCCCCN(CCCCCCCCCC(=O)OC(CCCCCC)CCCCCC)CCN(C)C] |
| 32 | 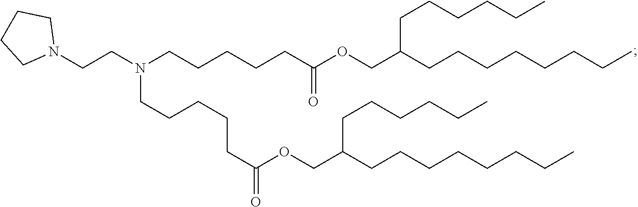 [SMILES: CCCCCCCCC(CCCCCC)COC(=O)CCCCCN(CCCCCC(=O)OCC(CCCCCC)CCCCCCCC)CCN1CCCC1] |
| 33 | 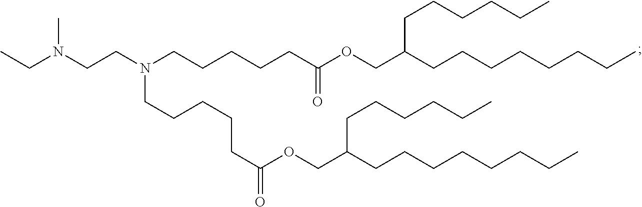 [SMILES: CCCCCCCCC(CCCCCC)COC(=O)CCCCCN(CCCCCC(=O)OCC(CCCCCC)CCCCCCCC)CCN(C)CC] |
| 34 | 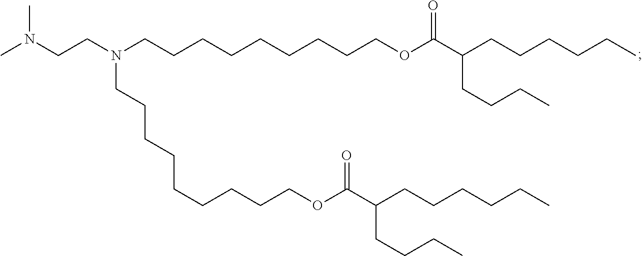 [SMILES: CCCCCCC(CCCC)C(=O)OCCCCCCCCCN(CCCCCCCCCOC(=O)C(CCCC)CCCCCC)CCN(C)C] |
| 35 | 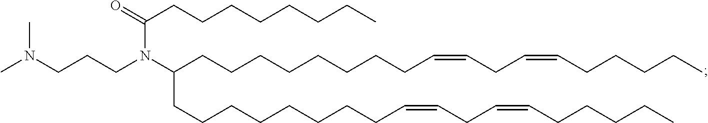 [SMILES: CCCCC/C=C\C/C=C\CCCCCCCCC(CCCCCCCC/C=C\C/C=C\CCCCC)N(CCCN(C)C)C(=O)CCCCCCCC] |
| 36 | 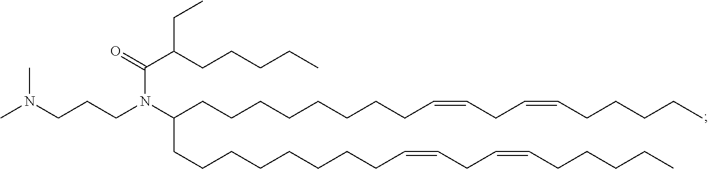 [SMILES: CCCCC/C=C\C/C=C\CCCCCCCCC(CCCCCCCC/C=C\C/C=C\CCCCC)N(CCCN(C)C)C(=O)C(CC)CCCCC] |
| 37 | 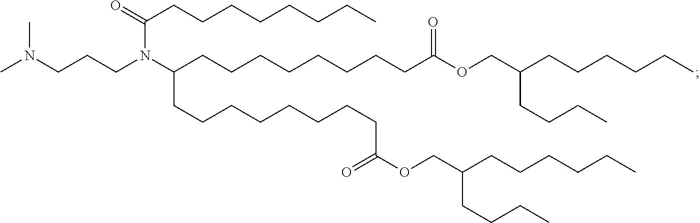 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] |
| 38 | 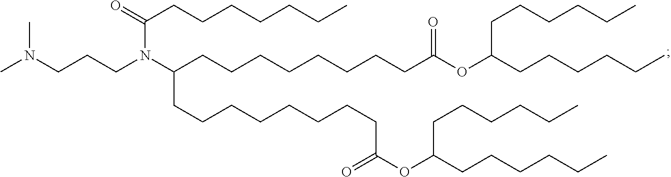 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |
| 39 | 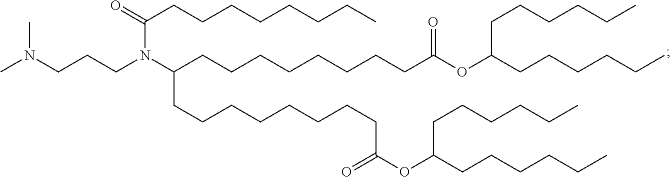 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC] |
| 40 | 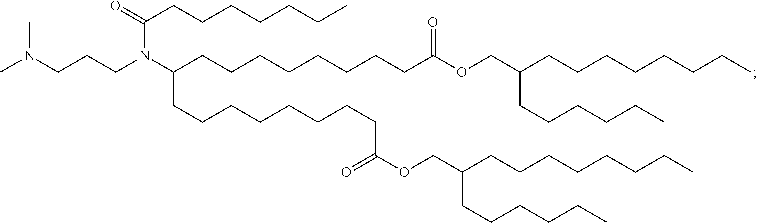 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] |
| 41 | 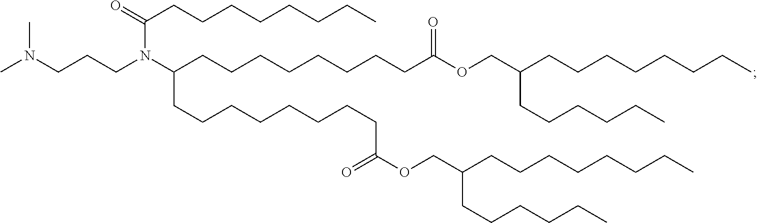 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] |
| 42 | 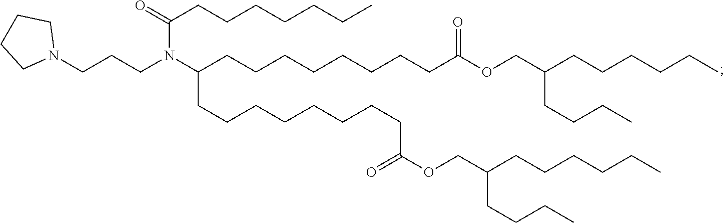 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] |
| 43 | 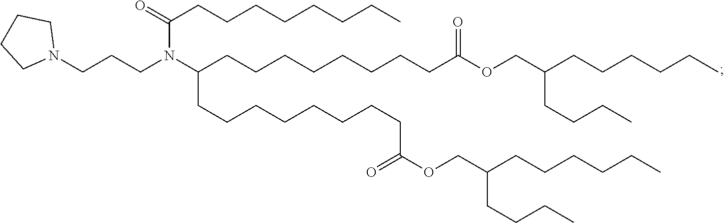 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] |
| 44 | 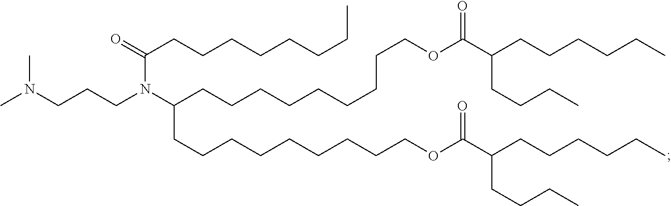 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] |
| 45 | 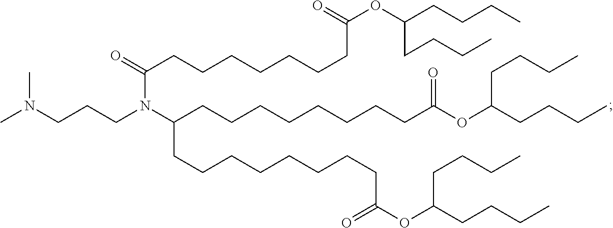 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] |
| 46 | 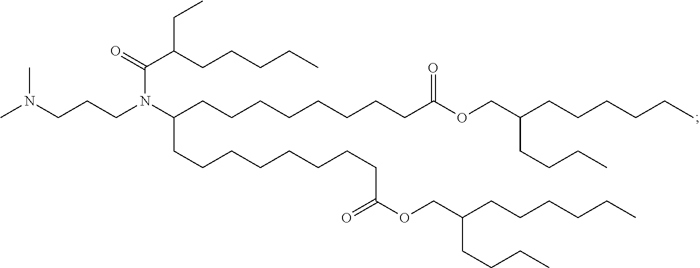 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC] |
| 47 | 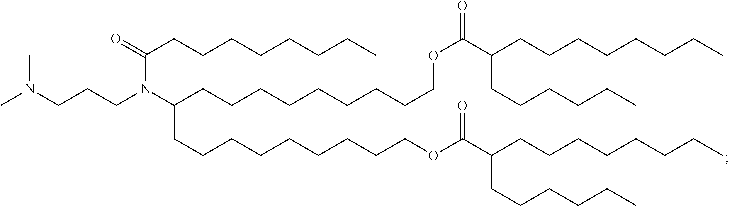 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC] |
| 48 | 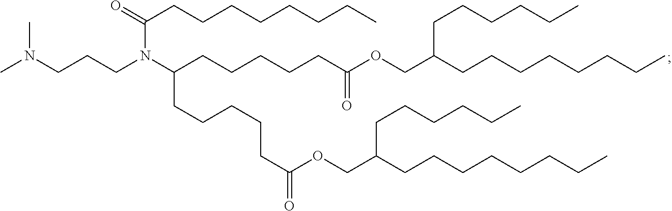 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC] |
| 49 | 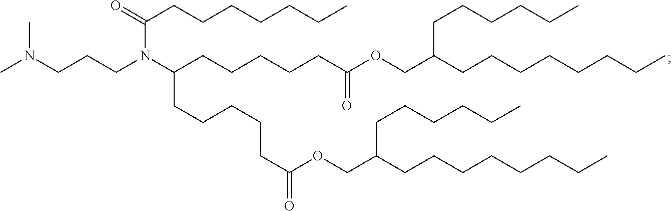 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC] |
| 50 | 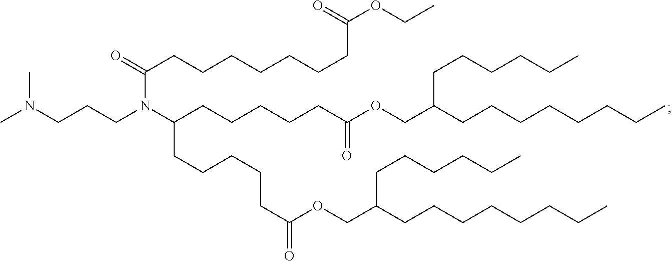 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC] |
| 51 | 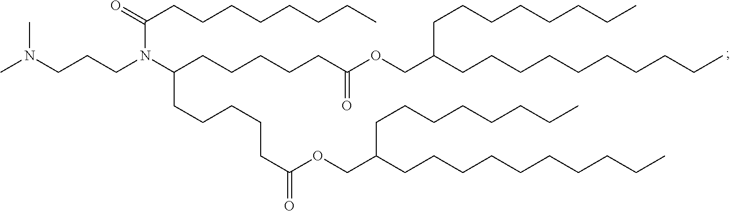 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC] |
| 52 | 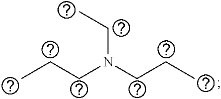 |
| 53 | 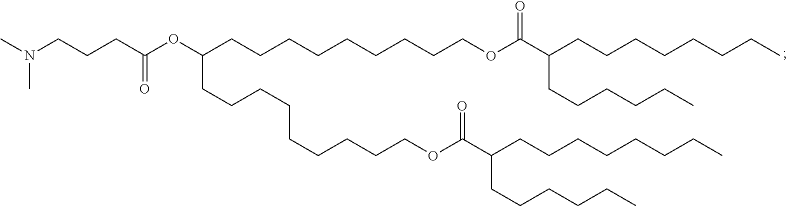 [SMILES: CCCCCCCCC(CCCCCC)C(=O)OCCCCCCCCCC(CCCCCCCCCOC(=O)C(CCCCCC)CCCCCCCC)OC(=O)CCCN(C)C] |
| 54 | 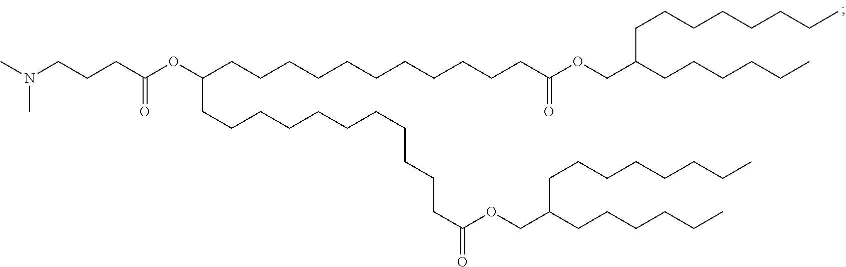 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C] |
| 55 | 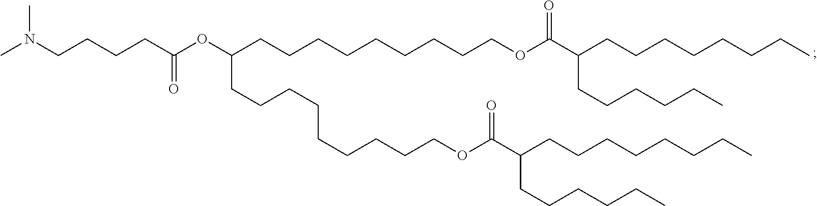 [SMILES: CCCCCCCCC(CCCCCC)C(=O)OCCCCCCCCCC(CCCCCCCCCOC(=O)C(CCCCCC)CCCCCCCC)OC(=O)CCCCN(C)C] |
| 56 | 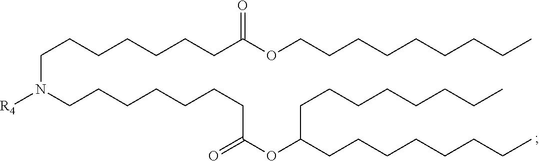 |
| 57 | 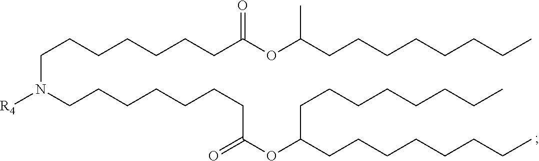 |
| 58 | 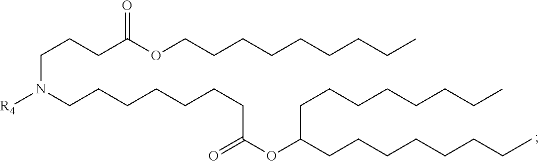 |
| 59 | 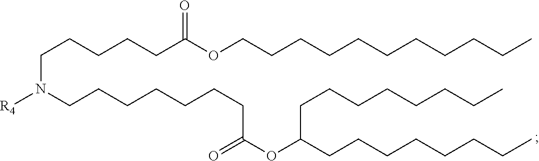 |
| 60 | 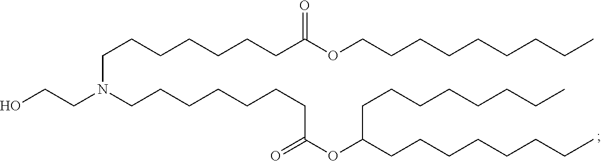 [SMILES: CCCCCCCCCOC(=O)CCCCCCCN(CCO)CCCCCCCC(=O)OC(CCCCCCCC)CCCCCCCC] |
| 61 | 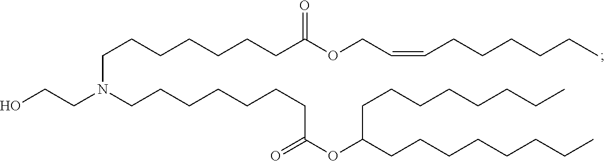 [SMILES: CCCCCC/C=C\COC(=O)CCCCCCCN(CCO)CCCCCCCC(=O)OC(CCCCCCCC)CCCCCCCC] |
| 62 | 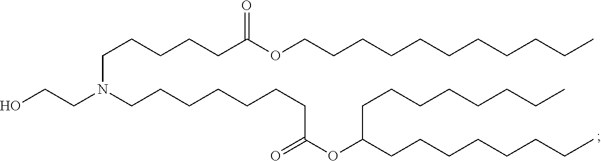 [SMILES: CCCCCCCCCCCOC(=O)CCCCCN(CCO)CCCCCCCC(=O)OC(CCCCCCCC)CCCCCCCC] |
| 63 | 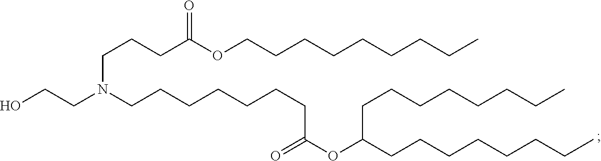 [SMILES: CCCCCCCCCOC(=O)CCCN(CCO)CCCCCCCC(=O)OC(CCCCCCCC)CCCCCCCC] |
| 64 | 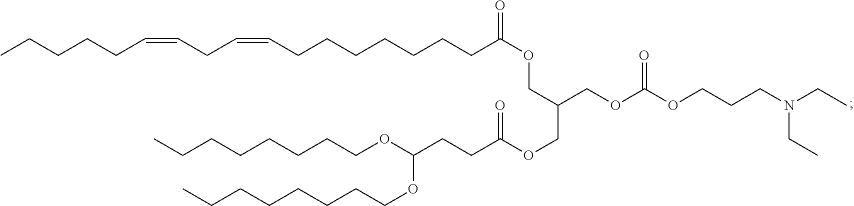 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 65 | 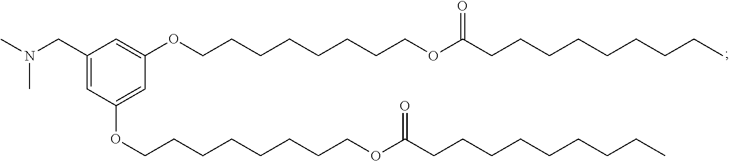 [SMILES: CCCCCCCCCC(=O)OCCCCCCCCOc1cc(CN(C)C)cc(OCCCCCCCCOC(=O)CCCCCCCCC)c1] |
| 66 | 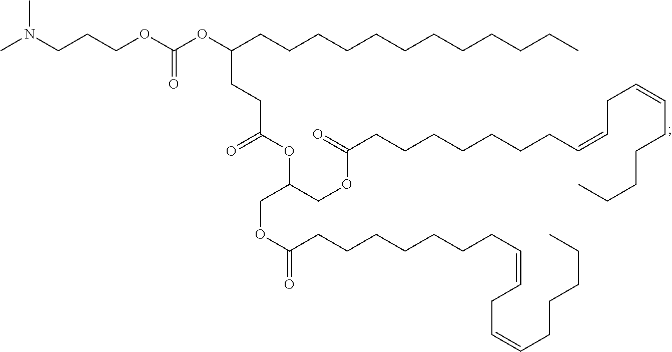 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C] |
| 67 | 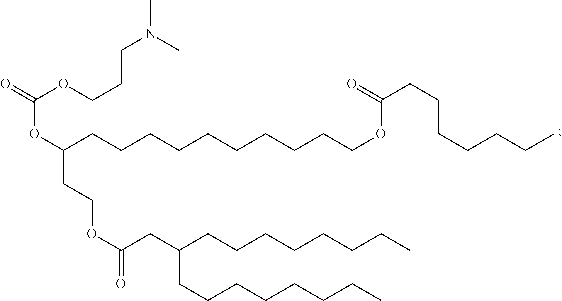 [SMILES: CCCCCCCCC(CCCCCCCC)CC(=O)OCCC(CCCCCCCCCCOC(=O)CCCCCCC)OC(=O)OCCCN(C)C] |
| 68 | 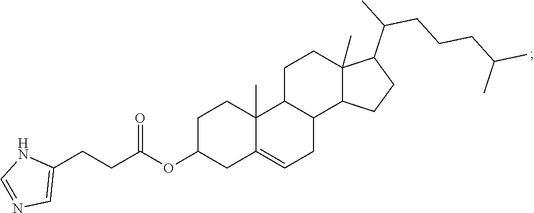 [SMILES: CC(C)CCCC(C)C1CCC2C3CC=C4CC(OC(=O)CCc5cnc[nH]5)CCC4(C)C3CCC12C] |
| 69 | 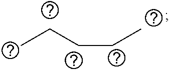 |
| 70 | 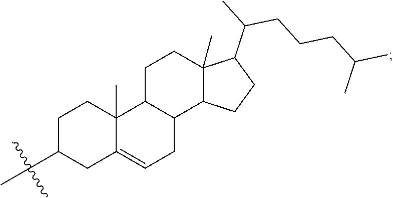 [SMILES: *C1CCC2(C)C(=CCC3C2CCC2(C)C(C(C)CCCC(C)C)CCC32)C1] |
| 71 | 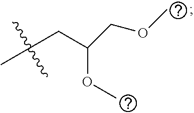 [SMILES: *CC(COC)OC] |
| 72 | 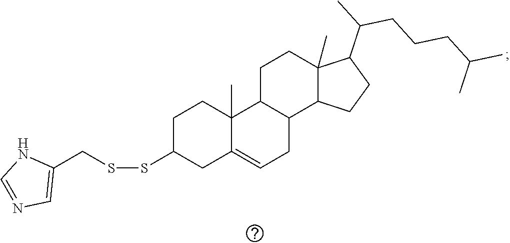 [SMILES: CC(C)CCCC(C)C1CCC2C3CC=C4CC(SSCc5cnc[nH]5)CCC4(C)C3CCC12C] |
| 73 | 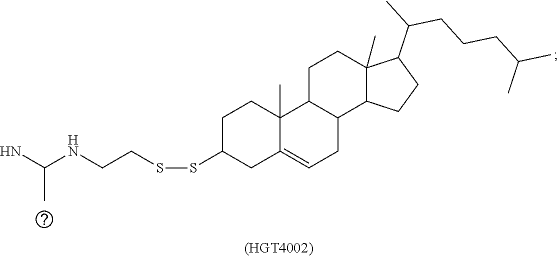 [SMILES: CC(C)CCCC(C)C1CCC2C3CC=C4CC(SSCCNC(C)N)CCC4(C)C3CCC12C] |
| 74 | 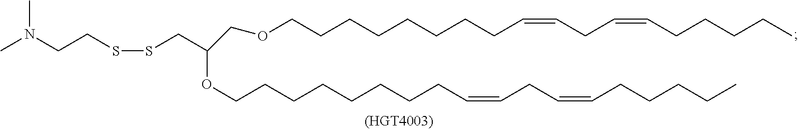 [SMILES: CCCCC/C=C\C/C=C\CCCCCCCCOCC(CSSCCN(C)C)OCCCCCCCC/C=C\C/C=C\CCCCC] |
| 75 | 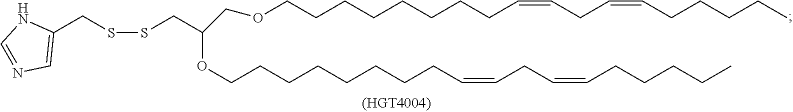 [SMILES: CCCCC/C=C\C/C=C\CCCCCCCCOCC(CSSCc1cnc[nH]1)OCCCCCCCC/C=C\C/C=C\CCCCC] |
| 76 | 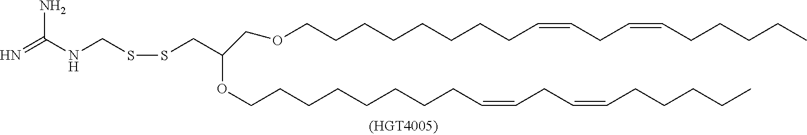 [SMILES: CCCCC/C=C\C/C=C\CCCCCCCCOCC(CSSCNC(=N)N)OCCCCCCCC/C=C\C/C=C\CCCCC] |

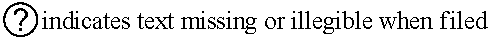

In some embodiments of the lipid composition of the present application, the ionizable cationic lipid is present in an amount from about from about 20 to about 23. In some embodiments, the molar percentage is from about 20, 20.5, 21, 21.5, 22, 22.5, to about 23 or any range derivable therein. In other embodiments, the molar percentage is from about 7.5 to about 20. In some embodiments, the molar percentage is from about 7.5, 8, 9, 10, 11, 12, 13, 14, 15, 16, 17, 18, 19, to about 20 or any range derivable therein.

In some embodiments of the lipid composition of the present application, said lipid composition comprises said ionizable cationic lipid at a molar percentage from about 5% to about 30%. In some embodiments of the lipid composition of the present application, said lipid composition comprises said ionizable cationic lipid at a molar percentage from about 10% to about 25%. In some embodiments of the lipid composition of the present application, said lipid composition comprises said ionizable cationic lipid at a molar percentage from about 15% to about 20%. In some embodiments of the lipid composition of the present application, said lipid composition comprises said ionizable cationic lipid at a molar percentage from about 10% to about 20%. In some embodiments of the lipid composition of the present application, said lipid composition comprises said ionizable cationic lipid at a molar percentage from about 20% to about 30%. In some embodiments of the lipid composition of the present application, said lipid composition comprises said ionizable cationic lipid at a molar percentage of at least (about) 5%, at least (about) 10%, at least (about) 15%, at least (about) 20%, at least (about) 25%, or at least (about) 30%. In some embodiments of the lipid composition of the present application, said lipid composition comprises said ionizable cationic lipid at a molar percentage of at most (about) 5%, at most (about) 10%, at most (about) 15%, at most (about) 20%, at most (about) 25%, or at most (about) 30%.

### Phospholipids or Other Zwitterionic Lipids

In some embodiments of the lipid composition of the present application, the lipid composition further comprises an additional lipid including but not limited to a steroid or a steroid derivative, a PEG lipid, and a phospholipid.

In some embodiments of the lipid composition of the present application, the lipid composition further comprises a phospholipid. In some embodiments, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some embodiments of the lipid compositions, the phospholipid is not an ethylphosphocholine.

In some embodiments of the lipid composition of the present application, the compositions may further comprise a molar percentage of the phospholipid to the total lipid composition from about 20 to about 23. In some embodiments, the molar percentage is from about 20, 20.5, 21, 21.5, 22, 22.5, to about 23 or any range derivable therein. In other embodiments, the molar percentage is from about 7.5 to about 60. In some embodiments, the molar percentage is from about 7.5, 8, 9, 10, 11, 12, 13, 14, 15, 16, 17, 18, 19, to about 20 or any range derivable therein.

In some embodiments of the lipid composition of the present application, said lipid composition comprises said phospholipid at a molar percentage from about 8% to about 23%. In some embodiments of the lipid composition of the present application, said lipid composition comprises said phospholipid at a molar percentage from about 10% to about 20%. In some embodiments of the lipid composition of the present application, said lipid composition comprises said phospholipid at a molar percentage from about 15% to about 20%. In some embodiments of the lipid composition of the present application, said lipid composition comprises said phospholipid at a molar percentage from about 8% to about 15%. In some embodiments of the lipid composition of the present application, said lipid composition comprises said phospholipid at a molar percentage from about 10% to about 15%. In some embodiments of the lipid composition of the present application, said lipid composition comprises said phospholipid at a molar percentage from about 12% to about 18%. In some embodiments of the lipid composition of the present application, said lipid composition comprises said phospholipid at a molar percentage of at least (about) 8%, at least (about) 10%, at least (about) 12%, at least (about) 15%, at least (about) 18%, at least (about) 20%, or at least (about) 23%. In some embodiments of the lipid composition of the present application, said lipid composition comprises said phospholipid at a molar percentage of at most (about) 8%, at most (about) 10%, at most (about) 12%, at most (about) 15%, at most (about) 18%, at most (about) 20%, or at most (about) 23%.

### Steroid or Steroid Derivatives

In some embodiments of the lipid composition of the present application, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

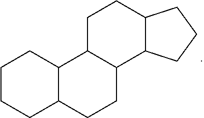 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

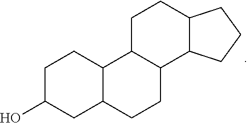 [SMILES: OC1CCC2C(CCC3C4CCCC4CCC23)C1]

In some embodiments of the present application, the steroid or steroid derivative is a cholestane or cholestane derivative. In a cholestane, the ring structure is further defined by the formula:

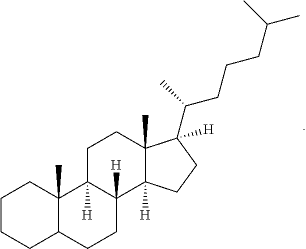

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

In some embodiments of the lipid composition, the compositions may further comprise a molar percentage of the steroid to the total lipid composition from about 40 to about 46. In some embodiments, the molar percentage is from about 40, 41, 42, 43, 44, 45, to about 46 or any range derivable therein. In other embodiments, the molar percentage of the steroid relative to the total lipid composition is from about 15 to about 40. In some embodiments, the molar percentage is 15, 16, 18, 20, 22, 24, 26, 28, 30, 32, 34, 36, 38, or 40, or any range derivable therein.

In some embodiments of the lipid composition of the present application, said lipid composition comprises said steroid or steroid derivative at a molar percentage from about 15% to about 46%. In some embodiments of the lipid composition of the present application, said lipid composition comprises said steroid or steroid derivative at a molar percentage from about 20% to about 40%. In some embodiments of the lipid composition of the present application, said lipid composition comprises said steroid or steroid derivative at a molar percentage from about 25% to about 35%. In some embodiments of the lipid composition of the present application, said lipid composition comprises said steroid or steroid derivative at a molar percentage from about 30% to about 40%. In some embodiments of the lipid composition of the present application, said lipid composition comprises said steroid or steroid derivative at a molar percentage from about 20% to about 30%. In some embodiments of the lipid composition of the present application, said lipid composition comprises said steroid or steroid derivative at a molar percentage of at least (about) 15%, of at least (about) 20%, of at least (about) 25%, of at least (about) 30%, of at least (about) 35%, of at least (about) 40%, of at least (about) 45%, or of at least (about) 46%. In some embodiments of the lipid composition of the present application, said lipid composition comprises said steroid or steroid derivative at a molar percentage of at most (about) 15%, of at most (about) 20%, of at most (about) 25%, of at most (about) 30%, of at most (about) 35%, of at most (about) 40%, of at most (about) 45%, or of at most (about) 46%.

### Polymer-Conjugated Lipids

In some embodiments of the lipid composition of the present application, the lipid composition further comprises a polymer conjugated lipid. In some embodiments, the polymer conjugated lipidis a PEG lipid. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid.

In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present application are taught by U.S. Pat. No. 5,820,873, WO 2010/141069, or U.S. Pat. No. 8,450,298, which is incorporated herein by reference.

In some embodiments of the lipid composition of the present application, the PEG lipid has a structural formula:

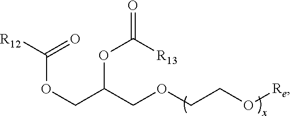

wherein: R₁₂ and R₁₃ are each independently alkyl_{(C≤24)}, alkenyl_{(C≤24)}, or a substituted version of either of these groups; Rₑ is hydrogen, alkyl_{(C≤18)}, or substituted alkyl_{(C≤18)}; and x is 1-250. In some embodiments, Rₑ is alkyl_{(C≤18)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments of the lipid composition of the present application, the PEG lipid has a structural formula:

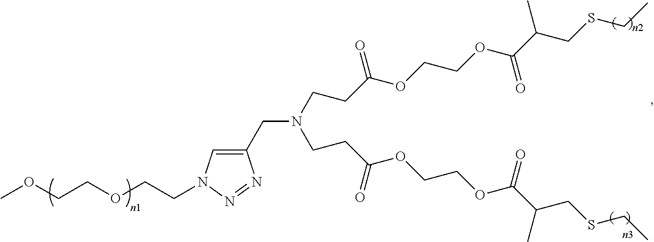

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments of the lipid composition of the present application, the compositions may further comprise a molar percentage of the PEG lipid to the total lipid composition from about 4.0 to about 4.6. In some embodiments, the molar percentage is from about 4.0, 4.1, 4.2, 4.3, 4.4, 4.5, to about 4.6 or any range derivable therein. In other embodiments, the molar percentage is from about 1.5 to about 4.0. In some embodiments, the molar percentage is from about 1.5, 1.75, 2, 2.25, 2.5, 2.75, 3, 3.25, 3.5, 3.75, to about 4.0 or any range derivable therein.

In some embodiments of the lipid composition of the present application, said lipid composition comprises said polymer-conjugated lipid at a molar percentage from about 0.5% to about 10%. In some embodiments of the lipid composition of the present application, said lipid composition comprises said polymer-conjugated lipid at a molar percentage from about 1% to about 8%. In some embodiments of the lipid composition of the present application, said lipid composition comprises said polymer-conjugated lipid at a molar percentage from about 2% to about 7%. In some embodiments of the lipid composition of the present application, said lipid composition comprises said polymer-conjugated lipid at a molar percentage from about 3% to about 5%. In some embodiments of the lipid composition of the present application, said lipid composition comprises said polymer-conjugated lipid at a molar percentage from about 5% to about 10%. In some embodiments of the lipid composition of the present application, said lipid composition comprises said polymer-conjugated lipid at a molar percentage of at least (about) 0.5%, at least (about) 1%, at least (about) 1.5%, at least (about) 2%, at least (about) 2.5%, at least (about) 3%, at least (about) 3.5%, at least (about) 4%, at least (about) 4.5%, at least (about) 5%, at least (about) 5.5%, at least (about) 6%, at least (about) 6.5%, at least (about) 7%, at least (about) 7.5%, at least (about) 8%, at least (about) 8.5%, at least (about) 9%, at least (about) 9.5%, or at least (about) 10%. In some embodiments of the lipid composition of the present application, said lipid composition comprises said polymer-conjugated lipid at a molar percentage of at most (about) 0.5%, at most (about) 1%, at most (about) 1.5%, at most (about) 2%, at most (about) 2.5%, at most (about) 3%, at most (about) 3.5%, at most (about) 4%, at most (about) 4.5%, at most (about) 5%, at most (about) 5.5%, at most (about) 6%, at most (about) 6.5%, at most (about) 7%, at most (about) 7.5%, at most (about) 8%, at most (about) 8.5%, at most (about) 9%, at most (about) 9.5%, or at most (about) 10%.

### Selective ORgan Targeting (SORT) Lipids

In some embodiments of the lipid composition of the present application, the lipid (e.g., nanoparticle) composition is preferentially delivered to a target organ. In some embodiments, the target organ is a lung, a lung tissue or a lung cell. As used herein, the term “preferentially delivered” is used to refer to a composition, upon being delivered, which is delivered to the target organ (e.g., lung), tissue, or cell in at least 25% (e.g., at least 30%, 35%, 40%, 45%, 50%, 55%, 60%, 65%, 70%, or 75%) of the amount administered.

In some embodiments of the lipid composition, the lipid composition comprises one or more selective organ targeting (SORT) lipid which leads to the selective delivery of the composition to a particular organ. In some embodiments, the SORT lipid may have two or more alkyl or alkenyl chains of C₆-C₂₄.

In some embodiments of the lipid compositions, the SORT lipid comprises permanently positively charged moiety. The permanently positively charged moiety may be positively charged at a physiological pH such that the SORT lipid comprises a positive charge upon delivery of a polynucleotide to a cell. In some embodiments the positively charged moiety is quaternary amine or quaternary ammonium ion. In some embodiments, the SORT lipid comprises, or is otherwise complexed to or interacting with, a counterion.

In some embodiments of the lipid compositions, the SORT lipid is a permanently cationic lipid (i.e., comprising one or more hydrophobic components and a permanently cationic group). The permanently cationic lipid may contain a group which has a positive charge regardless of the pH. One permanently cationic group that may be used in the permanently cationic lipid is a quaternary ammonium group. The permanently cationic lipid may comprise a structural formula:

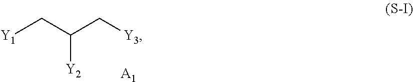

wherein:
Y₁, Y₂, or Y₃ are each independently X₁C(O)R₁ or X₂N⁺R₃R₄R₅;
provided at least one of Y₁, Y₂, and Y₃ is X₂N⁺R₃R₄R₅;
R₁ is C₁-C₂₄ alkyl, C₁-C₂₄ substituted alkyl, C₁-C₂₄ alkenyl, C₁-C₂₄ substituted alkenyl;
X₁ is O or NR^{a}, wherein Rₐ is hydrogen, C₁-C₄ alkyl, or C₁-C₄ substituted alkyl;
X₂ is C₁-C₆ alkanediyl or C₁-C₆ substituted alkanediyl; R₃, R₄, and R₅ are each independently C₁-C₂₄ alkyl, C₁-C₂₄ substituted alkyl, C₁-C₂₄ alkenyl, C₁-C₂₄ substituted alkenyl; and
A₁ is an anion with a charge equal to the number of X₂N⁺R₃R₄R₅ groups in the compound.

In some embodiments of the SORT lipids, the permanently cationic SORT lipid has a structural formula:

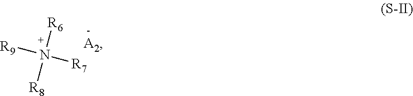

wherein:
R₆-R₉ are each independently C₁-C₂₄ alkyl, C₁-C₂₄ substituted alkyl, C₁-C₂₄ alkenyl, C₁-C₂₄ substituted alkenyl; provided at least one of R₆-R₉ is a group of C₈-C₂₄; and
A₂ is a monovalent anion.

In some embodiments of the lipid compositions, the SORT lipid comprises a head group of a particular structure. In some embodiments, the SORT lipid comprises a headgroup having a structural formula:

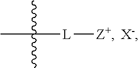

wherein L is a linker; Z⁺ is positively charged moiety and X is a counterion. In some embodiment, the linker is a biodegradable linker. The biodegradable linker may be degradable under physiological pH and temperature. The biodegradable linker may be degraded by proteins or enzymes from a subject. In some embodiments, the positively charged moiety is a quaternary ammonium ion or quaternary amine.

In some embodiments of the lipid compositions, the SORT lipid has a structural formula:

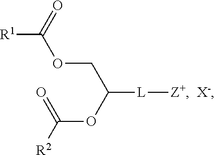

wherein R¹ and R² are each independently an optionally substituted C₆-C₂₄ alkyl, or an optionally substituted C₆-C₂₄ alkenyl.

In some embodiments of the lipid compositions, the SORT lipid has a structural formula:

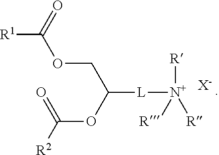

In some embodiments of the lipid compositions, the SORT lipid comprises a Linker (L). In some embodiments, L is

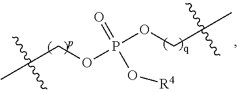

wherein:
p and q are each independently 1, 2, or 3; and
R⁴ is an optionally substituted C₁-C₆ alkyl

In some embodiments of the lipid compositions, the SORT lipid has a structural formula:

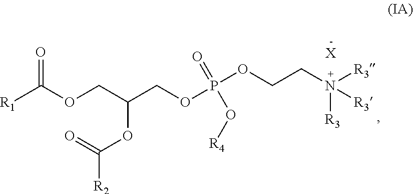

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
R₄ is alkyl_{(C≤6)} or substituted alkyl_{(C≤6)}; and
X⁻ is a monovalent anion.

In some embodiments of the lipid compositions, the SORT lipid is a phosphotidylcholine (e.g., 14:0 EPC). In some embodiments, the phophotidylcholine compound is further defined as:

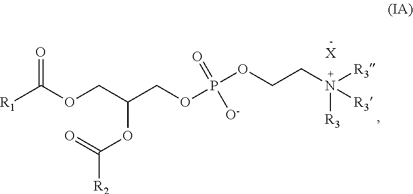

wherein:
R₁ and R₂ are each independently alkyl_{(C8-C24)}, alkenyl_{(C8-C24)}, or a substituted version of either group;
R₃, R₃′, and R₃^{″} are each independently alkyl_{(C≤6)} or substituted alkyl_{(C≤6)}; and
X⁻ is a monovalent anion.

In some embodiments of the lipid compositions, the SORT lipid is a phosphocholine lipid. In some embodiments, the SORT lipid is an ethylphosphocholine. The ethylphosphocholine may be, by way of example, without being limited to, 1,2-dimyristoleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, 1-palmitoyl-2-oleoyl-sn-glycero-3-ethylphosphocholine.

In some embodiments of the lipid compositions, the SORT lipid has a structural formula:

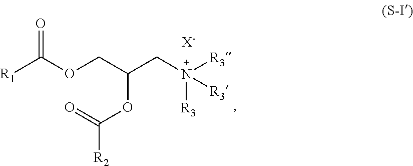

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
X⁻ is a monovalent anion.

By way of example, and without being limited thereto, a SORT lipid of the structural formula of the immediately preceding paragraph is 1,2-dioleoyl-3-trimethylammonium-propane (18:1 DOTAP) (e.g., chloride salt).

In some embodiments of the lipid compositions, the SORT lipid has a structural formula:

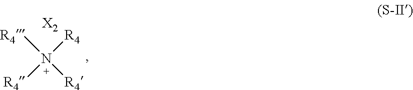

wherein:
R₄ and R₄′ are each independently alkyl_{(C6-C24)}, alkenyl_{(C6-C24)}, or a substituted version of either group;
R₄″ is alkyl_{(C≤24)}, alkenyl_{(C≤24)}, or a substituted version of either group;
R₄′″ is alkyl_{(C1-C8)}, alkenyl_{(C2-C8)}, or a substituted version of either group; and
X₂ is a monovalent anion.

By way of example, and without being limited thereto, a SORT lipid of the structural formula of the immediately preceding paragraph is dimethyldioctadecylammonium (DDAB) (e.g., bromide salt).

In some embodiments of the lipid compositions, the SORT lipid comprises one or more selected from the lipids set forth in Table 8.

TABLE 8
Example SORT lipids

| Lipid Name | Structure |
|---|---|
| 1,2-Dioleoyl-3-dimethylammonium- propane (18:1 DODAP) | 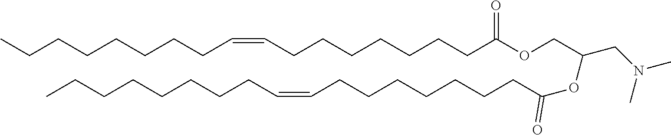 [SMILES: CCCCCCCC/C=C\CCCCCCCC(=O)OCC(CN(C)C)OC(=O)CCCCCCC/C=C\CCCCCCCC] |
| 1,2-Dioleoyl-3-trimethylammonium- propane (18:1 DOTAP) (e.g., chloride salt) | 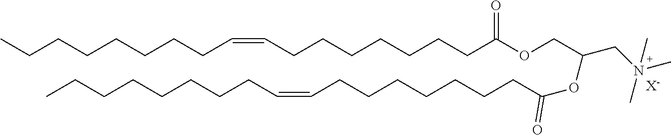 |
| 1,2-Di-O-octadecenyl-3- trimethylammonium propane (DOTMA) (e.g., chloride salt) | 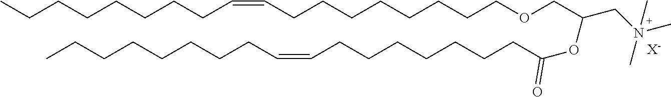 |
| Dimethyldioctadecylammonium (DDAB) (e.g., bromide salt) | 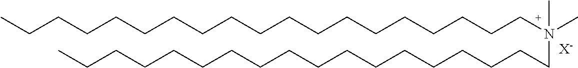 |
| 1,2-Dioleoyl-sn-glycero-3- ethylphosphocholine (14:0 EPC) (e.g., chloride salt) | 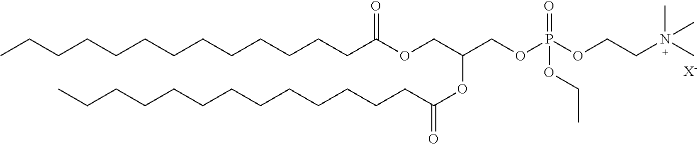 |

X⁻ is a counterion (e.g., Cl⁻, Br⁻, etc.)

In some embodiments of the lipid composition of the present application, said lipid composition comprises said SORT lipid at a molar percentage from about 20% to about 65%. In some embodiments of the lipid composition of the present application, said lipid composition comprises said SORT lipid at a molar percentage from about 25% to about 60%. In some embodiments of the lipid composition of the present application, said lipid composition comprises said SORT lipid at a molar percentage from about 30% to about 55%. In some embodiments of the lipid composition of the present application, said lipid composition comprises said SORT lipid at a molar percentage from about 20% to about 50%. In some embodiments of the lipid composition of the present application, said lipid composition comprises said SORT lipid at a molar percentage from about 30% to about 60%. In some embodiments of the lipid composition of the present application, said lipid composition comprises said SORT lipid at a molar percentage from about 25% to about 60%. In some embodiments of the lipid composition of the present application, said lipid composition comprises said SORT lipid at a molar percentage of at least (about) 25%, at least (about) 30%, at least (about) 35%, at least (about) 40%, at least (about) 45%, at least (about) 50%, at least (about) 55%, at least (about) 60%, or at least (about) 65%. In some embodiments of the lipid composition of the present application, said lipid composition comprises said SORT lipid at a molar percentage of at most (about) 25%, at most (about) 30%, at most (about) 35%, at most (about) 40%, at least (about) 45%, at most (about) 50%, at most (about) 55%, at most (about) 60%, or at most (about) 65%.

### SORT Formulations

In some embodiments, the lipid composition of the present disclosure comprises (i) an ionizable cationic lipid, (ii) a phospholipid, and (iii) a selective organ targeting (SORT) lipid separate from the ionizable cationic lipid and the phospholipid. In some embodiments, the lipid composition of the present disclosure comprises (i) an ionizable cationic lipid, (ii) a phospholipid, (iii) a selective organ targeting (SORT) lipid separate from the ionizable cationic lipid and the phospholipid, and (iv) a steroid or a steroid derivative thereof or a polymer-conjugated lipid. In some embodiments, the lipid composition of the present disclosure comprises (i) an ionizable cationic lipid, (ii) a phospholipid, (iii) a selective organ targeting (SORT) lipid separate from the ionizable cationic lipid and the phospholipid, (iv) a steroid or a steroid derivative thereof, and (v) a polymer-conjugated lipid. In some embodiments, the ionizable cationic lipid is a dendrimer or dendron. In some embodiments, the ionizable cationic lipid comprises an ammonium group which is positively charged at physiological pH and contains at least two hydrophobic groups. In some embodiments, the ammonium group is positively charged at a pH from about 6 to about 8. In some embodiments, the ionizable cationic lipid is a dendrimer or dendron. In some embodiments, the ionizable cationic lipid comprises at least two C₆-C₂₄ alkyl or alkenyl groups. In some embodiments of the SORT formulations, the phospholipid is not an ethylphosphocholine.

In some embodiments of the SORT formulations, the selective organ targeting (SORT) compound is present in the composition in a molar ratio from about 2% to about 70%, or any range derivable therein.

In some embodiments, the components of the (e.g., pharmaceutical) composition or the lipid composition are present at a particular molar percentage or range of molar percentages. In some embodiments, a component of the lipid composition is present at a molar percentage of at least 5%, 10%, 15, 20%, 25%, 30%, 35%, 40%, 45%, 50%, 55%, 60%, 65%, 70%, 75%, 80%, 85%, 90%, 95%, or more. In some embodiments, a component of the lipid composition is present at a molar percentage of at no more than 1%, 5%, 10%, 15, 20%, 25%, 30%, 35%, 40%, 45%, 50%, 55%, 60%, 65%, 70%, 75%, 80%, 85%, 90%, 95%, or less. In some embodiments, the lipid composition comprises the SORT lipid at a molar percentage from about 20% to about 65%. In some embodiments, the lipid composition comprises said ionizable cationic lipid at a molar percentage from about 5% to about 30%. In some embodiments, the lipid composition comprises a phospholipid at a molar percentage from about 8% to about 23%.

In some embodiments, the lipid composition comprises a steroid or steroid derivative. In some embodiments, the steroid or steroid derivative is at a molar percentage of about 15%. In some embodiments, the steroid or steroid derivative is at a molar percentage from about 15% to about 46%. In some embodiments, the steroid or steroid derivative is at a molar percentage of 15% or greater. In some embodiments, the steroid or steroid derivative is at a molar percentage of 46% or less. In some embodiments, the lipid composition further comprises a polymer-conjugated lipid. In some embodiments, the polymer-conjugated lipid is a poly(ethylene glycol) (PEG)-conjugated lipid). In some embodiments, the polymer-conjugated lipid is at a molar percentage of about 0.5%. In some embodiments, the polymer-conjugated lipid is at a molar percentage of about 10%. In some embodiments, the polymer-conjugated lipid is at a molar percentage from about 0.5% to 10%. In some embodiments, the polymer-conjugated lipid is at a molar percentage of 0.5% or greater. In some embodiments, the polymer-conjugated lipid is at a molar percentage of 10% or less.

Provided herein are (e.g., pharmaceutical) composition s that comprise components that allow for an improved efficacy or outcome based on the delivery of the polynucleotide. The compositions described elsewhere herein may be more effective at delivery to a particular cell, cell type, organ, or bodily region as compared to a reference composition or compound. The compositions described elsewhere herein may be more effective at generating increase expression of a corresponding polypeptide of a delivered polynucleotide. The compositions described elsewhere herein may be more effective at generating a larger number of cells that express a corresponding polypeptide of a delivered polynucleotide. The compositions described elsewhere herein may result in an increase uptake of the polynucleotide as compared to a reference polynucleotide. The increased uptake may be result of improved stability of polynucleotide or an improved targeting of the composition to a particular cell type or organ. In some embodiments, the SORT lipid is present in an amount in the lipid composition to effect a greater expression or activity of the polynucleotide (or corresponding polypeptide of the polynucleotide) in a cell compared to that achieved with a reference lipid composition comprising 13,16,20-tris(2-hydroxydodecyl)-13,16,20,23-tetraazapentatricontane-11,25-diol (“LF92”), a phospholipid, cholesterol, and a PEG-lipid. In some embodiments, the SORT lipid is present in an amount in the lipid composition to effect at least a 1.1 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 2-fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 5 fold greater expression or activity of the polynucleotide (or corresponding polypeptide of the polynucleotide) in a cell compared to that achieved with a reference lipid composition comprising LF92, a phospholipid, cholesterol, and a PEG-lipid. In some embodiments, the SORT lipid is present in an amount in the lipid composition to effect at least a 10-fold greater expression or activity of the polynucleotide (or corresponding polypeptide of the polynucleotide) in a cell compared to that achieved with a reference lipid composition comprising LF92, a phospholipid, cholesterol, and a PEG-lipid.

In some embodiments, the SORT lipid is present in an amount in said lipid composition to effect an expression or activity of said polynucleotide (or corresponding polypeptide of the polynucleotide) in a greater plurality of cells compared to that achieved with a reference lipid composition comprising LF92, a phospholipid, cholesterol, and a PEG-lipid. In some embodiments, the SORT lipid is present in an amount in said lipid composition to effect an expression or activity of said polynucleotide (or corresponding polypeptide of the polynucleotide) in at least a 1.1-fold greater plurality of cells compared to that achieved with a reference lipid composition comprising LF92, a phospholipid, cholesterol, and a PEG-lipid. In some embodiments, the SORT lipid is present in an amount in said lipid composition to effect an expression or activity of said polynucleotide (or corresponding polypeptide of the polynucleotide) in at least a 2-fold greater plurality of cells compared to that achieved with a reference lipid composition comprising LF92, a phospholipid, cholesterol, and a PEG-lipid. In some embodiments, the SORT lipid is present in an amount in said lipid composition to effect an expression or activity of said polynucleotide (or corresponding polypeptide of the polynucleotide) in at least a 5-fold greater plurality of cells compared to that achieved with a reference lipid composition comprising LF92, a phospholipid, cholesterol, and a PEG-lipid. In some embodiments, the SORT lipid is present in an amount in said lipid composition to effect an expression or activity of said polynucleotide (or corresponding polypeptide of the polynucleotide) in at least a 10-fold greater plurality of cells compared to that achieved with a reference lipid composition comprising LF92, a phospholipid, cholesterol, and a PEG-lipid.

In some embodiments, the SORT lipid is present in an amount in said lipid composition to effect an uptake of the polynucleotide in a greater plurality of cells compared to that achieved with a reference lipid composition comprising LF92, a phospholipid, cholesterol, and a PEG-lipid. In some embodiments, the SORT lipid is present in an amount in said lipid composition to effect an uptake of the polynucleotide in a greater amount to a cell compared to that achieved with a reference lipid composition comprising LF92, a phospholipid, cholesterol, and a PEG-lipid.

### Pharmaceutical Compositions

Some embodiments of the (e.g., pharmaceutical) composition disclosed herein comprise a particular molar ratio of the components or atoms. In some embodiments, the (e.g., pharmaceutical) composition comprises a particular molar ratio of nitrogen in the lipid composition to the phosphate in the polynucleotide (N/P ratio). In some embodiments, the molar ratio of nitrogen in the lipid composition to phosphate in said polynucleotide (N/P ratio) is no more than about 20:1. In some embodiments, the N/P ratio is from about 5:1 to about 50:1.

In some embodiments, composition comprises a particular molar ratio of said polynucleotide to total lipids of said lipid composition. In some embodiments, the molar ratio of said polynucleotide to total lipids of said lipid composition is no more than about 1:1, 1:10, 1:50, or 1:100.

In some embodiments, the lipid composition comprises a plurality of particles. The plurality of particles may be characterized by a particular size. For example, the plurality of particles may have an average size. In some embodiments the lipid composition comprises a plurality of particles characterized by a size (e.g. average size) of 100 nanometers (nm) or less. The plurality of particles may be characterized by a size of no more than 10 nm, 20 nm, 30 nm, 40 nm, 50 nm, 60 nm, 70 nm, 80 nm, 90 nm, 100 nm or less. The plurality of particles may be characterized by a size of at least 10 nm, 20 nm, 30 nm, 40 nm, 50 nm, 60 nm, 70 nm, 80 nm, 90 nm, 100 nm or more. The plurality of particles may be characterized by a size of any one of the following values or within a range of any two of the following values: 10 nm, 20 nm, 30 nm, 40 nm, 50 nm, 60 nm, 70 nm, 80 nm, 90 nm, and 100 nm.

In some embodiments, the plurality of particles may be characterized by a particular polydispersity index (PDI) In some embodiments, the lipid composition comprises a plurality of particles characterized by a polydispersity index (PDI) of no more than about 0.2.

In some embodiments, the plurality of particles may be characterized by a particular negative zeta potential. In some embodiments, the lipid composition comprises a plurality of particles characterized by a negative zeta potential of −10 millivolts (mV) to 10 mV.

The particles of the lipid composition may encapsulate other components of the (e.g., pharmaceutical) composition. In some embodiments, the polynucleotide is encapsulated in particles of the lipid composition.

In some embodiments (especially of the SORT formulations), the lipid composition (with or without polynucleotide(s) coupled therewith) comprises particular physical characteristic(s). For example, the lipid composition may comprise an apparent ionization constant (pKa). In some embodiments, the lipid composition has an (pKa) is of about 8 or higher. In some embodiments, the lipid composition has an (pKa) is within a range of 8 to 13. In some embodiments, the lipid composition has an (pKa) is of 13 or less.

In some embodiments, the (e.g., pharmaceutical) composition comprises one or more pharmaceutically acceptable excipients.

In some embodiments, the (e.g., pharmaceutical) composition can be administered subcutaneously, orally, intramuscularly, or intravenously. In one embodiment, the (e.g., pharmaceutical) composition is administered at a therapeutically effective dose.

### Kits

Provided herein, in some embodiments, is a kit comprising a (e.g., pharmaceutical) composition described herein, a container, and a label or package insert on or associated with the container.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A (e.g., unsaturated) dendrimer of a generation (g) having a structural formula:

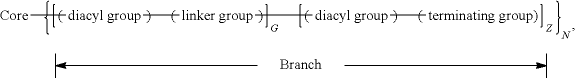

or a pharmaceutically acceptable salt thereof, wherein:
(a) the core comprises a structural formula (X_{Core}):

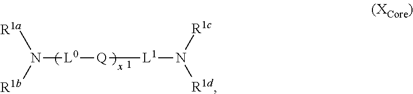

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}).

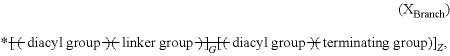

wherein:
   * indicates a point of attachment of the branch to the core;
   g is 1, 2, 3, or 4;
   Z=2^{(g-1)};
   G=0, when g=1; or G=Σᵢ₌₀^{i=g-2}2ⁱ, when g≠1;
(c) each diacyl group independently comprises a structural formula

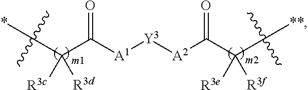

wherein:
   *indicates a point of attachment of the diacyl group at the proximal end thereof,
   ** indicates a point of attachment of the diacyl group at the distal end thereof,
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein: R⁴ is hydrogen or optionally substituted (e.g., C₁-C₆) alkyl;
   m¹ and m² are each independently at each occurrence 1, 2, or 3; and
   R^{3c}, R^{3d}, R^{3e}, and R^{3f} are each independently at each occurrence hydrogen or an optionally substituted (e.g., C₁-C₈) alkyl; and
(d) each linker group independently comprises a structural formula

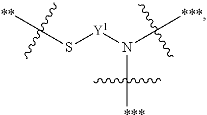

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is R independently at each occurrence selected from C₆-C₂₂ alkenyl (e.g., linear or branched) (e.g., having one or more double bounds), C₆-C₂₂ alkadienyl, and C₆-C₂₂ alkatrienyl.

Embodiment 2. The dendrimer of Embodiment 1, wherein x¹ is 0, 1, 2, or 3.

Embodiment 3. The dendrimer of Embodiment 1 or 2, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl

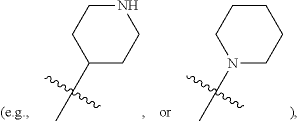 [SMILES: *C1CCNCC1 ; *N1CCCCC1]

N—(C₁-C₃ alkyl)-piperidinyl

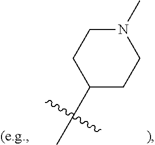 [SMILES: *C1CCN(C)CC1]

piperazinyl

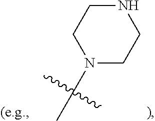 [SMILES: *N1CCNCC1]

N—(C₁-C₃ alkyl)-piperadizinyl

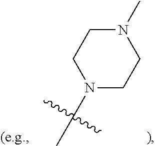 [SMILES: *N1CCN(C)CC1]

morpholinyl

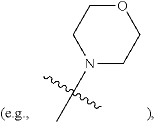 [SMILES: *N1CCOCC1]

### N-pyrrolidinyl

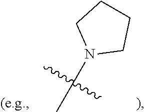 [SMILES: *N1CCCC1]

pyrrolidinyl

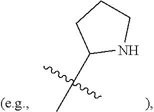 [SMILES: *C1CCCN1]

or N—(C₁-C₃ alkyl)-pyrrolidinyl

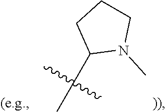 [SMILES: *C1CCCN1C]

(e.g., C₆-C₁₀) aryl, and C₃-C₅ heteroaryl (e.g., imidazolyl

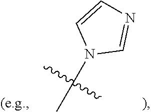 [SMILES: *n1ccnc1]

or pyridinyl

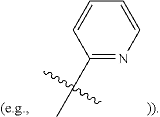 [SMILES: *c1ccccn1]

Embodiment 4. The dendrimer of Embodiment 3, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

Embodiment 5. The dendrimer of any one of Embodiments 1-4, wherein R^{3a} and R^{3b} are each independently at each occurrence hydrogen.

Embodiment 6. The dendrimer of any one of Embodiments 1-5, wherein the plurality (N) of branches comprises at least 2 (e.g., at least 3, at least 4, at least 5, or at least 6) branches.

Embodiment 7. The dendrimer of any one of Embodiments 1-5, wherein the plurality (N) of branches comprises from 2 to 6 (e.g., from 3 to 6, or from 4 to 6) branches.

Embodiment 8. The dendrimer of any one of Embodiments 1-7, wherein g=1; G=0; and Z=1.

Embodiment 9. The dendrimer of Embodiment 8, wherein each branch of the plurality of branches comprises a structural formula * [symbol] diacyl group [symbol] terminating group)

Embodiment 10. The dendrimer of any one of Embodiments 1-7, wherein g=2; G=1; and Z=2.

Embodiment 11. The dendrimer of Embodiment 10, wherein each branch of the plurality of branches comprises a structural formula

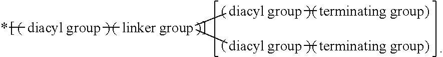

Embodiment 12. The dendrimer of any one of Embodiments 1-7, wherein g=3; G=3; and Z=4.

Embodiment 13. The dendrimer of Embodiment 12, wherein each branch of the plurality of branches comprises a structural formula

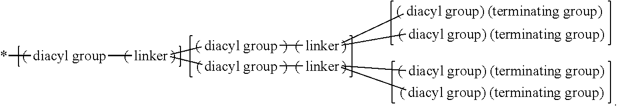

Embodiment 14. The dendrimer of any one of Embodiments 1-7, wherein g=4; G=7; and Z=8.

Embodiment 15. The dendrimer of Embodiment 14, wherein each branch of the plurality of branches comprises a structural formula:

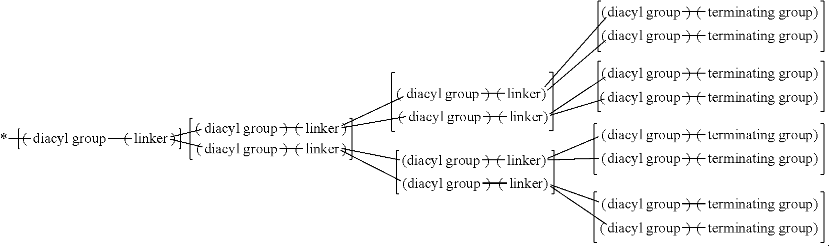

Embodiment 16. The dendrimer of any one of Embodiments 1-15, wherein the core comprises a structural formula:

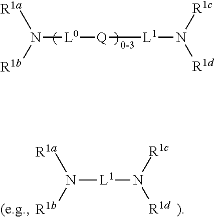

Embodiment 17. The dendrimer of any one of Embodiments 1-15, wherein the core comprises a structural formula:

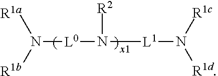

Embodiment 18. The dendrimer of Embodiment 17, wherein the core comprises a structural formula:

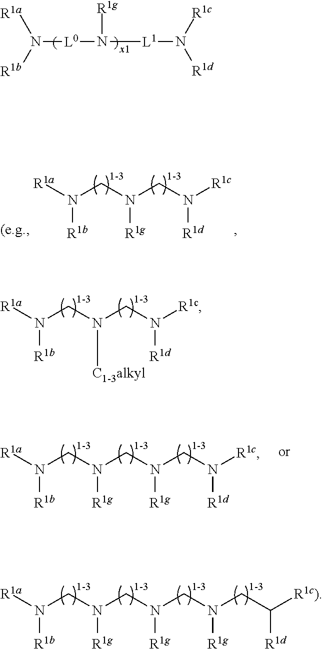

Embodiment 19. The dendrimer of Embodiment 17, wherein the core comprises a structural formula:

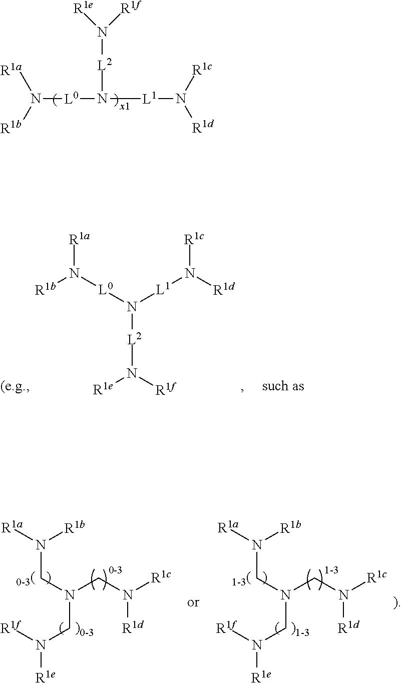

Embodiment 20. The dendrimer of any one of Embodiments 1-15, wherein the core comprises a structural formula:

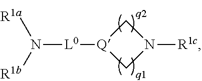

wherein Q′ is —NR²— or —CR^{3a}R^{3b}—; q¹ and q² are each independently 1 or 2.

Embodiment 21. The dendrimer of Embodiment 20, wherein the core comprises a structural formula:

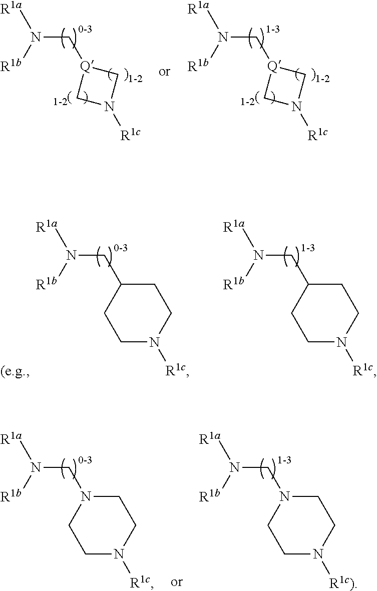

Embodiment 22. The dendrimer of any one of Embodiments 1-15, wherein the core comprises a structural formula

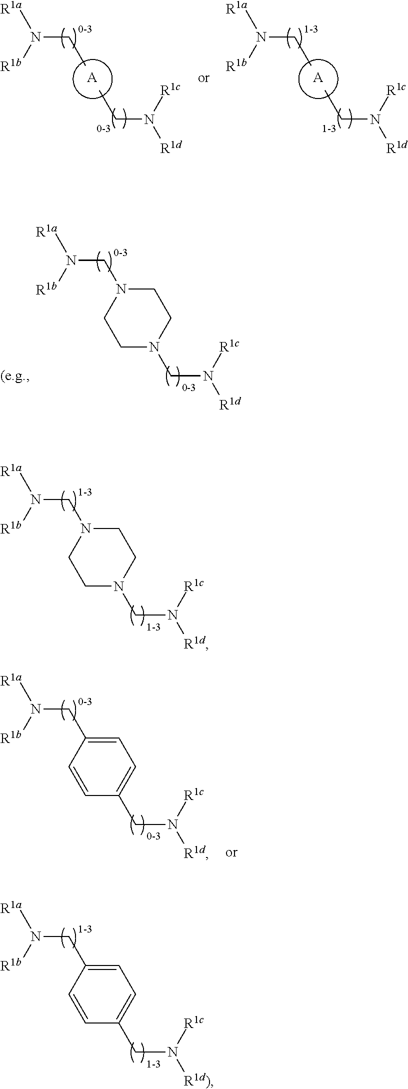

wherein ring A is an optionally substituted aryl or an optionally substituted (e.g., C₃-C₁₂, such as C₃-C₅) heteroaryl.

Embodiment 23. The dendrimer of any one of Embodiments 1-15, wherein the core comprises a structural formal

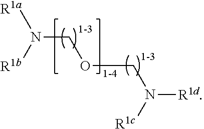

Embodiment 24. The dendrimer of any one of Embodiments 1-15, wherein the core comprises a structural formula selected from the group consisting of:

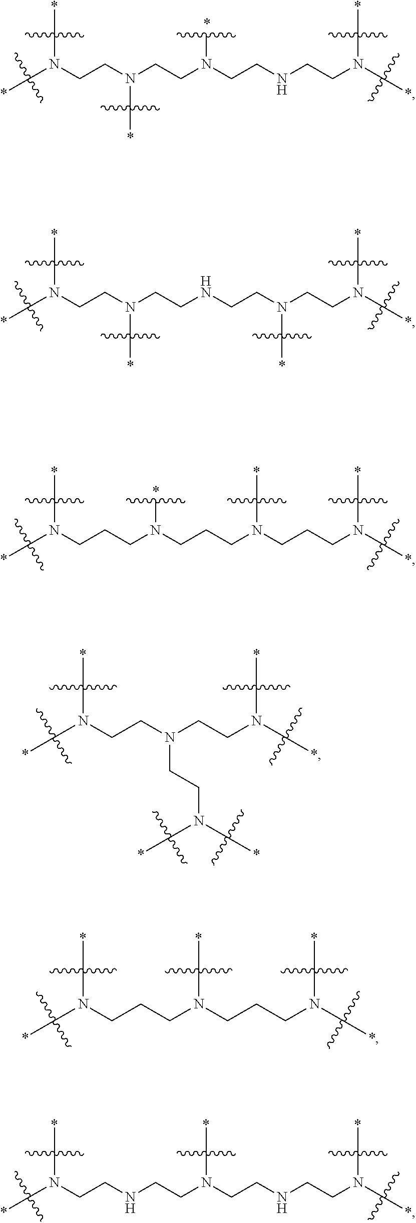

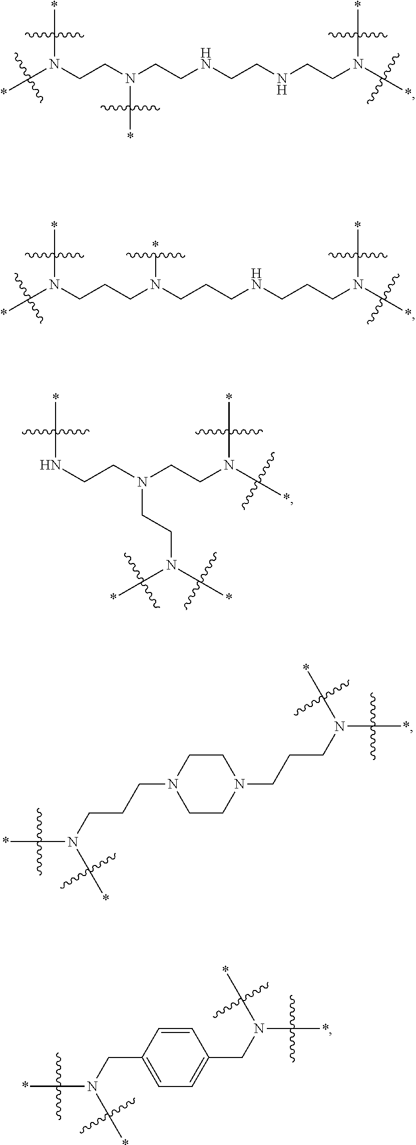

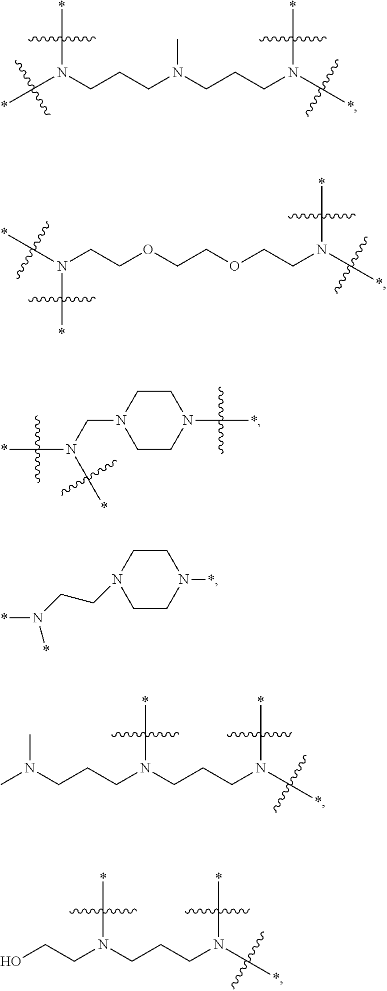

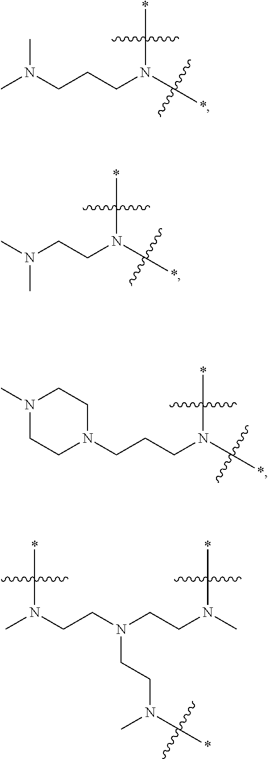

and pharmaceutically acceptable salts thereof, wherein * indicates a point of attachment of the core to a branch of the plurality of branches.

Embodiment 25. The dendrimer of Embodiment 24, wherein the core comprises a structural formula selected from the group consisting of:

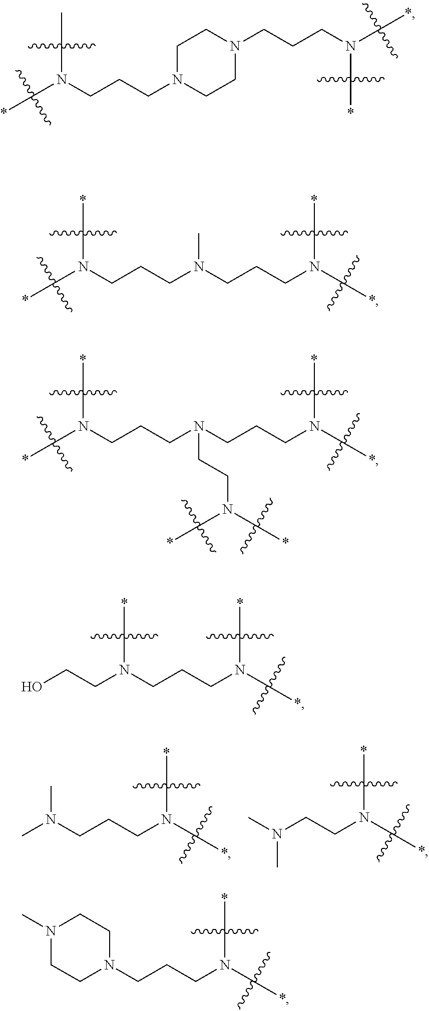

and pharmaceutically acceptable salts thereof, wherein * indicates a point of attachment of the core to a branch of the plurality of branches.

Embodiment 26. The dendrimer of Embodiment 24, wherein the core comprises a structural formula selected from the group consisting of:

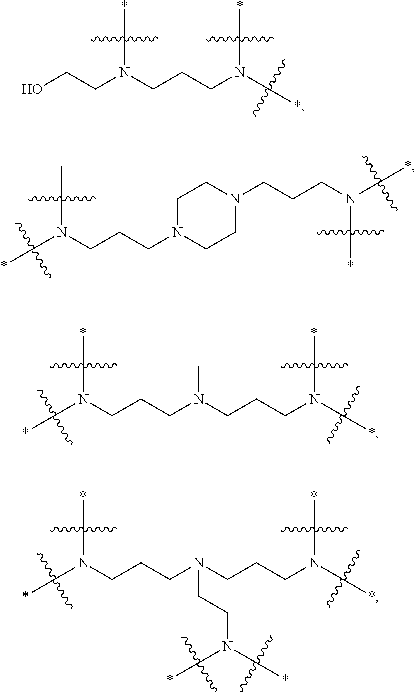

and pharmaceutically acceptable salts thereof, wherein * indicates a point of attachment of the core to a branch of the plurality of branches.

Embodiment 27. The dendrimer of Embodiment 24, wherein the core comprises a structural formula

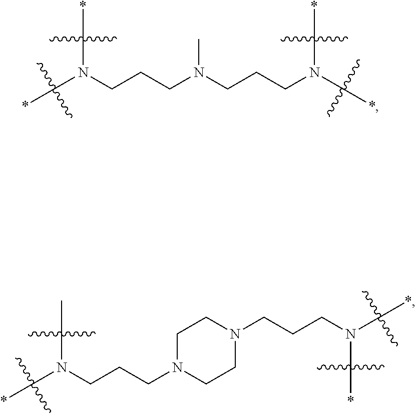

or a pharmaceutically acceptable salt thereof, wherein * indicates a point of attachment of the core to a branch of the plurality of branches.

Embodiment 28. The dendrimer of Embodiment 24, wherein the core comprises a structural formula

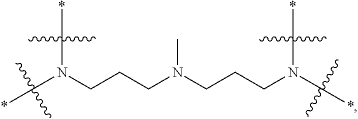

or a pharmaceutically acceptable salt thereof, wherein * indicates a point of attachment of the core to a branch of the plurality of branches.

Embodiment 29. The dendrimer of any one of Embodiments 1-28, wherein A¹ is —O— or —NH—.

Embodiment 30. The dendrimer of Embodiment 29, wherein A¹ is —O—.

Embodiment 31. The dendrimer of any one of Embodiments 1-30, wherein A² is —O— or —NH—.

Embodiment 32. The dendrimer of any Embodiment 31, wherein A² is —O—.

Embodiment 33. The dendrimer of any one of Embodiments 1-32, wherein Y³ is C₁-C₁₂ (e.g., C₁-C₆, such as C₁-C₃) alkylene.

Embodiment 34. The dendrimer of any one of Embodiments 1-33, wherein the diacyl group independently at each occurrence comprises a structural formula

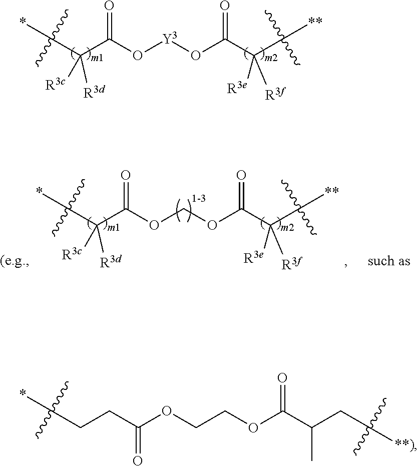

optionally wherein R^{3c}, R^{3d}, R^{3e}, and R^{3f} are each independently at each occurrence hydrogen or C₁-C₃ alkyl.

Embodiment 35. The dendrimer of any one of Embodiments 1-34, wherein L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

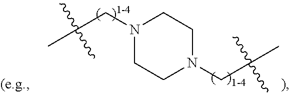

and [(C₁-C₄) alkylene]-phenylene-[(C₁-C₄) alkylene]

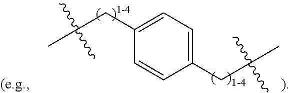

Embodiment 36. The dendrimer of Embodiment 35, wherein L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-.

Embodiment 37. The dendrimer of Embodiment 35, wherein L⁰, L¹, and L² are each independently at each occurrence C₁-C₆ alkylene (e.g., C₁-C₃ alkylene).

Embodiment 38. The dendrimer of Embodiment 35, wherein L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)).

Embodiment 39. The dendrimer of Embodiment 35, wherein L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

Embodiment 40. The dendrimer of any one of Embodiments 1-39, wherein R has a structural formula:

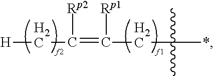

wherein:
   R^{p1} and R^{p2} are each independently H or C₁-C₆ (e.g., C₁-C₃) alkyl;
   f1 is 1, 2, 3, or 4; and
   f2 is 0, 1, 2, or 3.

Embodiment 41. The dendrimer of Embodiment 40, wherein —CR^{p2}=CR^{p1}— is a cis bond.

Embodiment 42. The dendrimer of Embodiment 40, wherein —CR^{p2}=CR^{p1}— is a trans bond.

Embodiment 43. The dendrimer of any one of Embodiments 40-42, wherein R^{p1} is H.

Embodiment 44. The dendrimer of any one of Embodiments 40-43, wherein R^{p2} is H.

Embodiment 45. The dendrimer of any one of Embodiments 40-44, wherein f1+f2≥3 (e.g., from 3 to 6, such as from 4 to 6).

Embodiment 46. The dendrimer of any one of Embodiments 1-39, wherein R has a structural formula:

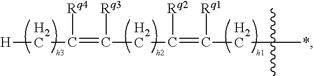

wherein:
R^{q1}, R^{q2}, R^{q3}, and R^{q4} are each independently H or C₁-C₆ (e.g., C₁-C₃) alkyl;
h1 is 1, 2, 3, or 4;
h2 is 1 or 2; and
h3 is 0, 1, 2, or 3.

Embodiment 47. The dendrimer of Embodiment 46, wherein —CR^{q2}=CR^{q1}— is a cis bond.

Embodiment 48. The dendrimer of Embodiment 46, wherein —CR^{q2}=CR^{q1}— is a trans bond.

Embodiment 49. The dendrimer of any one of Embodiments 46-49, wherein —CR^{q4}═CR^{q3}— is a cis bond.

Embodiment 50. The dendrimer of any one of Embodiments 46-49, wherein —CR^{q4}═CR^{q3}— is a trans bond.

Embodiment 51. The dendrimer of any one of Embodiments 46-50, wherein R^{q1} is H.

Embodiment 52. The dendrimer of any one of Embodiments 46-51, wherein R^{q2} is methyl or H.

Embodiment 53. The dendrimer of any one of Embodiments 46-52, wherein R^{q3} is H.

Embodiment 54. The dendrimer of any one of Embodiments 46-53, wherein R^{q4} is methyl or H.

Embodiment 55. The dendrimer of any one of Embodiments 46-54, wherein h1 is 1.

Embodiment 56. The dendrimer of any one of Embodiments 46-55, wherein h2 is 1 or 2.

Embodiment 57. The dendrimer of any one of Embodiments 46-56, wherein h3 is 1 or 2.

Embodiment 58. The dendrimer of any one of Embodiments 46-57, wherein h1+h2+h3≥3 (e.g., from 3 to 6, such as from 4 to 6).

Embodiment 59. The dendrimer of any one of Embodiments 1-39, wherein R has a structural formula:

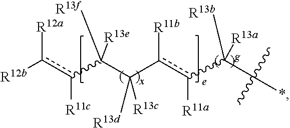

wherein:
   * indicates the point of attachment to the sulfur;
   e is 0, 1, 2, 3, 4, 5, or 6;
   g is 1, 2, or 3 (optionally g is 1);
   x is independently at each occurrence 0, 1, 2, or 3 (optionally x is 1); and
   R^{11a}, R^{11b}, R^{11c}, R^{12a}, R^{12b}, R^{13a}, R^{13b}, R^{13c}, R^{13d}, R^{13e}, and R^{13e} are each independently at each occurrence H or C₁-C₆ (e.g., C₁-C₃) alkyl.

Embodiment 60. The dendrimer of Embodiment 59, wherein R has a structural formula

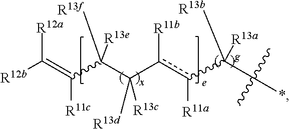

optionally

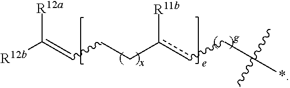

Embodiment 61. The dendrimer of Embodiment 59, wherein R has a structural formula

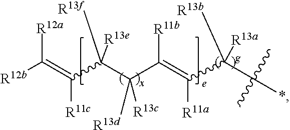

optionally

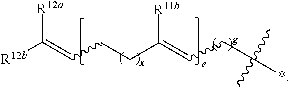

Embodiment 62. The dendrimer of Embodiment 59, wherein R has a structural formula

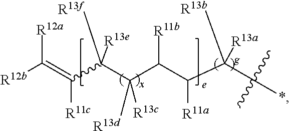

optionally

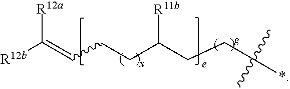

Embodiment 63. The dendrimer of any one of Embodiments 59-62, wherein e is 1, 2, 3, or 4 (optionally e is 1, 2, or 3).

Embodiment 64. The dendrimer of any one of Embodiments 59-63, wherein R^{11a} and R^{11c} are each H.

Embodiment 65. The dendrimer of any one of Embodiments 59-64, wherein R^{11b} is independently at each occurrence C₁-C₆ (e.g., C₁-C₃) alkyl.

Embodiment 66. The dendrimer of any one of Embodiments 59-65, wherein R^{12a} and R^{12b} are each independently C₁-C₆ (e.g., C₁-C₃) alkyl.

Embodiment 67. The dendrimer of any one of Embodiments 59-66, wherein R^{13a}, R^{13b}, R^{13c}, R^{13d}, R^{13e} and R^{13f} are each H.

Embodiment 68. The dendrimer of any one of Embodiments 1-39, wherein R is selected from the group consisting of:

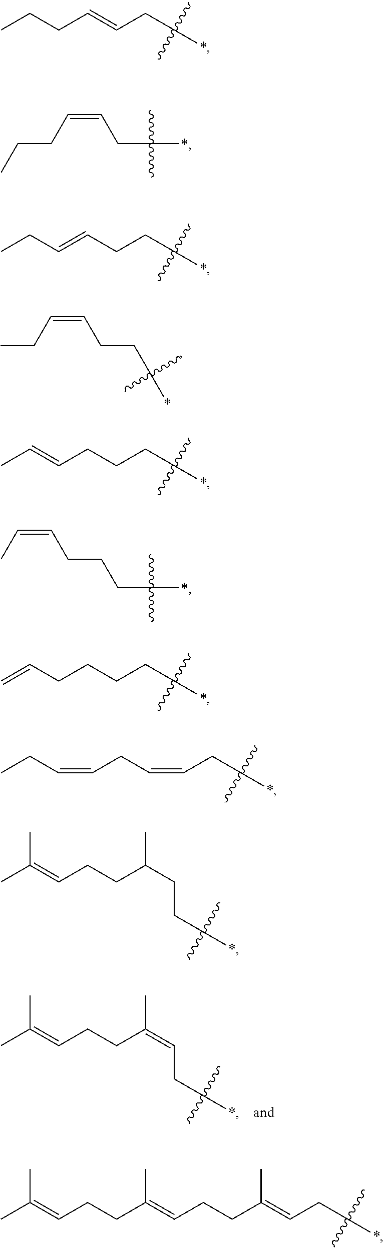

wherein * indicates the point of attachment to the sulfur.

Embodiment 69. The dendrimer of Embodiment 1, wherein the dendrimer is selected from the structures set forth in Table 6 and any pharmaceutically acceptable salt of any one of the structures set forth in Table 6.

Embodiment 70. The dendrimer of any one of Embodiments 1-69, wherein the dendrimer is characterized by an apparent acid dissociation constant (pKa) from 6.2 to 6.5 (e.g., as determined by in situ 6-p-toluidinyl-naphthalene-2-sulfonate (TNS) fluorescence titration).

Embodiment 71. The dendrimer of any one of Embodiments 1-70, wherein the dendrimer has a molecular weight (Mw) from 800 to 2,000 Da (e.g., as determined by mass spectrometry (MS) or by size exclusion chromatography (SEC)).

Embodiment 72. A lipid composition comprising:
an unsaturated dendrimer of any one of Embodiments 1-71; and
one or more lipids selected from an ionizable cationic lipid, a zwitterionic lipid, a phospholipid, a steroid or a steroid derivative thereof, and a polymer-conjugated (e.g., polyethylene glycol (PEG)-conjugated) lipid.

Embodiment 73. The lipid composition of Embodiment 72, wherein said unsaturated dendrimer is present in said lipid composition at a molar percentage of no more than about 60% (e.g., from about 5% to about 60%).

Embodiment 74. The lipid composition of Embodiment 72 or 73, wherein said one or more lipids comprises an ionizable cationic lipid separate from said unsaturated dendrimer.

Embodiment 75. The lipid composition of any one of Embodiments 72-74, wherein said ionizable cationic lipid is a fully saturated lipid.

Embodiment 76. The lipid composition of any one of Embodiments 72-74, wherein said ionizable cationic lipid is a fully saturated dendrimer of a generation (g) having the structural formula:

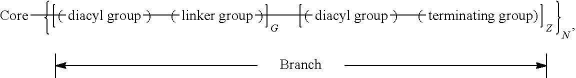

or a pharmaceutically acceptable salt thereof, wherein:
(a) the core comprises a structural formula (X_{Core}):

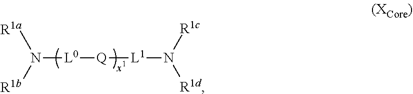

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

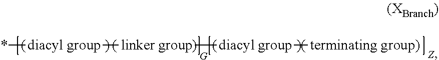

wherein:
   * indicates a point of attachment of the branch to the core;
   g is 1, 2, 3, or 4;
   Z=2^{(g-1)};
   G=0, when g=1; or G=Σᵢ₌₀^{i=g-2}2ⁱ, when g≠1;
(c) each diacyl group independently comprises a structural formula

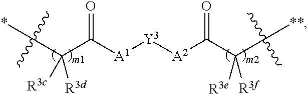

wherein:
   *indicates a point of attachment of the diacyl group at the proximal end thereof;
   ** indicates a point of attachment of the diacyl group at the distal end thereof,
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein: R⁴ is hydrogen or optionally substituted (e.g., C₁-C₆) alkyl;
   m¹ and m² are each independently at each occurrence 1, 2, or 3; and
   R^{3c}, R^{3d}, R^{3e}, and R^{3f} are each independently at each occurrence hydrogen or an optionally substituted (e.g., C₁-C₈) alkyl; and
(d) each linker group independently comprises a structural formula

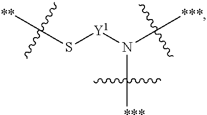

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-Cis, such as C₄-C₁₈) alkylthiol.

Embodiment 77. The lipid composition of any one of Embodiments 72-76, wherein said ionizable cationic lipid is present in said lipid composition at a molar ratio from about 1:1 to about 1:2 to said unsaturated dendrimer.

Embodiment 78. The lipid composition of any one of Embodiments 72-77, wherein said one or more lipids comprises a phospholipid, optionally selected from the group consisting of: 1,2-distearoyl-sn-glycero-3-phosphocholine (DSPC), and 1,2-dioleoyl-sn-glycero-3-phosphoethanolamine (DOPE).

Embodiment 79. The lipid composition of Embodiment 78, wherein said phospholipid is present in said lipid composition at a molar percentage from about 10% to about 50%.

Embodiment 80. The lipid composition of any one of Embodiments 72-79, wherein said one or more lipids comprises a polymer-conjugated (e.g., polyethylene glycol (PEG)-conjugated) lipid.

Embodiment 81. The lipid composition of Embodiment 80, wherein the polymer-conjugated (e.g., polyethylene glycol (PEG)-conjugated) lipid is present in said lipid composition at a molar percentage from about 0.25% to about 12.5%.

Embodiment 82. The lipid composition of any one of Embodiments 72-81, wherein said one or more lipids comprises a steroid or steroid derivative thereof.

Embodiment 83. The lipid composition of Embodiment 82, wherein said steroid or steroid derivative thereof is present in said lipid composition at a molar percentage from about 15% to about 60%.

Embodiment 84. The lipid composition of any one of Embodiments 72-83, further comprising a selective organ targeting (SORT) lipid that has a (e.g., permanently) positive net charge or a (e.g., permanently) negative net charge.

Embodiment 85. The lipid composition of Embodiment 84, wherein said SORT lipid has a (e.g., permanently) positive net charge.

Embodiment 86. The lipid composition of Embodiment 84, wherein said SORT lipid has a (e.g., permanently) negative net charge.

Embodiment 87. A pharmaceutical composition comprising a therapeutic agent coupled to a lipid composition comprising a dendrimer of any one of Embodiments 1-71.

Embodiment 88. A pharmaceutical composition comprising a therapeutic agent coupled to a lipid composition comprising a lipid composition of any one of Embodiments 72-86.

Embodiment 89. The pharmaceutical composition of Embodiment 87 or 88, wherein said therapeutic agent is a messenger ribonucleic acid (mRNA).

Embodiment 90. The pharmaceutical composition of Embodiment 89, wherein said mRNA is present in said pharmaceutical composition at a weight ratio from about 1:1 to about 1:100 with said cationic ionizable lipid.

Embodiment 91. The pharmaceutical composition of any one of Embodiments 87-90, further comprising a pharmaceutically acceptable excipient.

Embodiment 92. The pharmaceutical composition of any one of Embodiments 87-91, wherein the pharmaceutical composition is formulated for local or systemic administration.

Embodiment 93. The pharmaceutical composition of any one of Embodiments 87-91, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crémes, via a catheter, via a lavage, via continuous infusion, via infusion, via inhalation, via injection, via local delivery, or via localized perfusion.

Embodiment 94. The pharmaceutical composition of any one of Embodiments 87-93, comprising a SORT lipid in an amount sufficient to deliver said therapeutic agent to a liver cell (e.g., in a subject).

Embodiment 95. The pharmaceutical composition of any one of Embodiments 87-93, comprising a SORT lipid in an amount sufficient to deliver said therapeutic agent to a non-liver cell (e.g., in a subject).

Embodiment 96. The pharmaceutical composition of any one of Embodiments 87-93, wherein said unsaturated lipo-cationic dendrimer is present in said pharmaceutical composition in an amount sufficient to enhance a delivery potency of said therapeutic agent in a (e.g., liver) cell (e.g., in a subject).

Embodiment 97. A method for delivering a therapeutic agent into a cell, the method comprising:

contacting said cell with said therapeutic agent coupled to a lipid composition of any one of Embodiments 72-86, thereby delivering said therapeutic agent into said cell.

Embodiment 98. The method of Embodiment 97, wherein said contacting is ex vivo.

Embodiment 99. The method of Embodiment 97 or 98, wherein said contacting is in vivo.

Embodiment 100. The method of Embodiment 99, wherein said contacting comprises administering to a subject said therapeutic agent coupled to said lipid composition.

Embodiment 101. The method of any one of Embodiments 97-100, wherein said cell is in a (e.g., functionally compromised) tissue or organ of a subject.

Embodiment 102. The method of any one of Embodiments 97-101, further comprising repeating said contacting.

Embodiment 103. The method of any one of Embodiments 97-102, wherein said therapeutic agent is a heterologous messenger ribonucleic acid (mRNA).

Embodiment 104. The method of Embodiment 103, wherein, prior to said contacting, said cell exhibits an aberrant expression or activity of a protein encoded by said mRNA.

Embodiment 105. The method of Embodiment 104, wherein said aberrant expression or activity of said protein comprises an expression of a non-functional variant of said protein.

Embodiment 106. The method of Embodiment 104 or 105, wherein said aberrant expression or activity of said protein is associated with a genetic disease or disorder.

Embodiment 107. The method of any one of Embodiments 103-106, wherein said mRNA is expressed in said cell, upon said contacting, to produce a functional variant of said protein.

Embodiment 108. The method of any one of Embodiments 103-107, wherein an expression of said mRNA in said cell increases an amount of a functional variant of said protein as compared to an amount of said functional variant of said protein generated in absence of said contacting.

Embodiment 109. The method of any one of Embodiments 97-108, wherein said contacting comprises contacting a plurality of cells that comprises said cell.

Embodiment 110. The method of Embodiment 109, wherein said mRNA is expressed in at least 10% (e.g., at least 20%) of said plurality of cells, upon said contacting, to produce a functional variant of a protein encoded by said mRNA.

### EXAMPLES

### Example 1: Synthesis Procedure of Unsaturated Thiols from Alcohols

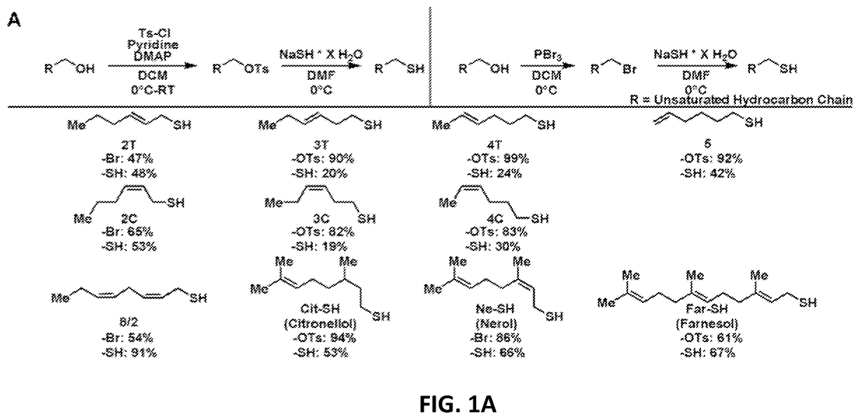

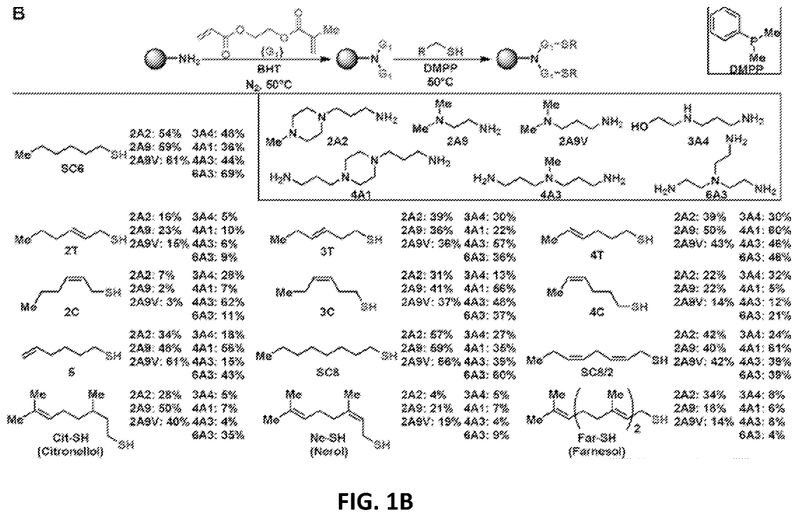

The overall scheme for the two main synthetic routes of synthesizing unsaturated thiols from alcohols are exemplified in FIG. 1. These two routes are abbreviated under corresponding unsaturated thiols as “-Br” and “OTs”.

“OTs” Route, —OH to -OTs

To a dry round-bottom flask with a stir bar, the alcohol (1 equiv.), CH₂Cl₂ (0.4M), 4-dimethylaminopyridine (0.2 equiv.), and pyridine (3 equiv.) were added. The solution was placed in an ice-bath and cooled to 0° C. Then, Ts-Cl (1 equiv.) was added portion-wise. The mixture was allowed to stir at 0° C. for 1 h, and then allowed to warm up to room temperature overnight under stirring. After 24 hours, the product was sequentially washed with 1M HCl and sat. NaHCO₃ solution, and the phases were separated. The aqueous solution was extracted 3× with DCM. The collected organic solution was washed with brine and dried over Na₂SO₄. The organic solution was then concentrated by rotary evaporation. The crude product was taken to the next step without further purification.

-OTs to -SAc

To a dry round-bottom flask with a stir bar, the tosylate (1 equiv.) and dimethylformamide (0.3M) were added and placed under N₂. Then, the KSAc (1.5 equiv.) was added and allowed to stir at 80° C. for 2 h. After 2 h, the solution was diluted with water, and the phases were separated. The aqueous solution was extracted 3× with Et₂O. Then, the collected organic solution was washed 3× with water and a final wash with brine. The organic solution was dried over MgSO₄ and concentrated by rotary evaporation. Column chromatography was performed using silica (10% EtOAc in hexanes).

-Sac to -SH

To a dry round-bottom flask with a stir bar placed under N₂, the thioacetate (1 equiv.) and dry THF (0.5M) were added. Then, it was cooled to 0° C. using an ice bath. LiAlH₄ (1.1 equiv.) was added portion-wise to the flask and allowed to stir on ice for 0.5 h and stir for 1.5 h as it warmed up to room temperature. Afterwards, the solution was cooled back to 0° C. and EtOAc and sat. solution of Rochelle Salt were slowly added. Once the phases were separated, the aqueous solution was extracted 3× with Et₂O. The collected organic solution were washed with brine and dried over MgSO₄. The organic solution was then concentrated by rotary evaporation. Column chromatography was performed using silica (100% hexanes).

“-Br” route, —OH to -Br

To a dry 250 mL round-bottom flask with a stir bar, the alcohol (1 equiv.) and CH₂Cl₂ (0.4M) were added and placed under N₂ and on ice-bath at 0° C. Once cooled, PBr₃ (0.5 equiv.) was added dropwise, and the solution was allowed to stir at 0° C. for 0.5 h. Then, the mixture was stirred as it warmed up to room temperature for 1.5 h. The reaction was quenched with sat. NaHCO₃ solution, and the phases were separated. The aqueous phase was extracted 3× with DCM. The collected organic solution was washed with brine and dried over Na₂SO₄. The organic solution was then concentrated by rotary evaporation. Column chromatography was performed using silica (100% hexanes).

-Br to -SH (NaSH)

To a dry round-bottom flask with a stir bar, the bromide (1 equiv.) and dimethylformamide (0.5M) were added and placed under N₂ and on ice-bath at 0° C. Then, NaSH·X H₂O (1.5 equiv.) was slowly added and allowed to stir on ice for 1 h. Then, the solution was stirred for 1 h as it warmed up to room temperature. Afterwards, the reaction was quenched with Et₂O and brine, and the phases were separated. The aqueous phase was extracted 3× with Et₂O, and the collected organic solution was washed 3× with brine. The organic solution was dried over MgSO₄ and concentrated by rotary evaporation. Column chromatography was performed using silica (100% hexanes).

Diyene Coupling Reactions were employed to synthesis diynes for resulting multiple double bonded unsaturated thiols.

Diyne Coupling

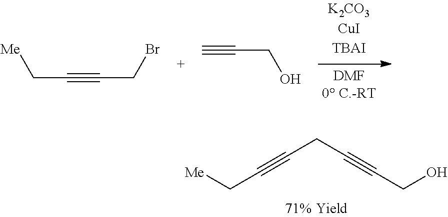

To a dry round-bottom flask with a stir bar, K₂CO₃ (1.5 equiv.), CuI (1 equiv.), tetra-n-butylammonium iodide (1 equiv.), and dimethylformamide (0.6M) were sequentially added and placed under N₂. The solution was cooled to 0° C. on an ice-bath, and the propargyl alcohol (1.2 equiv., portion-wise) and 1-bromo-2-pentyne (1 equiv.) were sequentially added. The solution was allowed to warm up to room temperature and stirred overnight. Afterwards, the solution was cooled to 0° C. on an ice-bath and quenched with Et₂O and water. The mixture was filtered through Celite, and the cake was rinsed with Et₂O 3×. The phases were then separated, and the aqueous layer was extracted 3× with Et₂O. The collected organic solution was washed 3× with water and a final wash with brine. Then, the organic solution was dried over MgSO₄ and concentrated by rotary evaporation. Column chromatography was performed using silica (100% hexanes>10% EtOAc/hexanes>20% EtOAc/hexanes).

(Z,Z) Diene Formation

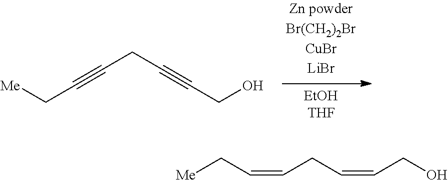 [SMILES: CCC#CCC#CCO ; CC/C=C\C/C=C\CO]

To a dry round-bottom flask with a stir bar, Zn powder (5.35 equiv.) and EtOH (⅔ of 1.33M) were added. Then half of the Br(CH₂)₂Br (½ of 0.46 equiv.) was added, place under N₂, and set to reflux for 10 min. Then, the second half of Br(CH₂)₂Br (½ of 0.46 equiv.) was added very slowly. The solution was set to reflux for 10 more min. Afterwards, the solution was cooled down to 50° C., and a mixture of CuBr (0.55 equiv.) and LiBr (1.35 equiv.) in THF (5.1M) were added. Finally, the diyne was diluted in EtOH (⅓ of 1.33M) and added. The solution was set to reflux at 140° C. overnight. Afterwards, the solution was cooled to room temperature and slowly quenched with sat. NH₄Cl. The mixture was filtered through Celite, and the cake was rinsed 3× with Et₂O. The collected organic solution was dried over MgSO₄ and concentrated by rotary evaporation. The crude product was taken to the next step without further purification.

After employing multiple strategies, including the Mitsunobu reaction/reduction and Bunte salt, two methods emerged as optimal based on the alcohol, reaction scale, and yield (Table. 9). For most allylic alcohols, conversion ofthe alcohol to a bromide and subsequent reaction with NaSH provided thiols at 48% to 91% yield. For non-allylic alcohols and farnesol, tosyl protection of the alcohol and subsequent treatment with NaSH afforded the desired thiols at 19% to 67% yields. Thiol nomenclature was based on the carbon chain length (6/8), position of unsaturation (2-5), configuration (cis/trans), and/or their natural product derivative (Citronellol, Nerol, and Farnesol). In tandem, seven ionizable amines were selected as candidates based on the established optimal pKa of 6.2-6.5 for ionizable lipids. The amines were prepared by reacting with an ester-based linker synthesized as previously described. With the amine cores and thiols in hand, appropriate equivalents of thiols per amine were combined with the modified amines and dimethyl phenyl pyridine as a catalyst to create the desired ionizable, unsaturated lipids. Optimization of the unsaturated thiol reactions were conducted. Example synthetic routes and their overall isolated yields can be seen in Table 9.

TABLE 9
Exemplary synthetic methods for terminating group / peripheries structures and yields

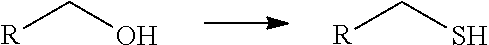

| Parent | Scale for Thiol | | Overall |
|---|---|---|---|
| Alcohol | Formation Step (mmol) | Method | Isolated Yield |
| Allylic (2T) | 5 | 1. Mitsunobu (CH₃COSH) | 14% |
| | | 2. K₂CO₃/MeOH | |
| Allylic (2T) | 5 | Lawesson's | N.P. |
| | | Reagent | |
| Allylic (2T) | 6 | 1. -OTs | N.P. |
| | | 2. KSAc | |
| Allylic (2T) | 3 | 1. -OTs | N.P. |
| | | 2. SiO₂-KSAc (adsorbed) | |
| Allylic (2T) | 3/7 | 1. -OTs | N.P. |
| | | 2. Thiourea (reflux) | |
| Allylic (2T) | 3 | 1. -OTs | N.P. |
| | | 2. KSAc | |
| Allylic (2C) | 40 | 1. PBr₃ | 26% |
| | | 2. NaSH | |
| Allylic (2C) | 12/80 | 1. -Br | N.P. |
| | | 2. Na₂S₂O₃ | |
| Allylic (2C) | 1 | 1. -Br | N.P. |
| | | 2. Thiourea | |
| Allylic (Far) | 5 | Lawsson's Reagent | N.P. |
| Non-Allylic (3T) | 5 | 1. Mitsunobu (CH₃COSH) | 22% |
| | | 2. K₂CO₃/MeOH | |
| Non-Allylic (4C) | 6 | Thiourea | N.P. |
| | | HBr in Acetic Acid | |
| | | Reflux | |
| Non-Allylic (4T) | 3 | 1. -OTs | 18% |
| | | 2. KSAc | |
| | | 3. NaOH | |
| Non-Allylic (4T) | 7.4 | 1. NaOCN/TFA | N.P. |
| | | 2. P₂S₅ | |
| Non-Allylic (5) | 10 | 1. -Br | N.P. |
| | | 2. Thiourea | |
| Non-Allylic (5) | 3 | 1. NaOCN/TFA | N.P. |
| | | 2. P₂S₅ | |
| Non-Allylic (5) | 46 | 1. -OTs | 28% |
| | | 2. KSAc | |
| | | 3. LiAlH₄ | |
| Non-Allylic (4T) | 0.4/15.2 | 1. -OTs | 56%/27% |
| | | 2. NaSH*H₂O | |
| Non-Allylic (5) | 17.2 | 1. -OTs | 42% |
| | | 2. NaSH*H₂O | |

An overall scheme of the dendrimer synthesis comprising an amine core, linker and unsaturated thiol is illustrated in FIG. 1B. The isolated overall isolated yield of each example unsaturated thiol with a corresponding core also be seen in FIG. 1B.

### Modified Amine Core

To a scintillation vial with a stir bar, the amine core of interest (1 equiv.) and BHT (0.088 equiv.) were added. Afterwards, the di-ester linker 2-(acryloyloxy)ethyl methacrylate (AEMA) (G1) was added (1.1 equiv. per arm) and set to stir at 50° C. for 24 hr or until complete conversion was observed by ¹H NMR. The crude product was taken to the next step without further purification.

### Cationic Lipid

To a scintillation vial with a stir bar, the modified amine core (1 equiv.), thiol of interest (1.1 equiv. per arm), and DMPP (0.45 equiv.) were added and set to stir at 50° C. for 24-48 hr. Column chromatography was performed using neutral Alumina. (2A2: 100% Hex.>5% EtOAc/Hex.>10% EtOAc/Hex.>15% EtOAc/Hex.) (2A9, 2A9V: 100% Hex.>20% EtOAc/Hex.>50% EtOAc/Hex.) (3A4, 4A1, 4A3: 100% Hex.>15% EtOAc/Hex.>50% EtOAc/Hex.) (6A3: 100% Hex.>12% EtOAc/Hex.>30% EtOAc/Hex.) Allylic thiols required excess DMPP (1.1 equiv. per arm) for completion of reaction.

### Example 2: In Vivo and In Vitro Assays

### Instrument and Materials

Reactions were performed in oven-dried round bottom flasks or borosilicate scintillation vials with a stir bar. Proton nuclear magnetic resonance (¹H NMR) spectra were recorded with a Bruker 400 MHz or Varian 500 MHz spectrometer. Peaks are reported in parts per million and are referenced from CDCl₃ (7.26).

Flash chromatography was performed on a Teledyne Isco CombiFlash Rf-200i chromatography system equipped with UV-Vis and evaporative light scattering detectors (ELSD) or manually using 60 Å, 40-63 um Silica (Sorbtech) or activated, neutral Aluminum Oxide (Sigma-Aldrich). Particle sizes and PDI were measured by Dynamic Light Scattering (DLS) using a Malvern Zetasizer Nano ZS (He-Ne laser, λ=632 nm). Confocal microscopy was conducted using a Zeiss LSM-710 laser scanning confocal microscope and data analyzed with the Zeiss LSM-710 software.

All commercial reagents were used as received (ACS reagent grade or higher) from Sigma-Aldrich unless otherwise noted. All solvents were purchased from Fisher Scientific and purified with a solvent purification system (Innovative Technology) with the exception of dimethylformamide (Sigma-Aldrich), chloroform (CHCl₃) (Sigma-Aldrich), and EtOH (Pharmco) being used as received. 1-(3-Aminopropyl)-4-methylpiperazine (2A2), 3-cis-hexenol, 3-trans-hexenol, and 2-cis-hexenol were purchased from Alfa Aesar. 4-trans-hexenol and ethanol (Et₂O) was purchased from Acros Organics. 1,4-Bis(3-aminopropyl)piperazine (4A1), 3,3′-diamino-N-methyldipropylamine (4A3), 4-cis-hexenol, 5-hexenol, and tertbutylammonium iodide were purchased from TCI. N-(2-Hydroxyethyl)-1,3-propanediamine (3A4) was purchased from Frontier Scientific.

Luciferase mRNA and Cyanine 5 (Cy5) Luciferase mRNA were purchased from TriLink Biotechnologies. DOPE, DOPC, DOPS, DSPC, NBD-PE, N-Rh-PE were all obtained from Avanti Polar Lipids. Cholesterol was purchased from Sigma-Aldrich. DMG-PEG2000 was purchased from NOF America. LysoTracker Green DND-26 was purchased from ThermoFisher Scientific.

### Cell Lines

IGROV-1 were obtained from ATTC. Cells were cultured in RPMI 1640 medium supplemented with 5% FBS and 50 U/mL penicillin/streptomycin. All cells were maintained at 37° C. and 5% CO₂.

### In Vitro Nanparticle Formulations

Ionizable lipid, DOPE, cholesterol, DMG-PEG2k were dissolved in ethanol as noted below (S2, base formulation). Firefly Luciferase (Luc) mRNA was diluted in 10 mM citric acid-sodium citrate buffer (pH 4.4). The lipid mixture and nucleic acid solution were rapidly combined at a volumetric ratio of 3:1 nucleic acid:lipid mix. Afterwards, the LNP formulations were diluted with 1×PBS to 1 ng/uL concentration.

### In Vitro Luciferase Expression and Cell Viability Tests

IGROV-1 cells were seeded into white 96-well plates at a density of 1×10⁴ cells per well the day before transfection with media. Day of transfection, the media was replaced with 200 μL of fresh media. Then, 25 μL of the Luc mRNA formulations were added with a fixed dose of 25 ng mRNA per well. After incubation for another 24 hours, ONE-Glo+ Tox kits (Promega) were used to detect Luc mRNA expression and cytotoxicity.

### In Vivo Nanoparticle Formulations

Ionizable lipid(s), DOPE, cholesterol, DMG-PEG2k were dissolved in ethanol as noted below in S2, and the Luc mRNA was diluted in 10 mM citric acid-sodium citrate buffer (pH 4.4) at the desired dosage. The lipid mixture and nucleic acid dilution were rapidly combined at a volumetric ratio of 3:1 nucleic acid:lipid mix. The nanoparticles were dialyzed against 1×PBS in Pur-A-Lyzer midi dialysis chambers (Sigma-Aldrich, WMCO 3.5 kDa) for 2 hours prior to injection.

In Vivo Luciferase mRNA Delivery

All experiments were approved by the Institutional Animal Care & Use Committee (IACUC) of The University of Texas Southwestern Medical Center and were consistent with local, state and federal regulations as applicable. Female C57BL/6 mice (18-20 g) were injected with LNP formulations via tail vein injection (5 ug of mRNA, 0.25 mg/kg). After 6 h, the luciferase expression was evaluated by live animal bioluminescence imaging. Animals were anesthetized under isofluorane, and D-Luciferin (150 mg/kg) was introduced by intraperitoneal injection. Then, the luciferase activity was imaged, and ex vivo imaging was performed on systemic organs after resection on an IVIS Lumina system (Perkin Elmer). The images were processed using Living Image analysis software (Perkin Elmer).

### LNP Characterization

To evaluate physicochemical properties of the formulations, Dynamic Light Scattering (DLS, Malvern, 1730 Scattering angle) was used. Size distribution and Polydispersity Index (PDI) were measured using 100 μL of nanoparticles (50 ng/uL of mRNA). To calculate encapsulation efficacy of mRNA, the Quant-iT RiboGreen RNA Assay was conducted based on its standard protocol (ThermoFisher).

### Lipid Fusion Assay

Endosome mimicking anionic liposomes were prepared by mixing DOPS:DOPC:DOPE:NBD-PE:N-Rh-PE (25:25:48:1:1 molar ratio) in CHCl₃ at a fixed total lipid concentration of 1 mM. Afterwards, the film was created by rotary evaporation of the solution, followed by 2 h of vacuum drying to produce a thin lipid film.

LNPs were formulated with the in vivo method. 100 uL PBS (pH 5.5) was added to each well in a black 96-well plate (n=3 per sample), and 1 μL of endosome mimicking anionic liposomes (1 mM) was added to each well. Endosome mimicking anionic liposomes in PBS served as the negative control (Fₘᵢₙ), and the endosome mimic lipids incubated with 2% Triton-X in PBS solutions were set as positive control (Fₘₐₓ). 10 μL of LNPs being tested were added to the wells (n=3). After incubating at 37° C. for 5 min., fluorescence measurements (F) were conducted on a microplate reader at Ex/Em=465/520 nm. The lipid fusion (%) was calculated as (F−Fₘᵢₙ)/(Fₘₐₓ−Fₘᵢₙ)*100%.

### Confocal Imaging

LNPs were formulated with the in vivo method. 100 uL PBS (pH 5.5) was added to each well in a black 96-well plate (n=3 per sample), and 1 μL of endosome mimicking anionic liposomes (1 mM) was added to each well. Endosome mimicking anionic liposomes in PBS served as the negative control (Fₘᵢₙ), and the endosome mimic lipids incubated with 2% Triton-X in PBS solutions were set as positive control (Fₘₐₓ). 10 μL of LNPs being tested were added to the wells (n=3). After incubating at 37° C. for 5 min., fluorescence measurements (F) were conducted on a microplate reader at Ex/Em=465/520 nm. The lipid fusion (%) was calculated as (F−Fₘᵢₙ)/(Fₘₐₓ−Fₘᵢₙ)*100%.

### Statistical Analyses

Data, unless otherwise noted, is reported as mean±SD. Graph Pad Prism 7 was used to calculate statistical comparisons. Two-tailed t-tests were used to calculate p values. Not significant: P>0.05; * denotes P<0.05; ** denotes P<0.005; *** denotes P<0.0005.

### Results

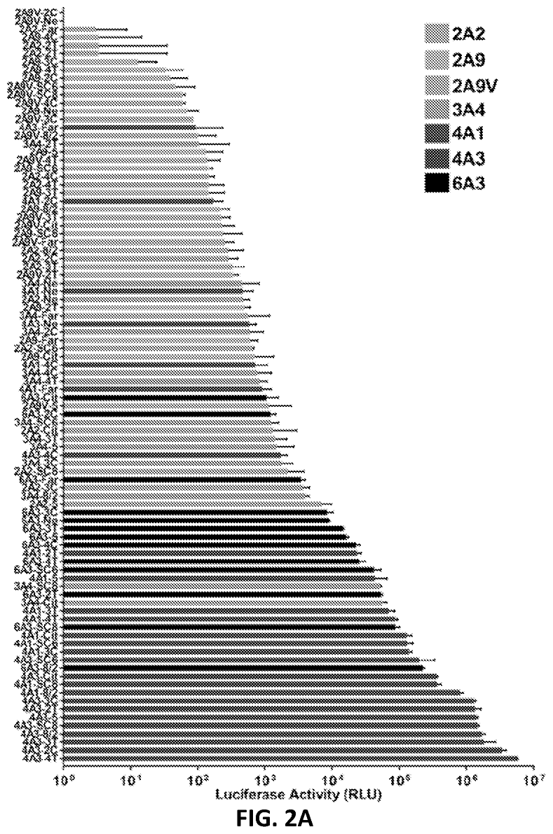

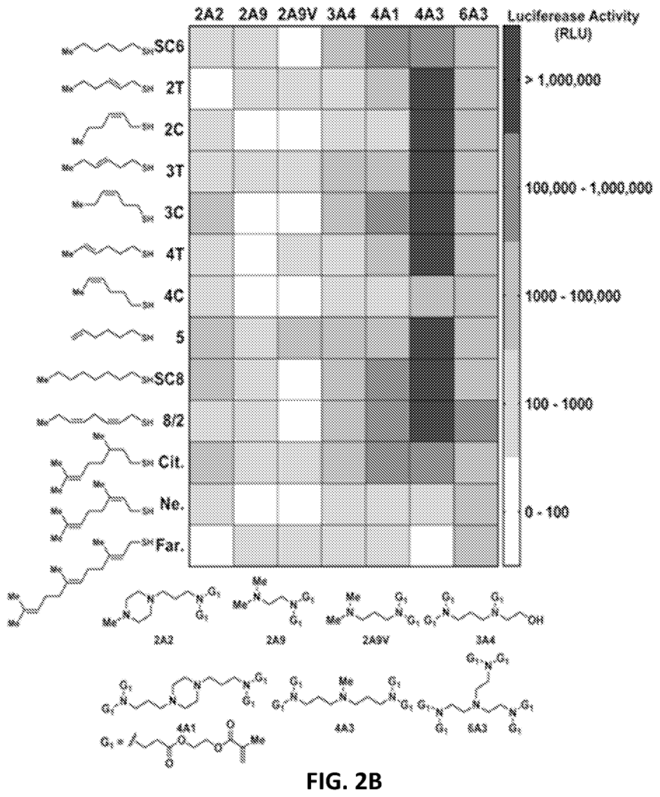

The new lipids were formulated into LNPs using a mix of the synthesized lipid, DOPE, cholesterol, and DMG-PEG2k (15:15:30:3, mol:mol), encapsulating Firefly Luciferase (Luc) mRNA. FIG. 2 shows the evaluation of the LNPs in IGROV-I cells (25 ng/well) for cell viability and Luc expression. Assessment of the heat map allowed for determination of SARs with respect to hydrophobic domain and amine core chemical structure. While introducing unsaturation in the tail produced some lipids with improved transfection over parent compounds, this chemical alteration was not a change that automatically and universally improved performance. Rather, the placement and cis/trans configuration of the unsaturation were important in determining whether in vitro mRNA delivery would be improved. For example, 8/2 showed comparable Luc expression to its saturated counterpart (SC8). Among all cores examined in the initial screen, 4A3 was the most efficacious. The 4A3 series was selected for further evaluation on its capabilities and study SAR with respect to the hydrophobic domain.

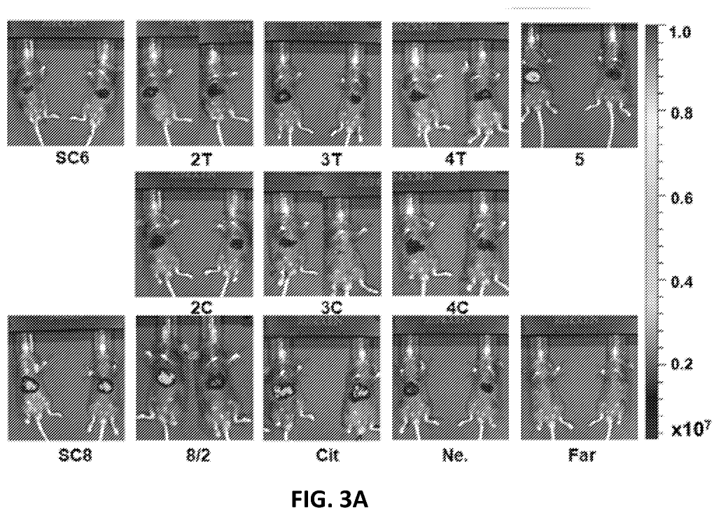

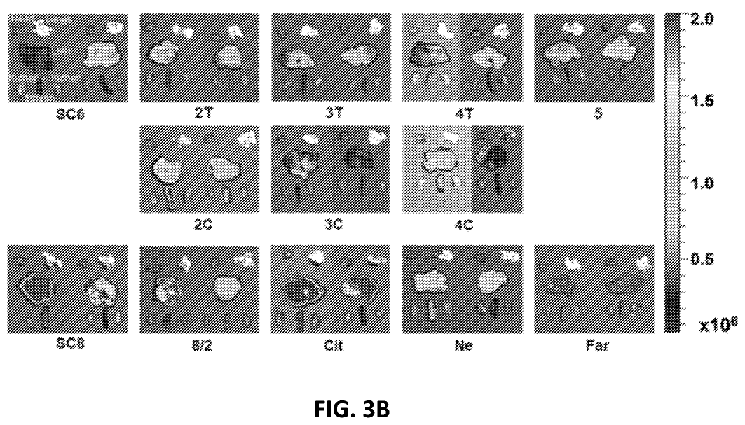

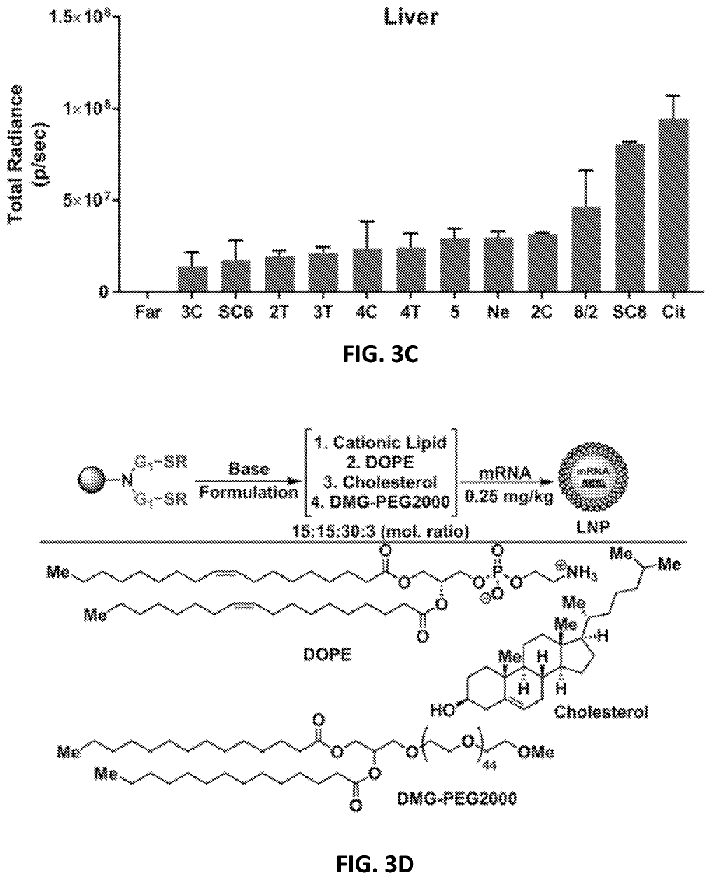

The 4A3 series was evaluated to assess how the unsaturation variations affected mRNA delivery in vivo, which is the more relevant setting for mRNA therapeutic testing. LNPs were formulated using the same components for in vitro testing in the same molar ratio. C57BL/6 mice were administered with each of the 4A3-based LNP series carrying Luc mRNA via tail-vein injections (FIG. 3). Clear differences from the in vitro data appeared. While the six-carbon chain series showed few significant differences amongst each other, the eight-carbon tail series exhibited striking distinctions.

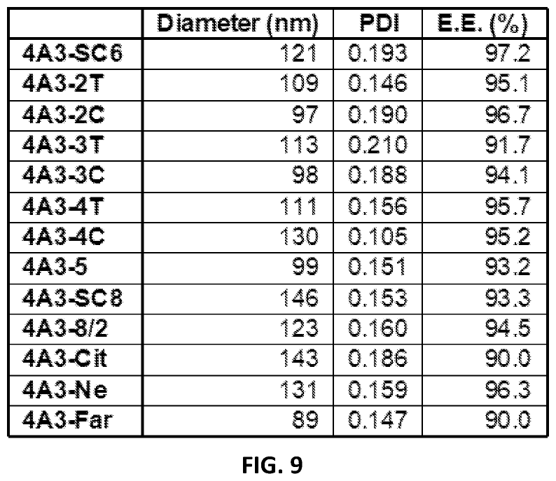

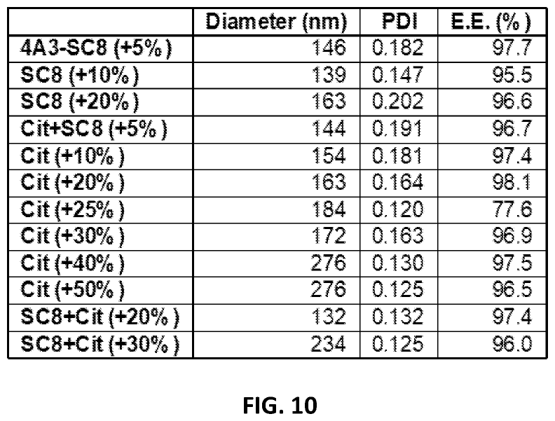

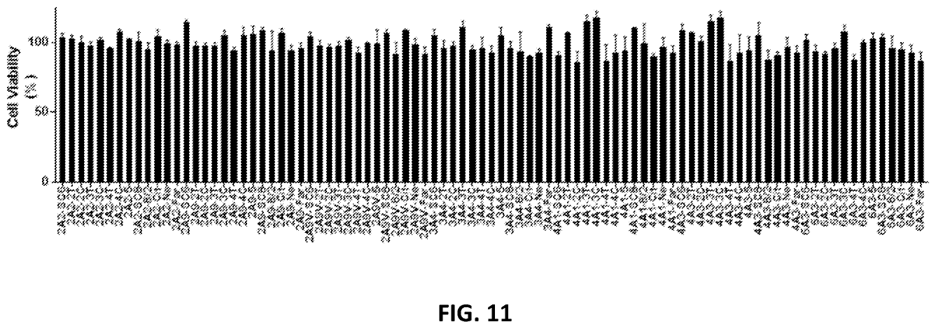

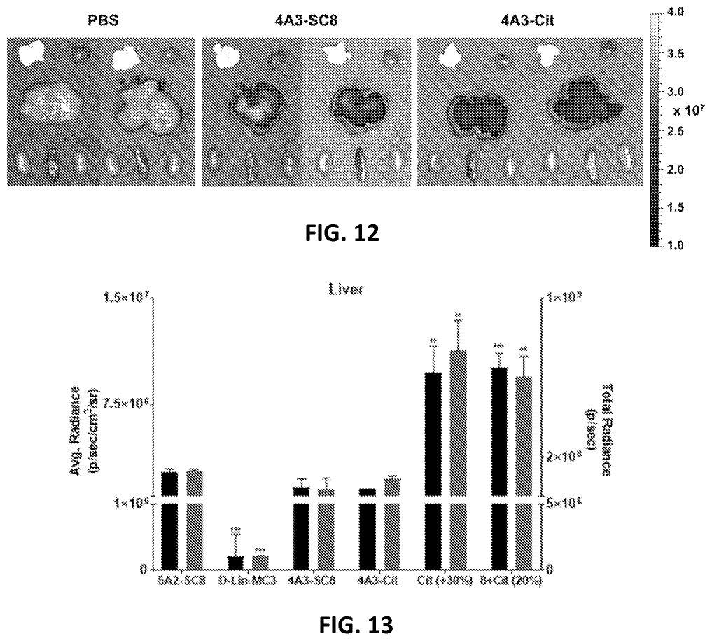

Despite being structurally similar, 4A3-Cit performed better than 4A3-Ne, differing only by a prenyl motif per tail. This disparity was likely due to the increased rigidity based on this structural difference. The comparison of the duo suggests that the “stiffness” of the components and its ability to promote membrane permeability may be an important factor in improving mRNA delivery when tailoring to its unsaturation. With 4A3-8/2, it exhibited lower Luc expression compared to its alkyl partner, suggesting again that the introduction of unsaturation alone was not sufficient to increase efficacy. Efficacy did not significantly correlate with size, PDI, or encapsulation efficiency (FIG. 9), suggesting that chemical structure was the major driver of efficacy. Moreover, the introduction of unsaturation did not change the biodistribution of 4A3-SC8 and saturated 4A3-Cit based LNPs (FIG. 12). As endosomal escape of LNPs remains a major challenge that correlates with amino lipid chemistry, these LNPs may differ based on their ability to escape the endosome.

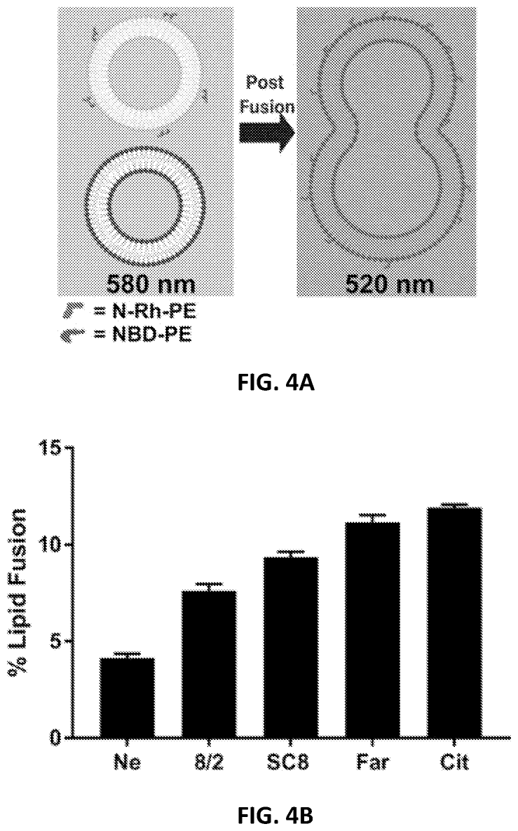

Fluorescence resonance energy transfer (FRET)-based assay was employed to determine the LNP's ability to disrupt and fuse with the endosomal membrane (FIG. 4). DOPE-conjugated FRET probes (NBD-PE and N—Rh-PE) were formulated into endosome-mimicking nanoparticles. NBD is normally quenched by the rhodamine, but the NBD signal would rise if disruption of the membrane occurred. In accordance with the in vivo data, 4A3-Cit proved to be efficacious. 4A3-Cit's unique structure may promotes better endosomal escape than the other LNPs.

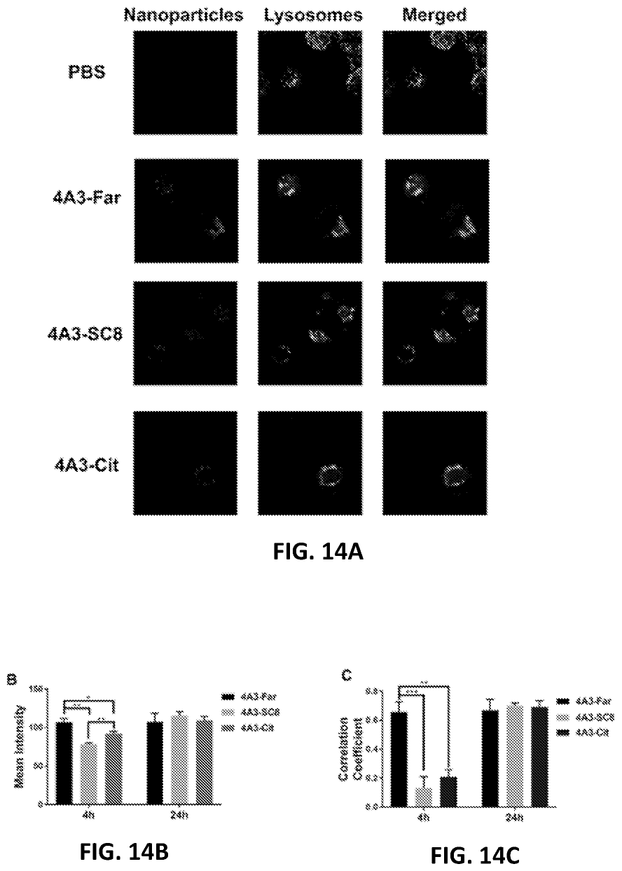

Cellular uptake and intracellular trafficking was further examined, in vitro experiments were performed using Cy5-labeled mRNA LNPs. The results showed that 4A3-SC8, 4A3-Far, and 4A3-Cit LNPs were all effectively internalized at 4 hours and 24 hours (FIG. 14A and FIG. 14B). There were differences at 4 hours, with the two unsaturated lipids (4A3-Far and 4A3-Cit) LNPs internalizing slightly faster than the saturated (4A3-SC8) LNPs. The progression of Cy5 mRNA was further tracked from the cell membrane to endosomes to lysosomes and quantified co-localization of Cy5 mRNA with lysosomes. Interestingly, there was significantly higher colocalization between 4A3-Far and lysosomes compared to 4A3-SC8 and 4A3-Cit. These results suggest that 4A3-Far LNPs lead to mRNA accumulation in lysosomes and unproductive delivery. Moreover, the reduced colocalization with lysosomes for 4A3-SC8 and 4A3-Cit LNPs may help explain why they were efficacious. The overall results connected chemical structure to factors including cellular uptake, endosomal escape, and intracellular trafficking.

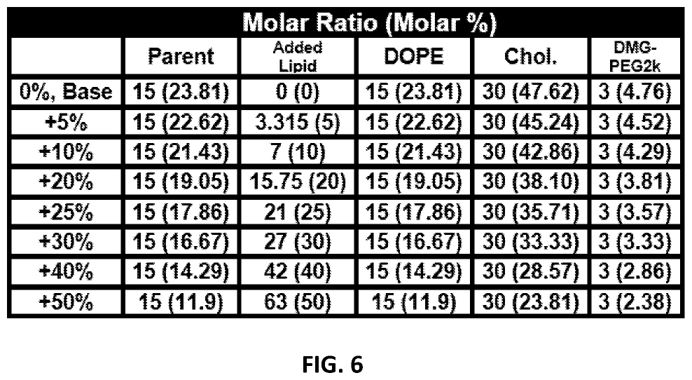

Building on the discovery of unsaturated 4A3-Cit and understanding of increased lipid fusion, 4A3-Cit could be used to enhance mRNA delivery in vivo to the liver. Selective Organ Targeting Nanoparticles (SORT) can have selective mRNA delivery to different tissues. In one application, mixing two ionizable lipids into a 5-component LNP increased delivery efficacy to the liver. 4A3-SC8 and 4A3-Cit could be combined into a SORT LNP to engineer improved lipid formulations for the liver. mRNA delivery efficacy and tissue tropism can be correlated with the chemical identity and percent incorporation of the added SORT molecule while keeping the molar amount of other molecules constant. Increasing the occupied percentage of the unsaturated lipid could promote endosomal escape and may improve mRNA delivery. Thus, the following primary formulations were devised: 4A3-SC8 with additional parent lipid, 4A3-Cit with additional parent lipid, and a mixture of the two lipids where one would serve as the parent lipid and the other served as additional supplemental SORT lipid (FIG. 6).

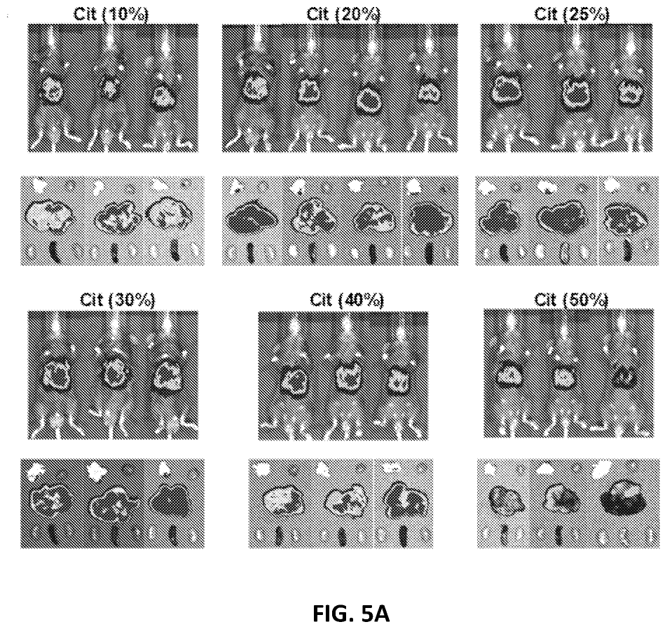

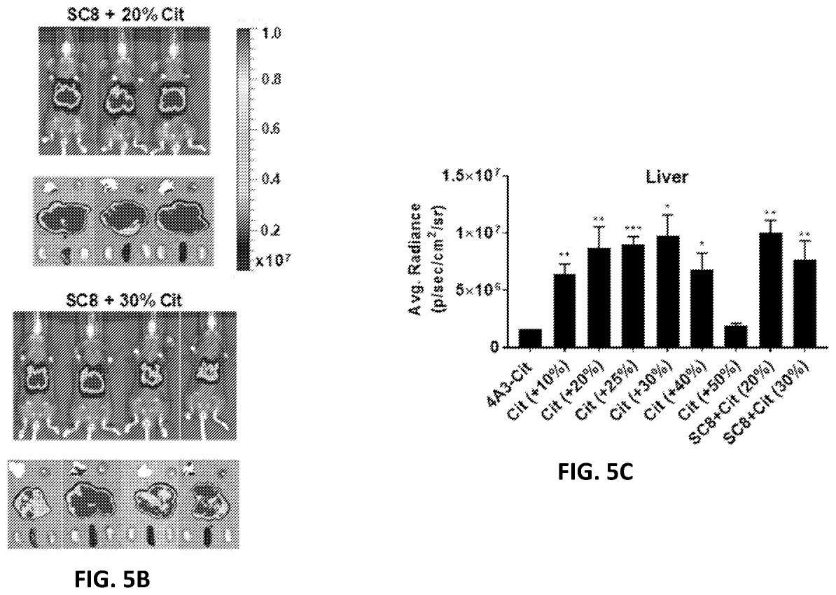

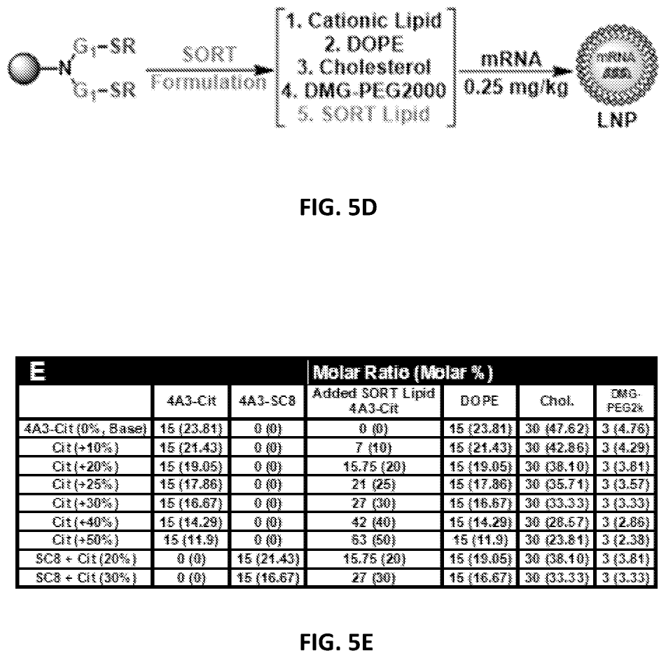

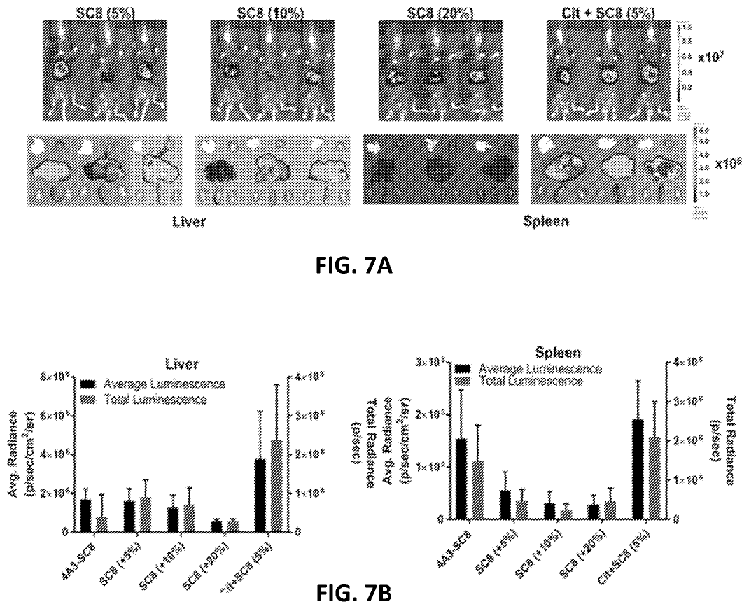

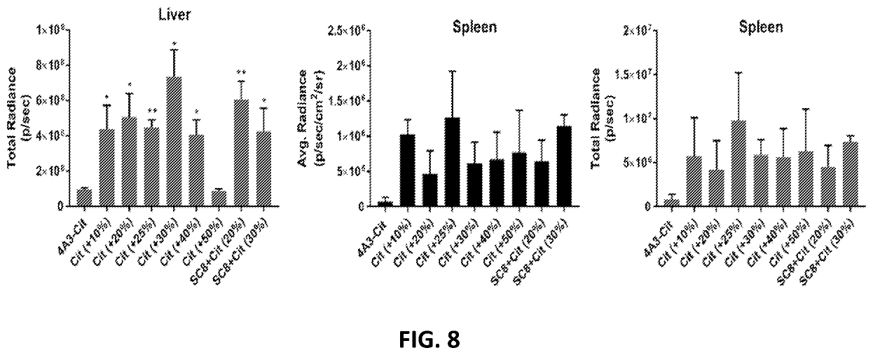

The SORT lipid required optimization to improvements in mRNA transfection by evaluating formulations across a range of SORT lipid amounts. Additional 4A3-SC8 yielded only mild improvements (FIG. 7), but formulations involving additional Cit achieved significant improvements (FIG. 5). Once the ideal percentages for the SORT lipids (+5% SC8 and +20/30% of Cit), the cross-over mixtures were tested. Cit+SC8 (5%) performed only slightly better than its parent formulation (FIG. 7), but SC8+Cit (20%) surpassed all others with an 18-fold increase of the average luminescence. SC8+Cit (20%) was also superior to its saturated parent lipid and established benchmarks including 5A2-SC8 LNPs and DLin-MC3-DMA LNPs (FIG. 13). Interestingly, 8+Cit (30%) performed worse than its Cit (+30%) counterpart, and the reverse was observed with its 20% variant. The data revealed that improvement was accessible by increasing occupancy of the 4A3-Cit, but there was a threshold for the additive effect of Cit. The comparison of the average luminescence suggests that there needs to be a careful balance of ionizable lipids for LNPs to achieve successful increased transfection.

In summary, the synthesized unsaturated thiols enabled access to a library of 91 ionizable lipids. In vitro mRNA delivery screening results revealed differences and SAR that correlated with the location/configuration of the unsaturated bond(s) rather than the simple presence of unsaturation. Selecting the 4A3 amine core to study in vivo, 4A3-Cit demonstrated the highest efficacy. Mechanistic studies using model endosomal membranes indicated that 4A3-Cit demonstrated the highest lipid fusion ability, suggesting its unique tail structure may enhance endosomal escape. 4A3-Cit further established its exceptional utility for mRNA delivery through application in SORT LNPs. SC8+Cit (20%) SORT LNPs improved mRNA delivery 18-fold over parent formulations. The findings from this work aid a deeper understanding of how unsaturation may promote mRNA delivery by increasing endosomal fusion, identify 4A3-Cit as a potent new lipid, and further expand the utility of SORT LNPs for efficacious mRNA delivery.

While preferred embodiments of the present disclosure have been shown and described herein, it will be obvious to those skilled in the art that such embodiments are provided by way of example only.

Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A (e.g., unsaturated) dendrimer of a generation (g) having a structural formula: 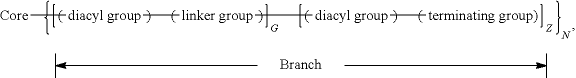
or a pharmaceutically acceptable salt thereof, wherein:
(a) the core comprises a structural formula (X_{Core}): 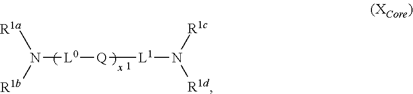
wherein:
Q is independently at each occurrence a covalent bond, —O—, —S—, —NR²—, or —CR^{3a}R^{3b}—;
R² is independently at each occurrence R^{1g} or -L²-NR^{1e}R^{1f};
R^{3a} and R^{3b} are each independently at each occurrence hydrogen or an optionally substituted (e.g., C₁-C₆, such as C₁-C₃) alkyl;
R^{1a}, R^{1b}, R^{1c}, R^{1d}, R₁ₑ, R^{1f}, and R^{1g} (if present) are each independently at each occurrence a point of connection to a branch, hydrogen, or an optionally substituted (e.g., C₁-C₁₂) alkyl;
L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
x¹ is 0, 1, 2, 3, 4, 5, or 6; and
(b) each branch of the plurality (N) of branches independently comprises a structural formula (X_{Branch}): 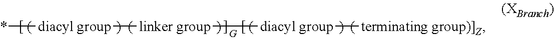
wherein:
* indicates a point of attachment of the branch to the core;
g is 1, 2, 3, or 4;
Z=2^{(g-1)};
G=0, when g=1; or G=ᵢ₌₀^{i=g-2}2ⁱ, when g≠1;
(c) each diacyl group independently comprises a structural formula 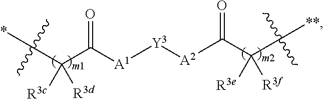
wherein:
* indicates a point of attachment of the diacyl group at the proximal end thereof;
** indicates a point of attachment of the diacyl group at the distal end thereof;
Y³ is independently at each occurrence an optionally substituted (e.g., C₁-C₁₂);
alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR⁴—, wherein: R⁴ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula 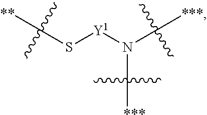
wherein:
** indicates a point of attachment of the linker to a proximal diacyl group;
*** indicates a point of attachment of the linker to a distal diacyl group; and
Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is R independently at each occurrence selected from C₆-C₂₂ alkenyl, C₆-C₂₂ alkadienyl, and C₆-C₂₂ alkatrienyl.

2. The dendrimer of claim 1, wherein x¹ is 0, 1, 2, or 3.

3. The dendrimer of claim 1, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl 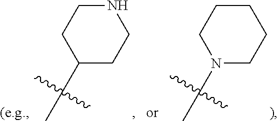 [SMILES: *C1CCNCC1 ; *N1CCCCC1]
N—(C₁-C₃ alkyl)-piperidinyl 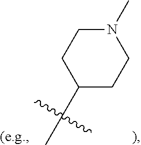 [SMILES: *C1CCN(C)CC1]
piperazinyl 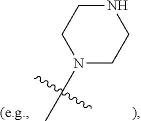 [SMILES: *N1CCNCC1]
N—(C₁-C₃ alkyl)-piperadizinyl 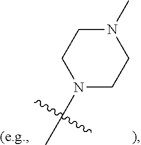 [SMILES: *N1CCN(C)CC1]
morpholinyl 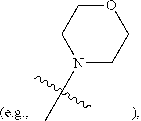 [SMILES: *N1CCOCC1]
N-pyrrolidinyl 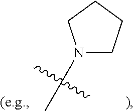 [SMILES: *N1CCCC1]
pyrrolidinyl 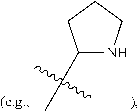 [SMILES: *C1CCCN1]
or N—(C₁-C₃ alkyl)-pyrrolidinyl 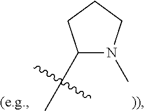 [SMILES: *C1CCCN1C]
(e.g., C₆-C₁₀) aryl, and C₃-C₅ heteroaryl 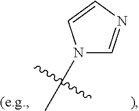 [SMILES: *n1ccnc1]
or pyridinyl

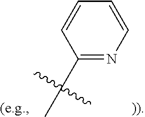 [SMILES: *c1ccccn1]

4. The dendrimer of claim [00253], wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

5. The dendrimer of claim 1, wherein R^{3a} and R^{3b} are each independently at each occurrence hydrogen.

6. The dendrimer of claim 1, wherein the plurality (N) of branches comprises at least 2 (e.g., at least 3, at least 4, at least 5, or at least 6) branches.

7. The dendrimer of claim 1, wherein the plurality (N) of branches comprises from 2 to 6 (e.g., from 3 to 6, or from 4 to 6) branches.

8. The dendrimer of claim 1, wherein g=1; G=0; and Z=1.

9. The dendrimer of claim 8, wherein each branch of the plurality of branches comprises a structural formula * [symbol] diacyl group [symbol] terminating group)

10. The dendrimer of claim 1, wherein g=2; G=1; and Z=2.

11. The dendrimer of claim 10, wherein each branch of the plurality of branches comprises a structural formula

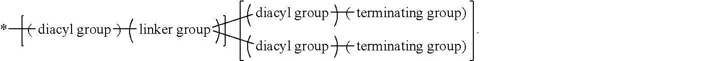

12. The dendrimer of claim 1, wherein g=3; G=3; and Z=4.

13. The dendrimer of claim 12, wherein each branch of the plurality of branches comprises a structural formula

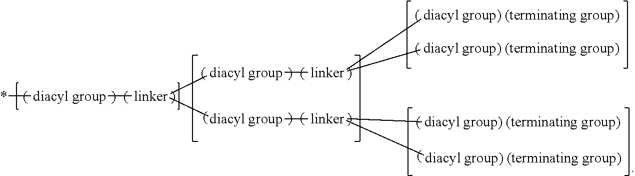

14. The dendrimer of claim 1, wherein g=4; G=7; and Z=8.

15. The dendrimer of claim 14, wherein each branch of the plurality of branches comprises a structural formula:

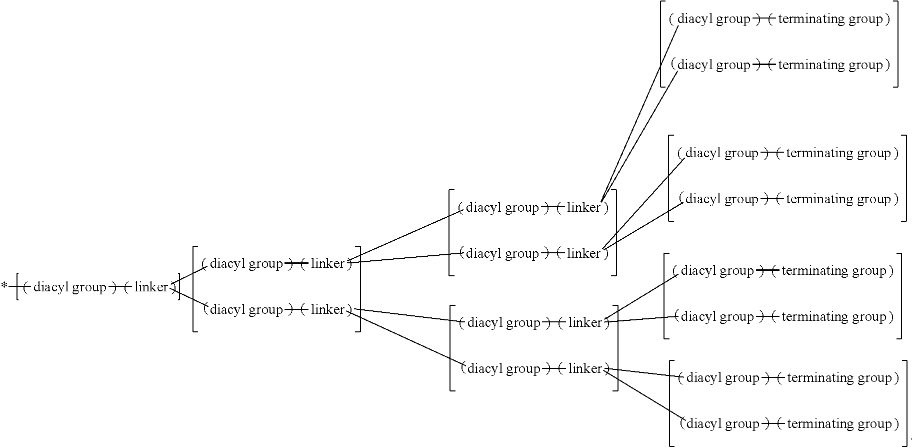

16. The dendrimer of claim 1, wherein the core comprises a structural formula:

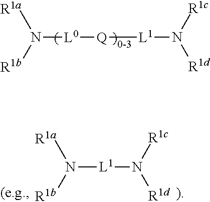

17. The dendrimer of claim 1, wherein the core comprises a structural formula:

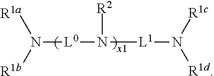

18. The dendrimer of claim 17, wherein the core comprises a structural formula:

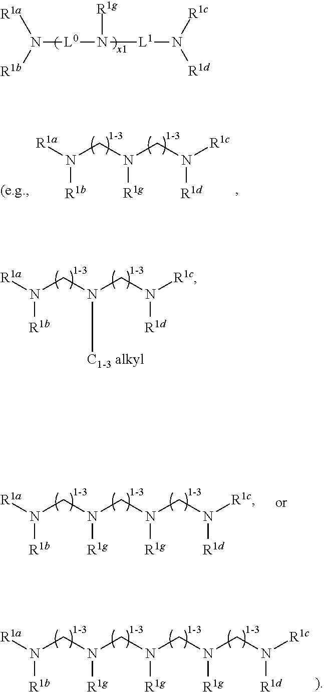

19. The dendrimer of claim 17, wherein the core comprises a structural formula:

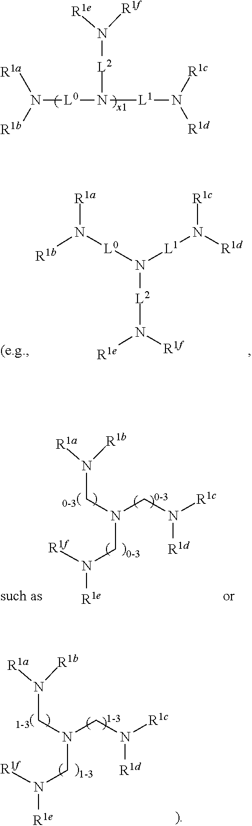

20. The dendrimer of claim 1, wherein the core comprises a structural formula: 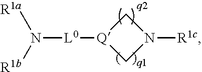
wherein Q′ is —NR²— or —CR^{3a}R^{3b}—; q¹ and q² are each independently 1 or 2.

21. The dendrimer of claim 20, wherein the core comprises a structural formula:

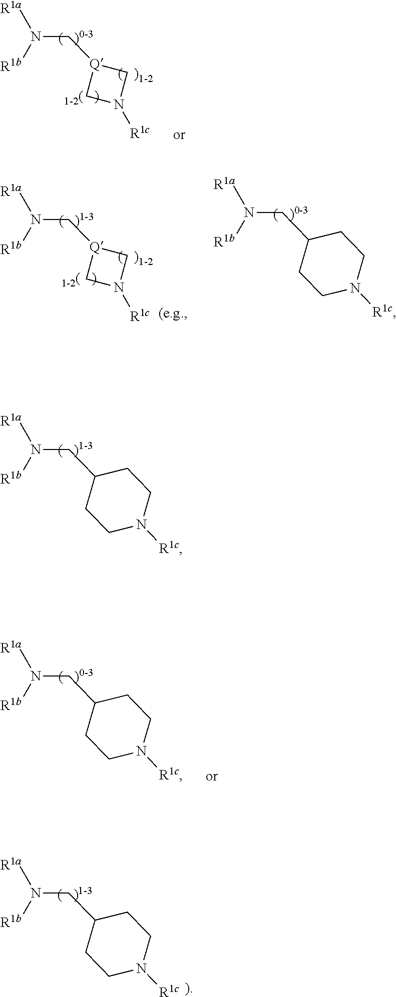

22. The dendrimer of claim 1, wherein the core comprises a structural formula 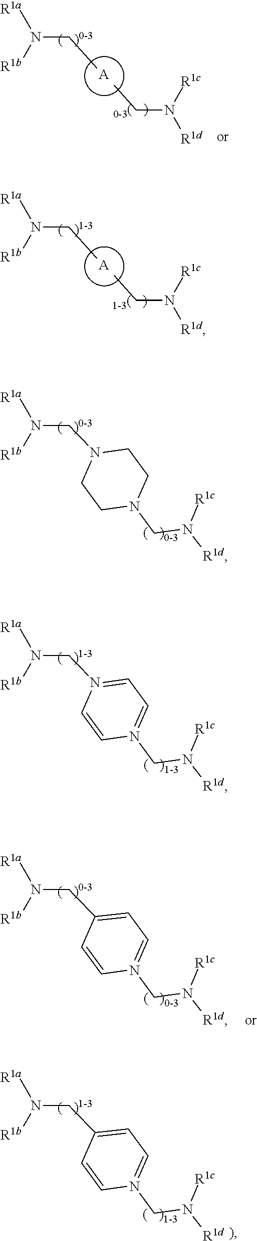
wherein ring A is an optionally substituted aryl or an optionally substituted (e.g., C₃-C₁₂, such as C₃-C₅) heteroaryl.

23. The dendrimer of claim 1, wherein the core comprises a structural formula

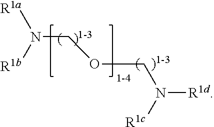

24. The dendrimer of claim 1, wherein the core comprises a structural formula selected from the group consisting of: 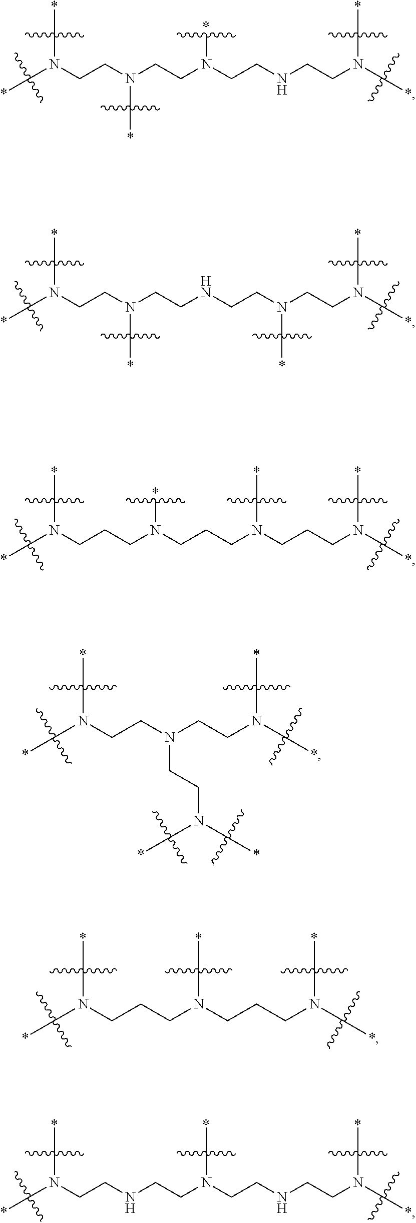 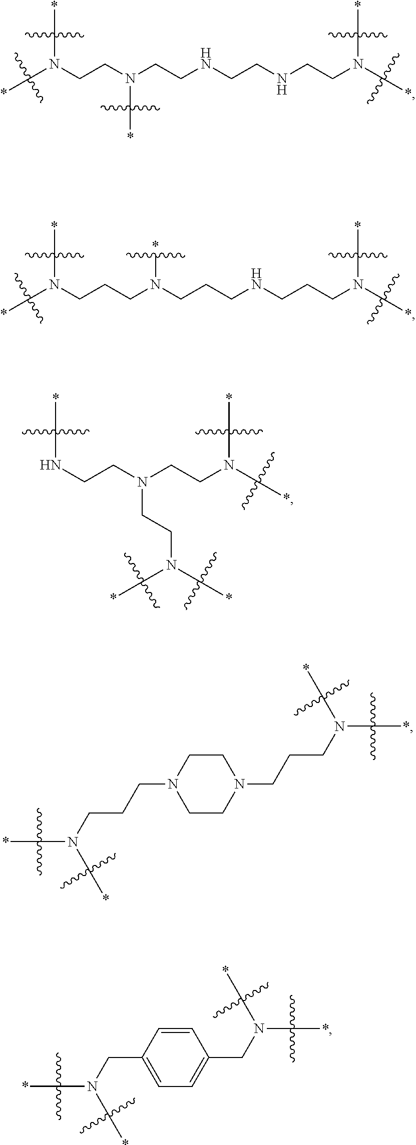 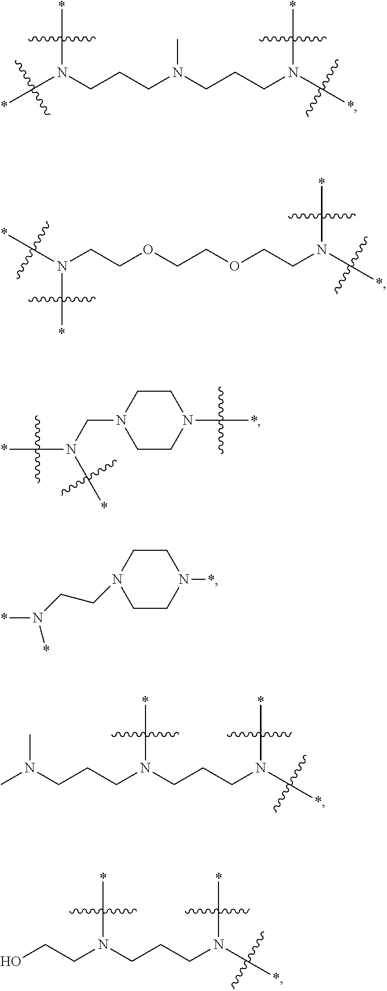 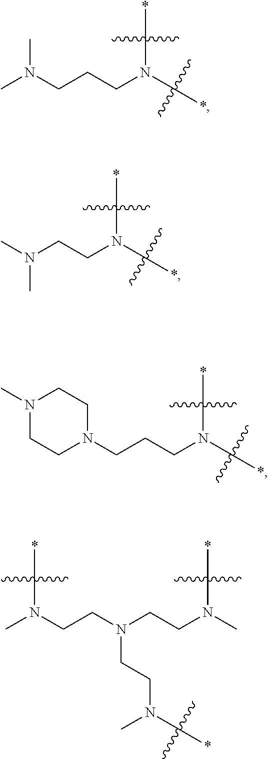
and pharmaceutically acceptable salts thereof, wherein * indicates a point of attachment of the core to a branch of the plurality of branches.

25. The dendrimer of claim 24, wherein the core comprises a structural formula selected from the group consisting of: 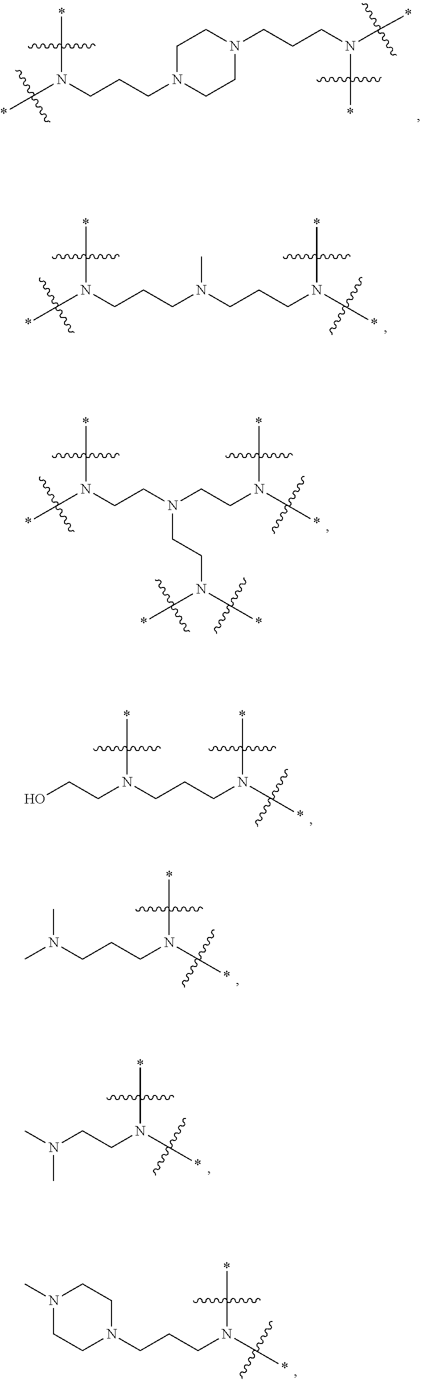
and pharmaceutically acceptable salts thereof, wherein * indicates a point of attachment of the core to a branch of the plurality of branches.

26. The dendrimer of claim 24, wherein the core comprises a structural formula selected from the group consisting of: 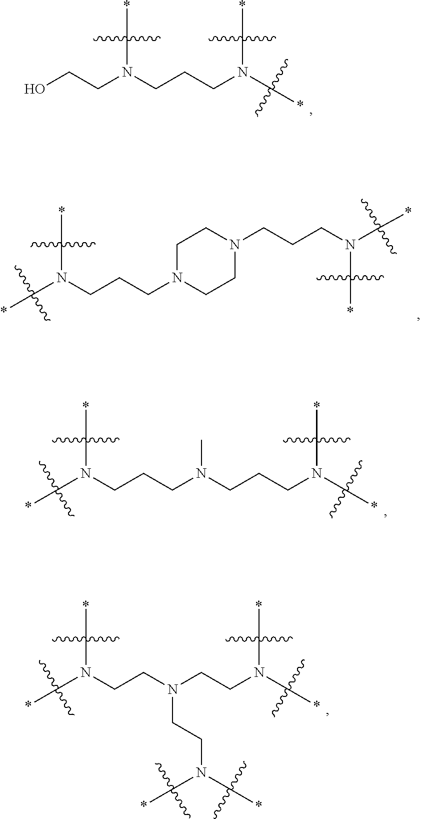
and pharmaceutically acceptable salts thereof, wherein * indicates a point of attachment of the core to a branch of the plurality of branches.

27. The dendrimer of claim 24, wherein the core comprises a structural formula 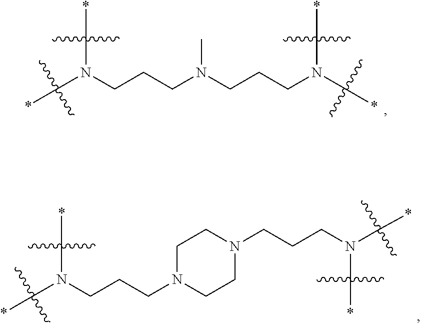
or a pharmaceutically acceptable salt thereof, wherein * indicates a point of attachment of the core to a branch of the plurality of branches.

28. The dendrimer of claim 24, wherein the core comprises a structural formula 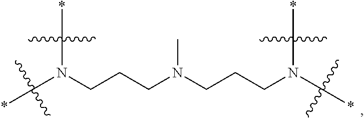
or a pharmaceutically acceptable salt thereof, wherein * indicates a point of attachment of the core to a branch of the plurality of branches.

29. The dendrimer of claim 1, wherein A¹ is —O— or —NH—.

30. The dendrimer of claim 29, wherein A¹ is —O—.

31. The dendrimer of claim 1, wherein A² is —O— or —NH—.

32. The dendrimer of claim 31, wherein A² is —O—.

33. The dendrimer of claim 1, wherein Y³ is C₁-C₁₂ (e.g., C₁-C₆, such as C₁-C₃) alkylene.

34. The dendrimer of claim 1, wherein the diacyl group independently at each occurrence comprises a structural formula 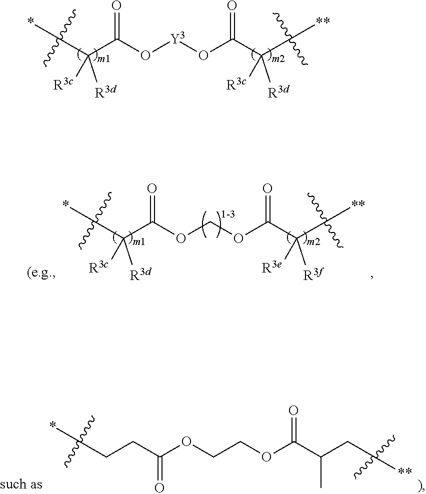
optionally wherein R^{3c}, R^{3d}, R^{3e}, and R^{3f} are each independently at each occurrence hydrogen or C₁-C₃ alkyl.

35. The dendrimer of claim 1, wherein L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene] 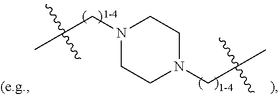
and [(C₁-C₄) alkylene]-phenylene-[(C₁-C₄) alkylene]

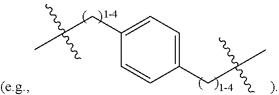

36. The dendrimer of claim 35, wherein L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-.

37. The dendrimer of claim 35, wherein L⁰, L¹, and L² are each independently at each occurrence C₁-C₆ alkylene (e.g., C₁-C₃ alkylene).

38. The dendrimer of claim 35, wherein L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)).

39. The dendrimer of claim 35, wherein L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

40. The dendrimer of claim 1, wherein R has a structural formula: 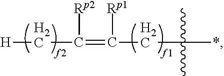
wherein:
R^{p1} and R^{p2} are each independently H or C₁-C₆ (e.g., C₁-C₃) alkyl;
f1 is 1, 2, 3, or 4; and
f2 is 0, 1, 2, or 3.

41. The dendrimer of claim 40, wherein —CR^{p2}═CR^{p1}— is a cis bond.

42. The dendrimer of claim 40, wherein —CR^{p2}═CR^{p1}— is a trans bond.

43. The dendrimer of claim 40, wherein R^{p1} is H.

44. The dendrimer of claim 40, wherein R^{p2} is H.

45. The dendrimer of claim 40, wherein f1+f2≥3 (e.g., from 3 to 6, such as from 4 to 6).

46. The dendrimer of claim [00251], wherein R has a structural formula: 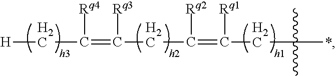
wherein:
R^{q1}, R^{q2}, R^{q3}, and R^{q4} are each independently H or C₁-C₆ (e.g., C₁-C₃) alkyl;
h1 is 1, 2, 3, or 4;
h2 is 1 or 2; and
h3 is 0, 1, 2, or 3.

47. The dendrimer of claim [00296], wherein —CR^{p2}═CR^{q1}— is a cis bond.

48. The dendrimer of claim 46, wherein —CR^{q2}═CR^{q1}— is a trans bond.

49. The dendrimer of claim 46, wherein —CR^{q4}═CR^{q3}— is a cis bond.

50. The dendrimer of claim 46, wherein —CR^{q4}═CR^{q3}— is a trans bond.

51. The dendrimer of claim 46, wherein R^{q1} is H.

52. The dendrimer of claim 46, wherein R^{q2} is methyl or H.

53. The dendrimer of claim 46, wherein R^{q3} is H.

54. The dendrimer of claim 46, wherein R^{q4} is methyl or H.

55. The dendrimer of claim 46, wherein h1 is 1.

56. The dendrimer of claim 46, wherein h2 is 1 or 2.

57. The dendrimer of claim 46, wherein h3 is 1 or 2.

58. The dendrimer of claim 46, wherein h1+h2+h3≥3 (e.g., from 3 to 6, such as from 4 to 6).

59. The dendrimer of claim 1, wherein R has a structural formula: 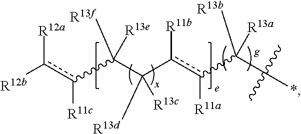
wherein:
indicates the point of attachment to the sulfur;
e is 0, 1, 2, 3, 4, 5, or 6;
g is 1, 2, or 3 (optionally g is 1);
x is independently at each occurrence 0, 1, 2, or 3 (optionally x is 1); and
R^{11a}, R^{11b}, R^{11c}, R^{12a}, R^{12b}, R^{13a}, R^{13b}, R^{13c}, R^{13d}, R^{13e}, and R^{13e} are each independently at each occurrence H or C₁-C₆ (e.g., C₁-C₃) alkyl.

60. The dendrimer of claim 59, wherein R has a structural formula 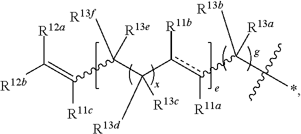
optionally

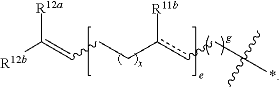

61. The dendrimer of claim 59, wherein R has a structural formula 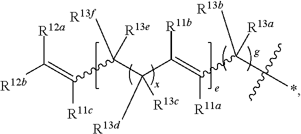
optionally

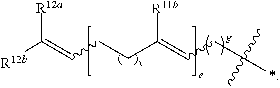

62. The dendrimer of claim 59, wherein R has a structural formula 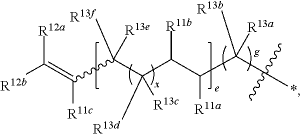
optionally

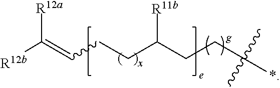

63. The dendrimer of claim 59, wherein e is 1, 2, 3, or 4 (optionally e is 1, 2, or 3).

64. The dendrimer of claim 59, wherein R^{11a} and R^{11b} are each H.

65. The dendrimer of claim 59, wherein R^{11b} is independently at each occurrence C₁-C₆ (e.g., C₁-C₃) alkyl.

66. The dendrimer of claim 59, wherein R^{12a} and R^{12b} are each independently C₁-C₆ (e.g., C₁-C₃) alkyl.

67. The dendrimer of claim 59, wherein R^{13a}, R^{13b}, R^{13c}, R^{13d}, R^{13e} and R^{13f} are each H.

68. The dendrimer of claim [00251], wherein R is selected from the group consisting of: 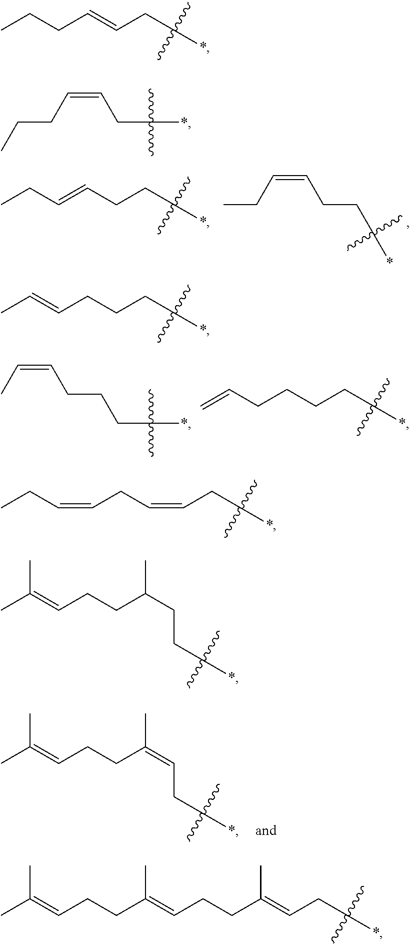
wherein * indicates the point of attachment to the sulfur.

69. The dendrimer of claim [00251], wherein the dendrimer is selected from the structures set forth in Table 6 and any pharmaceutically acceptable salt of any one of the structures set forth in Table 6.

70. The dendrimer of claim 1, wherein the dendrimer is characterized by an apparent acid dissociation constant (pKa) from 6.2 to 6.5 (e.g., as determined by in situ 6-p-toluidinyl-naphthalene-2-sulfonate (TNS) fluorescence titration).

71. The dendrimer of claim 1, wherein the dendrimer has a molecular weight (Mw) from 800 to 2,000 Da (e.g., as determined by mass spectrometry (MS) or by size exclusion chromatography (SEC)).

72. A lipid composition comprising:
an unsaturated dendrimer of any one of claims [00251]-71; and
one or more lipids selected from an ionizable cationic lipid, a zwitterionic lipid, a phospholipid, a steroid or a steroid derivative thereof, and a polymer-conjugated (e.g., polyethylene glycol (PEG)-conjugated) lipid.

73. The lipid composition of claim 72, wherein said unsaturated dendrimer is present in said lipid composition at a molar percentage of no more than about 60% (e.g., from about 5% to about 60%).

74. The lipid composition of claim 72, wherein said one or more lipids comprises an ionizable cationic lipid separate from said unsaturated dendrimer.

75. The lipid composition of claim 72, wherein said ionizable cationic lipid is a fully saturated lipid.

76. The lipid composition of claim 72, wherein said ionizable cationic lipid is a fully saturated dendrimer of a generation (g) having the structural formula: 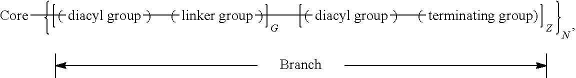
or a pharmaceutically acceptable salt thereof, wherein:
(a) the core comprises a structural formula (X_{Core}): 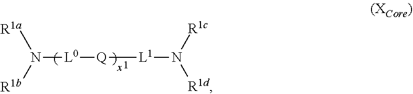
wherein:
Q is independently at each occurrence a covalent bond, —O—, —S—, —NR²—, or —CR^{3a}R^{3b}—;
R² is independently at each occurrence Rig or -L²-NR^{1e}R^{1f};
R^{3a} and R^{3b} are each independently at each occurrence hydrogen or an optionally substituted (e.g., C₁-C₆, such as C₁-C₃) alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
x¹ is 0, 1, 2, 3, 4, 5, or 6; and
(b) each branch of the plurality (N) of branches independently comprises a structural formula (X_{Branch}): 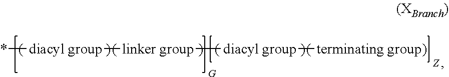
wherein:
* indicates a point of attachment of the branch to the core;
g is 1, 2, 3, or 4;
Z=2^{(g-1)};
G=0, when g=1; or G=Σᵢ₌₀^{i=g-2}2ⁱ, when g≠1;
(c) each diacyl group independently comprises a structural formula 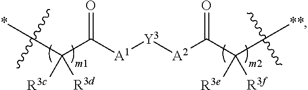
wherein:
* indicates a point of attachment of the diacyl group at the proximal end thereof,
** indicates a point of attachment of the diacyl group at the distal end thereof,
Y³ is independently at each occurrence an optionally substituted (e.g., C₁-C₁₂);
alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR⁴—, wherein: R⁴ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula 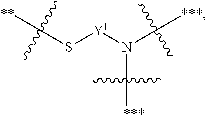
wherein:
** indicates a point of attachment of the linker to a proximal diacyl group;
*** indicates a point of attachment of the linker to a distal diacyl group; and
Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol.

77. The lipid composition of claim 72, wherein said ionizable cationic lipid is present in said lipid composition at a molar ratio from about 1:1 to about 1:2 to said unsaturated dendrimer.

78. The lipid composition of claim 72, wherein said one or more lipids comprises a phospholipid, optionally selected from the group consisting of: 1,2-distearoyl-sn-glycero-3-phosphocholine (DSPC), and 1,2-dioleoyl-sn-glycero-3-phosphoethanolamine (DOPE).

79. The lipid composition of claim 78, wherein said phospholipid is present in said lipid composition at a molar percentage from about 10% to about 50%.

80. The lipid composition of claim 72, wherein said one or more lipids comprises a polymer-conjugated (e.g., polyethylene glycol (PEG)-conjugated) lipid.

81. The lipid composition of claim 80, wherein the polymer-conjugated (e.g., polyethylene glycol (PEG)-conjugated) lipid is present in said lipid composition at a molar percentage from about 0.25% to about 12.5%.

82. The lipid composition of claim 72, wherein said one or more lipids comprises a steroid or steroid derivative thereof.

83. The lipid composition of claim 82, wherein said steroid or steroid derivative thereof is present in said lipid composition at a molar percentage from about 15% to about 60%.

84. The lipid composition of claim 72, further comprising a selective organ targeting (SORT) lipid that has a (e.g., permanently) positive net charge or a (e.g., permanently) negative net charge.

85. The lipid composition of claim 84, wherein said SORT lipid has a (e.g., permanently) positive net charge.

86. The lipid composition of claim 84, wherein said SORT lipid has a (e.g., permanently) negative net charge.

87. A pharmaceutical composition comprising a therapeutic agent coupled to a lipid composition comprising a dendrimer of any one of claims 1-71.

88. A pharmaceutical composition comprising a therapeutic agent coupled to a lipid composition comprising a lipid composition of any one of claims 72-86.

89. The pharmaceutical composition of claim 87 or 88, wherein said therapeutic agent is a messenger ribonucleic acid (mRNA).

90. The pharmaceutical composition of claim 89, wherein said mRNA is present in said pharmaceutical composition at a weight ratio from about 1:1 to about 1:100 with said cationic ionizable lipid.

91. The pharmaceutical composition of any one of claims 87-90, further comprising a pharmaceutically acceptable excipient.

92. The pharmaceutical composition of any one of claims 87-91, wherein the pharmaceutical composition is formulated for local or systemic administration.

93. The pharmaceutical composition of any one of claims 87-91, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crémes, via a catheter, via a lavage, via continuous infusion, via infusion, via inhalation, via injection, via local delivery, or via localized perfusion.

94. The pharmaceutical composition of any one of claims 87-93, comprising a SORT lipid in an amount sufficient to deliver said therapeutic agent to a liver cell (e.g., in a subject).

95. The pharmaceutical composition of any one of claims 87-93, comprising a SORT lipid in an amount sufficient to deliver said therapeutic agent to a non-liver cell (e.g., in a subject).

96. The pharmaceutical composition of any one of claims 87-93, wherein said unsaturated lipo-cationic dendrimer is present in said pharmaceutical composition in an amount sufficient to enhance a delivery potency of said therapeutic agent in a (e.g., liver) cell (e.g., in a subject).

97. A method for delivering a therapeutic agent into a cell, the method comprising:
contacting said cell with said therapeutic agent coupled to a lipid composition of any one of claims 72-86, thereby delivering said therapeutic agent into said cell.

98. The method of claim 97, wherein said contacting is ex vivo or in vivo.

99. The method of claim 97, wherein said contacting comprises administering to a subject said therapeutic agent coupled to said lipid composition.

100. The method of claim 97, wherein said cell is in a (e.g., functionally compromised) tissue or organ of a subject.
